(19) United States
(12) Patent Application Publication
(10) Pub. No.: US 2026/0090188 A1
(43) Pub. Date: Mar. 26, 2026

(54) HETEROCYCLIC COMPOUND, ORGANIC LIGHT EMITTING DEVICE COMPRISING SAME AND COMPOSITION FOR ORGANIC MATERIAL LAYER OF ORGANIC LIGHT EMITTING DEVICE

(71) Applicant: LT MATERIALS CO.,LTD., Yongin-si (KR)

(72) Inventors: Eui-Jeong CHOI, Yongin-si (KR); Hyun-Joo LEE, Yongin-si (KR); Young-Seok NO, Yongin-si (KR); Dong-Jun KIM, Yongin-si (KR); Dae-Hyuk CHOI, Yongin-si (KR)

(21) Appl. No.: 19/334,963

(22) Filed: Sep. 22, 2025

(30) Foreign Application Priority Data
Sep. 25, 2024 (KR) 10-2024-0129959

(51) Int. Cl. H10K 50/11; C07D 209/86; H10K 85/40; H10K 85/60

(52) U.S. Cl. CPC H10K 50/11; C07D 209/86; H10K 85/40; H10K 85/622; H10K 85/626; H10K 85/654; H10K 85/6572; H10K 85/6574; H10K 85/6576

(57) ABSTRACT

Disclosed are a heterocyclic compound of Chemical Formula 1, an organic light emitting device including the same and a composition for an organic material layer of an organic light emitting device. When used in an organic light emitting device, the heterocyclic compound described in the present specification can lower the driving voltage of the device, improve the light emitting efficiency, and improve the service life characteristics of the device.

### CROSS-REFERENCE TO RELATED APPLICATIONS

This application claims priority to and the benefit of Korean Patent Application No. 10-2024-0129959 filed in the Korean Intellectual Property Office on Sep. 25, 2024, the entire contents of which are incorporated herein by reference.

### TECHNICAL FIELD

The present specification relates to a heterocyclic compound, an organic light emitting device including the same and a composition for an organic material layer of the organic light emitting device.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENTS

### Patent Documents

(Patent Document 1) U.S. Pat. No. 4,356,429

### SUMMARY OF THE INVENTION

The present invention has been made in an effort to provide a heterocyclic compound, an organic light emitting device including the same and a composition for an organic material layer of an organic light emitting device.

An exemplary embodiment of the present invention provides a heterocyclic compound of the following Chemical Formula 1.

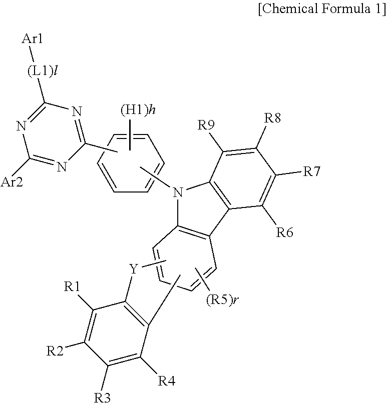

In Chemical Formula 1,
L1 is a direct bond; a substituted or unsubstituted C6 to C60 arylene group; or a substituted or unsubstituted C2 to C60 heteroarylene group,
1 is an integer from 1 to 3, and when 1 is 2 or greater, L1 is the same or different,
Y is O; S; C(R10)(R11) or N(R12),
H1 is hydrogen; or deuterium,
h is an integer from 1 to 4, and when h is 2 or greater, H1 is the same or different,
R1 to R12 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
at least one of R1 to R9 is deuterium,
r is 1 or 2, and when r is 2, R5 is the same or different,
Ar1 is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
Ar2 is a group represented by the following Chemical Formula A or B,

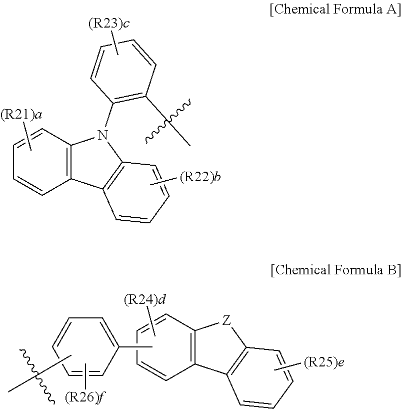

in Chemical Formulae A and B,
[symbol] is a moiety bonded to Chemical Formula 1,
Z is O; S; or C(R27)(R28),
R21 to R28 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or bonded to an adjacent group to form a substituted or unsubstituted aromatic hydrocarbon ring; or a substituted or unsubstituted aromatic hetero ring, and
a, b, c, e, and f are each an integer from 0 to 4, d is an integer from 0 to 3, and when a to f are each 2 or greater, substituents in the parenthesis are the same or different.

Another exemplary embodiment provides an organic light emitting device including: a first electrode; a second electrode; and an organic material layer having one or more layers provided between the first electrode and the second electrode, in which one or more layers of the organic material layer include one or more of the heterocyclic compounds.

Yet another exemplary embodiment provides a composition for an organic material layer of an organic light emitting device, including the heterocyclic compound.

When used in an organic light emitting device, the heterocyclic compound described in the present specification can lower the driving voltage of the device, improve the light emitting efficiency, and improve the service life characteristics of the device.

Specifically, the heterocyclic compound of the present invention is characterized by including disubstituted benzene (Feature 1), a substituent Ar2 (Feature 2), a deuterium-containing carbazole derivative (Feature 3), and triazine (Feature 4), and has an effect of further facilitating intramolecular electron transfer by including the disubstituted benzene as the core structure to adjust the appropriate overlap of the HOMO electron cloud and the LUMO electron cloud compared to other structures such as trisubstituted benzene.

Furthermore, the heterocyclic compound of the present invention includes a substituent with a specific structure as the substituent Ar2, and in particular, N-carbazole (bonded to the nitrogen of carbazole) is substituted at the ortho position of a phenylene group, which is a linker, and by having the substitution structure as described above, the spatial positions of the HOMO and LUMO become closer to each other, enabling intramolecular through-space charge transfer, so that the heterocyclic compound of the present invention has an effect of lowering the driving voltage and improving the light emitting efficiency.

Finally, the heterocyclic compound of the present invention has an effect of lowering the vibrational energy and rotational energy of the molecule and enhancing the stability of the molecule by including one or more deuterium atoms in the carbazole derivative.

### BRIEF DESCRIPTION OF THE DRAWINGS

FIGS. 1 to 3 are views each exemplarily illustrating a stacking structure of an organic light emitting device according to an exemplary embodiment of the present specification.

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

In the present specification, “N to N′” means N or more and N′ or less.

In the present specification, [symbol] of a chemical formula means a position to which a constituent element is bonded.

The term “substitution” means that a hydrogen atom bonded to a carbon atom or nitrogen atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; a cyano group; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; a silyl group; a phosphine oxide group; and an amine group, or a substituent to which two or more substituents selected among the substituents are linked.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the deuterium content may be 0% to 100%, and the deuterium content may be expressed as a deuterium substitution rate.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and may also be interpreted as an element which has the same number of protons, but different number of neutrons.

In an exemplary embodiment of the present application, when the total number of substituents of a basic compound is defined as T1 and the number of specific substituents among the substituents is defined as T2, the substitution rate T % of the specific substituent may be defined as T2/T1×100=T %.

That is, in an example, a deuterium substitution rate of 20% in a phenyl group represented by

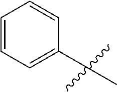 [SMILES: *c1ccccc1]

may be represented by 20% when the total number of substituents that the phenyl group can have is 5 (T1 in the formula) and the number of deuterium atoms among the substituents is 1 (T2 in the formula). That is, a deuterium substitution rate of 20% in the phenyl group may be represented by the following structural formula.

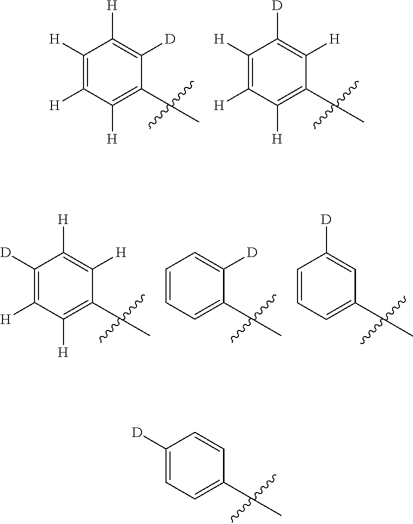 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium substitution rate of 0%” may mean a phenyl group that does not include a deuterium atom as a substituent, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes 0, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

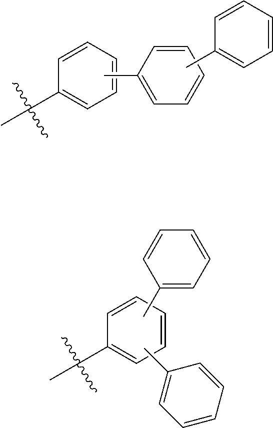

In the present specification, the fluorenyl group may be substituted, and adjacent substituents may be bonded to each other to form a ring.

When the fluorenyl group is substituted, the substituent may be the following structures, but is not limited thereto.

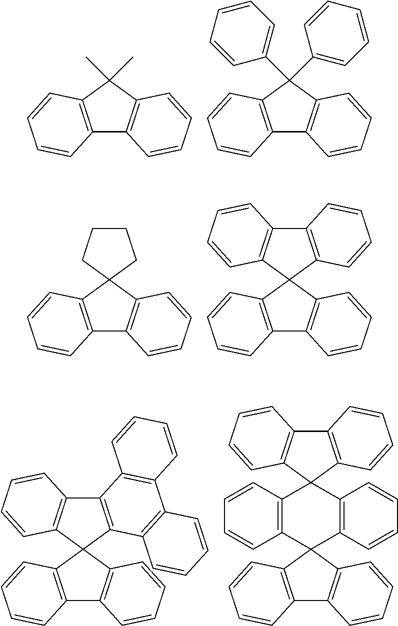

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole), a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, a benzocarbazole group may be any one of the following structures.

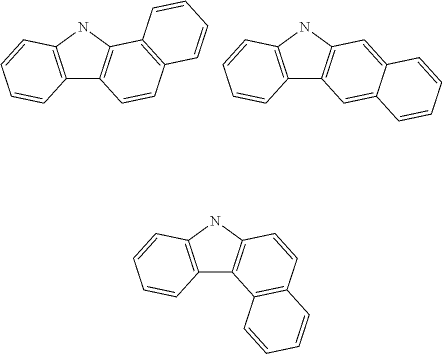

In the present specification, a dibenzocarbazole group may be any one of the following structures.

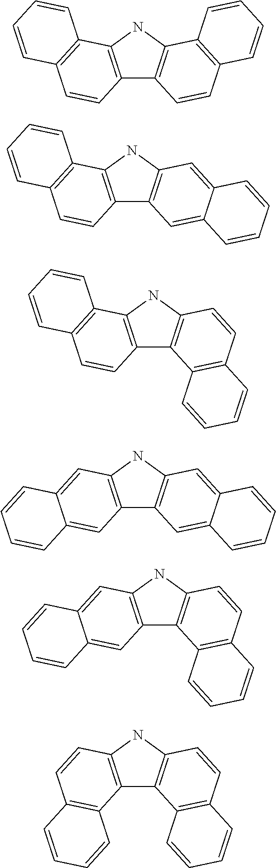

In the present specification, when the substituent is a carbazole group, a benzocarbazole group, or a dibenzocarbazole group, it means being bonded to the nitrogen or carbon of the carbazole group, the benzocarbazole group, or the dibenzocarbazole group.

In the present specification, when a carbazole group, a benzocarbazole group, or a dibenzocarbazole group is substituted, an additional substituent may be substituted at the nitrogen or carbon of the carbazole group, the benzocarbazole group, or the dibenzocarbazole group.

In the present specification, a naphthobenzofuran group may be any one of the following structures.

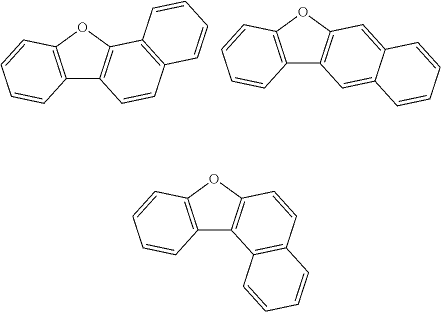

In the present specification, a naphthobenzothiophene group may be any one of the following structures.

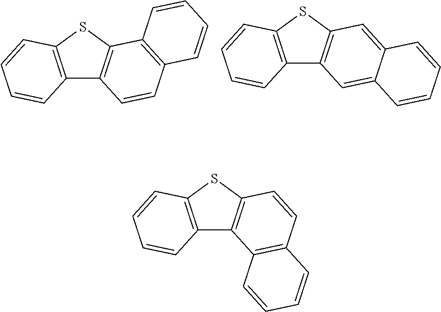

In the present specification, a silyl group includes Si and is a substituent to which the Si atom is directly linked as a radical, and is represented by —Si(R107)(R108)(R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

The silyl group may include an alkylsilyl group, an arylsilyl group, a heteroarylsilyl group, an alkylarylsilyl group, an arylheteroarylsilyl group, and the like, according to the substituent bonded to the Si element. An alkylsilyl group, an arylsilyl group, or a heteroarylsilyl group means that an alkyl group, an aryl group, or a heteroaryl group is substituted with the Si element of a silyl group, respectively, an alkylarylsilyl group means that an alkyl group and an aryl group are substituted with the Si element of a silyl group, and an arylheteroarylsilyl group means that an aryl group and a heteroaryl group are substituted with the Si element of a silyl group.

In the present, a triarylsilyl group means a silyl group substituted with three aryl groups. The number of carbon atoms of the aryl group may be 6 to 60, 6 to 30, or 6 to 20, and the number of carbon atoms of the triarylsilyl group may be 18 to 180, 18 to 90, or 18 to 40.

Specific examples of the silyl group include the following structures, but are not limited thereto.

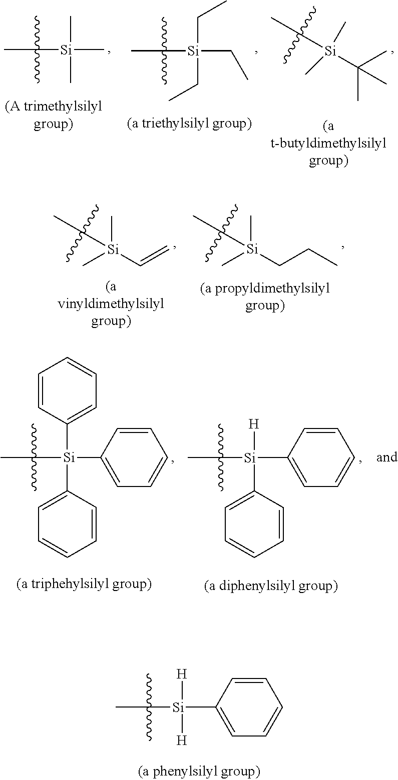

In the present specification, a phosphine oxide group is represented by —P(═O)(R110)(R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112)(R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the number of carbon atoms may be represented by Cn (n: an integer of 1 or greater). For example, when the number of carbon atoms is 1 to 10, the number of carbon atoms may be represented by C1 to C10.

An exemplary embodiment of the present specification provides a heterocyclic compound of the following Chemical Formula 1.

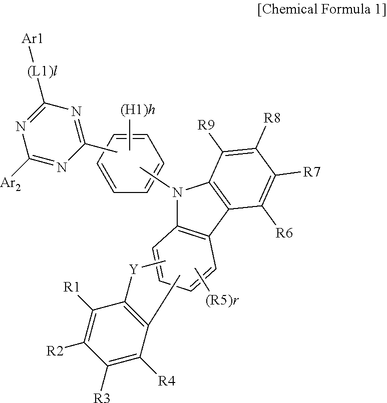

The definition of each substituent of Chemical Formula 1 is the same as that described above.

In an exemplary embodiment of the present specification, Chemical Formula 1 may be represented by any one of the following Chemical Formulae 1-1 to 1-6.

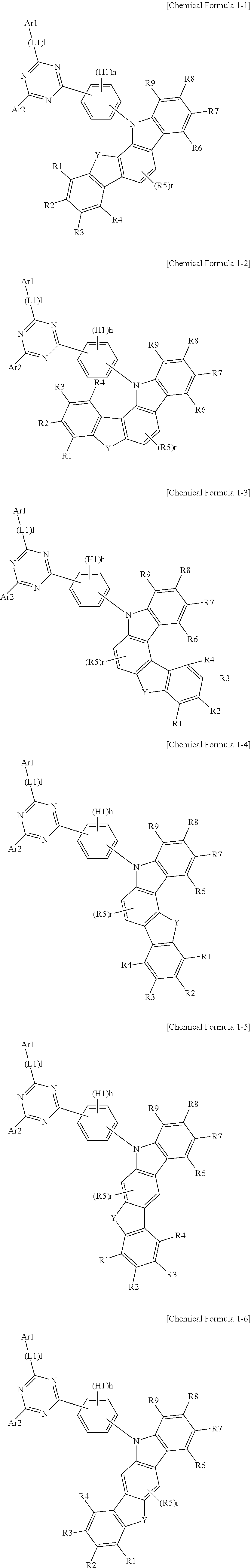

In Chemical Formulae 1-1 to 1-6,
the definition of each substituent is the same as the definition in Chemical Formula 1.

In an exemplary embodiment of the present specification, Chemical Formula 1 may be represented by any one of the following Chemical Formulae 1-P, 1-M and 1-O.

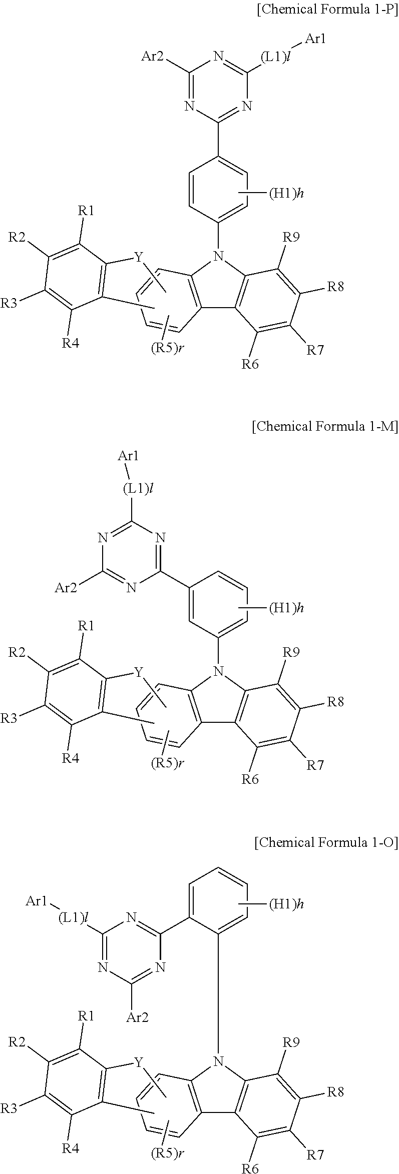

In Chemical Formulae 1-P, 1-M and 1-O,
the definition of each substituent is the same as the definition in Chemical Formula 1.

In an exemplary embodiment of the present specification, L1 is a direct bond; a substituted or unsubstituted C6 to C60 arylene group; or a substituted or unsubstituted C2 to C60 heteroarylene group.

In an exemplary embodiment of the present specification, L1 may be a direct bond; a substituted or unsubstituted C6 to C30 arylene group; or a substituted or unsubstituted C2 to C30 heteroarylene group.

In an exemplary embodiment of the present specification, L1 may be a direct bond; or a substituted or unsubstituted C6 to C60 arylene group.

In an exemplary embodiment of the present specification, L1 may be a direct bond; or a substituted or unsubstituted C6 to C30 arylene group.

In an exemplary embodiment of the present specification, L1 may be a direct bond; or a substituted or unsubstituted C6 to C15 arylene group.

In an exemplary embodiment of the present specification, L1 may be a direct bond; or a C6 to C60 arylene group.

In an exemplary embodiment of the present specification, L1 may be a direct bond; or a C6 to C30 arylene group.

In an exemplary embodiment of the present specification, L1 may be a direct bond; or a C6 to C15 arylene group.

In an exemplary embodiment of the present specification, L1 may be a direct bond; or a substituted or unsubstituted phenylene group.

In an exemplary embodiment of the present specification, L1 may be a direct bond; or a phenylene group.

In an exemplary embodiment of the present specification, L1 may be a direct bond.

In an exemplary embodiment of the present specification, 1 is an integer from 1 to 3, and when 1 is 2 or greater, Lis are the same or different.

In an exemplary embodiment of the present specification, when 1 is 2, L1 is represented by -L1-L1′-, and the definition of L1 is the same as that of L1.

In an exemplary embodiment of the present specification, 1 may be 1.

In an exemplary embodiment of the present specification, Y is O; S; C(R10)(R11) or N(R12).

In an exemplary embodiment of the present specification, Y may be O.

In an exemplary embodiment of the present specification, Y may be S.

In an exemplary embodiment of the present specification, Y may be C(R10)(R11).

In an exemplary embodiment of the present specification, Y may be N(R12).

In an exemplary embodiment of the present specification, R10 and R11 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R10 and R11 may be each independently a substituted or unsubstituted C1 to C60 alkyl group; or a substituted or unsubstituted C6 to C60 aryl group.

In an exemplary embodiment of the present specification, R10 and R11 may be each independently a substituted or unsubstituted C1 to C60 alkyl group.

In an exemplary embodiment of the present specification, R10 and R11 may be each independently a substituted or unsubstituted C1 to C30 alkyl group.

In an exemplary embodiment of the present specification, R10 and R11 may be each independently a substituted or unsubstituted C1 to C10 alkyl group.

In an exemplary embodiment of the present specification, R10 and R11 may be each independently a substituted or unsubstituted methyl group.

In an exemplary embodiment of the present specification, R10 and R11 may be each independently a C1 to C10 alkyl group unsubstituted or substituted with deuterium.

In an exemplary embodiment of the present specification, R10 and R11 may be each independently a methyl group unsubstituted or substituted with deuterium.

In an exemplary embodiment of the present specification, R10 and R11 may be each independently a C1 to C10 alkyl group.

In an exemplary embodiment of the present specification, R10 and R11 may be a methyl group.

In an exemplary embodiment of the present specification, R10 and R11 may be the same.

In an exemplary embodiment of the present specification, R12 is hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R12 may be a substituted or unsubstituted C1 to C60 alkyl group; or a substituted or unsubstituted C6 to C60 aryl group.

In an exemplary embodiment of the present specification, R12 may be a substituted or unsubstituted C6 to C60 aryl group.

In an exemplary embodiment of the present specification, R12 may be a substituted or unsubstituted C6 to C30 aryl group.

In an exemplary embodiment of the present specification, R12 may be a substituted or unsubstituted C6 to C20 aryl group.

In an exemplary embodiment of the present specification, R12 may be a substituted or unsubstituted phenyl group.

In an exemplary embodiment of the present specification, R12 may be a C6 to C30 aryl group unsubstituted or substituted with deuterium.

In an exemplary embodiment of the present specification, R12 may be a C6 to C20 aryl group unsubstituted or substituted with deuterium.

In an exemplary embodiment of the present specification, R12 may be a phenyl group unsubstituted or substituted with deuterium.

In an exemplary embodiment of the present specification, Chemical Formula 1 may be represented by any one of the following Chemical Formulae 1-a to 1-d.

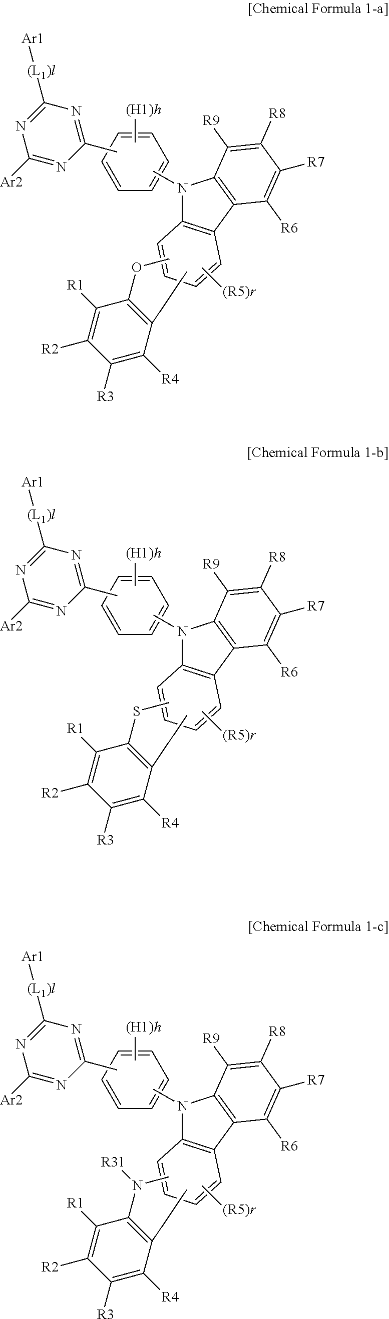

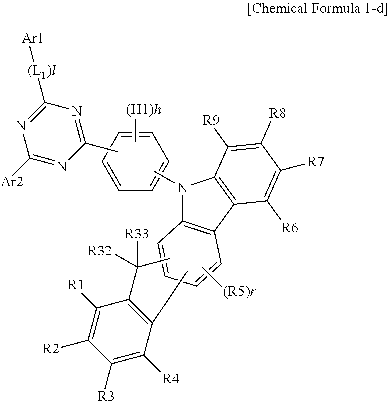

In Chemical Formulae 1-a to 1-d,
R31 is a substituted or unsubstituted C6 to C60 aryl group,
R32 and R33 are each independently a substituted or unsubstituted C1 to C60 alkyl group, and
the definitions of the other substituents are the same as the definitions in Chemical Formula 1.

In an exemplary embodiment of the present specification, H1 is hydrogen; or deuterium.

In an exemplary embodiment of the present specification, h is an integer from 1 to 4, and when h is 2 or greater, H1s are the same or different.

In an exemplary embodiment of the present specification, h means the number of substituents H1, and when h is 1, 2 or 3, the number of substituents H1 is 1, 2 or 3, and the remaining carbon atoms of the benzene ring may be bonded to hydrogen.

In an exemplary embodiment of the present specification, when h is 2, 3 or 4, the substituent H1 may be one or two types.

In an exemplary embodiment of the present specification, h may be 4, and H1 may be hydrogen.

In an exemplary embodiment of the present specification, h may be 4, and H1 may be deuterium.

In an exemplary embodiment of the present specification, h may be 4, and 4 H1s may be hydrogen, deuterium, deuterium, and deuterium.

In an exemplary embodiment of the present specification, h may be 4, and 4 H1s may be hydrogen, hydrogen, deuterium, and deuterium.

In an exemplary embodiment of the present specification, h may be 4, and 4 H1s may be hydrogen, hydrogen, hydrogen, and deuterium.

In an exemplary embodiment of the present specification, h may be 3, and H1 may be deuterium. In this case, the benzene ring of Chemical Formula 1 includes hydrogen, deuterium, deuterium, and deuterium.

In an exemplary embodiment of the present specification, h may be 2, and H1 may be deuterium. In this case, the benzene ring of Chemical Formula 1 includes hydrogen, hydrogen, deuterium, and deuterium.

In an exemplary embodiment of the present specification, h may be 1, and H1 may be deuterium. In this case, the benzene ring of Chemical Formula 1 includes hydrogen, hydrogen, hydrogen, and deuterium.

In an exemplary embodiment of the present specification, R1 to R9 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and at least one of R1 to R9 is deuterium.

In an exemplary embodiment of the present specification, R1 to R9 may be each independently hydrogen; deuterium; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group, and at least one of R1 to R9 is deuterium.

In an exemplary embodiment of the present specification, R1 to R9 may be each independently hydrogen; deuterium; a substituted or unsubstituted C1 to C60 alkyl group; a substituted or unsubstituted C3 to C60 cycloalkyl group; or a substituted or unsubstituted C6 to C60 aryl group, and at least one of R1 to R9 is deuterium.

In an exemplary embodiment of the present specification, R1 to R9 may be each independently hydrogen; deuterium; or a substituted or unsubstituted C1 to C60 alkyl group, and at least of R1 to R9 is deuterium.

In an exemplary embodiment of the present specification, R1 to R9 may be each independently hydrogen; or deuterium, and at least one of R1 to R9 is deuterium.

In an exemplary embodiment of the present specification, Ar1 is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, Ar1 may be a substituted or unsubstituted C6 to C60 aryl group; or a substituted or unsubstituted C2 to C60 heteroaryl group.

In an exemplary embodiment of the present specification, Ar1 may be a substituted or unsubstituted C6 to C30 aryl group; or a substituted or unsubstituted C2 to C30 heteroaryl group.

In an exemplary embodiment of the present specification, Ar1 may be a substituted or unsubstituted C6 to C60 aryl group.

In an exemplary embodiment of the present specification, Ar1 may be a substituted or unsubstituted C6 to C30 aryl group.

In an exemplary embodiment of the present specification, Ar1 may be a C6 to C60 aryl group.

In an exemplary embodiment of the present specification, Ar1 may be a C6 to C30 aryl group.

In an exemplary embodiment of the present specification, Ar1 may be a substituted or unsubstituted phenyl group; or a substituted or unsubstituted biphenyl group.

In an exemplary embodiment of the present specification, Ar1 may be a phenyl group; or a biphenyl group.

In an exemplary embodiment of the present specification, the deuterium substitution rate of Ar1 may be 0%.

In an exemplary embodiment of the present specification, Ar2 is a group represented by the following Chemical Formula A or B.

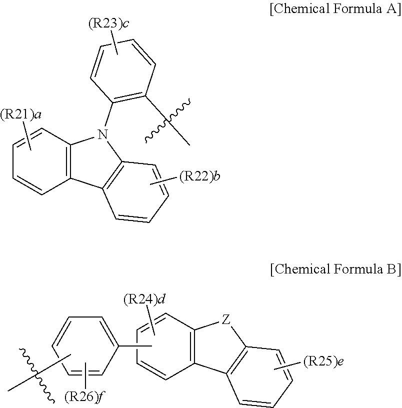

In Chemical Formulae A and B,
[symbol] is a moiety bonded to Chemical Formula 1,
Z is O; S; or C(R27)(R28),
R21 to R28 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or bonded to an adjacent group to form a substituted or unsubstituted aromatic hydrocarbon ring; or a substituted or unsubstituted aromatic hetero ring, and
a, b, c, e, and f are each an integer from 0 to 4, d is an integer from 0 to 3, and when a to f are each 2 or greater, substituents in the parenthesis are the same or different.

In an exemplary embodiment of the present specification, Chemical Formula 1 may be represented by the following Chemical Formula 1-A or 1-B.

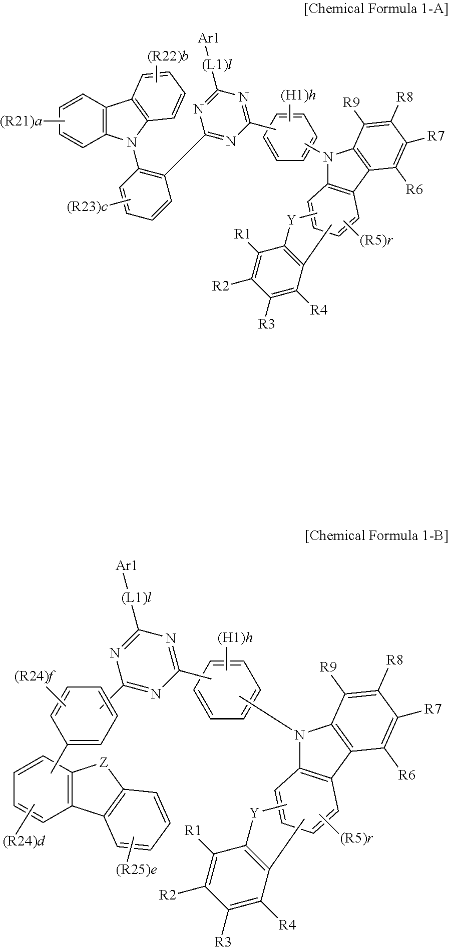

In Chemical Formulae 1-A and 1-B,
the definition of each substituent is the same as the definition in Chemical Formula 1.

In an exemplary embodiment of the present specification, Z may be O.

In an exemplary embodiment of the present specification, Z may be S.

In an exemplary embodiment of the present specification, Z may be C(R27)(R28).

In an exemplary embodiment of the present specification, R21 to R26 may be each independently hydrogen; deuterium; a substituted or unsubstituted C1 to C60 alkyl group; a substituted or unsubstituted C6 to C60 aryl group; or a substituted or unsubstituted C2 to C60 heteroaryl group, or may be bonded to an adjacent group to form a substituted or unsubstituted aromatic hydrocarbon ring; or a substituted or unsubstituted aromatic hetero ring.

In an exemplary embodiment of the present specification, R21 to R26 may be each independently hydrogen; deuterium; or a substituted or unsubstituted C6 to C60 aryl group, or may be bonded to an adjacent group to form a substituted or unsubstituted aromatic hydrocarbon ring; or a substituted or unsubstituted aromatic hetero ring.

In an exemplary embodiment of the present specification, R21 to R26 may be each independently hydrogen; deuterium; or a substituted or unsubstituted C6 to C30 aryl group, or may be bonded to an adjacent group to form a substituted or unsubstituted C6 to C30 aromatic hydrocarbon ring; or a substituted or unsubstituted C2 to C30 aromatic hetero ring.

In an exemplary embodiment of the present specification, R21 to R26 may be each independently hydrogen; deuterium; or a substituted or unsubstituted C6 to C30 aryl group, or may be bonded to an adjacent group to form a substituted or unsubstituted C6 to C30 aromatic hydrocarbon ring; or a substituted or unsubstituted C2 to C30 aromatic hetero ring including O or S.

In an exemplary embodiment of the present specification, R21 to R26 may be each independently hydrogen; deuterium; or a substituted or unsubstituted phenyl group, or may be bonded to an adjacent group to form a substituted or unsubstituted indene ring; a substituted or unsubstituted benzofuran ring; or a substituted or unsubstituted benzothiophene ring.

In an exemplary embodiment of the present specification, R21 and R22 may be each independently hydrogen; deuterium; a substituted or unsubstituted C1 to C60 alkyl group; a substituted or unsubstituted C6 to C60 aryl group; or a substituted or unsubstituted C2 to C60 heteroaryl group, or may be bonded to an adjacent group to form a substituted or unsubstituted aromatic hydrocarbon ring; or a substituted or unsubstituted aromatic hetero ring.

In an exemplary embodiment of the present specification, R21 and R22 may be each independently hydrogen; deuterium; or a substituted or unsubstituted C6 to C60 aryl group, or may be bonded to an adjacent group to form a substituted or unsubstituted aromatic hydrocarbon ring; or a substituted or unsubstituted aromatic hetero ring.

In an exemplary embodiment of the present specification, R21 and R22 may be each independently hydrogen; deuterium; or a substituted or unsubstituted C6 to C30 aryl group, or may be bonded to an adjacent group to form a substituted or unsubstituted C6 to C30 aromatic hydrocarbon ring; or a substituted or unsubstituted C2 to C30 aromatic hetero ring.

In an exemplary embodiment of the present specification, R21 and R22 may be each independently hydrogen; deuterium; or a substituted or unsubstituted C6 to C30 aryl group, or may be bonded to an adjacent group to form a substituted or unsubstituted C6 to C30 aromatic hydrocarbon ring; or a substituted or unsubstituted C2 to C30 aromatic hetero ring including O or S.

In an exemplary embodiment of the present specification, R21 and R22 may be each independently hydrogen; deuterium; or a substituted or unsubstituted phenyl group, or may be bonded to an adjacent group to form a substituted or unsubstituted indene ring; a substituted or unsubstituted benzofuran ring; or a substituted or unsubstituted benzothiophene ring.

In an exemplary embodiment of the present specification, R21 and R22 may be each independently hydrogen; deuterium; or a C6 to C30 aryl group unsubstituted or substituted with deuterium, or may be bonded to an adjacent group to form a C6 to C30 aromatic hydrocarbon ring unsubstituted or substituted with one or more substituents of deuterium and an alkyl group; or a C2 to C30 aromatic hetero ring unsubstituted or substituted with deuterium and including O or S.

In an exemplary embodiment of the present specification, R21 and R22 may be each independently hydrogen; deuterium; or a phenyl group unsubstituted or substituted with deuterium, or may be bonded to an adjacent group to form an indene ring unsubstituted or substituted with one or more substituents of deuterium and an alkyl group; a benzofuran ring unsubstituted or substituted with deuterium; or a benzothiophene ring unsubstituted or substituted with deuterium.

In an exemplary embodiment of the present specification, R24 and R25 may be each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In an exemplary embodiment of the present specification, R24 and R25 may be each independently hydrogen; deuterium; or a substituted or unsubstituted C6 to C60 aryl group.

In an exemplary embodiment of the present specification, R24 and R25 may be each independently hydrogen; deuterium; or a substituted or unsubstituted C6 to C30 aryl group.

In an exemplary embodiment of the present specification, R24 and R25 may be each independently hydrogen; deuterium; or a substituted or unsubstituted phenyl group.

In an exemplary embodiment of the present specification, R23 and R26 may be each independently hydrogen; or deuterium.

In an exemplary embodiment of the present specification, R27 and R28 may be each independently a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R27 and R28 may be each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R27 and R28 may be each independently a substituted or unsubstituted C1 to C60 alkyl group; or a substituted or unsubstituted C6 to C60 aryl group.

In an exemplary embodiment of the present specification, R27 and R28 may be each independently a substituted or unsubstituted C1 to C60 alkyl group.

In an exemplary embodiment of the present specification, R27 and R28 may be each independently a substituted or unsubstituted C1 to C30 alkyl group.

In an exemplary embodiment of the present specification, R27 and R28 may be each independently a substituted or unsubstituted C1 to C10 alkyl group.

In an exemplary embodiment of the present specification, R27 and R28 may be each independently a C1 to C30 alkyl group.

In an exemplary embodiment of the present specification, R27 and R28 may be each independently a C1 to C10 alkyl group.

In an exemplary embodiment of the present specification, R27 and R28 may be each independently a substituted or unsubstituted methyl group.

In an exemplary embodiment of the present specification, R27 and R28 may be each independently a methyl group.

In an exemplary embodiment of the present specification, Ar2 is a group represented by any one of the following Chemical Formulae A-1, A-2, A-3, B-1, B-2, B-3 and B-4.

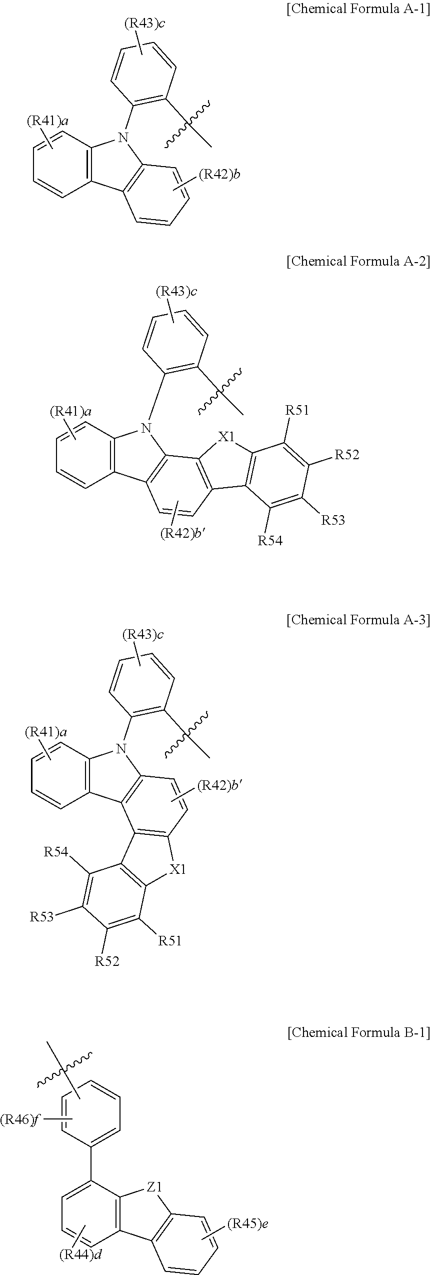

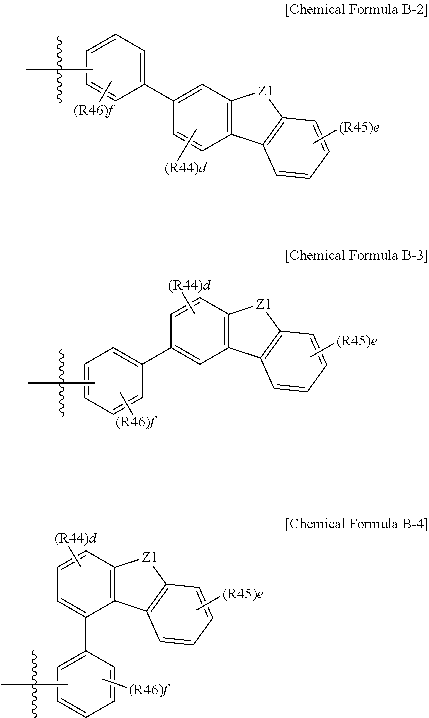

In Chemical Formulae A-1, A-2, A-3, B-1, B-2, B-3 and B-4,
[symbol] is a moiety bonded to Chemical Formula 1,
X1 and Z1 are each independently O; S; or C(R55)(R56),
R41 to R46 and R51 to R56 are each independently hydrogen; deuterium; a substituted or unsubstituted C1 to C60 alkyl group; or a substituted or unsubstituted C6 to C60 aryl group, and
a, b, c, e and f are each an integer from 0 to 4, d is an integer from 0 to 3, b′ is an integer from 0 to 2, and when a to f are each 2 or greater or b′ is 2, substituents in the parenthesis are the same or different.

In an exemplary embodiment of the present specification, R41 and R45 may be each independently hydrogen; deuterium; or a substituted or unsubstituted C6 to C60 aryl group.

In an exemplary embodiment of the present specification, R41 and R45 may be each independently hydrogen; deuterium; or a substituted or unsubstituted C6 to C30 aryl group.

In an exemplary embodiment of the present specification, R41 and R45 may be each independently hydrogen; deuterium; or a substituted or unsubstituted phenyl group.

In an exemplary embodiment of the present specification, R41 and R45 may be each independently hydrogen; deuterium; or a C6 to C60 aryl group unsubstituted or substituted with deuterium.

In an exemplary embodiment of the present specification, R41 and R45 may be each independently hydrogen; deuterium; or a C6 to C30 aryl group unsubstituted or substituted with deuterium.

In an exemplary embodiment of the present specification, R41 and R45 may be each independently hydrogen; deuterium; or a phenyl group unsubstituted or substituted with deuterium.

In an exemplary embodiment of the present specification, R42 to R44 and R46 may be each independently hydrogen; or deuterium.

In an exemplary embodiment of the present specification, R51 to R54 may be each independently hydrogen; or deuterium.

In an exemplary embodiment of the present specification, R55 and R56 may be each independently a substituted or unsubstituted C1 to C10 alkyl group.

In an exemplary embodiment of the present specification, R55 and R56 may be each independently a substituted or unsubstituted methyl group.

In an exemplary embodiment of the present specification, R55 and R56 may be each independently a C1 to C10 alkyl group.

In an exemplary embodiment of the present specification, R55 and R56 may be each independently a methyl group.

In an exemplary embodiment of the present specification, a, b, c, e and f may be each an integer from 1 to 4.

In an exemplary embodiment of the present specification, d may be an integer from 1 to 3.

In an exemplary embodiment of the present specification, U may be 1 or 2.

In an exemplary embodiment of the present specification, Chemical Formula 1 may be represented by the following Chemical Formula 1-D.

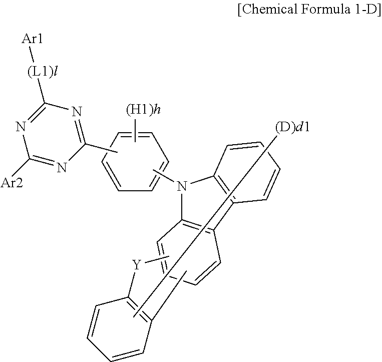

In Chemical Formula 1-D,
D is deuterium,
d1 is an integer from 1 to 10, and
the definitions of the other substituents are the same as the definitions in Chemical Formula 1.

In an exemplary embodiment of the present specification, the deuterium substitution rate of the

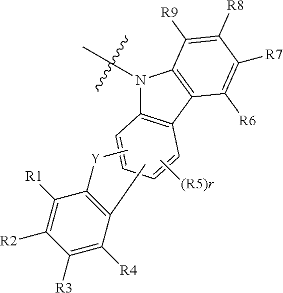

structure in Chemical Formula 1 may be 10% to 100%.

In an exemplary embodiment of the present specification, the deuterium substitution rate of the

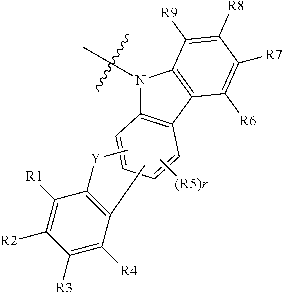

structure in Chemical Formula 1 may be 20% to 100%.

In an exemplary embodiment of the present specification, the deuterium substitution rate of the

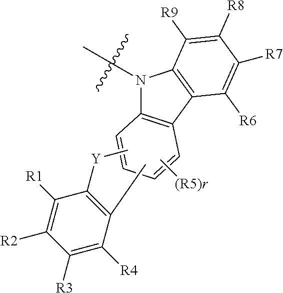

structure in Chemical Formula 1 may be 30% to 100%.

In an exemplary embodiment of the present specification, the deuterium substitution rate of the

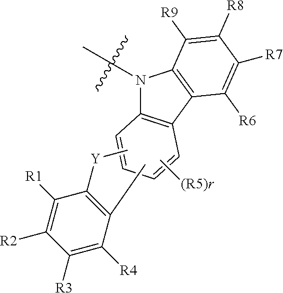

structure in Chemical Formula 1 may be 50% to 100%.

In an exemplary embodiment of the present specification, the deuterium substitution rate of the

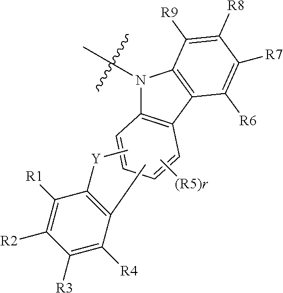

structure in Chemical Formula 1 may be 10% or more and less than 100%.

In an exemplary embodiment of the present specification, the deuterium substitution rate of the

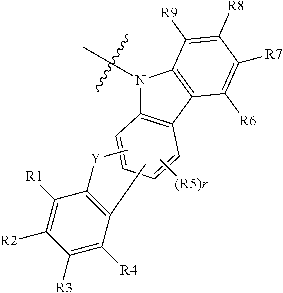

structure in Chemical Formula 1 may be 20% or more and less than 100%.

In an exemplary embodiment of the present specification, the deuterium substitution rate of the

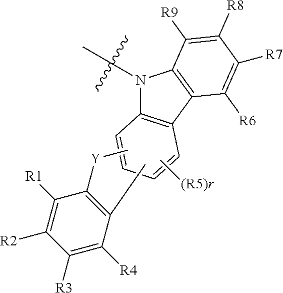

structure in Chemical Formula 1 may be 30% or more and less than 100%.

In an exemplary embodiment of the present specification, the deuterium substitution rate of the

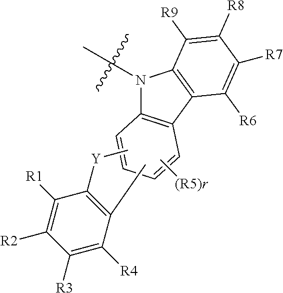

structure in Chemical Formula 1 may be 50% or more and less than 100%.

In an exemplary embodiment of the present specification, the deuterium substitution rate of the

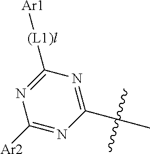

structure in Chemical Formula 1 may be 0%, or 10% to 100%.

In an exemplary embodiment of the present specification, the deuterium substitution rate of the

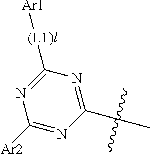

structure in Chemical Formula 1 may be 0%, or 20% to 100%.

In an exemplary embodiment of the present specification, the deuterium substitution rate of the

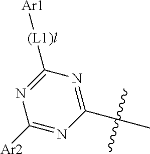

structure in Chemical Formula 1 may be 0%, or 10% or more and less than 100%.

In an exemplary embodiment of the present specification, the deuterium substitution rate of the

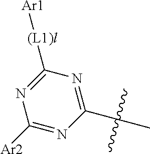

structure in Chemical Formula 1 may be 0%, or 20% or more and less than 100%.

In an exemplary embodiment of the present specification, the deuterium substitution rate based on the total hydrogen and deuterium in Chemical Formula 1 may be more than 0% and 100% or less.

In an exemplary embodiment of the present specification, the deuterium substitution rate of Chemical Formula 1 may be 5% to 100%.

In an exemplary embodiment of the present specification, the deuterium substitution rate of Chemical Formula 1 may be 10% to 100%.

In an exemplary embodiment of the present specification, the deuterium substitution rate of Chemical Formula 1 may be 15% to 100%.

In an exemplary embodiment of the present specification, the deuterium substitution rate of Chemical Formula 1 may be 20% to 100%.

In an exemplary embodiment of the present specification, the deuterium substitution rate of Chemical Formula 1 may be more than 0% and less than 100%. In an exemplary embodiment of the present specification, the deuterium substitution rate of Chemical Formula 1 may be 5% or more and less than 100%.

In an exemplary embodiment of the present specification, the deuterium substitution rate of Chemical Formula 1 may be 10% or more and less than 100%.

In an exemplary embodiment of the present specification, the deuterium substitution rate of Chemical Formula 1 may be 15% or more and less than 100%.

In an exemplary embodiment of the present specification, the deuterium substitution rate of Chemical Formula 1 may be 20% or more and less than 100%.

In an exemplary embodiment of the present specification, the deuterium substitution rate of Chemical Formula 1 may be 5% to 90%.

In an exemplary embodiment of the present specification, the deuterium substitution rate of Chemical Formula 1 may be 10% to 90%.

In an exemplary embodiment of the present specification, the deuterium substitution rate of Chemical Formula 1 may be 15% to 90%.

In an exemplary embodiment of the present specification, the deuterium substitution rate of Chemical Formula 1 may be 20% to 90%.

In the present specification, the deuterium substitution rate refers to the ratio of the number of deuterium atoms to the total number of hydrogen and deuterium atoms included in a specific structure

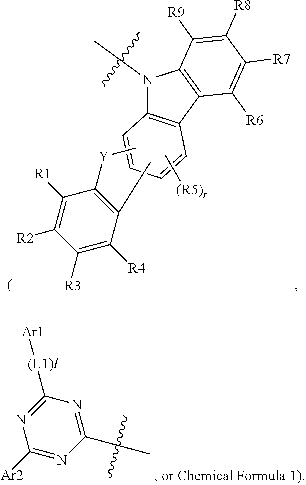

For example, when a particular structure includes 20 hydrogen atoms and 20 deuterium atoms, the deuterium substitution rate is 50% because the ratio of the 20 deuterium atoms to the total of 40 hydrogen and deuterium atoms is 50%.

In an exemplary embodiment of the present specification, the deuterium substitution rate of the heterocyclic compound of Chemical Formula 1 satisfies the above range, the photochemical characteristics of a compound which includes deuterium and a compound which does not include deuterium are almost similar, but when deposited on a thin film, the deuterium-containing material tends to be packed with a narrower intermolecular distance.

Accordingly, when an electron only device (EOD) and a hole only device (HOD) are manufactured and the current density thereof according to voltage is confirmed, it can be confirmed that the heterocyclic compound of Chemical Formula 1 of the present invention exhibits much more balanced charge transport characteristics than a compound which does not include deuterium in the same structure. In particular, even though deuterium is included compared to the same structure, when deuterium is included in the

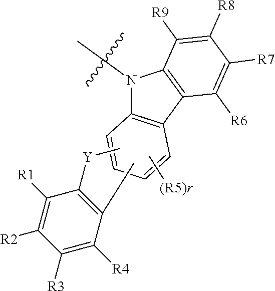

structure as in the present invention, it is possible to provide excellent performance as a material for an organic light emitting device compared to when deuterium is included in a structure such as

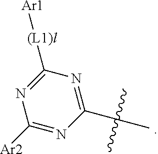

Further, when the surface of a thin film is observed using an atomic force microscope (AFM), it can be confirmed that the thin film made of a compound including deuterium is deposited with a more uniform surface without any aggregated portion.

Additionally, since the single bond dissociation energy of carbon and deuterium is higher than the single bond dissociation energy of carbon and hydrogen, in the case of the heterocyclic compound of Chemical Formula 1 of the present invention, the stability of the total molecules is enhanced, so that there is an effect of improving the service life of the device.

In an exemplary embodiment of the present specification, Chemical Formula 1 may be represented by any one of the following compounds.

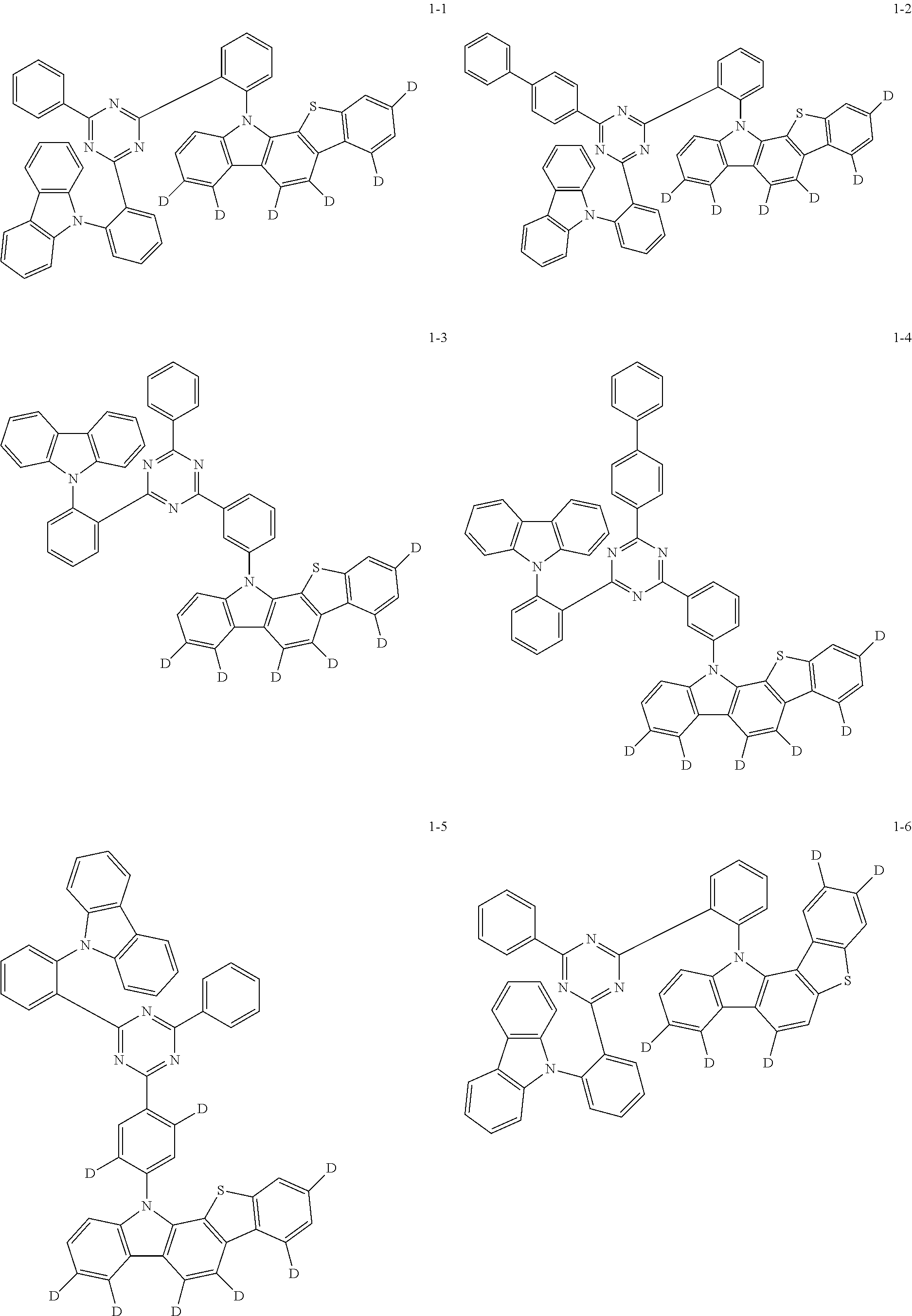

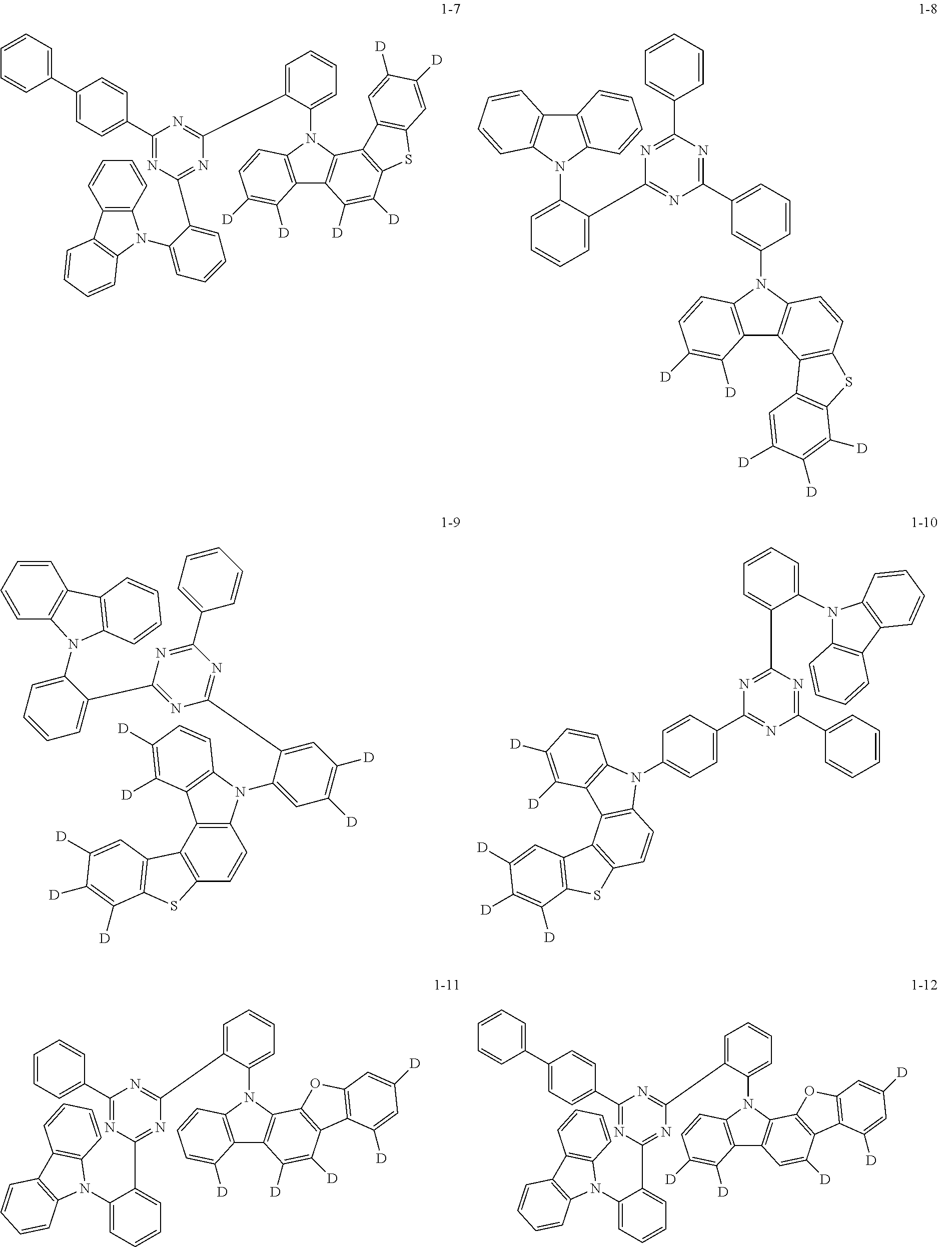

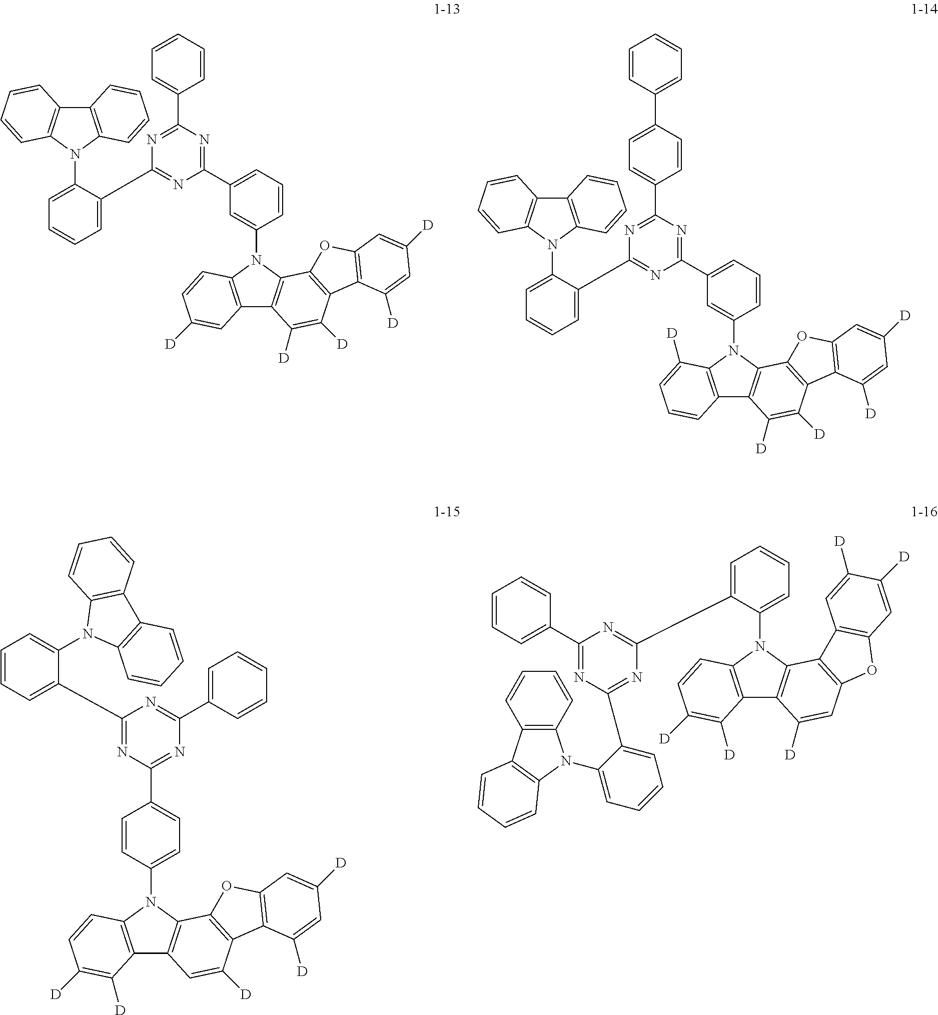

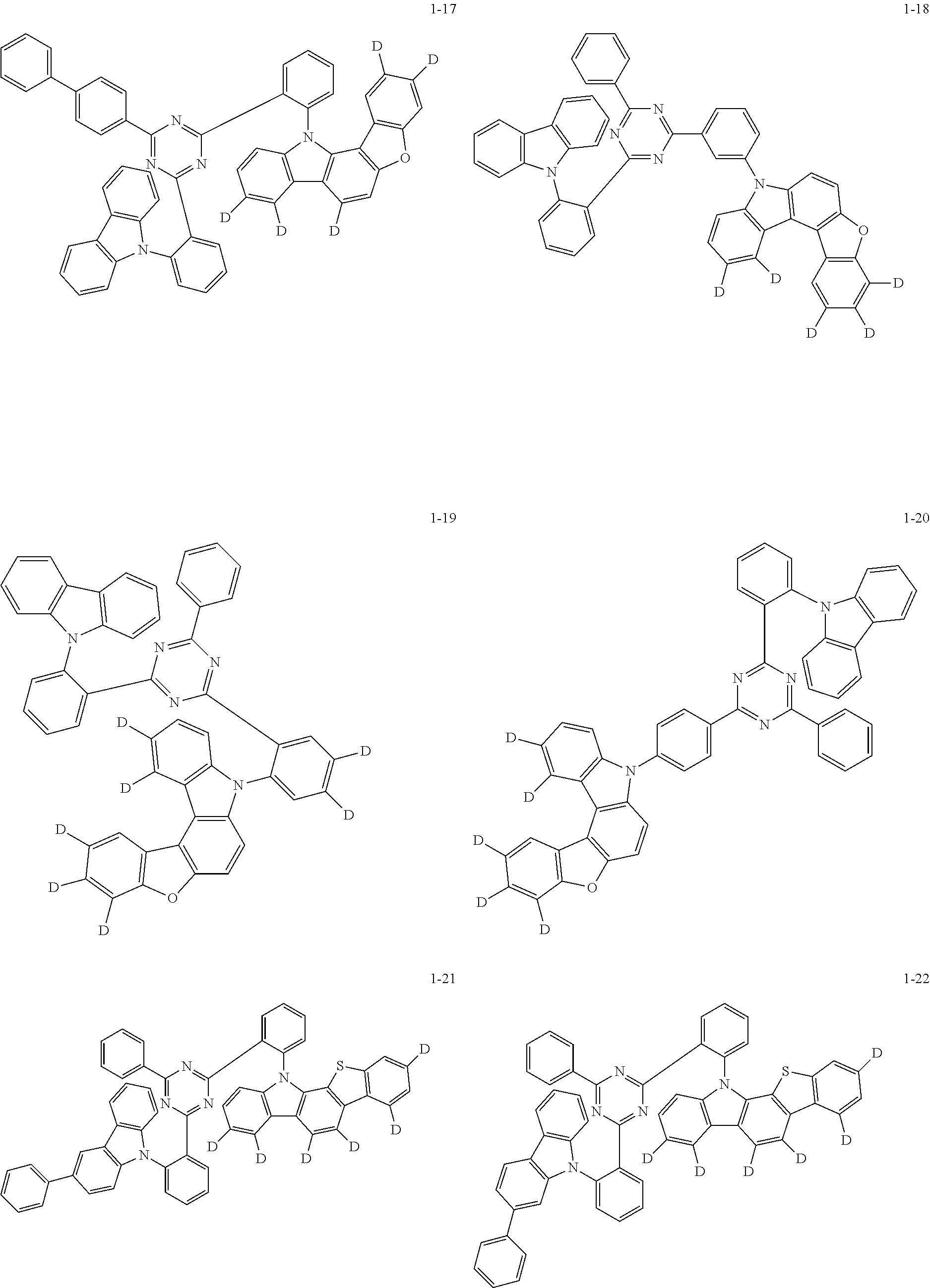

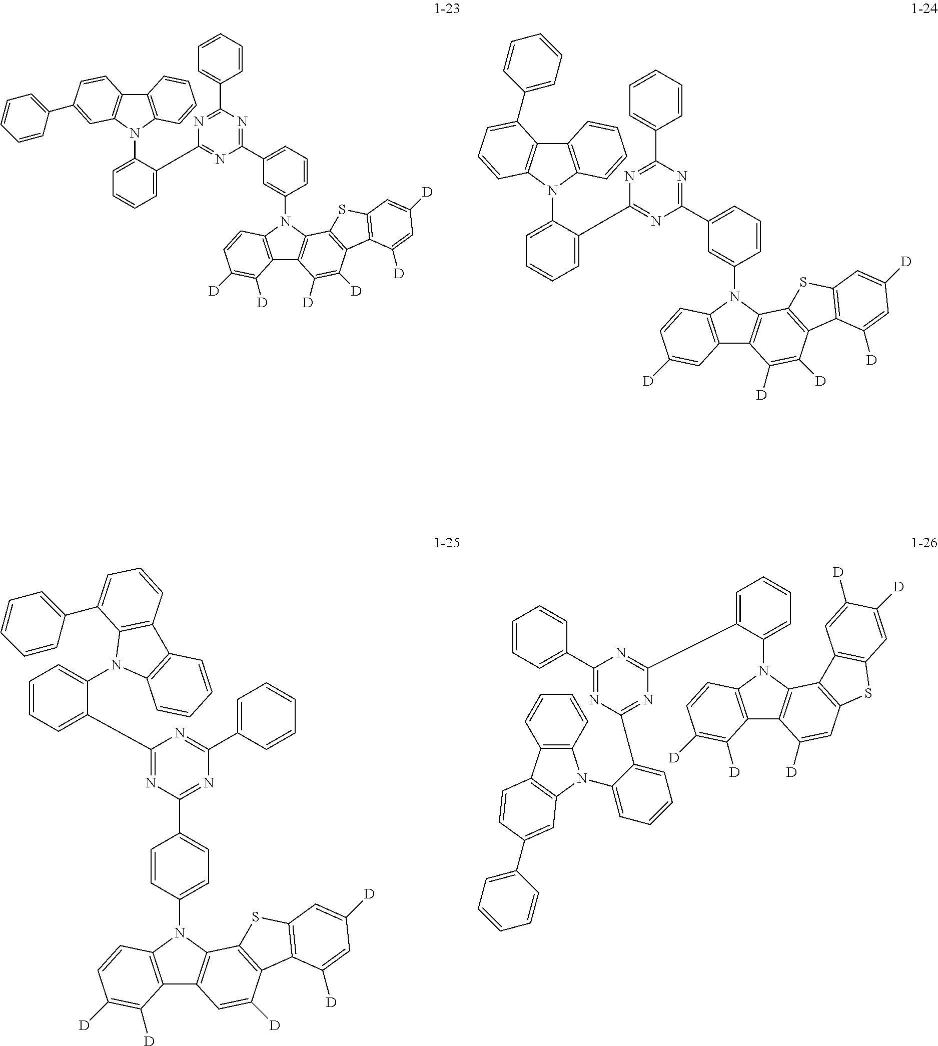

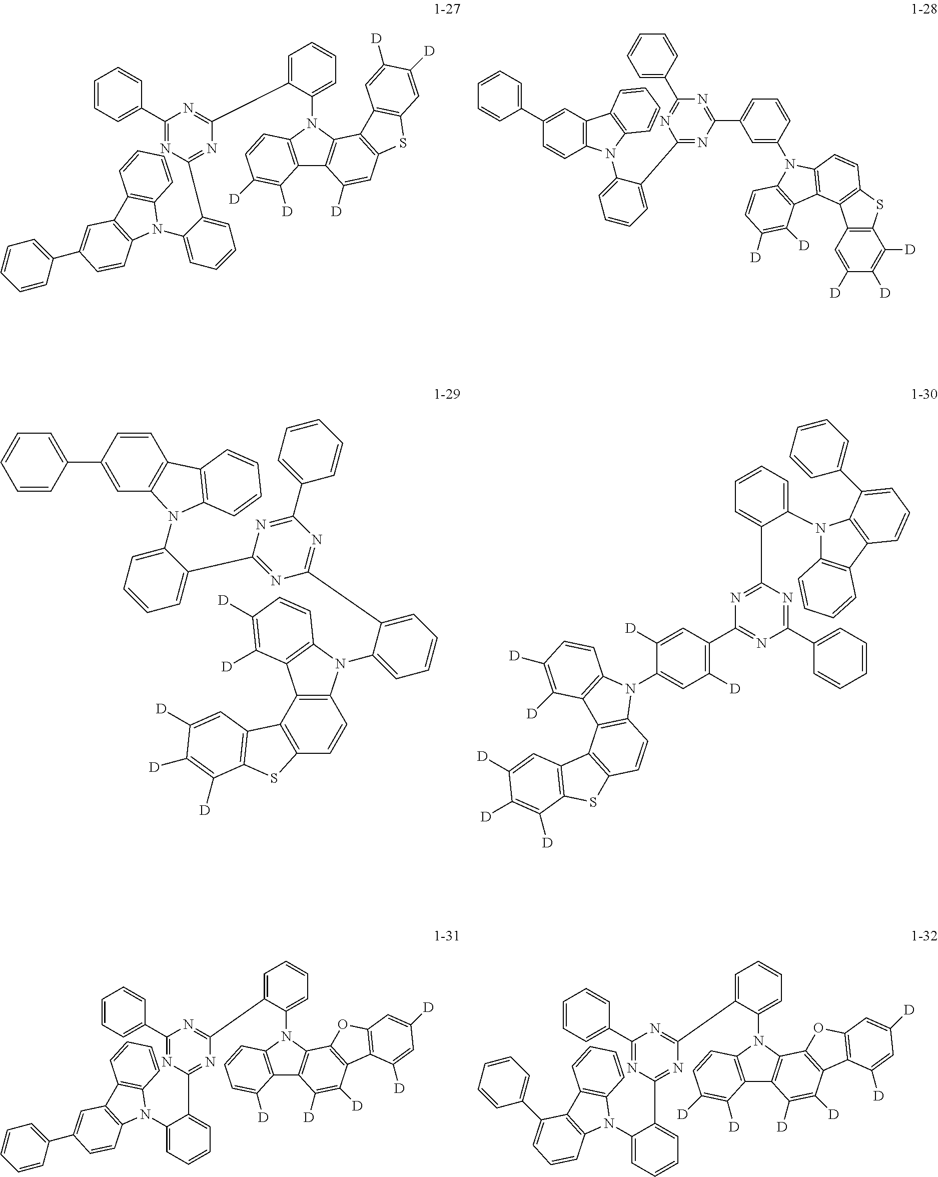

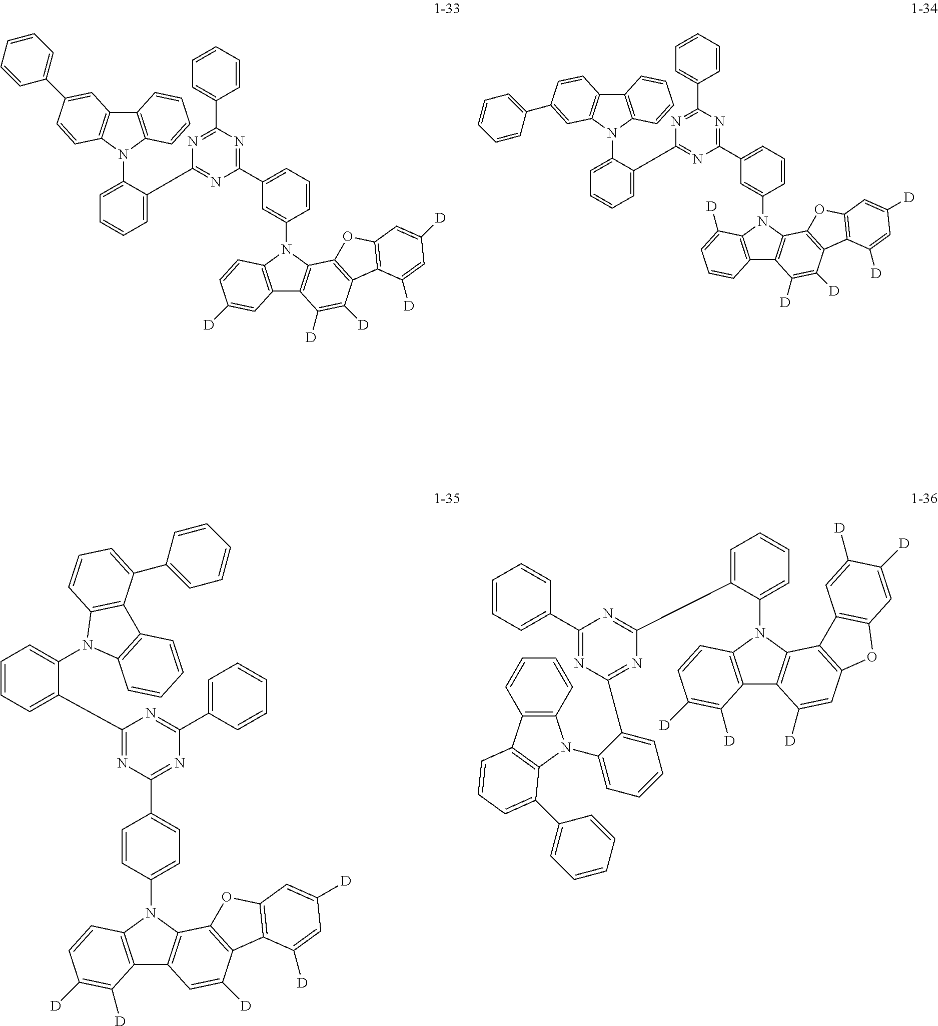

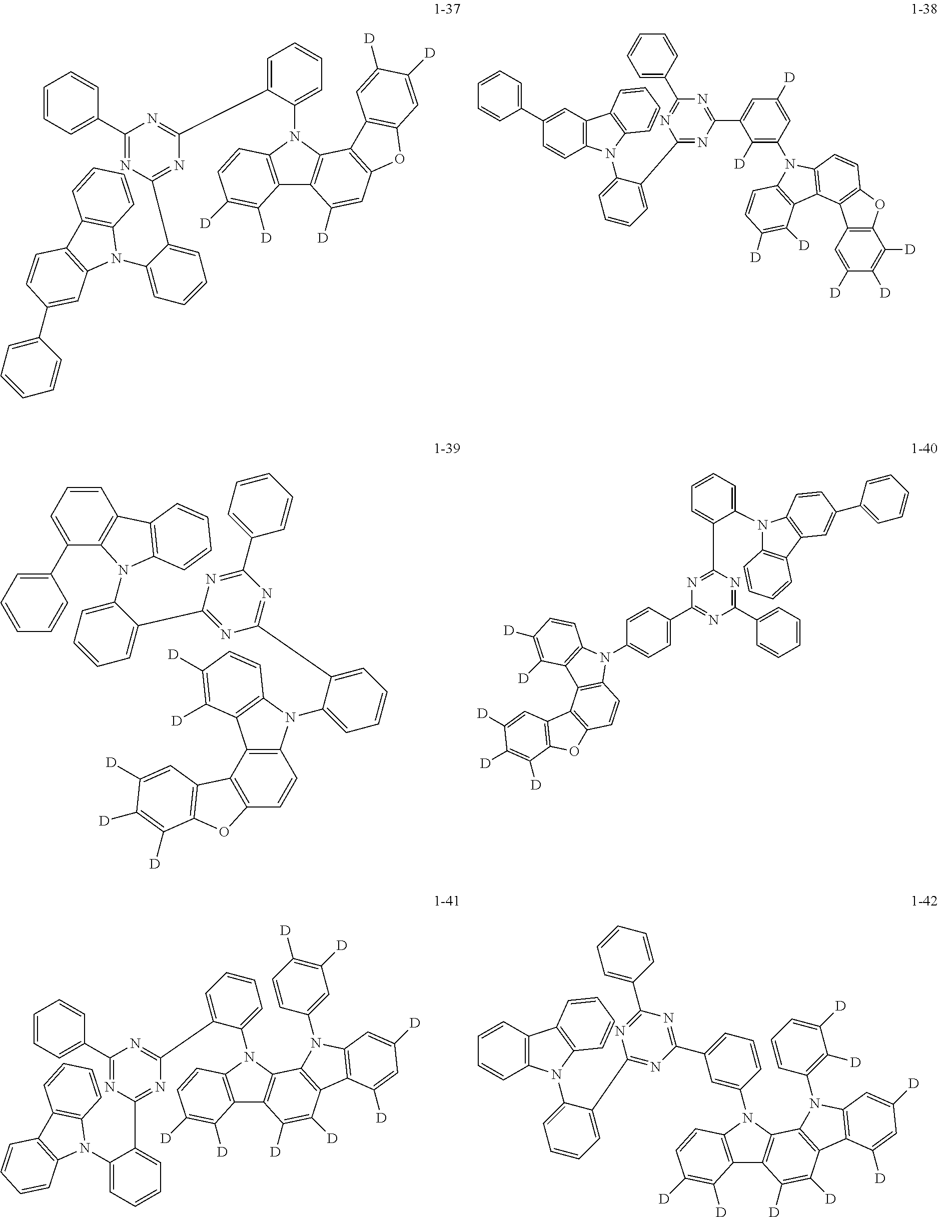

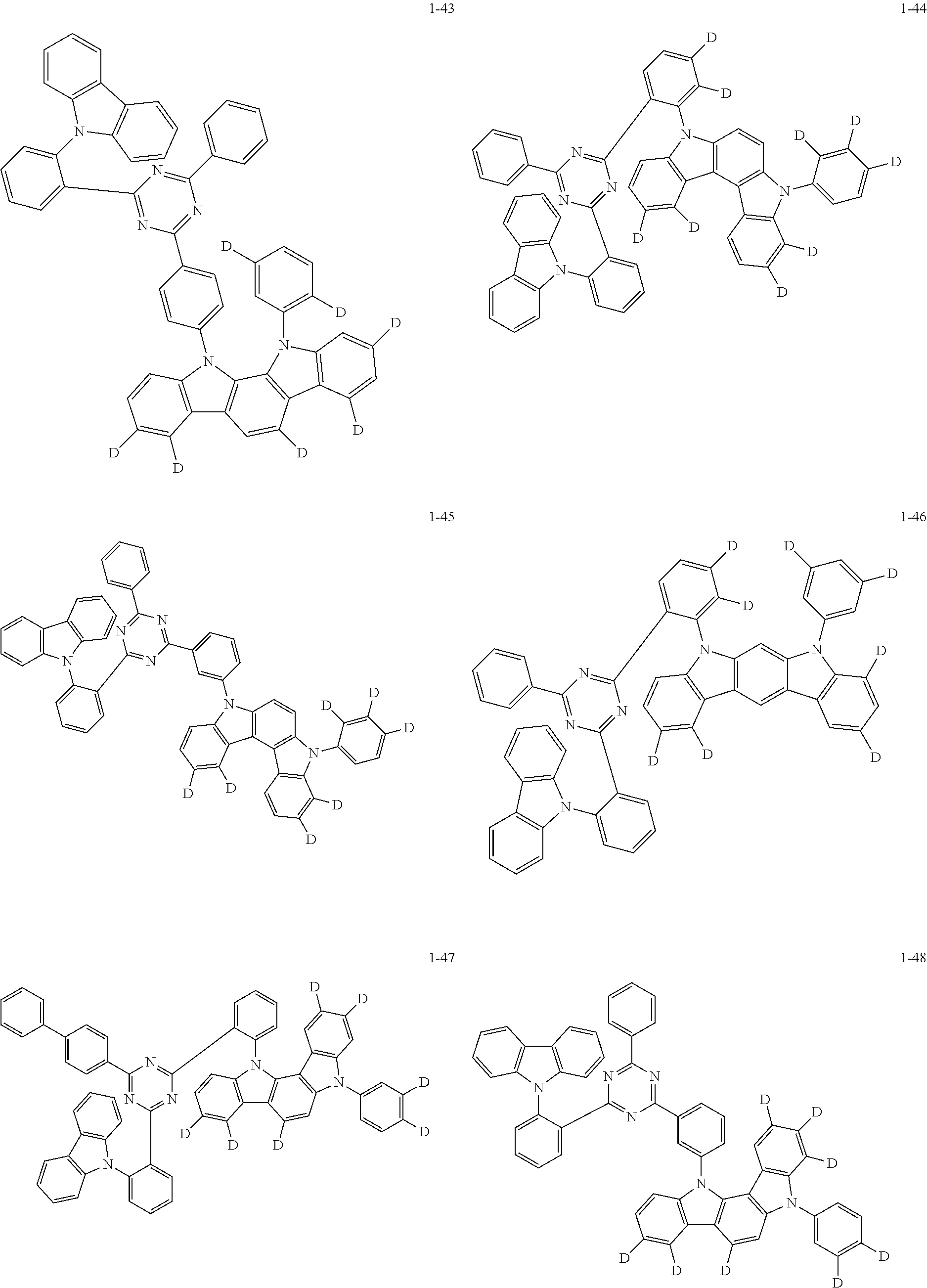

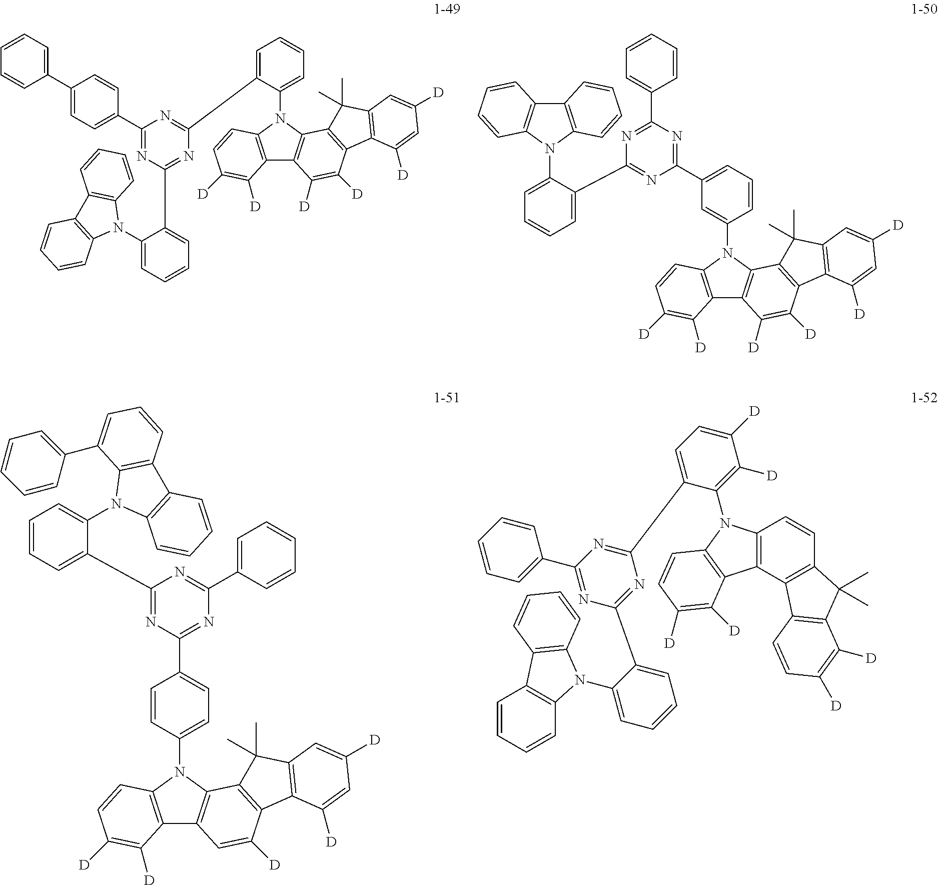

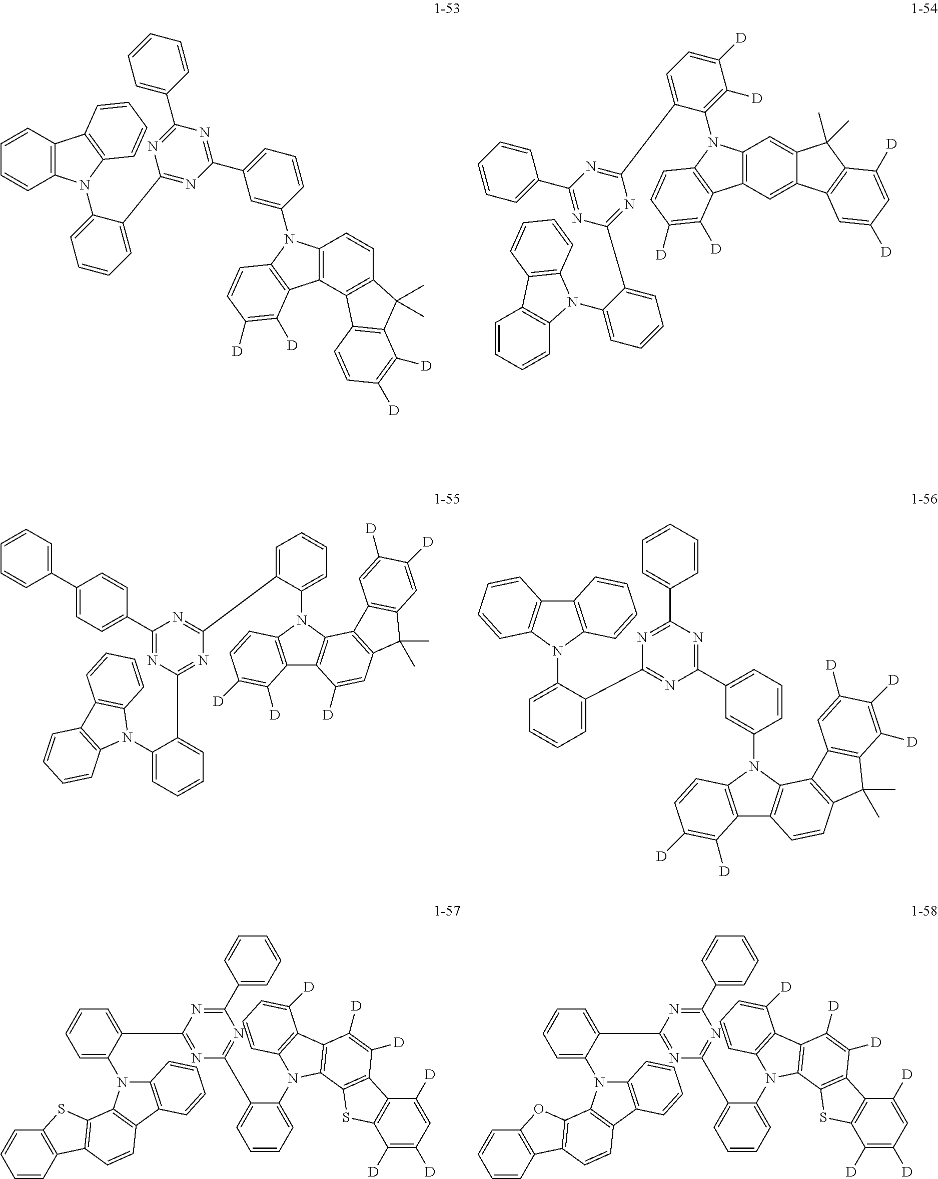

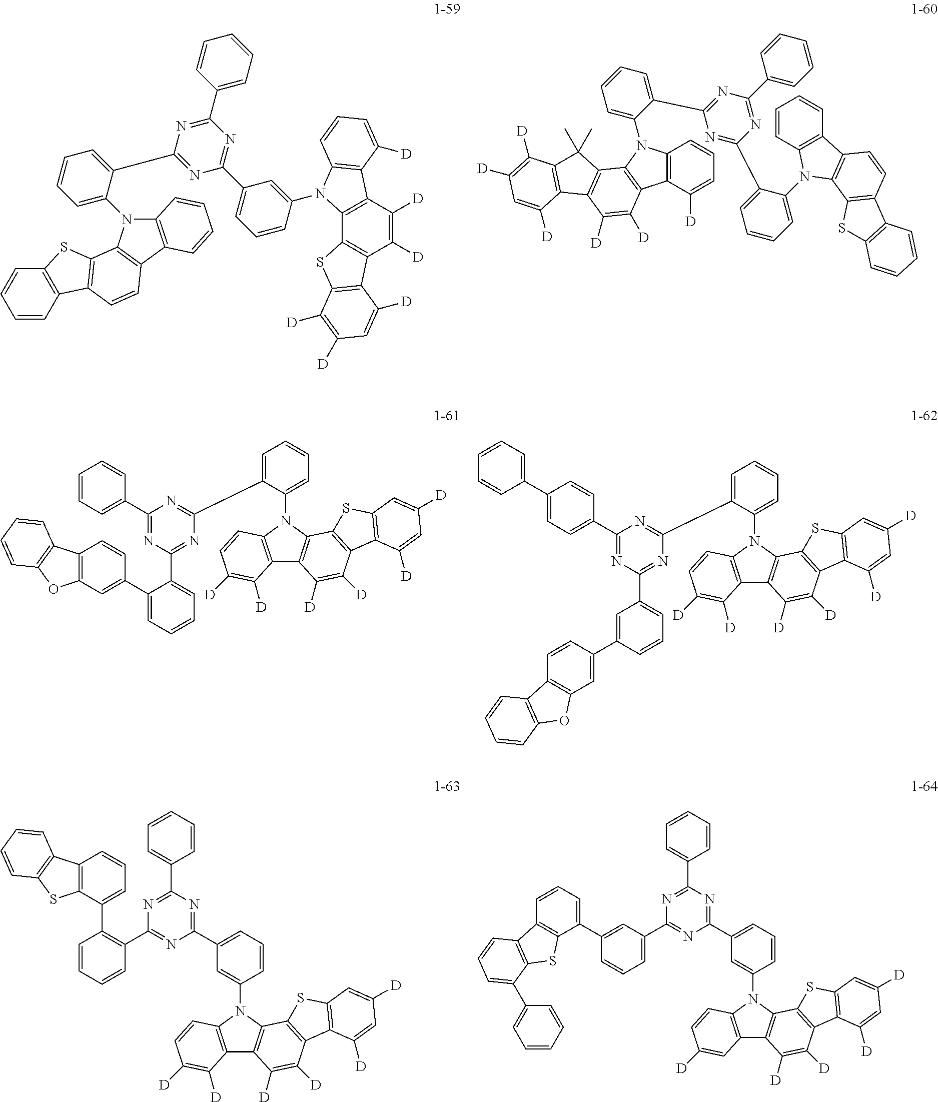

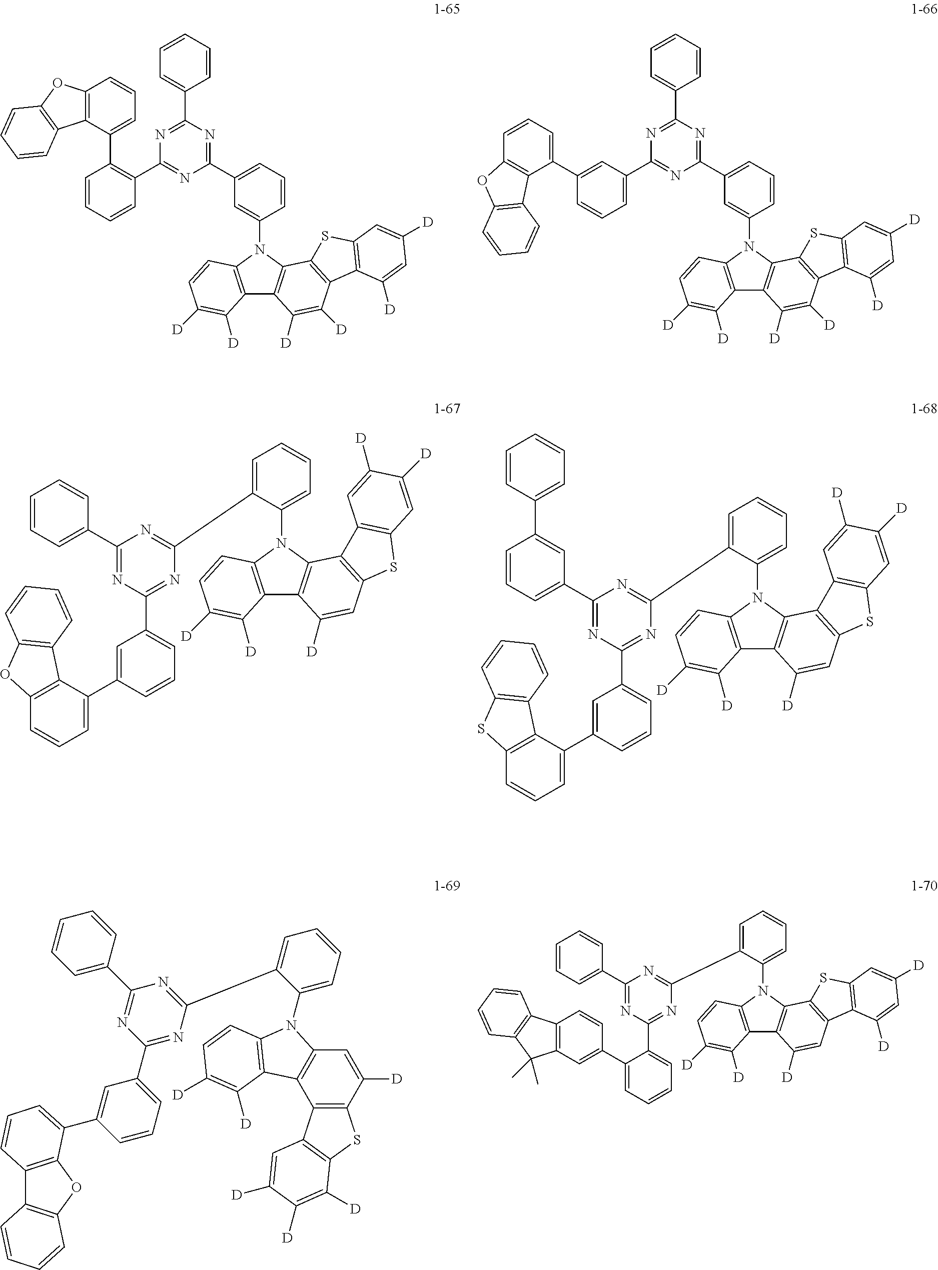

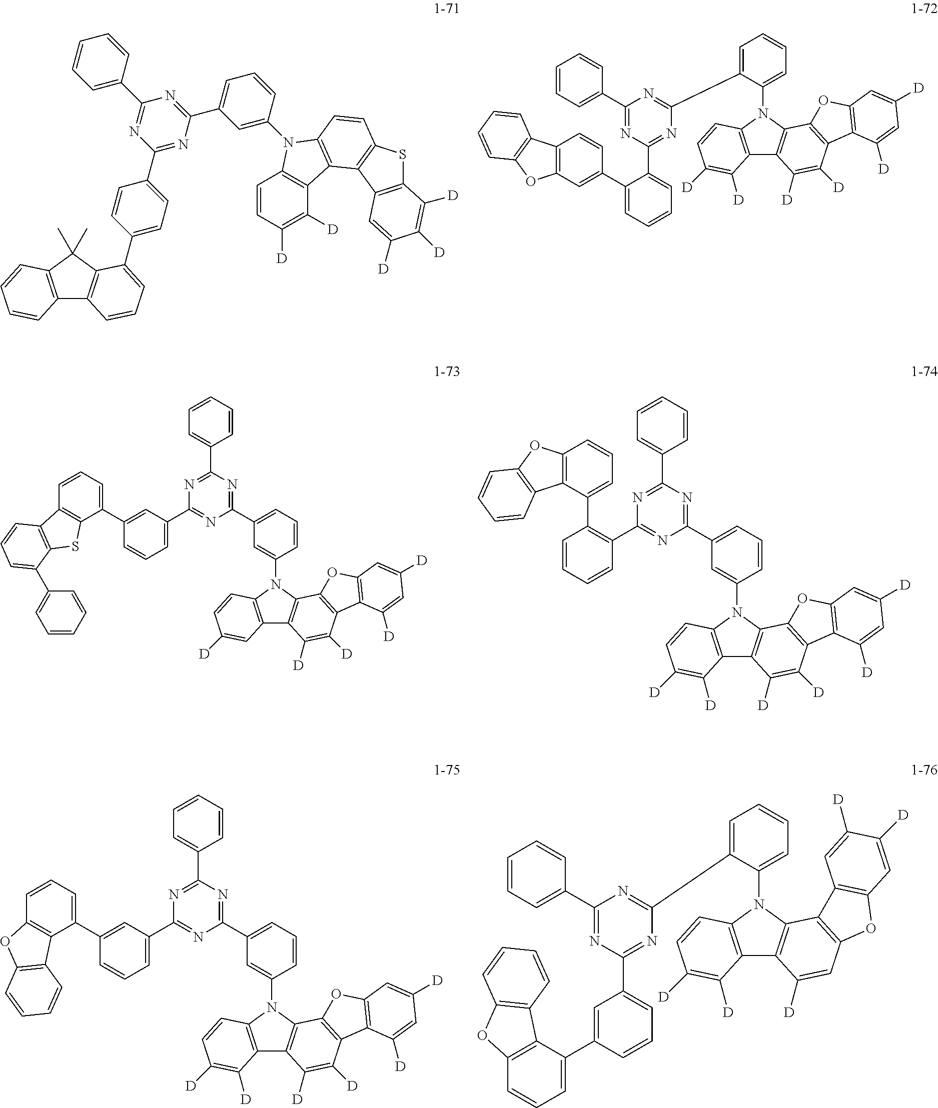

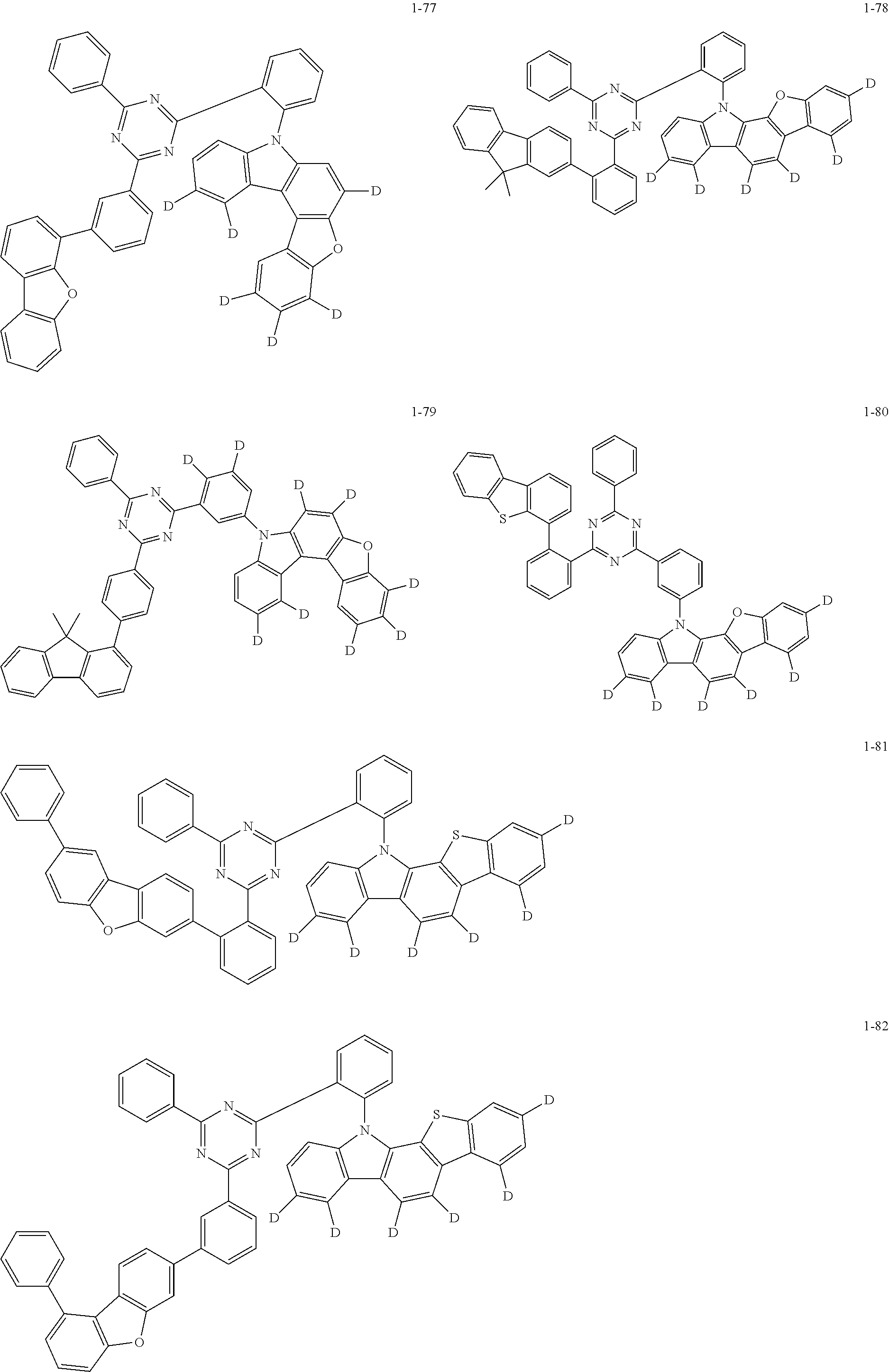

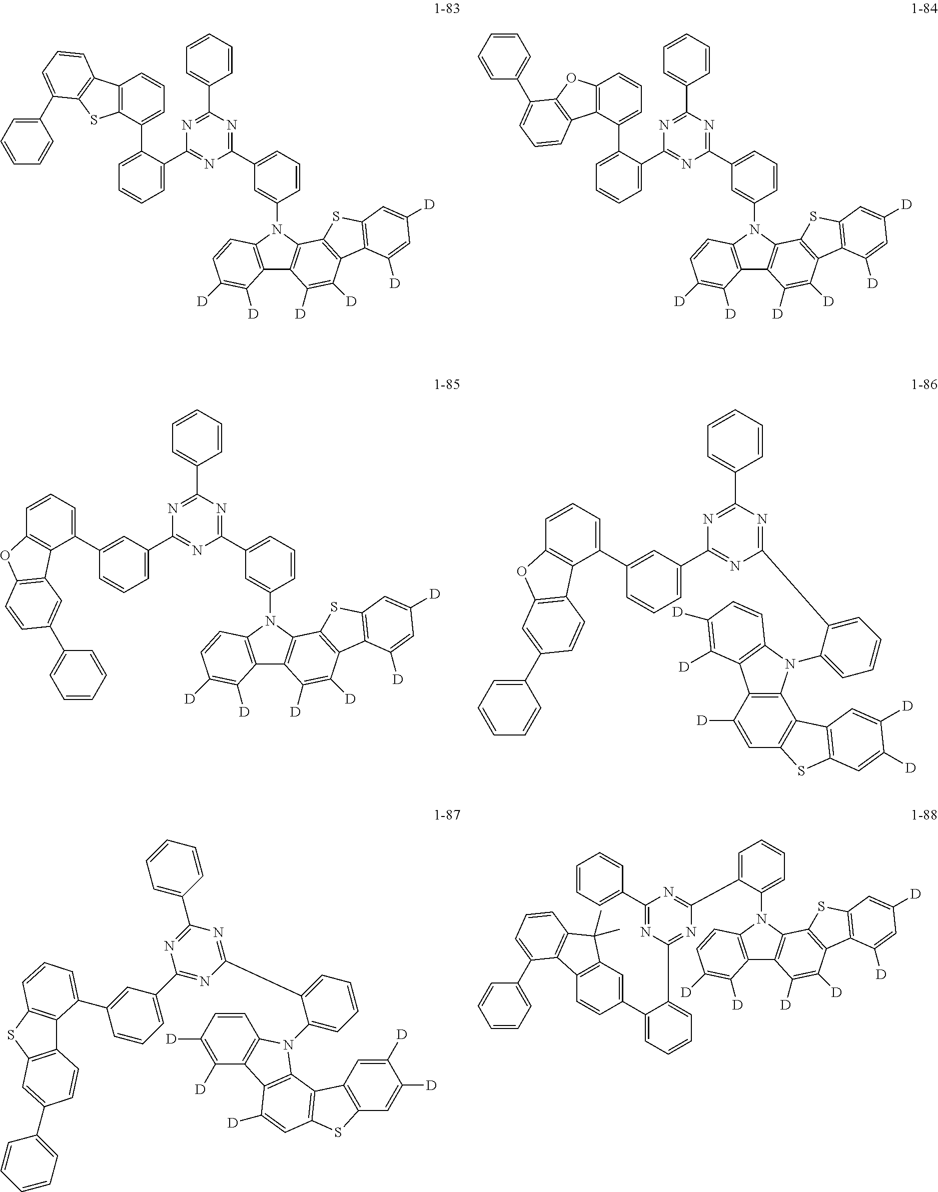

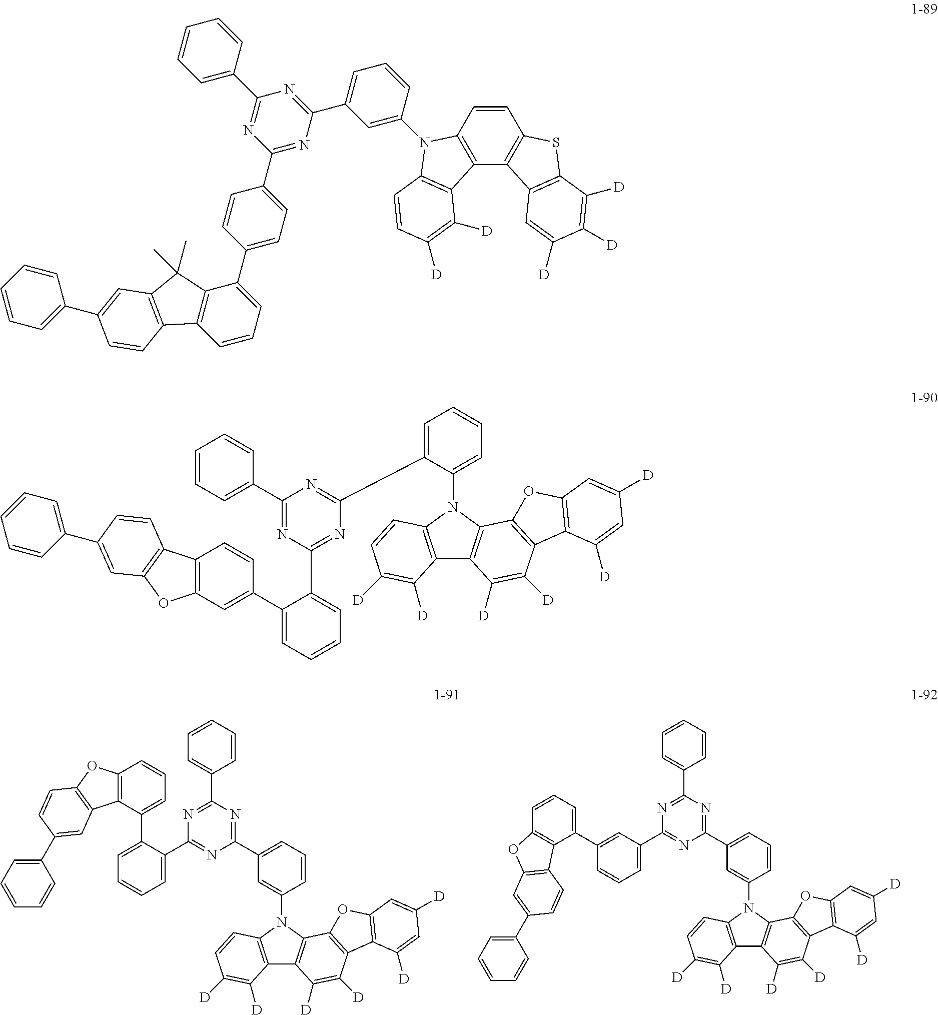

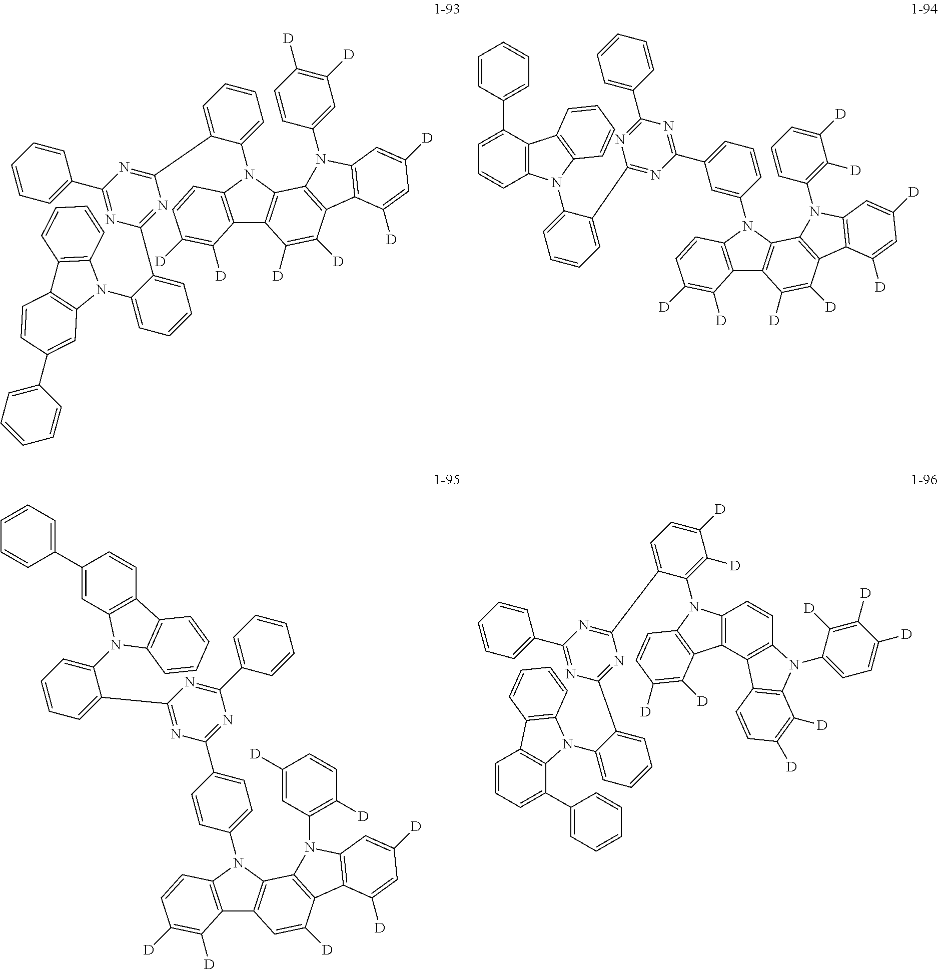

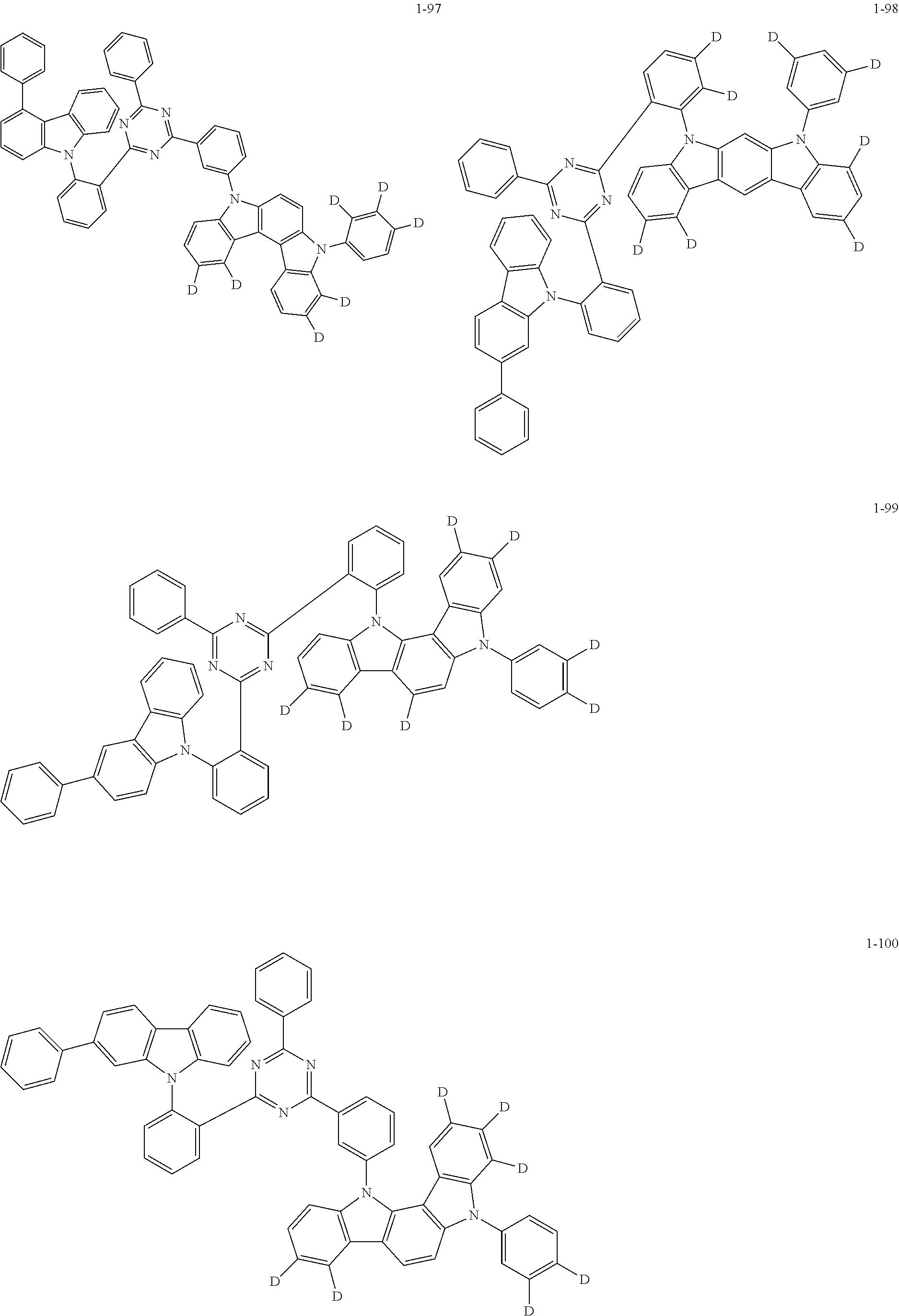

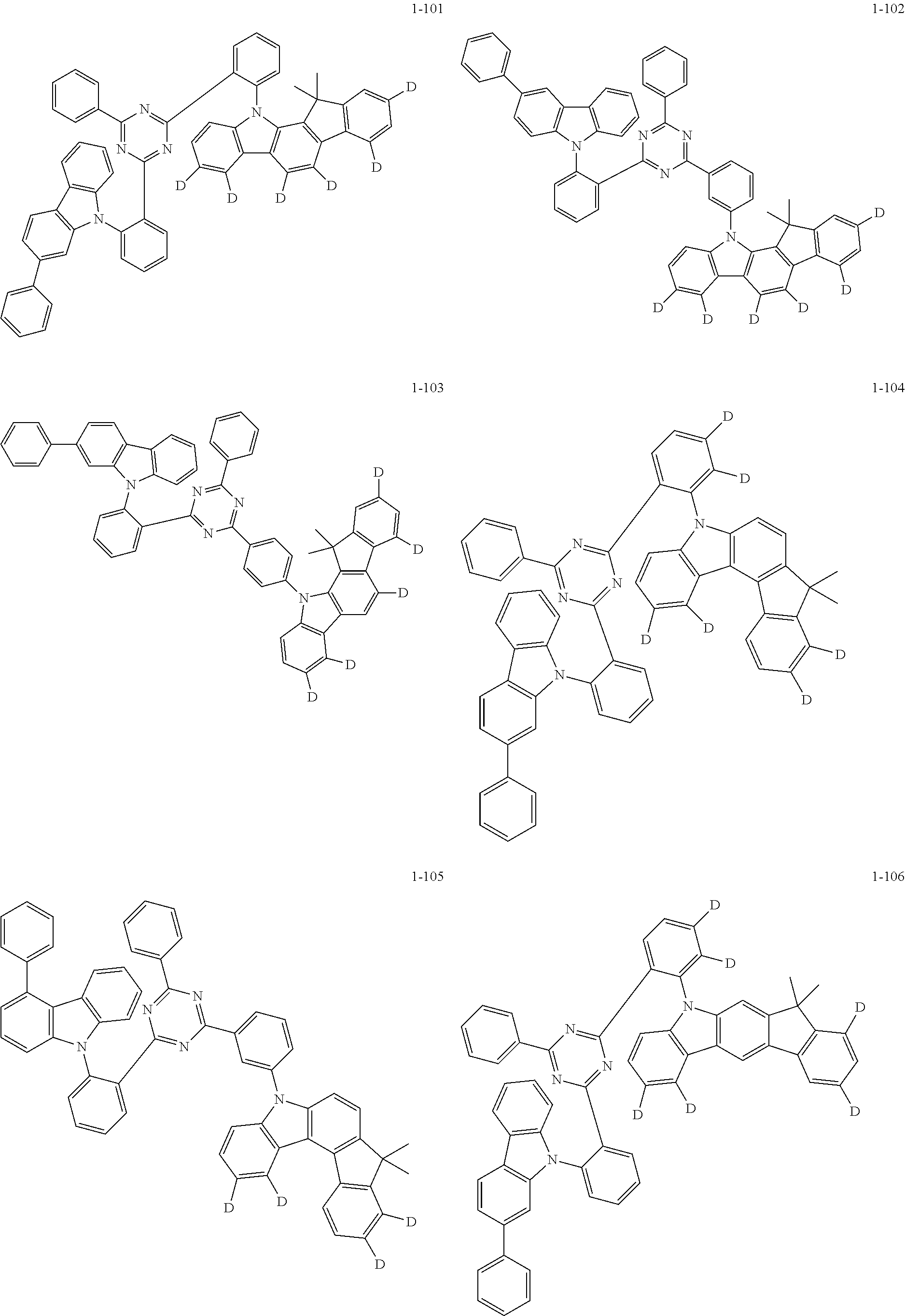

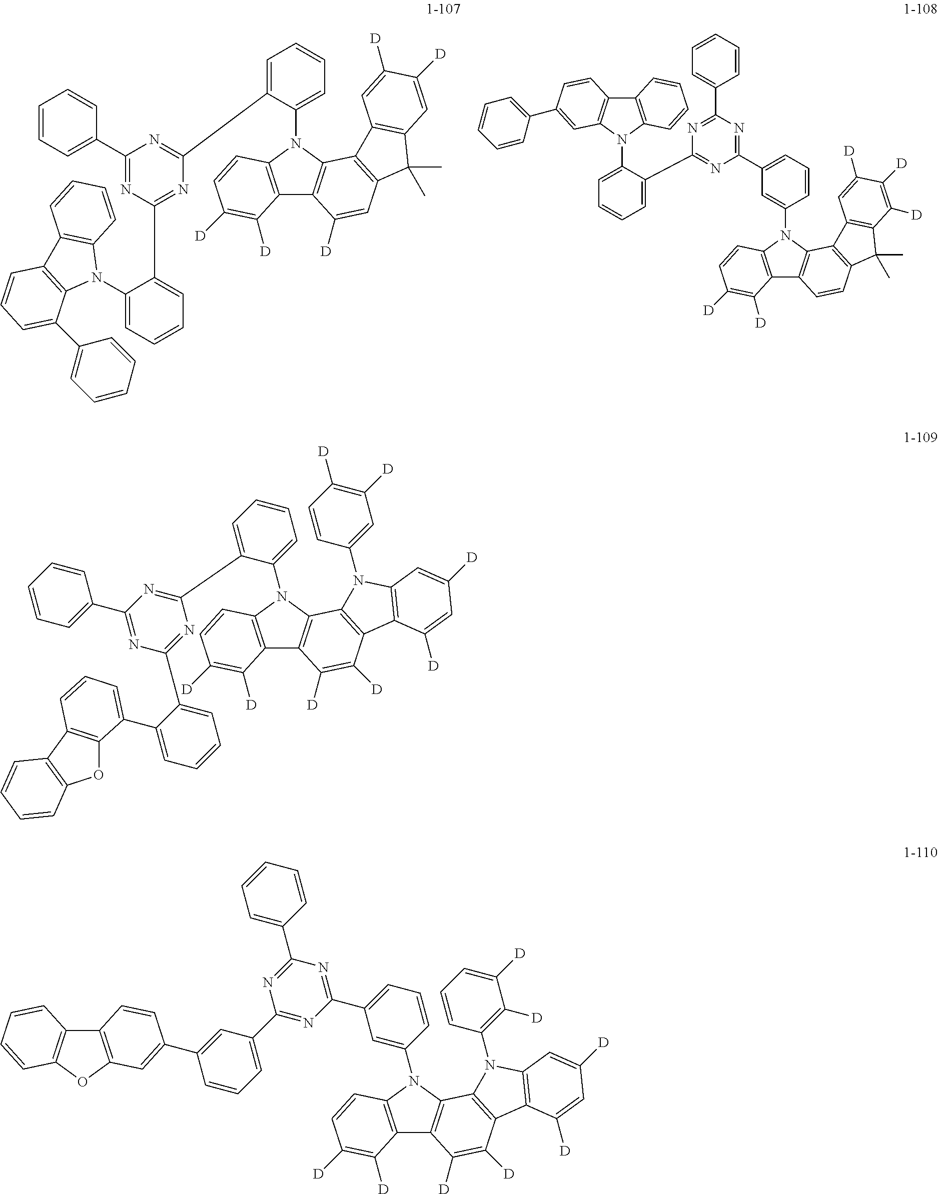

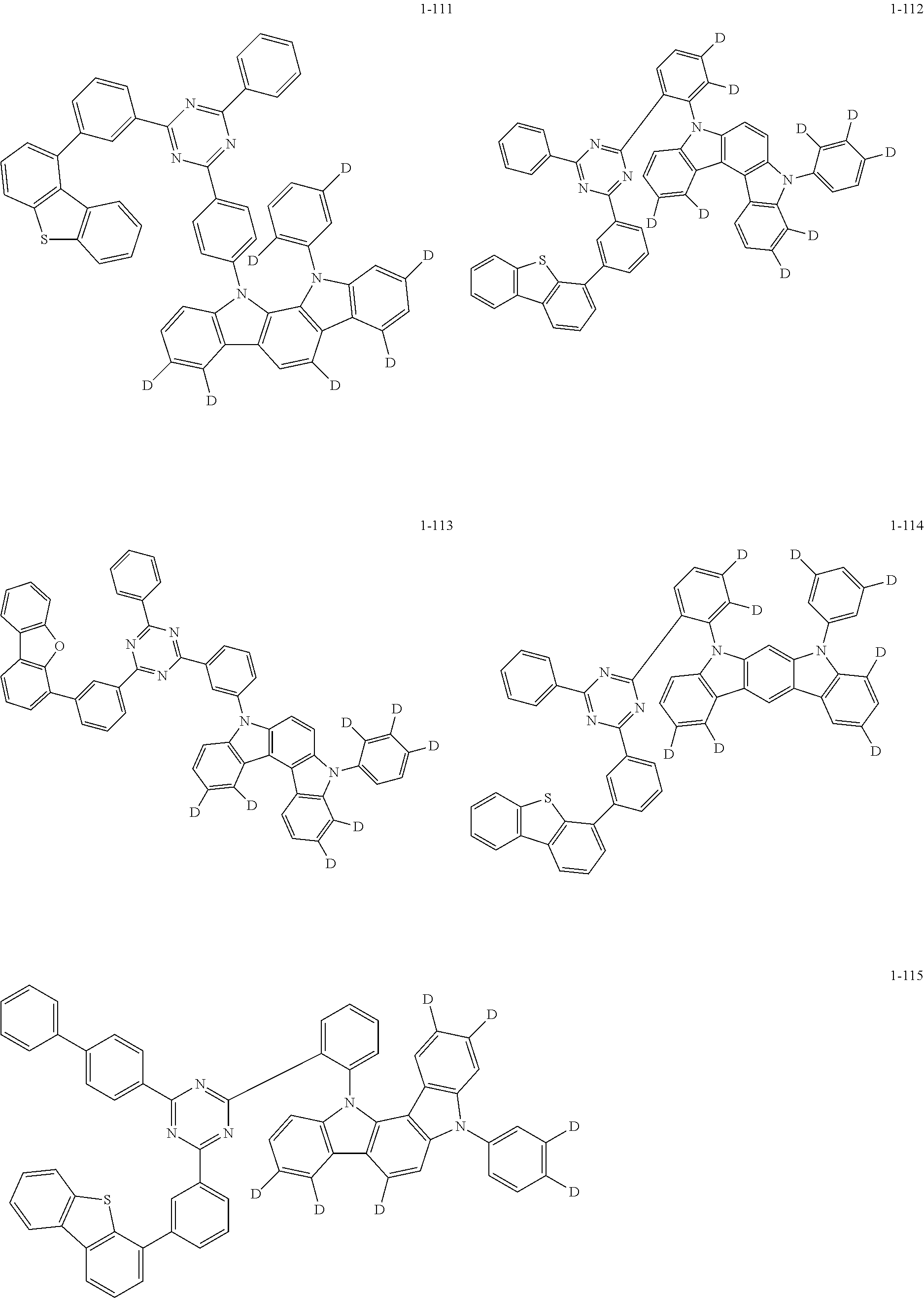

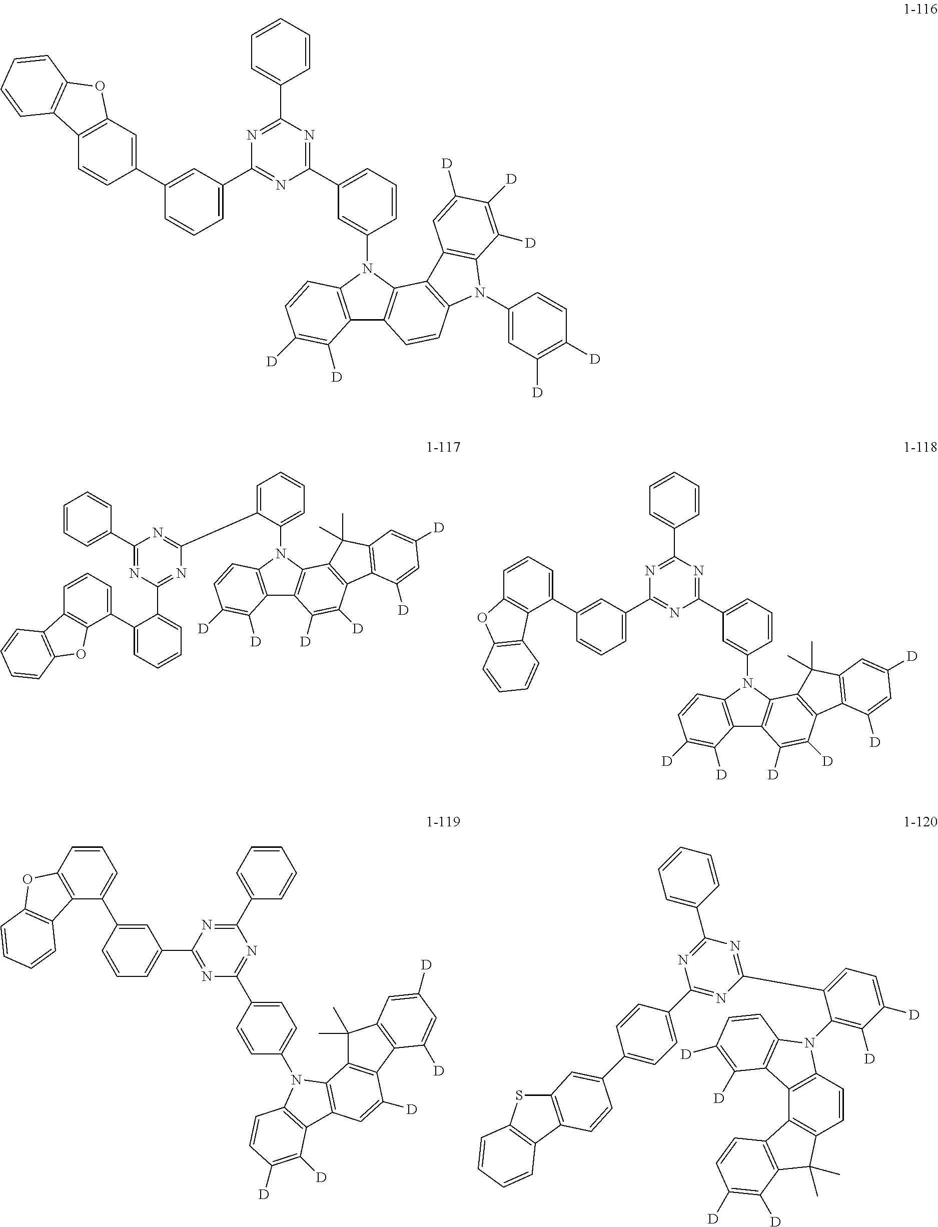

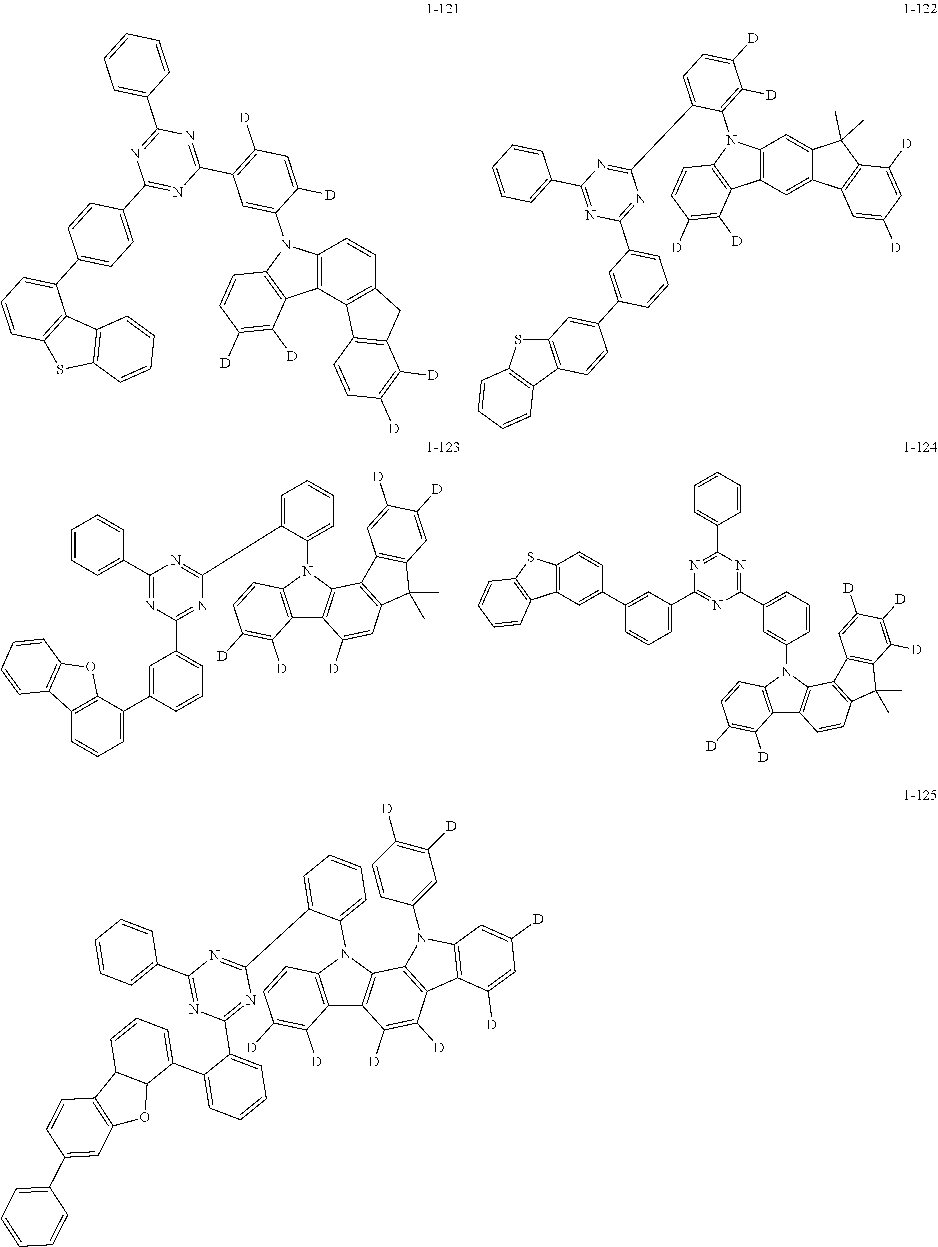

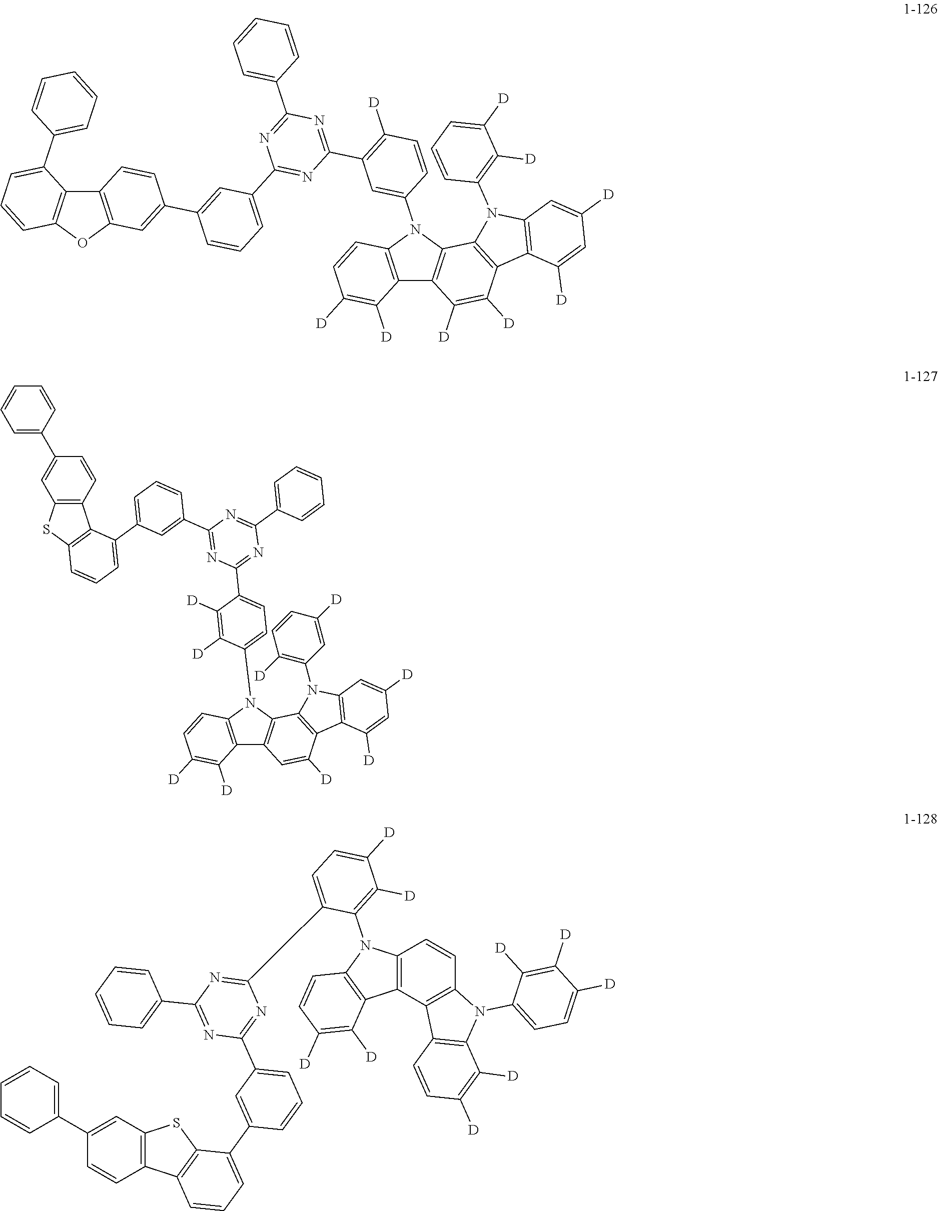

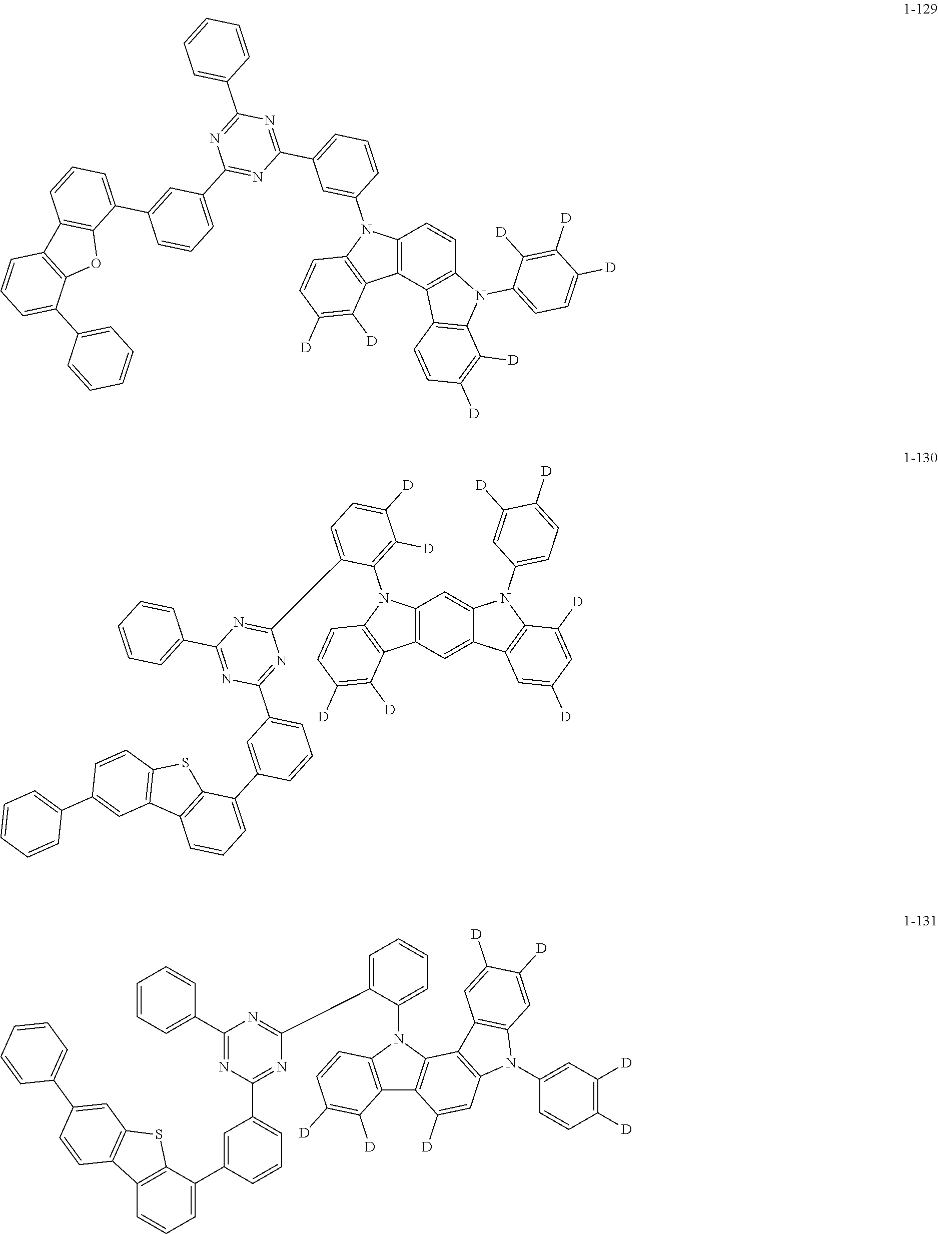 [SMILES: [2H]c1ccc(-n2c3ccc4c(c5c([2H])c([2H])ccc5n4-c4cccc(-c5nc(-c6ccccc6)nc(-c6cccc(-c7cccc8c7oc7c(-c9ccccc9)cccc78)c6)n5)c4)c3c3ccc([2H])c([2H])c32)c([2H])c1[2H] ; [2H]c1cc([2H])c2c(c1)c1cc3c4c([2H])c([2H])ccc4n(-c4c(-c5nc(-c6ccccc6)nc(-c6cccc(-c7cccc8c7sc7ccc(-c9ccccc9)cc78)c6)n5)ccc([2H])c4[2H])c3cc1n2-c1ccc([2H])c([2H])c1 ; [2H]c1ccc(-n2c3cc([2H])c([2H])cc3c3c2cc([2H])c2c4c([2H])c([2H])ccc4n(-c4ccccc4-c4nc(-c5ccccc5)nc(-c5cccc(-c6cccc7c6sc6cc(-c8ccccc8)ccc67)c5)n4)c23)cc1[2H]]

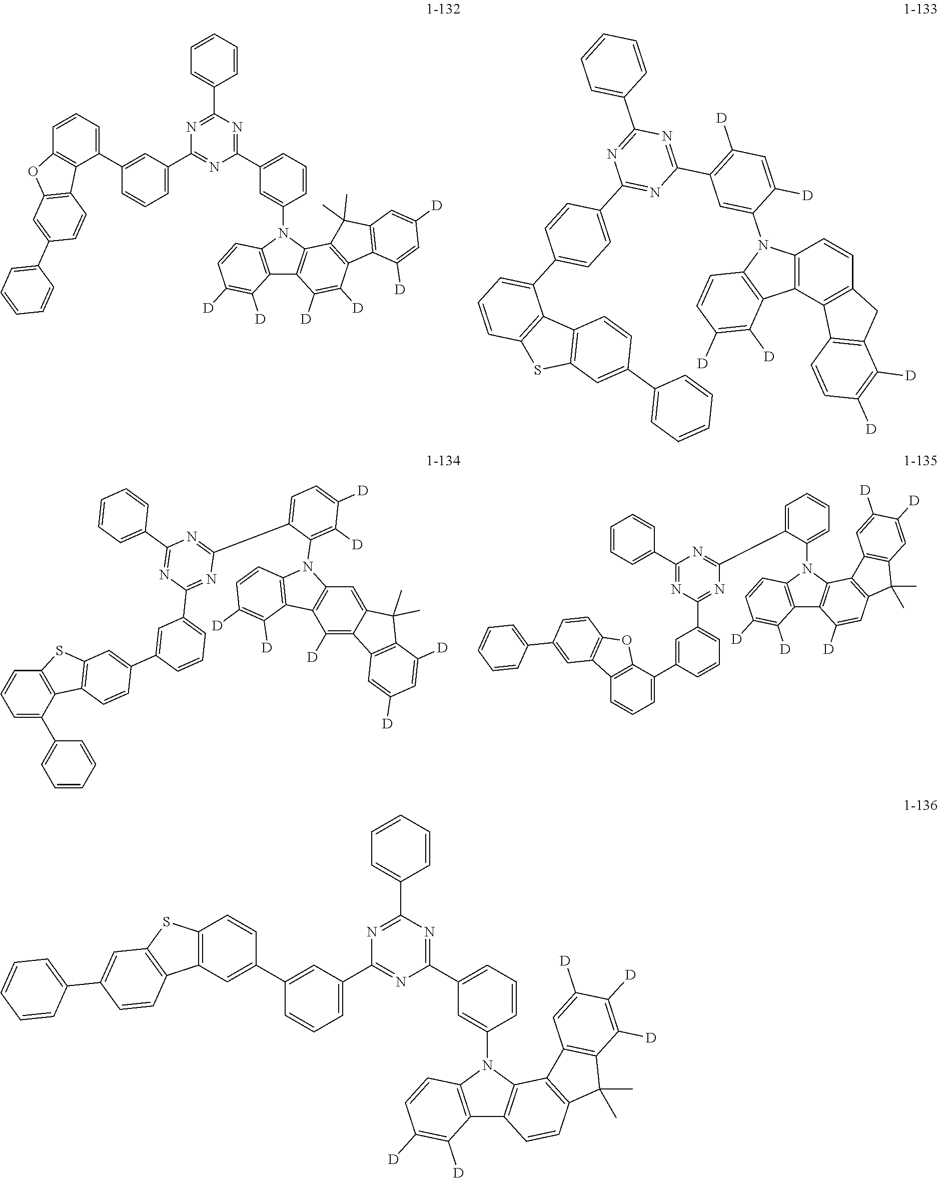

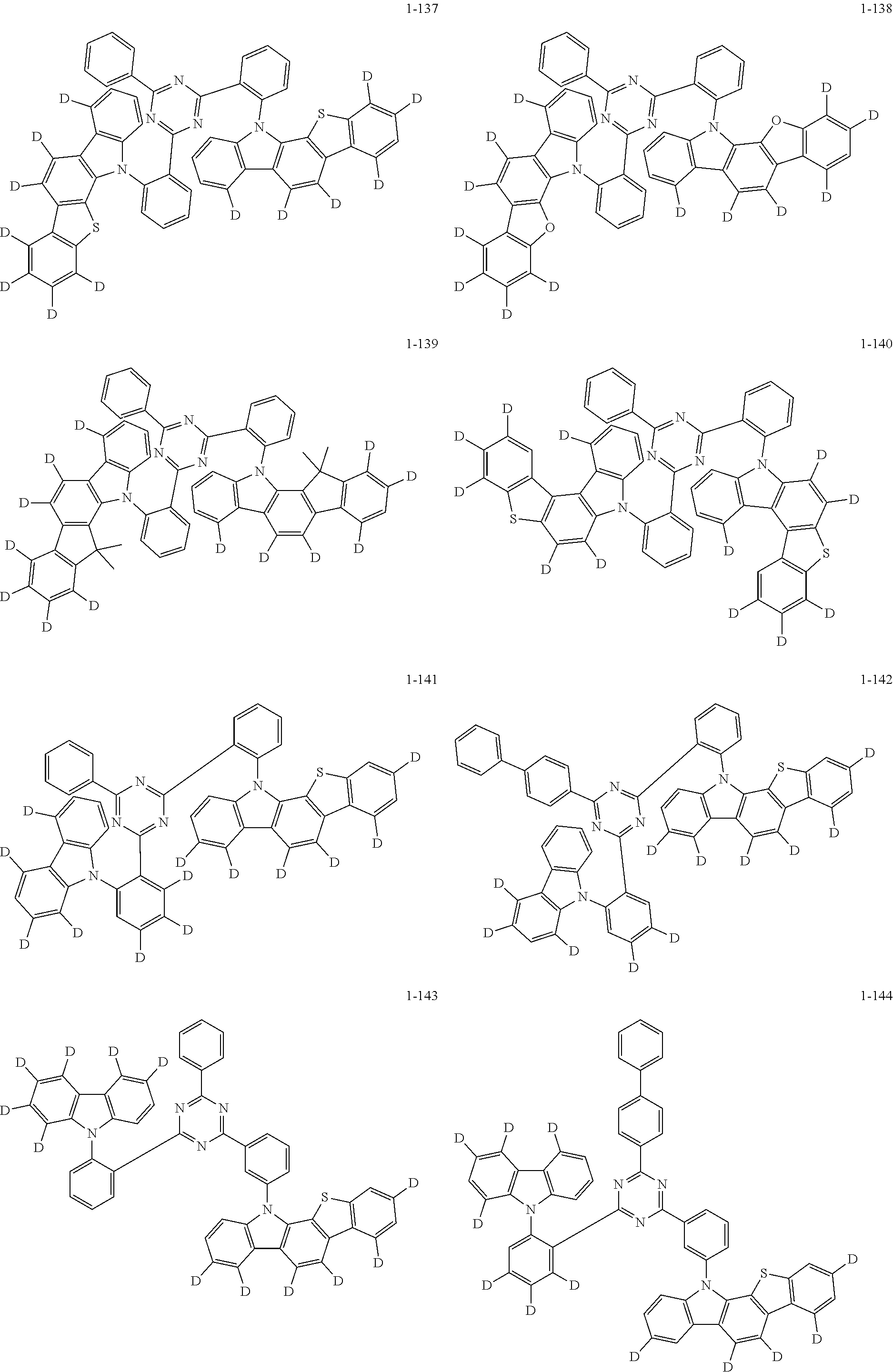

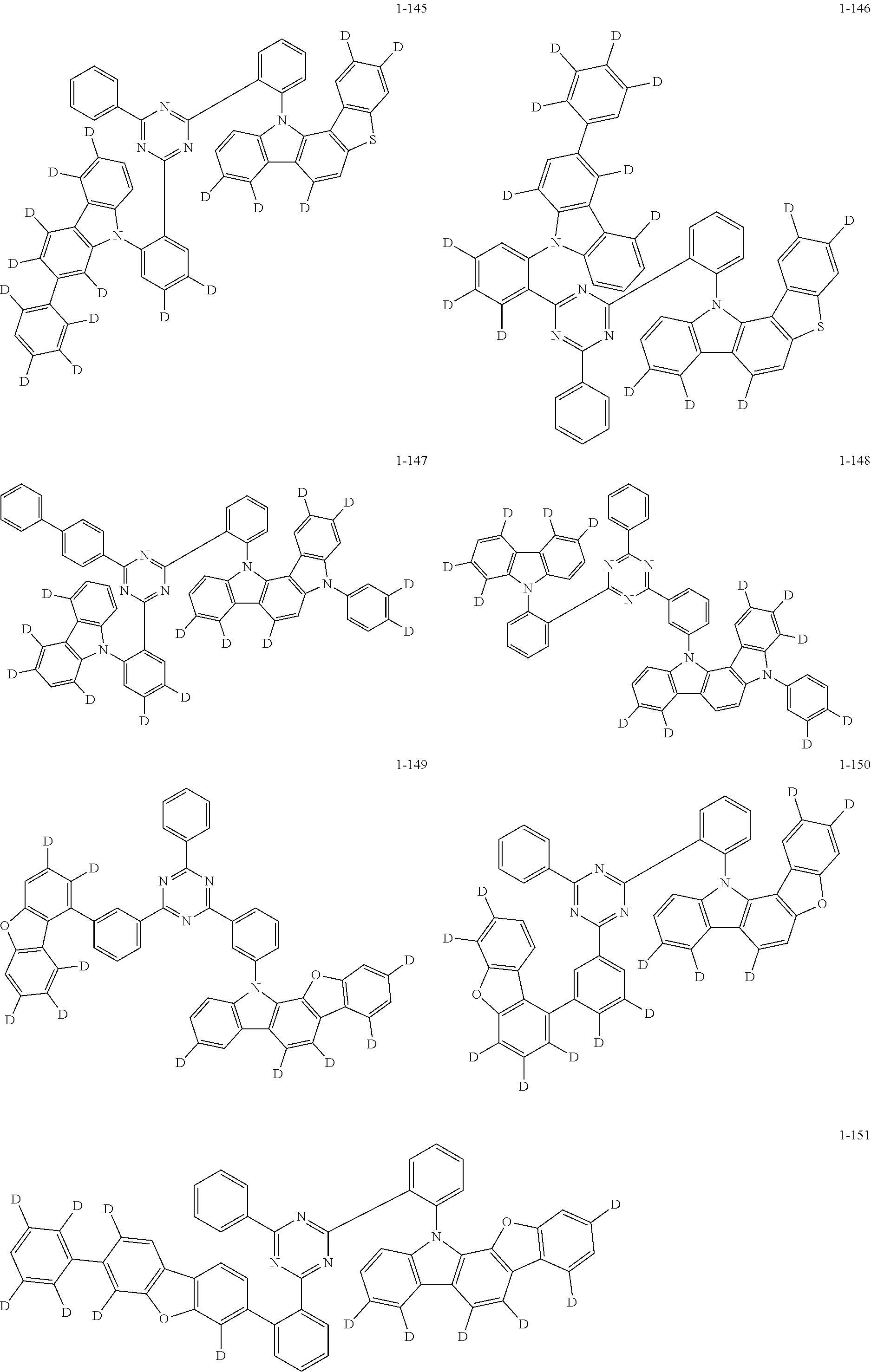

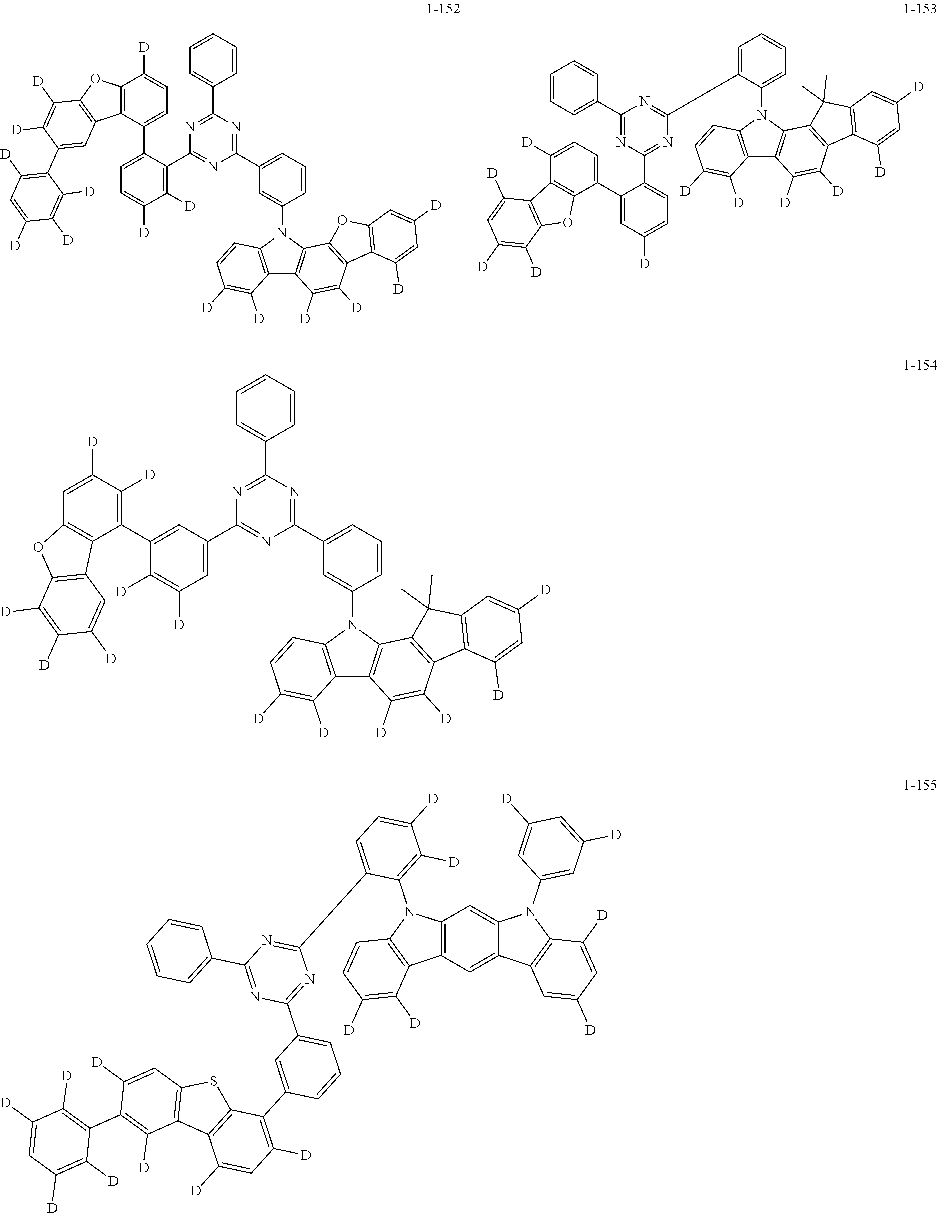

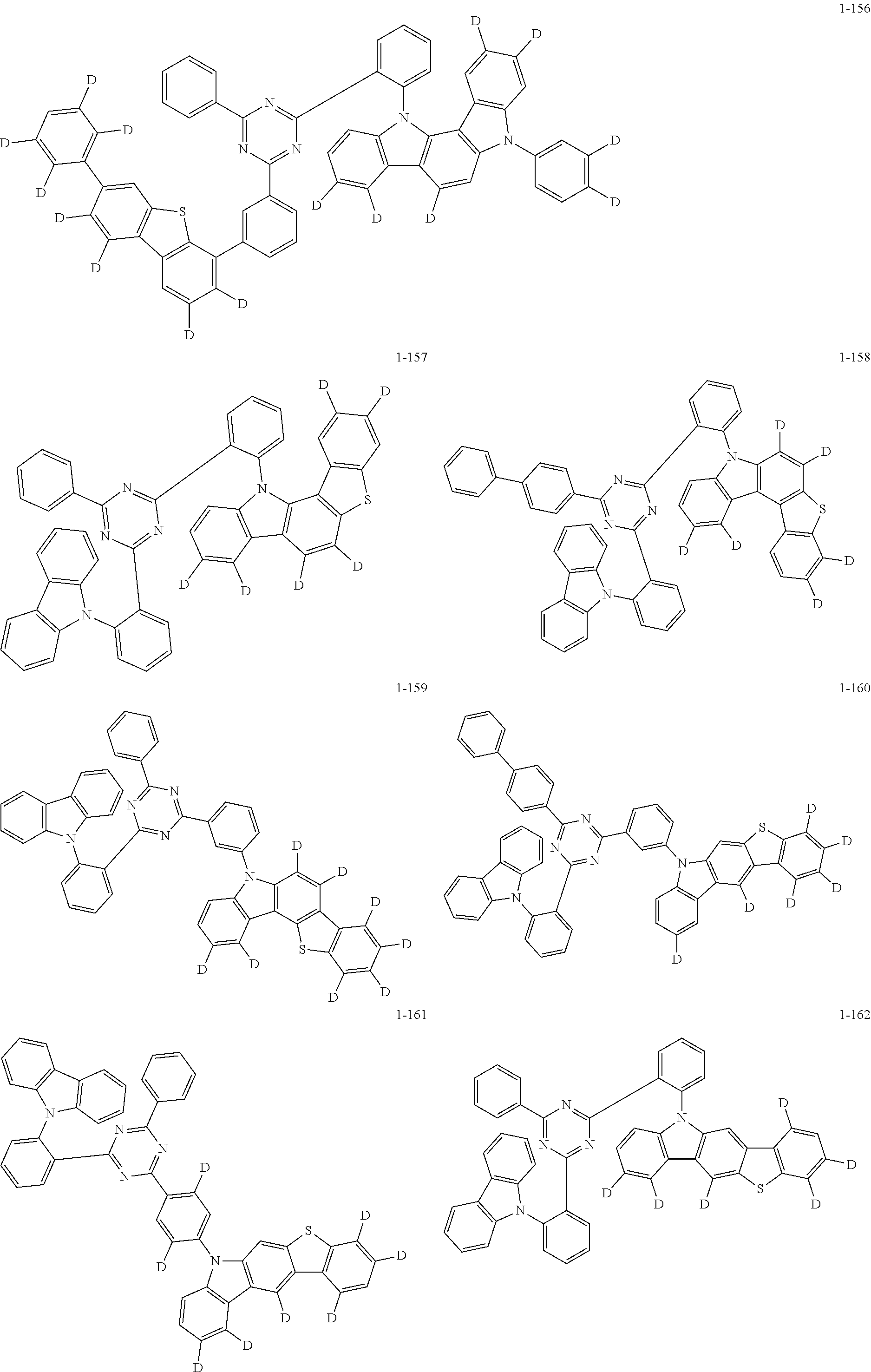

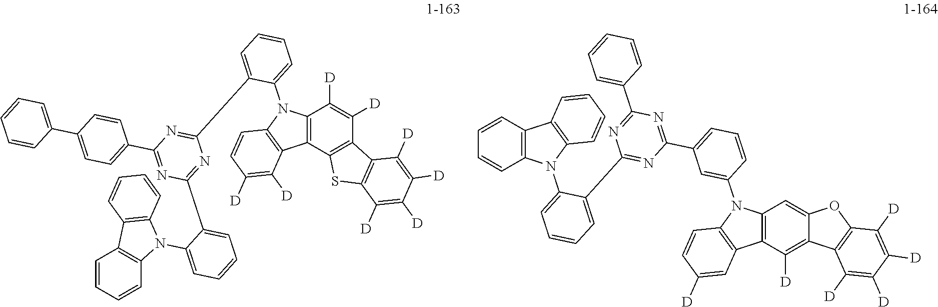 [SMILES: [2H]c1ccc2c(c1[2H])c1c3sc4c([2H])c([2H])c([2H])c([2H])c4c3c([2H])c([2H])c1n2-c1ccccc1-c1nc(-c2ccc(-c3ccccc3)cc2)nc(-c2ccccc2-n2c3ccccc3c3ccccc32)n1 ; [2H]c1ccc2c(c1)c1c([2H])c3c(cc1n2-c1cccc(-c2nc(-c4ccccc4)nc(-c4ccccc4-n4c5ccccc5c5ccccc54)n2)c1)oc1c([2H])c([2H])c([2H])c([2H])c13]

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a hole transport layer material, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

In another exemplary embodiment of the present specification, provided is an organic light emitting device including: a first electrode; a second electrode; and an organic material layer having one or more layers provided between the first electrode and the second electrode, in which one or more layers of the organic material layer include the one or more heterocyclic compounds of Chemical Formula 1.

In an exemplary embodiment of the present specification, the organic material layer includes a light emitting layer, and the light emitting layer may include one or more of the heterocyclic compounds.

In an exemplary embodiment of the present specification, the organic material layer includes a light emitting layer, and the light emitting layer may include one of the heterocyclic compounds.

In an exemplary embodiment of the present specification, the organic material layer includes a light emitting layer, the light emitting layer includes a host, and the host may include one or more of the heterocyclic compounds.

In an exemplary embodiment of the present specification, the organic material layer includes a light emitting layer, the light emitting layer includes a host, and the host may include one of the heterocyclic compounds.

In an exemplary embodiment of the present specification, the organic material layer includes a light emitting layer, the light emitting layer includes a host, the host includes a green host, and the green host may include one or more of the heterocyclic compounds.

In an exemplary embodiment of the present specification, the organic material layer includes a light emitting layer, the light emitting layer includes a host, the host includes a red host, and the red host may include one or more of the heterocyclic compounds.

In an exemplary embodiment of the present specification, the organic material layer includes a light emitting layer, the light emitting layer includes a host, the host includes a blue host, and the blue host may include one or more of the heterocyclic compounds.

In an exemplary embodiment of the present specification, the organic material layer includes a light emitting layer, and the light emitting layer may include the heterocyclic compound as an N-type host.

In an exemplary embodiment of the present specification, an organic material layer including the heterocyclic compound may further include a compound of the following Chemical Formula 2 or 3.

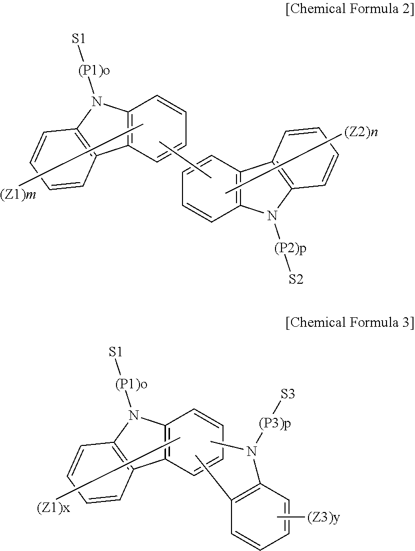

In Chemical Formulae 2 and 3,
P1 to P3 are each independently a direct bond; a substituted or unsubstituted C6 to C60 arylene group; or a substituted or unsubstituted C2 to C60 heteroarylene group,
S1 to S3 are each independently a cyano group; a substituted or unsubstituted triarylsilyl group; a substituted or unsubstituted C6 to C60 aryl group; or a substituted or unsubstituted C2 to C60 heteroaryl group,
Z1 to Z3 are each independently hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
m and n are each an integer of 1 to 7,
x is an integer from 1 to 6, and
y is an integer from 1 to 4,
o, p, and q are each an integer from 1 to 3, and
when m, n, x, y, o, p, and g are each 2 or greater, substituents in the parenthesis are the same as or different from each other.

In an exemplary embodiment of the present specification, the organic material layer includes a light emitting layer, and the light emitting layer may further include the compound of Chemical Formula 2 or 3.

In an exemplary embodiment of the present specification, the organic material layer includes a light emitting layer, the light emitting layer includes a host, and the host may further include the compound of Chemical Formula 2 or 3.

In an exemplary embodiment of the present specification, the light emitting layer may further include the compound of Chemical Formula 2 or 3 as a P-type host.

In an exemplary embodiment of the present specification, P1 to P3 may be each independently a direct bond; a substituted or unsubstituted C6 to C40 arylene group; or a substituted or unsubstituted C2 to C40 heteroarylene group.

In an exemplary embodiment of the present specification, P1 to P3 may be each independently a direct bond; a substituted or unsubstituted C6 to C20 arylene group; or a substituted or unsubstituted C2 to C20 heteroarylene group.

In an exemplary embodiment of the present specification, P1 to P3 may be each independently a direct bond; a substituted or unsubstituted C6 to C20 arylene group; a substituted or unsubstituted C2 to C20 heteroarylene group including O or S.

In an exemplary embodiment of the present specification, P1 to P3 may be each independently a direct bond; a substituted or unsubstituted phenylene group; a substituted or unsubstituted divalent dibenzofuran group; or a substituted or unsubstituted divalent dibenzothiophene group.

In an exemplary embodiment of the present specification, P1 to P3 may be each independently a direct bond; a C6 to C40 arylene group unsubstituted or substituted with deuterium; or a C2 to C40 heteroarylene group unsubstituted or substituted with deuterium.

In an exemplary embodiment of the present specification, P1 to P3 may be each independently a direct bond; a C6 to C20 arylene group unsubstituted or substituted with deuterium; or a C2 to C20 heteroarylene group unsubstituted or substituted with deuterium.

In an exemplary embodiment of the present specification, P1 to P3 may be each independently a direct bond; a C6 to C20 arylene group unsubstituted or substituted with deuterium; or a C2 to C20 heteroarylene group unsubstituted or substituted with deuterium and including O or S.

In an exemplary embodiment of the present specification, P1 to P3 may be each independently a direct bond; a phenylene group unsubstituted or substituted with deuterium; a divalent dibenzofuran group unsubstituted or substituted with deuterium; or a divalent dibenzothiophene group unsubstituted or substituted with deuterium.

In an exemplary embodiment of the present specification, P1 to P3 may be each independently a direct bond; or a substituted or unsubstituted C6 to C60 arylene group.

In an exemplary embodiment of the present specification, P1 to P3 may be each independently a direct bond; or a substituted or unsubstituted C6 to C40 arylene group.

In an exemplary embodiment of the present specification, P1 to P3 may be each independently a direct bond; or a substituted or unsubstituted C6 to C20 arylene group.

In an exemplary embodiment of the present specification, P1 to P3 may be each independently a direct bond; or a C6 to C60 arylene group unsubstituted or substituted with deuterium.

In an exemplary embodiment of the present specification, P1 to P3 may be each independently a direct bond; or a C6 to C40 arylene group unsubstituted or substituted with deuterium.

In an exemplary embodiment of the present specification, P1 to P3 may be each independently a direct bond; or a C6 to C20 arylene group unsubstituted or substituted with deuterium.

In an exemplary embodiment of the present specification, S1 to S3 may be each independently a cyano group; a substituted or unsubstituted C18 to C40 triarylsilyl group; a substituted or unsubstituted C6 to C40 aryl group; or a substituted or unsubstituted C2 to C40 heteroaryl group.

In an exemplary embodiment of the present specification, S1 to S3 may be each independently a cyano group; a substituted or unsubstituted C18 to C40 triarylsilyl group; a substituted or unsubstituted C6 to C40 aryl group; or a substituted or unsubstituted C2 to C40 heteroaryl group including O or S.

In an exemplary embodiment of the present specification, S1 to S3 may be each independently a cyano group; a substituted or unsubstituted triphenylsilyl group; a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted fluorenyl group; a substituted or unsubstituted dibenzofuran group; or a substituted or unsubstituted dibenzothiophene group.

In an exemplary embodiment of the present specification, S1 to S3 may be each independently a cyano group; a C18 to C60 triarylsilyl group unsubstituted or substituted with deuterium; a C6 to C60 aryl group unsubstituted or substituted with deuterium, an alkyl group, or an alkyl group substituted with deuterium; or a C2 to C60 heteroaryl group unsubstituted or substituted with deuterium, an aryl group, or an aryl group substituted with deuterium.

In an exemplary embodiment of the present specification, S1 to S3 may be each independently a cyano group; a C18 to C40 triarylsilyl group unsubstituted or substituted with deuterium; a C6 to C40 aryl group unsubstituted or substituted with deuterium, an alkyl group, or an alkyl group substituted with deuterium; or a C2 to C40 heteroaryl group unsubstituted or substituted with deuterium, an aryl group, or an aryl group substituted with deuterium.

In an exemplary embodiment of the present specification, S1 to S3 may be each independently a cyano group; a C18 to C40 triarylsilyl group unsubstituted or substituted with deuterium; a C6 to C40 aryl group unsubstituted or substituted with deuterium, an alkyl group, or an alkyl group substituted with deuterium; or a C2 to C40 heteroaryl group unsubstituted or substituted with deuterium, an aryl group, or an aryl group substituted with deuterium and including O or S.

In an exemplary embodiment of the present specification, S1 to S3 may be each independently a cyano group; a triphenylsilyl group unsubstituted or substituted with deuterium; a phenyl group unsubstituted or substituted with deuterium; a biphenyl group unsubstituted or substituted with deuterium; a terphenyl group unsubstituted or substituted with deuterium; a naphthyl group unsubstituted or substituted with deuterium; a triphenylene group unsubstituted or substituted with deuterium; a dimethylfluorenyl group unsubstituted or substituted with deuterium; a diphenylfluorenyl group unsubstituted or substituted with deuterium; a spirobifluorenyl group unsubstituted or substituted with deuterium; a dibenzofuran group unsubstituted or substituted with deuterium, an aryl group, or an aryl group substituted with deuterium; or a dibenzothiophene group unsubstituted or substituted with deuterium, an aryl group, or an aryl group substituted with deuterium.

In an exemplary embodiment of the present specification, S1 to S3 may be each independently a cyano group; a triphenylsilyl group unsubstituted or substituted with deuterium; a phenyl group unsubstituted or substituted with deuterium; a biphenyl group unsubstituted or substituted with deuterium; a terphenyl group unsubstituted or substituted with deuterium; a naphthyl group unsubstituted or substituted with deuterium; a triphenylene group unsubstituted or substituted with deuterium; a dimethylfluorenyl group unsubstituted or substituted with deuterium; a diphenylfluorenyl group unsubstituted or substituted with deuterium; a spirobifluorenyl group unsubstituted or substituted with deuterium; a dibenzofuran group unsubstituted or substituted with deuterium; or a dibenzothiophene group unsubstituted or substituted with deuterium.

In an exemplary embodiment of the present specification, Z1 to Z3 may be each independently hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; or a substituted or unsubstituted C2 to C60 heterocycloalkyl group.

In an exemplary embodiment of the present specification, Z1 to Z3 may be each independently hydrogen; deuterium; or a substituted or unsubstituted C1 to C60 alkyl group.

In an exemplary embodiment of the present specification, Z1 to Z3 may be each independently hydrogen; deuterium; or a substituted or unsubstituted C1 to C30 alkyl group.

In an exemplary embodiment of the present specification, Z1 to Z3 may be each independently hydrogen; deuterium; or a substituted or unsubstituted C1 to C10 alkyl group.

In an exemplary embodiment of the present specification, Z1 to Z3 may be each independently hydrogen; or deuterium.

In an exemplary embodiment of the present specification, m and n may be 7.

In an exemplary embodiment of the present specification, x may be 6.

In an exemplary embodiment of the present specification, y may be 4.

In an exemplary embodiment of the present specification, Chemical Formula 3 may be represented by any one of the following Chemical Formulae 3-1 to 3-5.

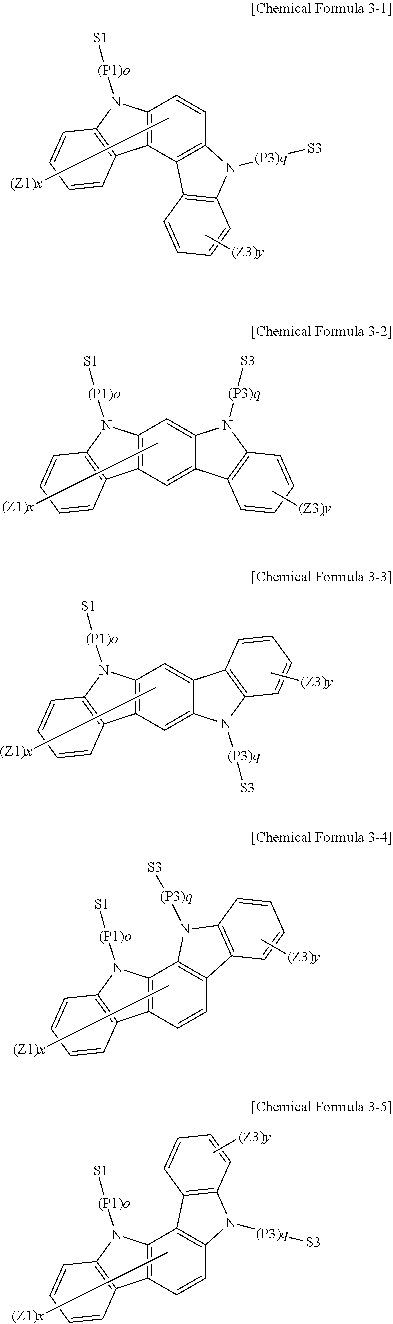

In Chemical Formulae 3-1 to 3-5, the definition of each substituent is the same as the definition in Chemical Formula 3.

In an exemplary embodiment of the present specification, the deuterium substitution rate based on the total hydrogen and deuterium in Chemical Formula 2 or 3 may be 0%, or more than 0% and 100% or less.

In an exemplary embodiment of the present specification, the deuterium substitution rate of Chemical Formula 2 or 3 may be 0%, or 10% to 100%.

In an exemplary embodiment of the present specification, the deuterium substitution rate of Chemical Formula 2 or 3 may be 0%, or 15% to 100%.

In an exemplary embodiment of the present specification, the deuterium substitution rate of Chemical Formula 2 or 3 may be 0%, or 20% to 100%.

In an exemplary embodiment of the present specification, Chemical Formula 2 may be selected from the following compounds.

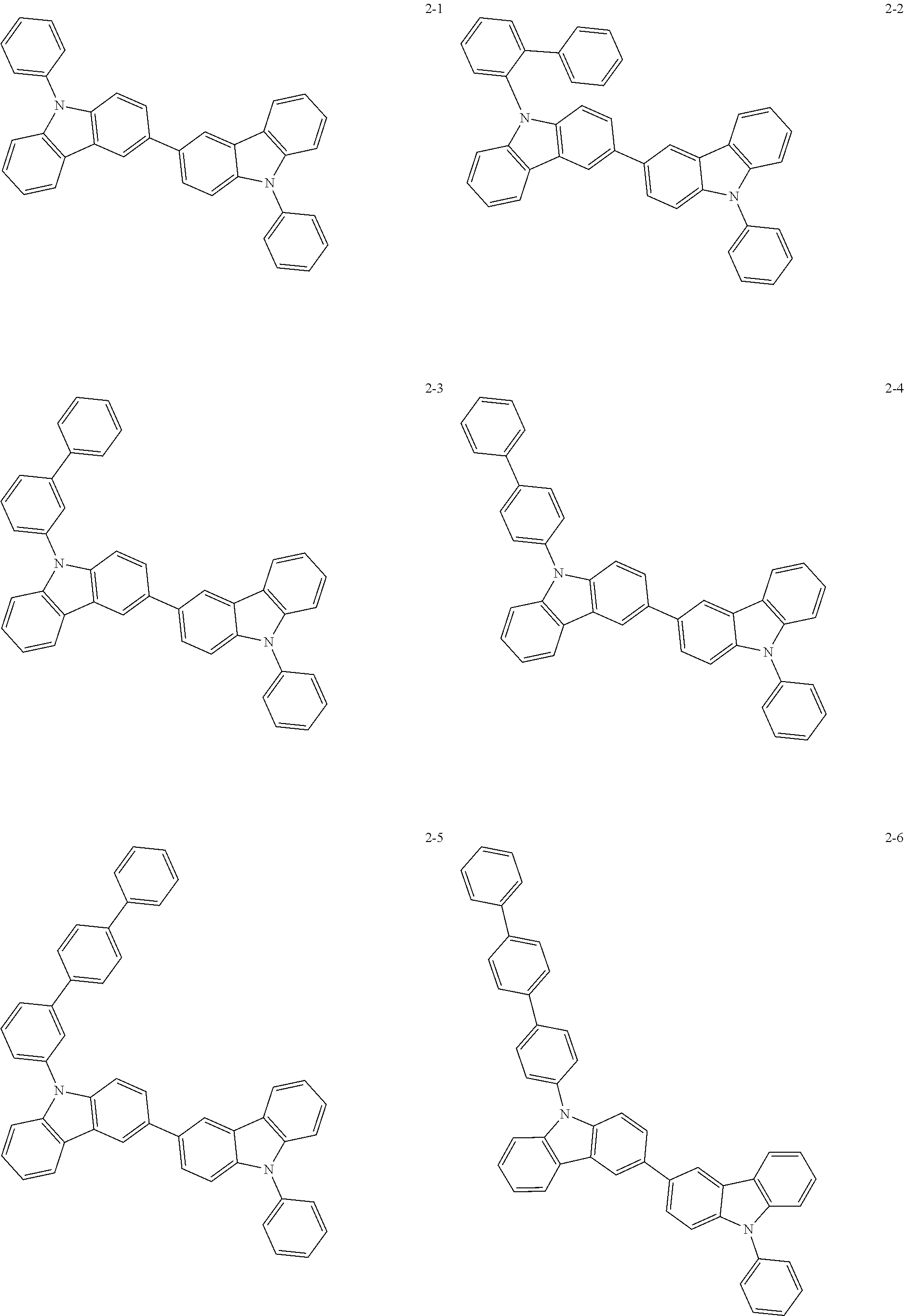 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

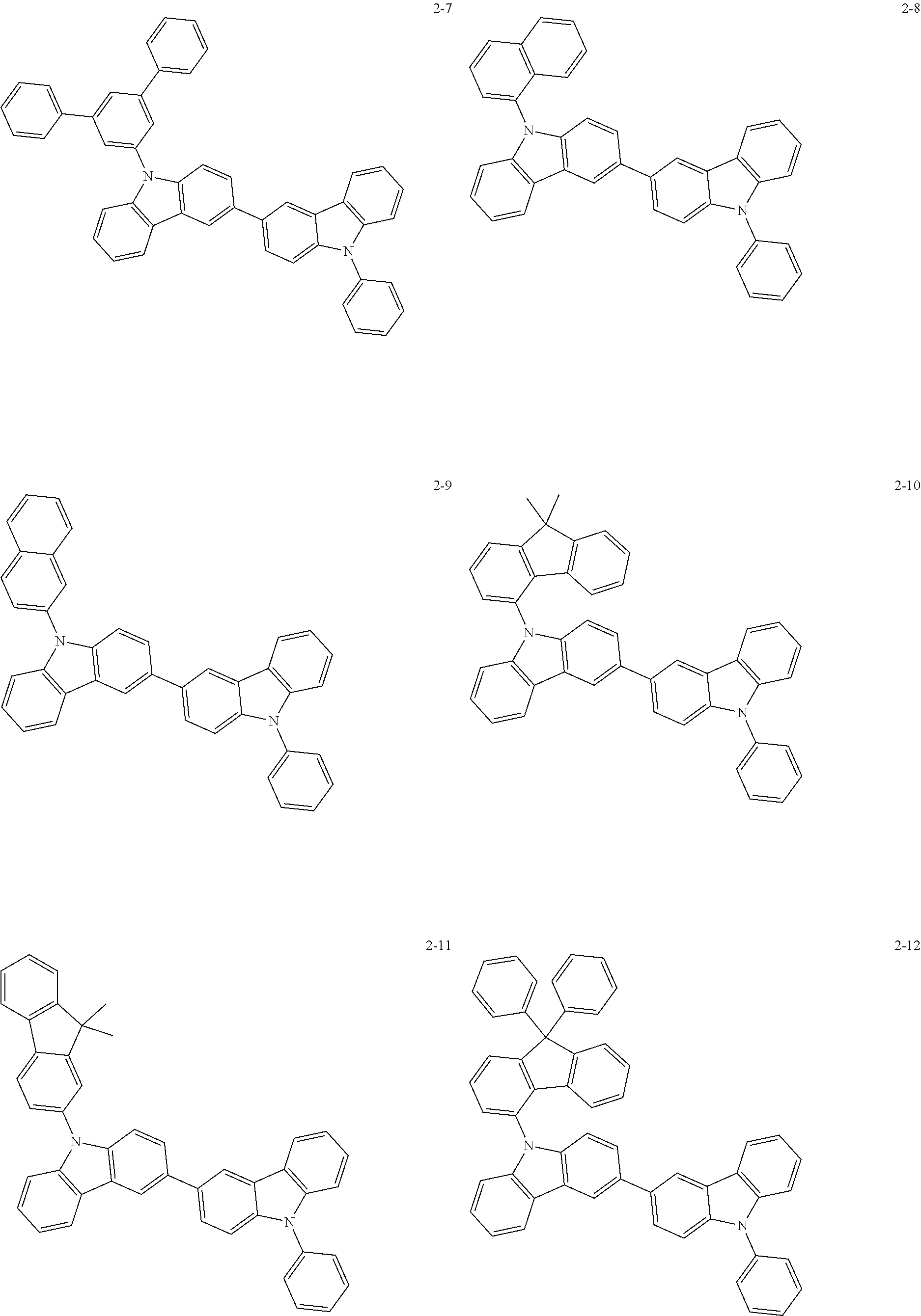 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

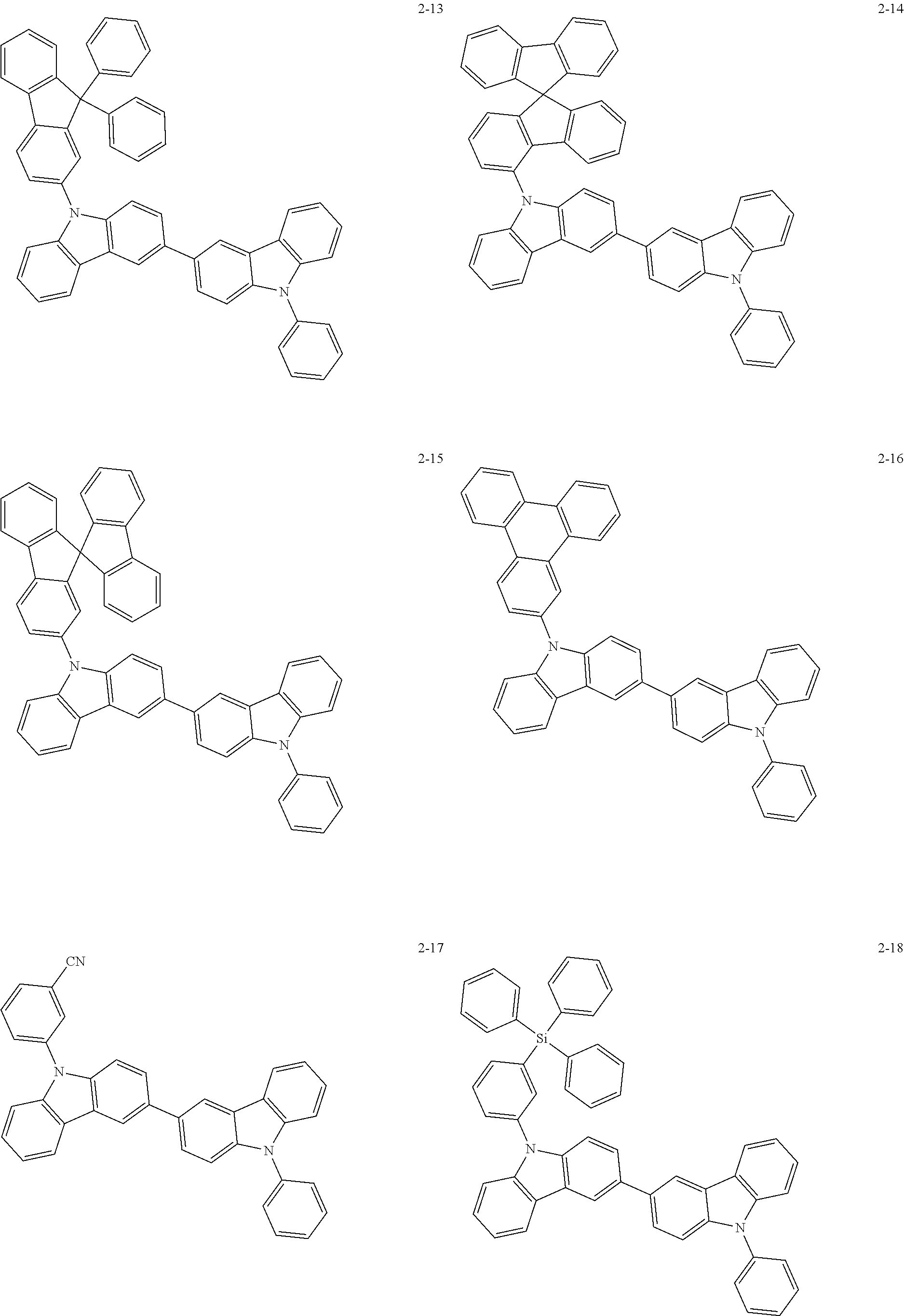

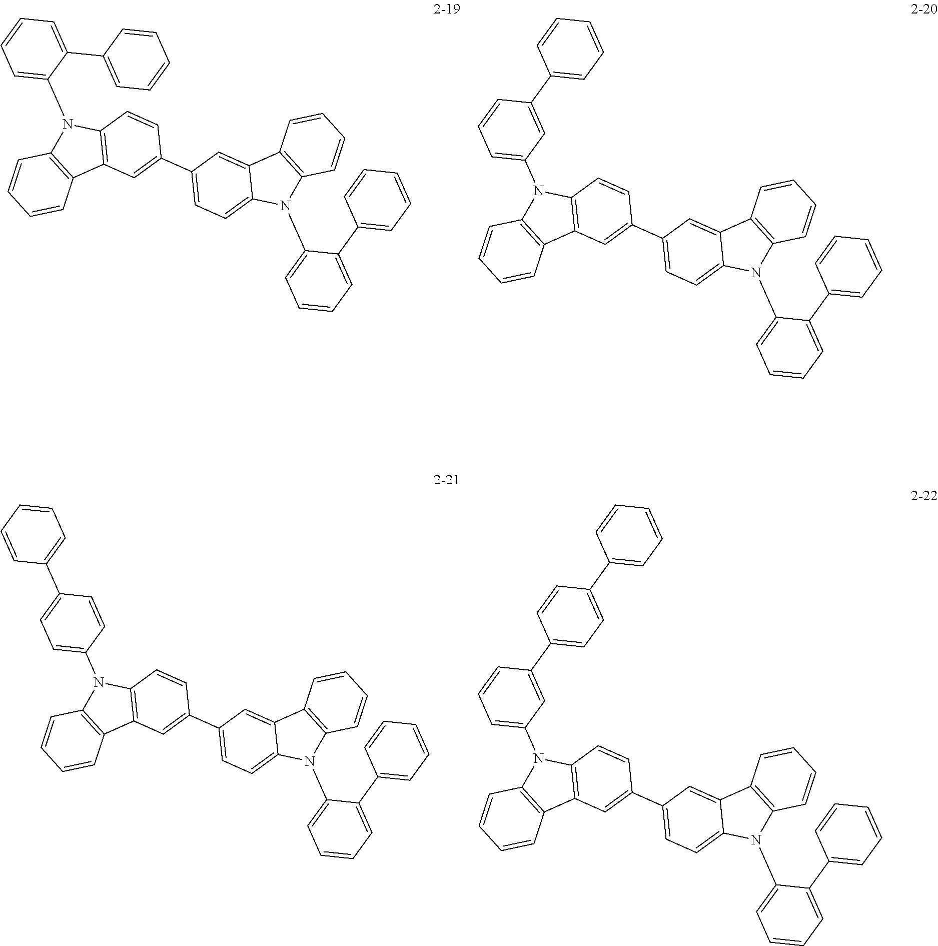 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-c3cccc(-n4c5ccccc5c5cc(-c6ccc7c(c6)c6ccccc6n7-c6ccccc6-c6ccccc6)ccc54)c3)cc2)cc1]

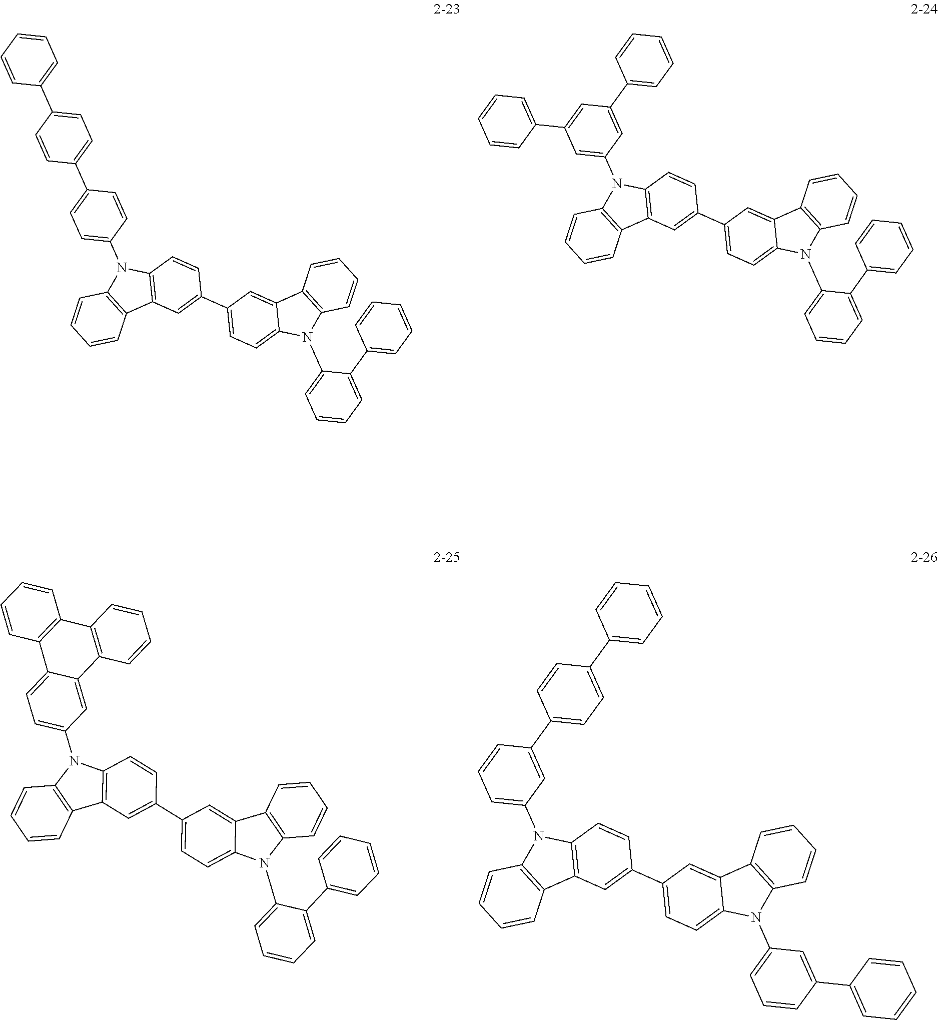 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4ccccc4n5-c4ccc5c6ccccc6c6ccccc6c5c4)ccc32)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1]

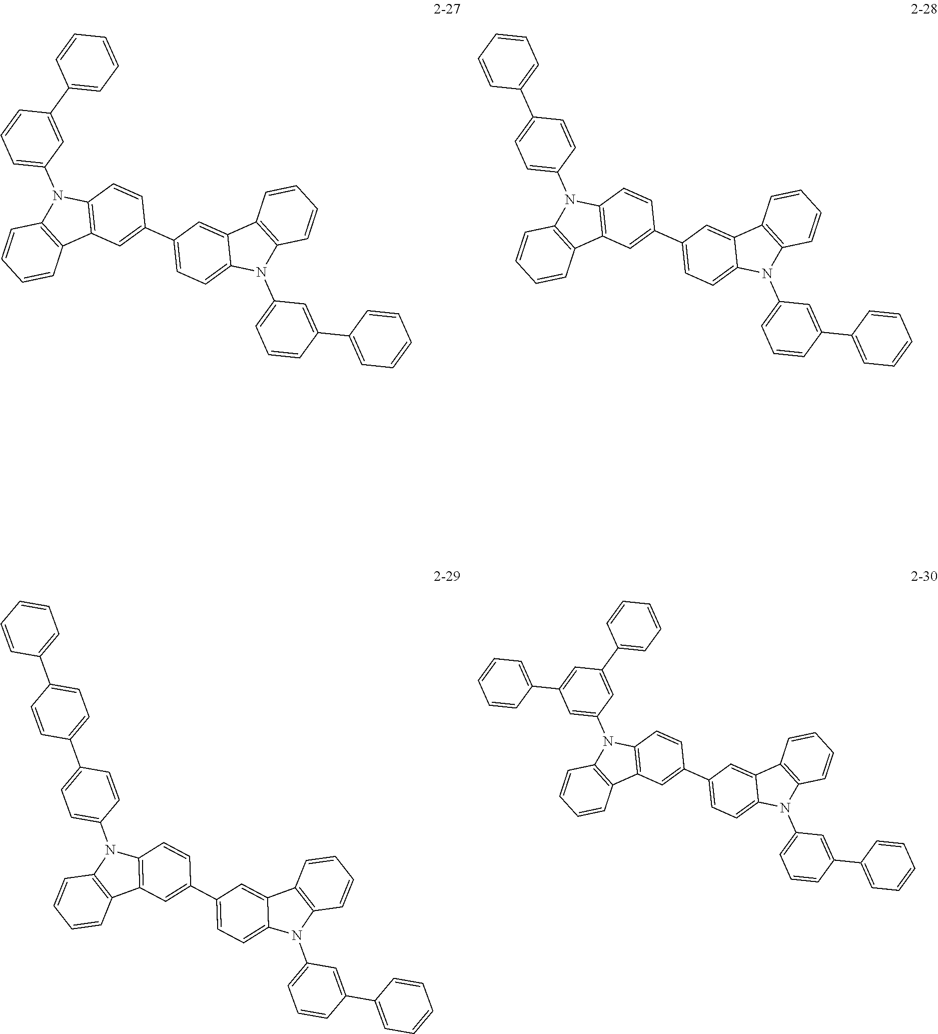 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

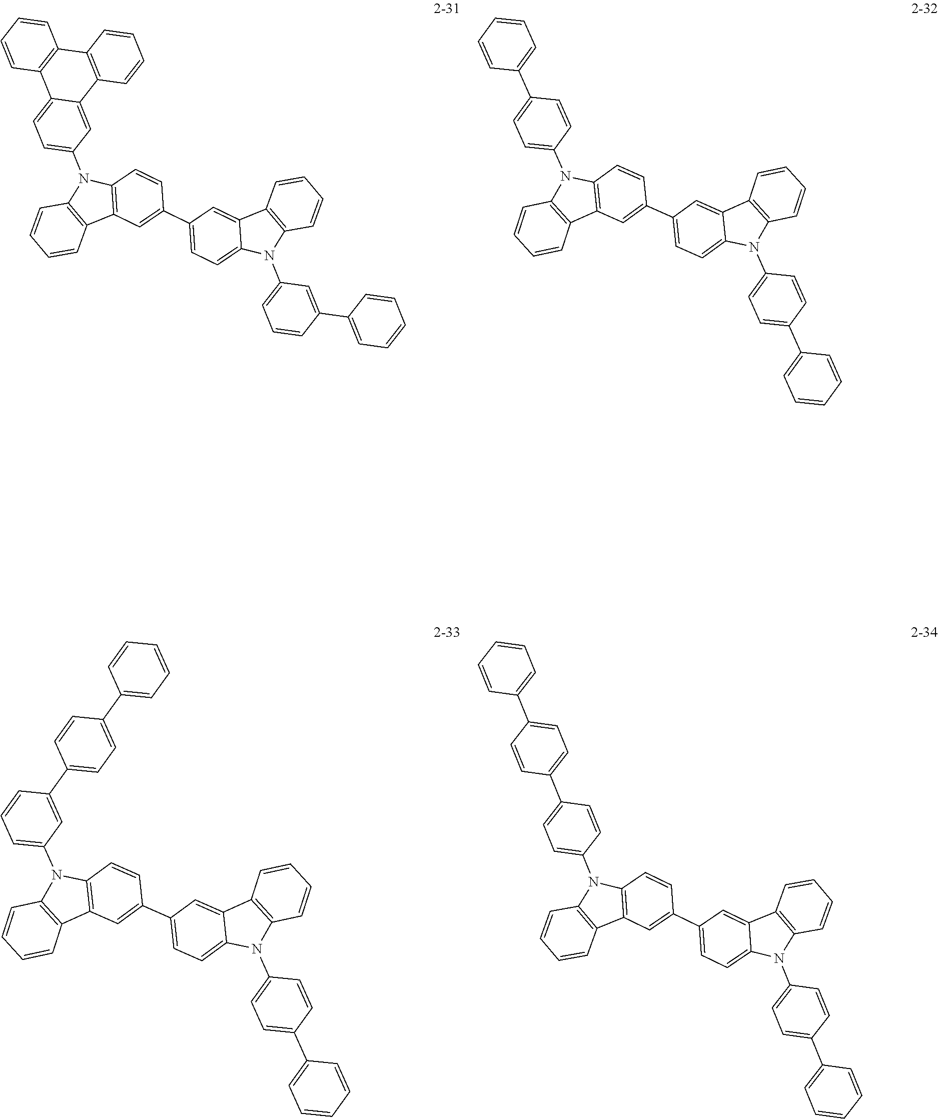 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

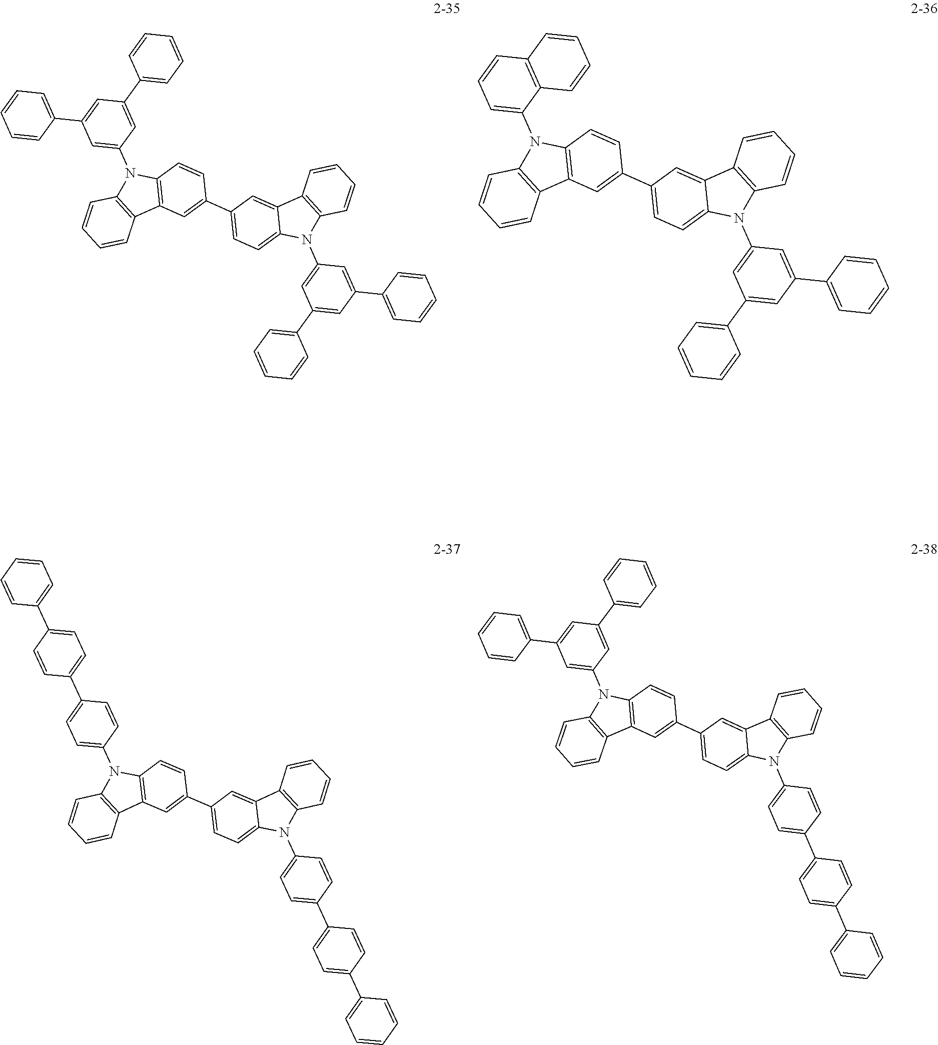 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

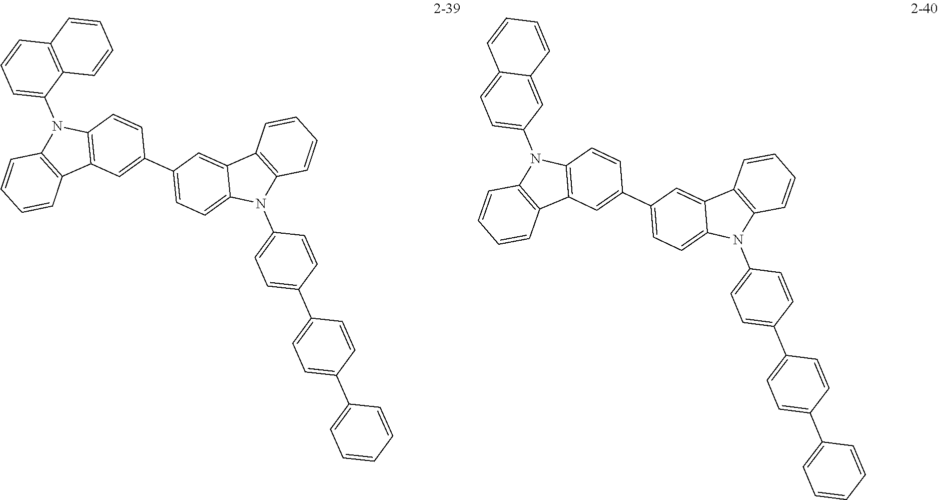 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

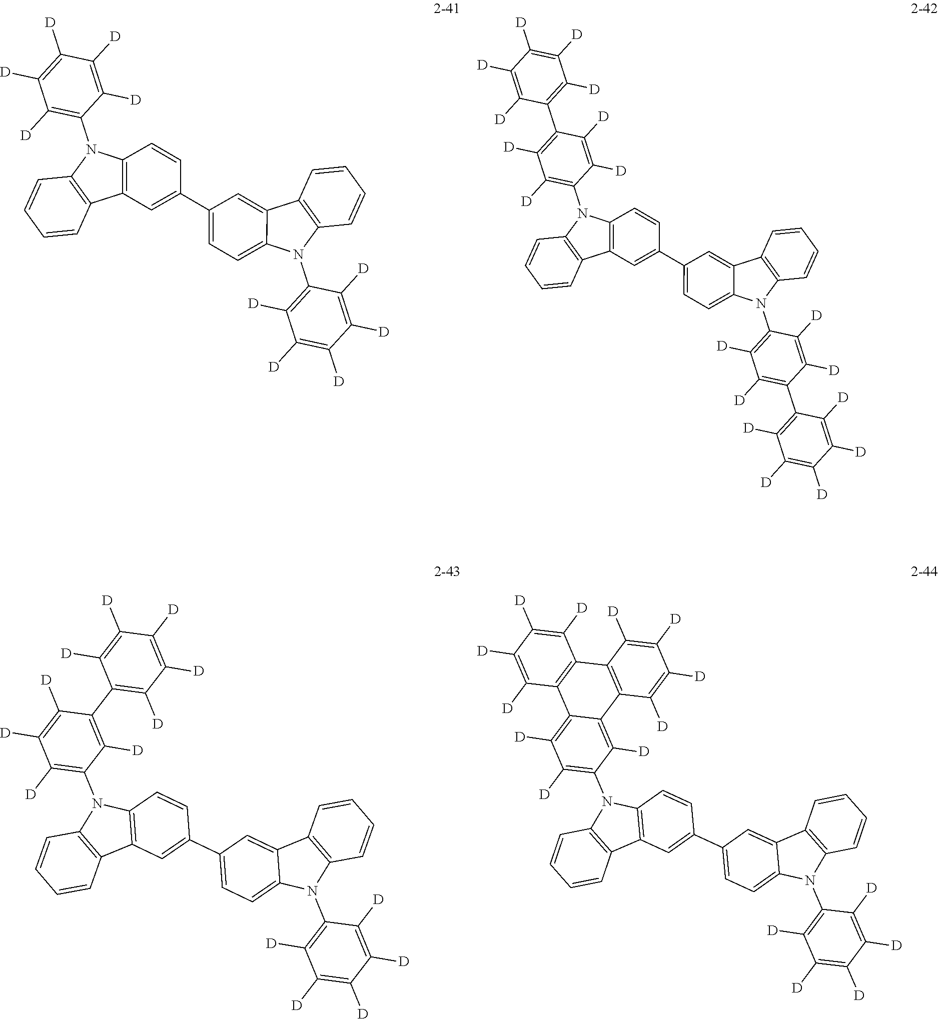

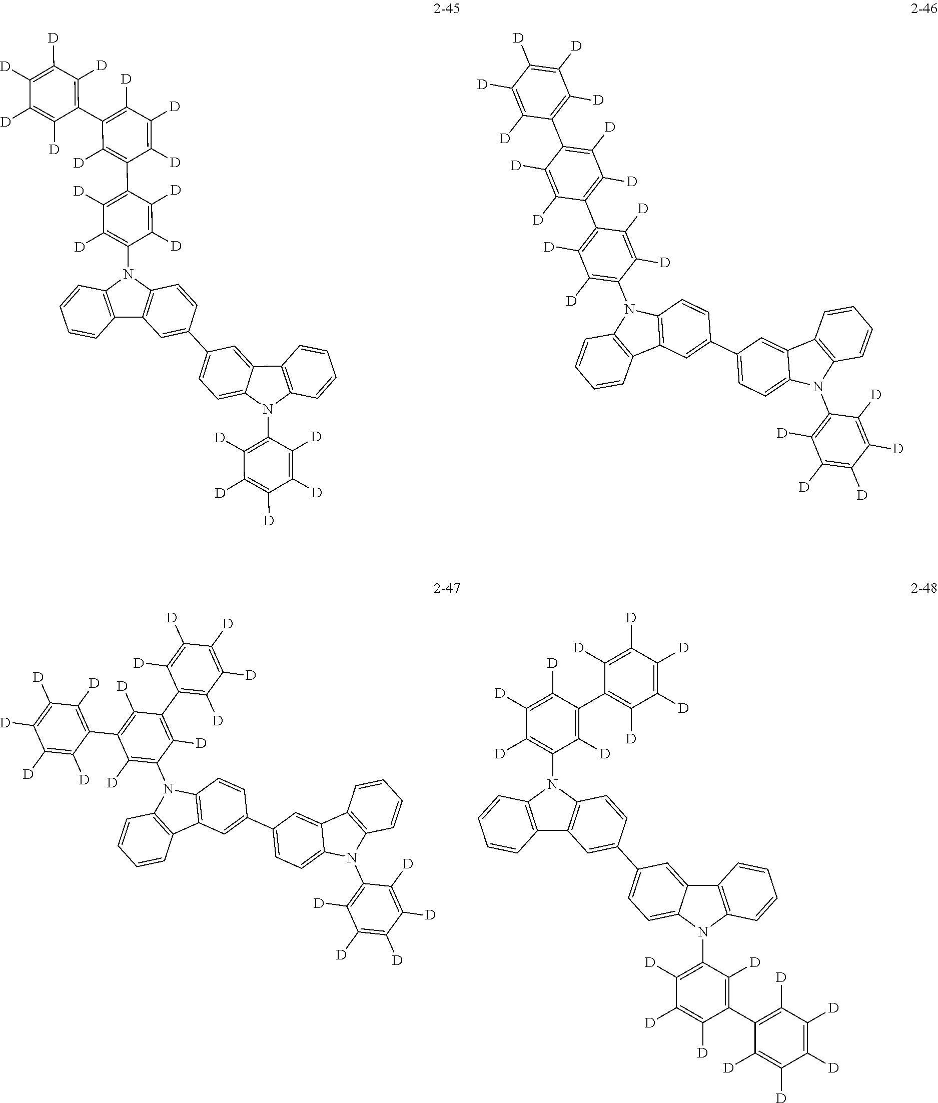

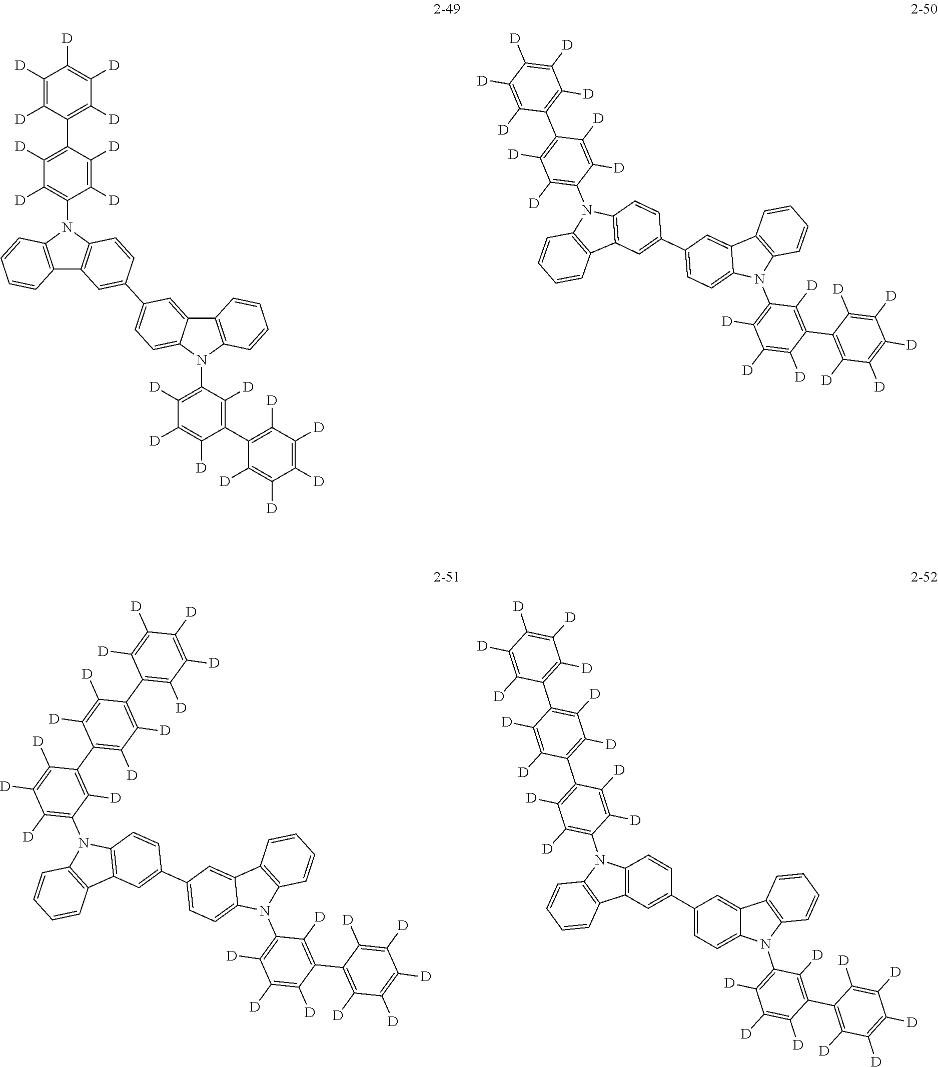

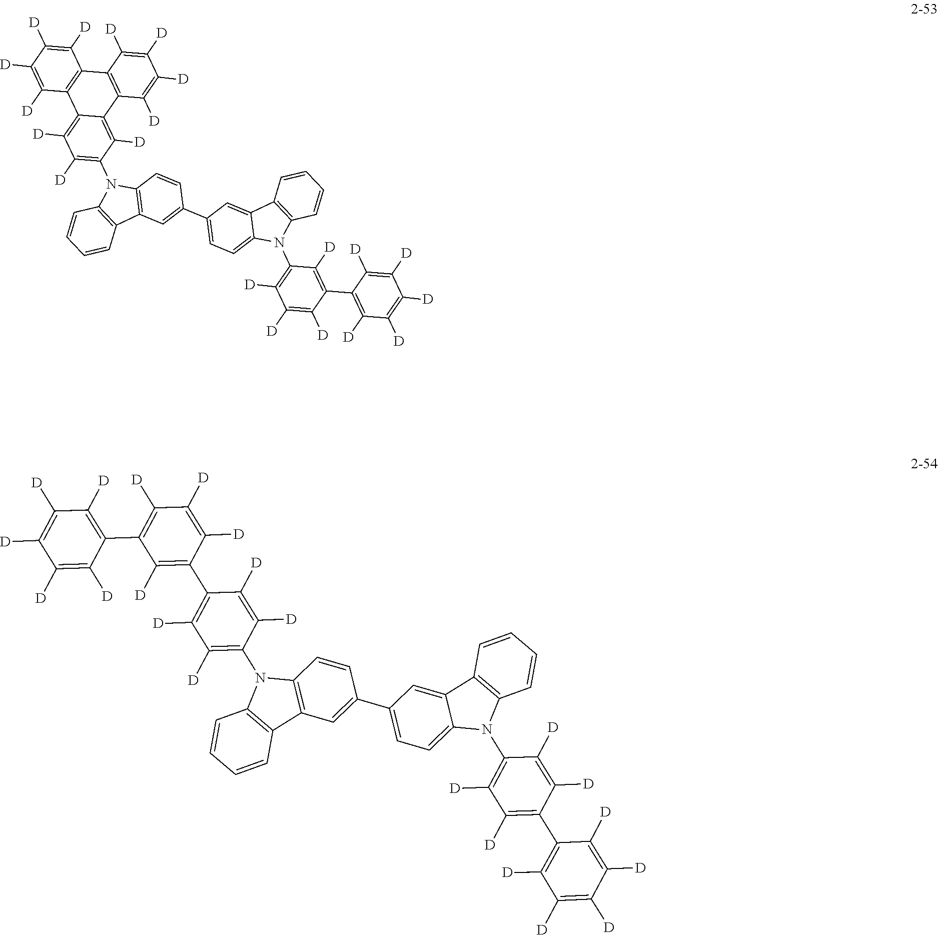 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c(-c7c([2H])c([2H])c([2H])c([2H])c7[2H])c6[2H])c([2H])c5[2H])ccc43)c([2H])c2[2H])c([2H])c1[2H]]

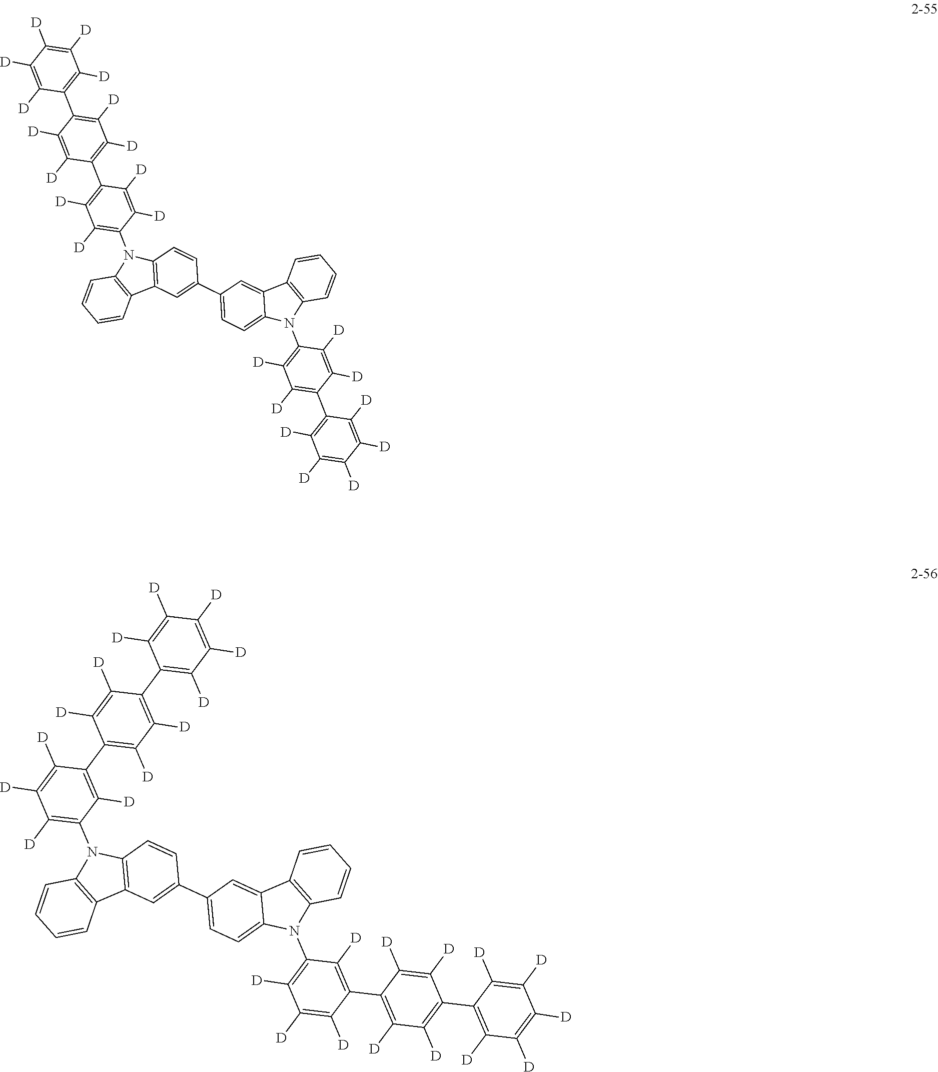 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c([2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c(-c8c([2H])c([2H])c([2H])c([2H])c8[2H])c([2H])c7[2H])c6[2H])ccc54)c3[2H])c([2H])c2[2H])c([2H])c1[2H]]

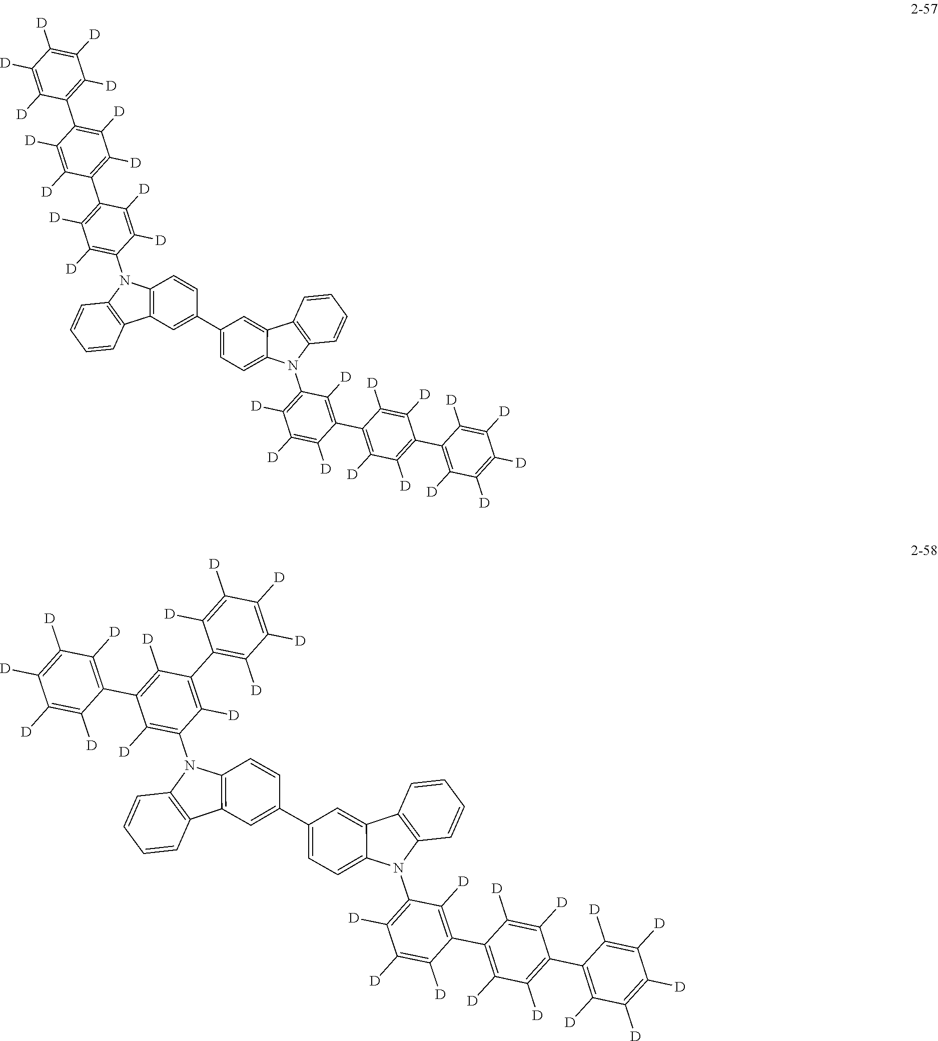

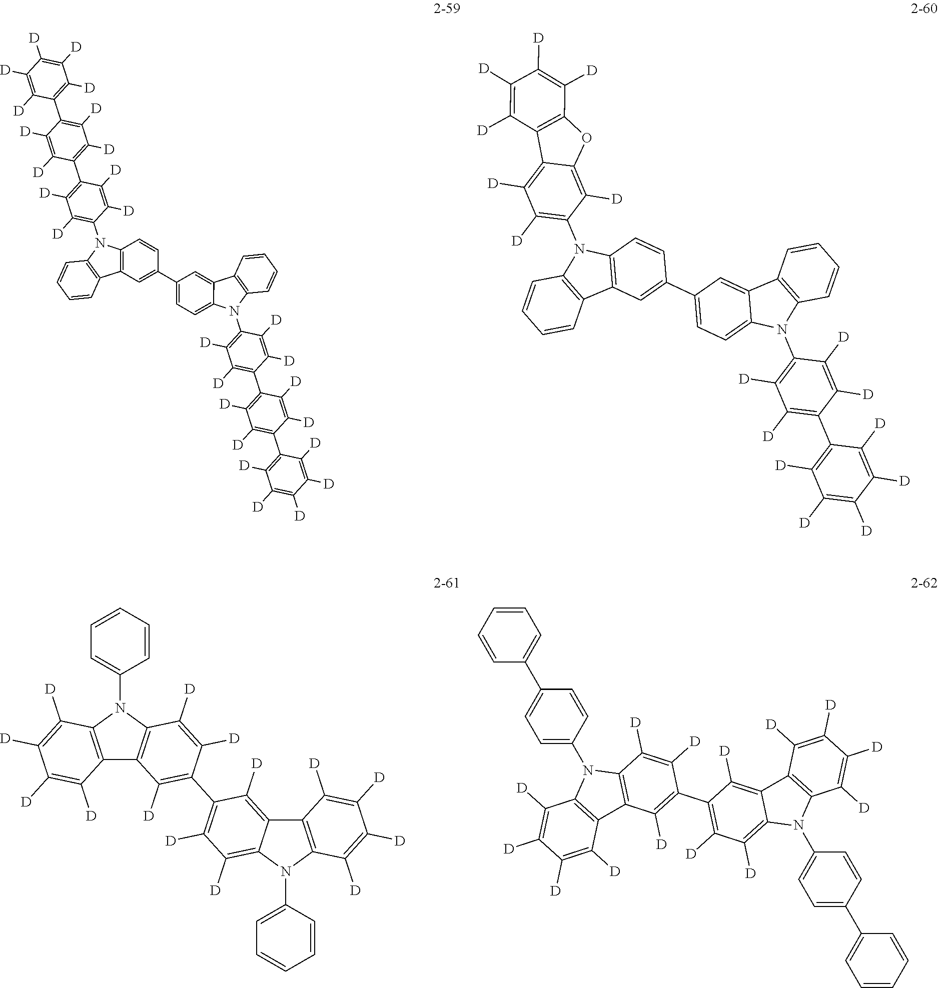

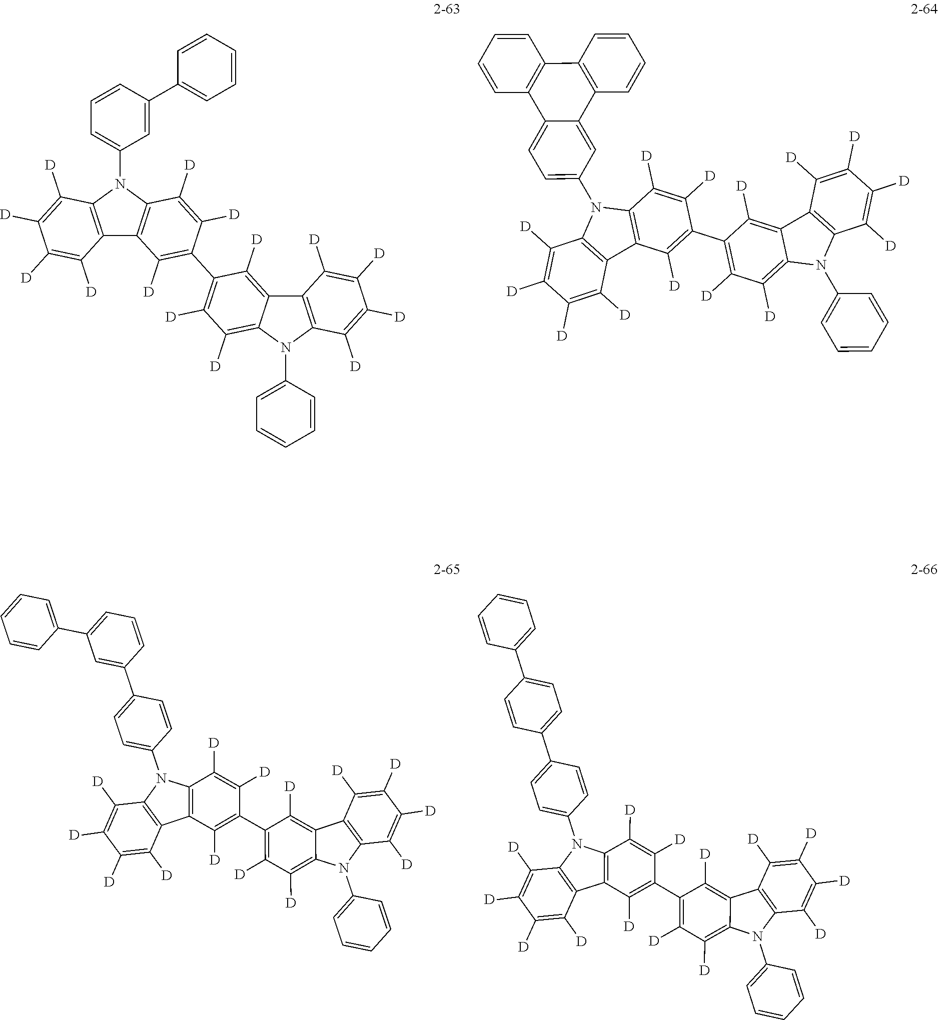

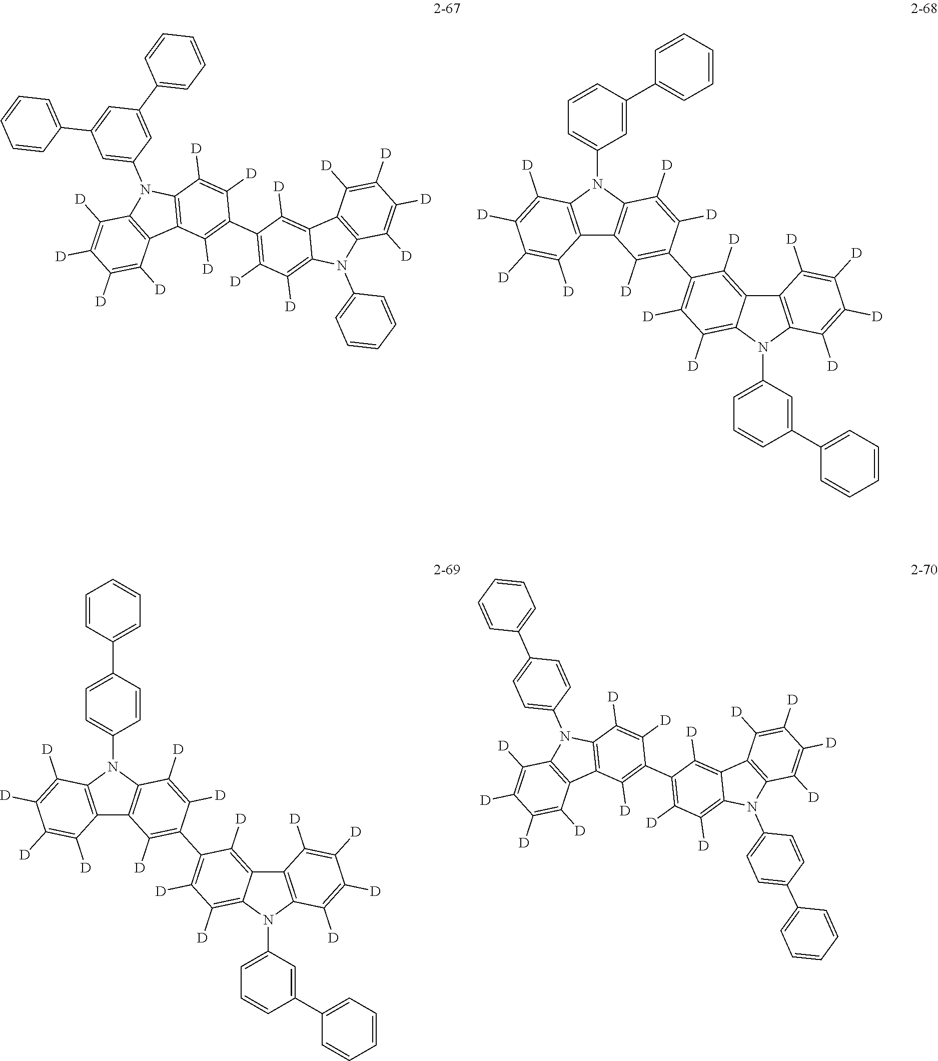

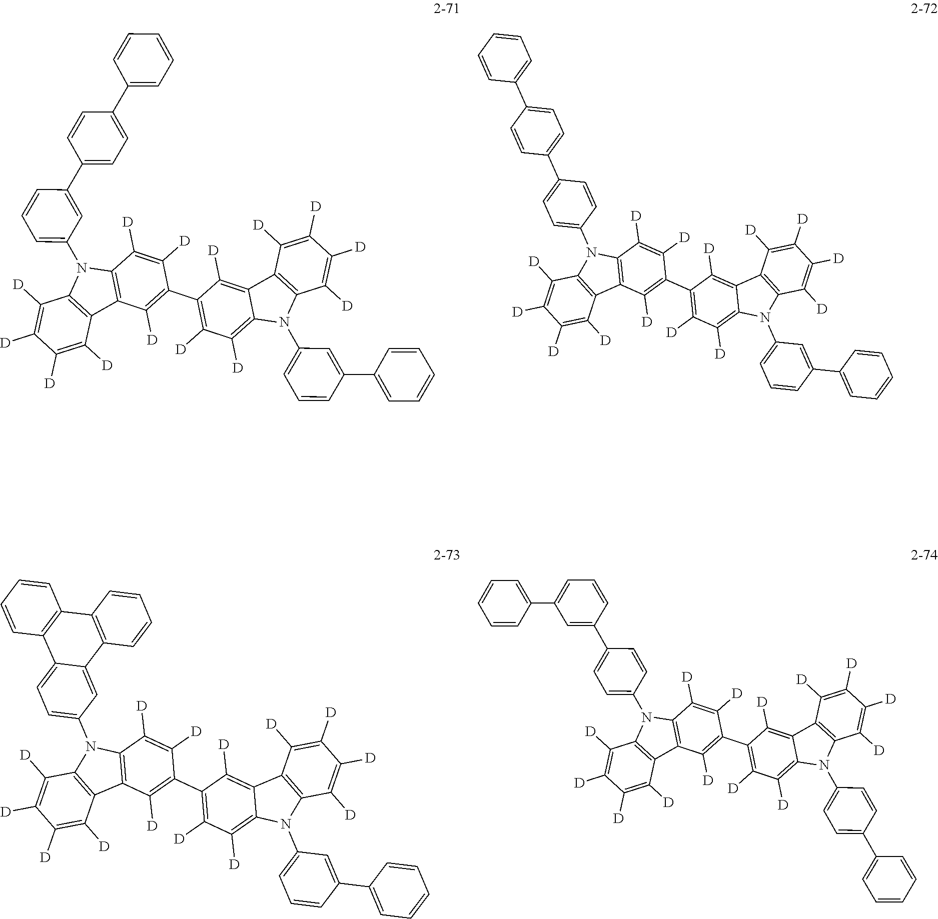

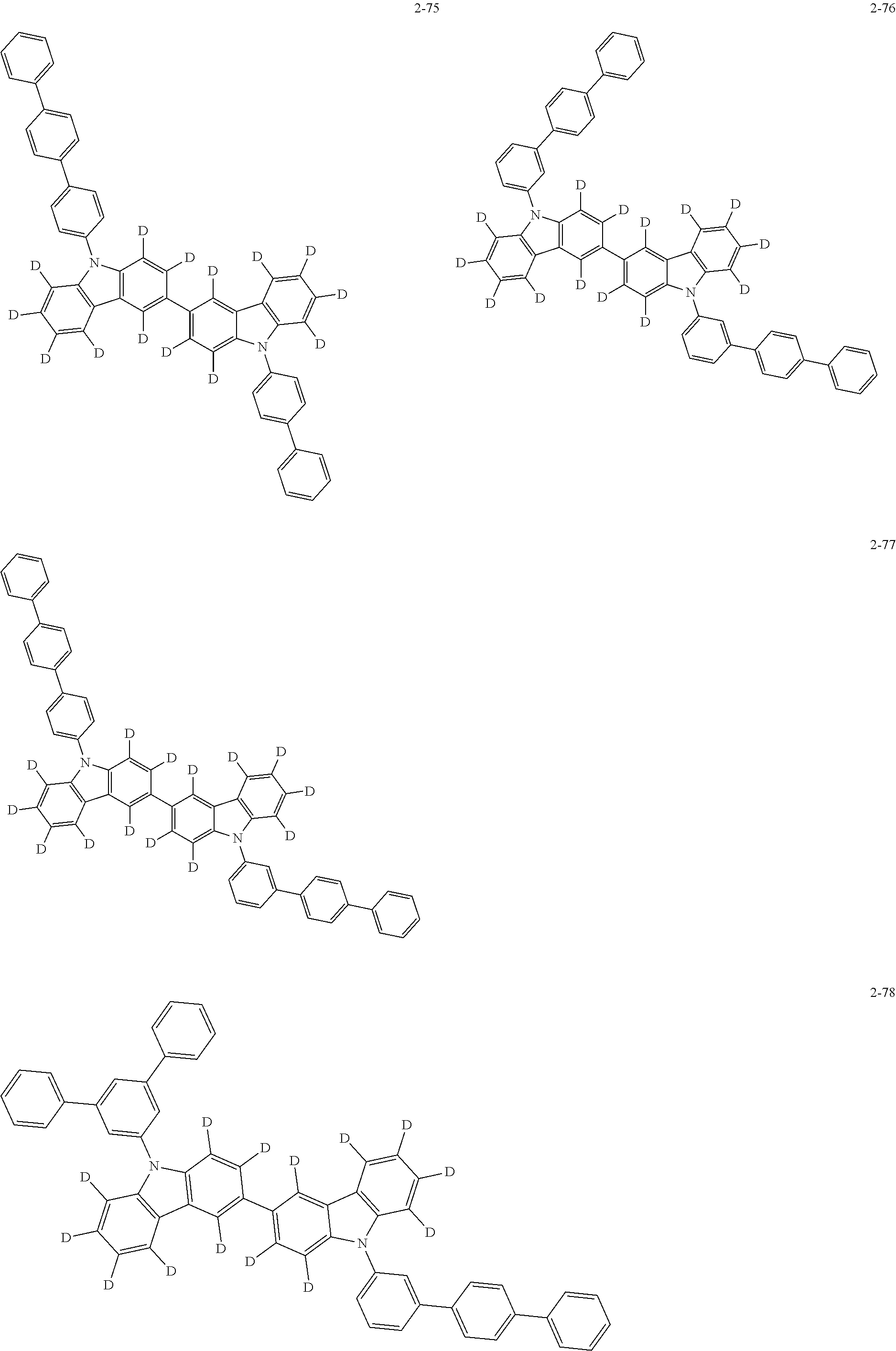

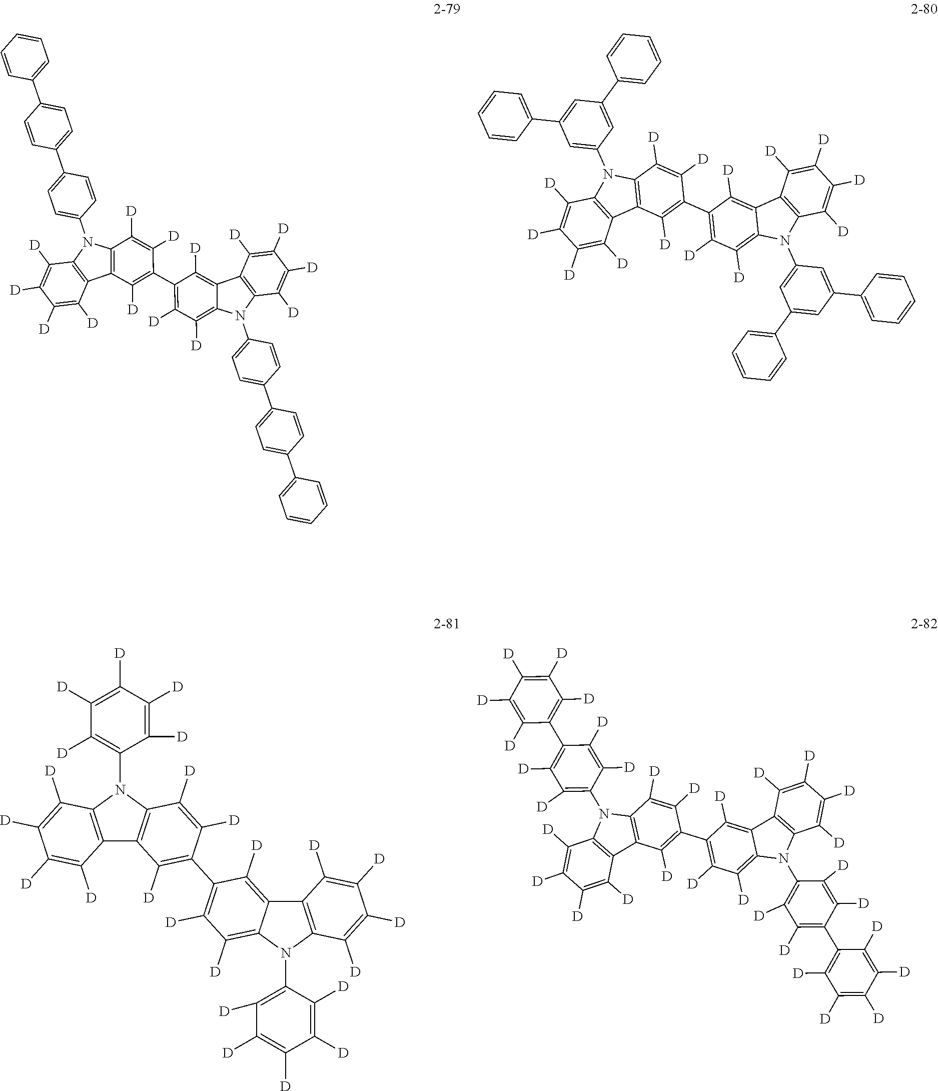

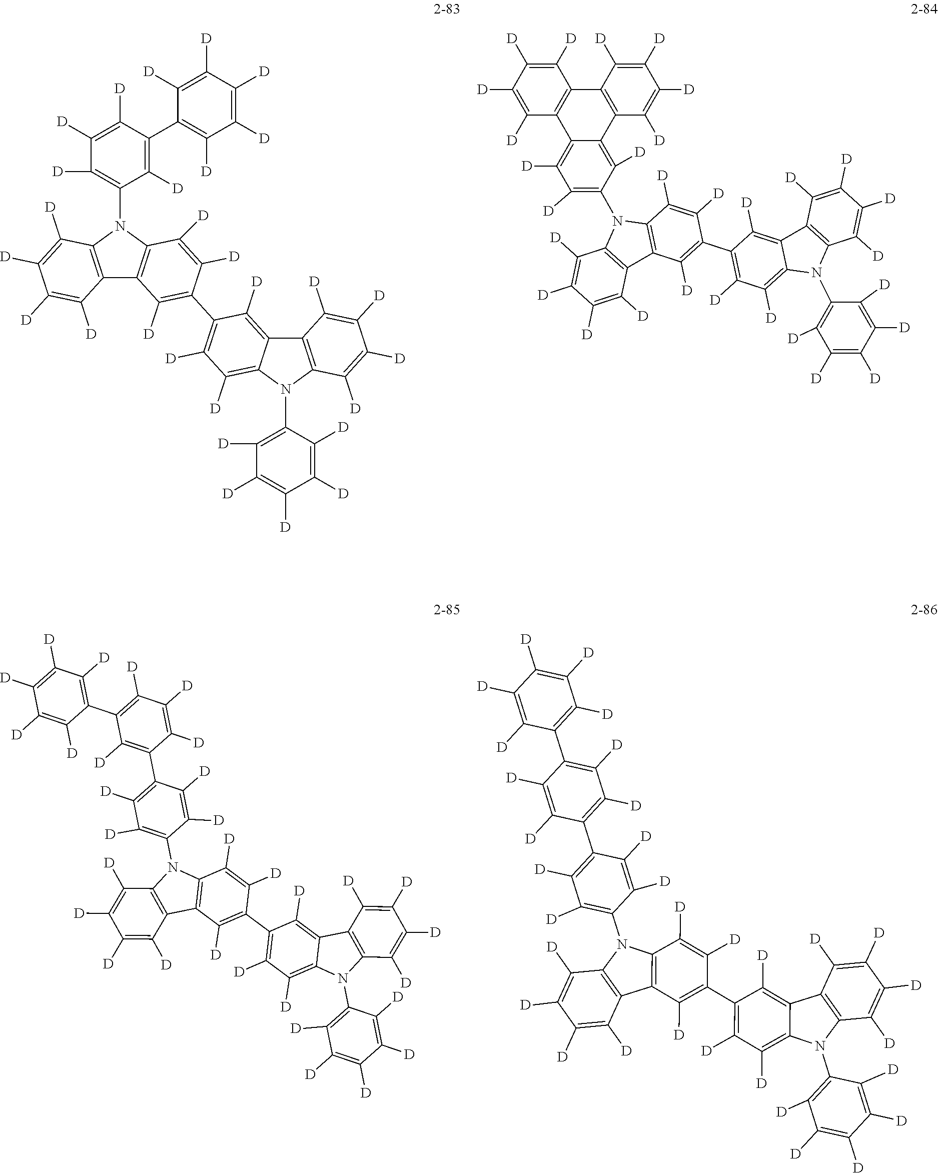

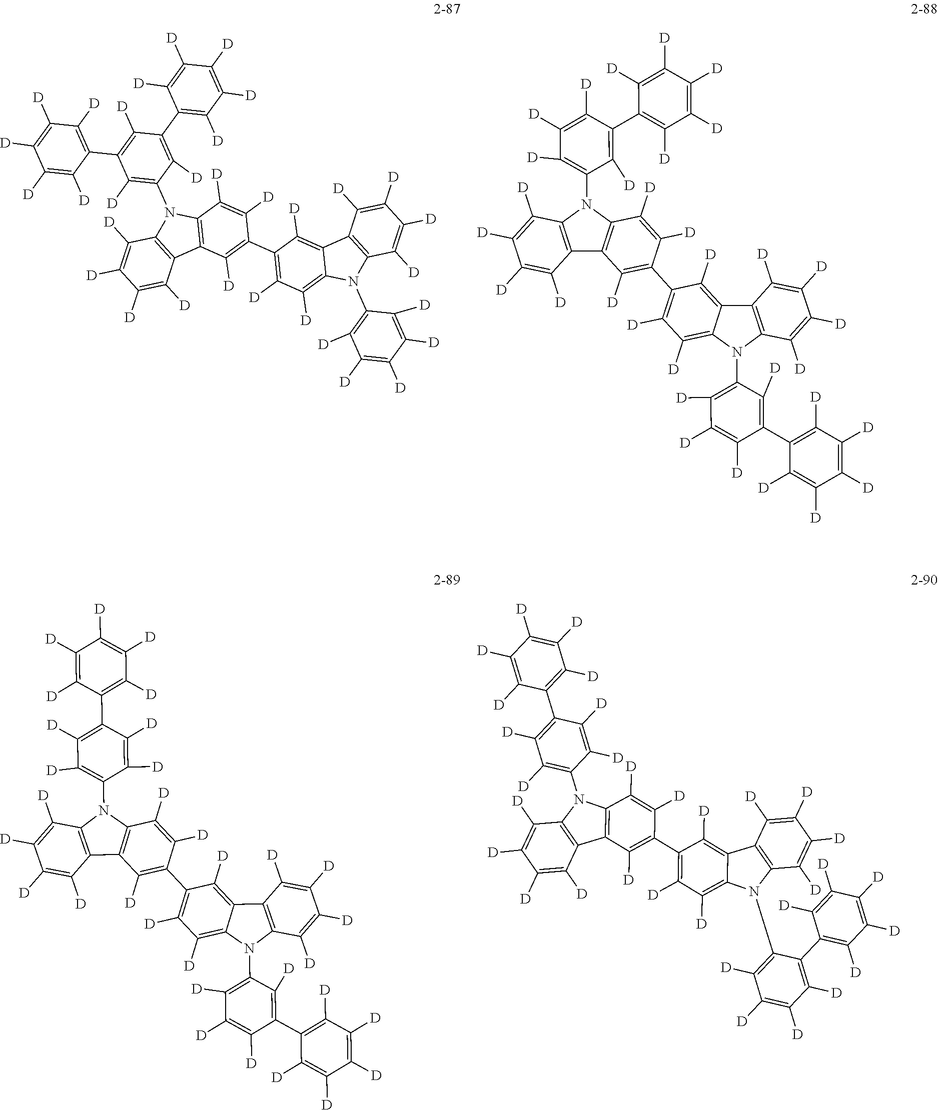

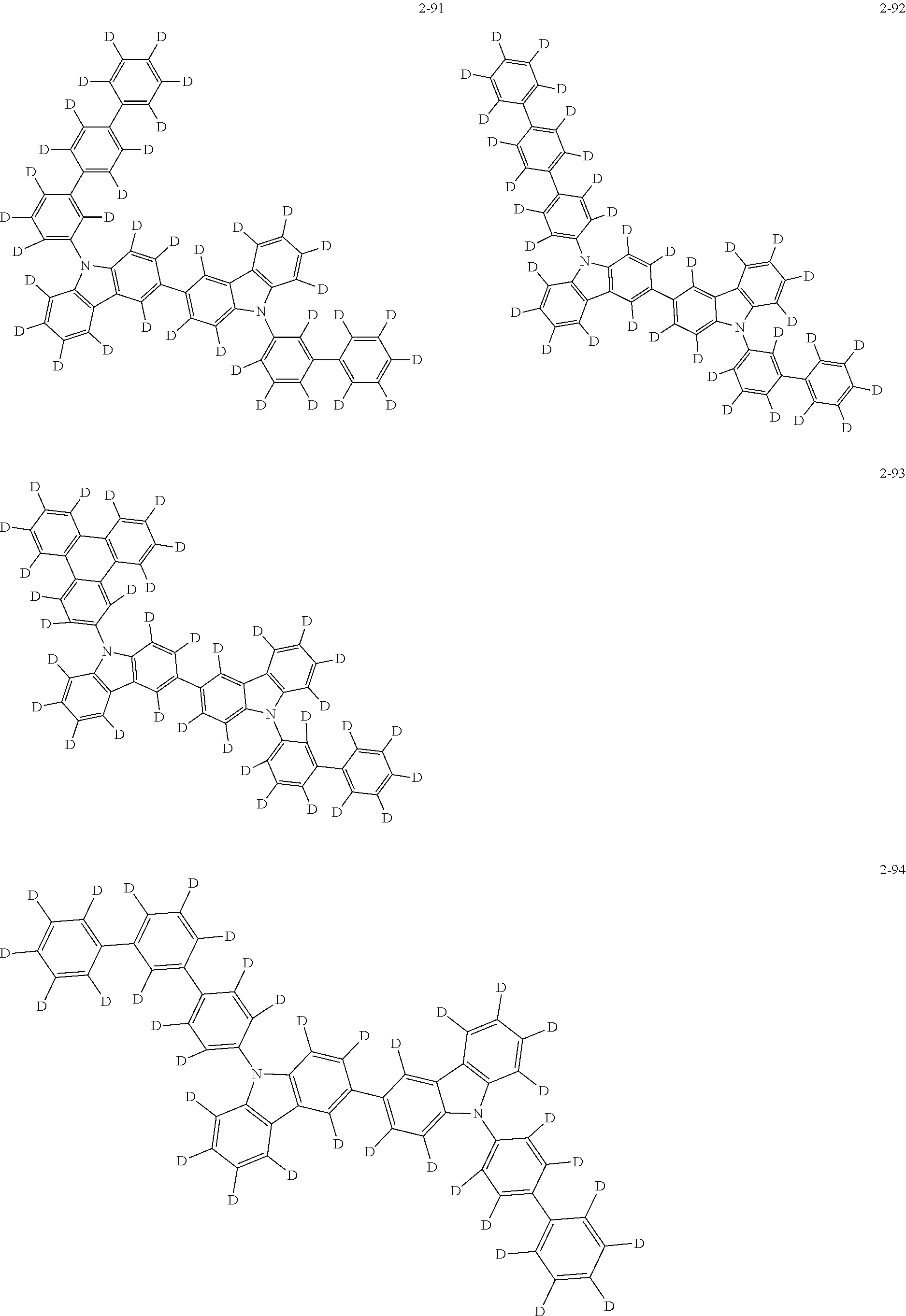

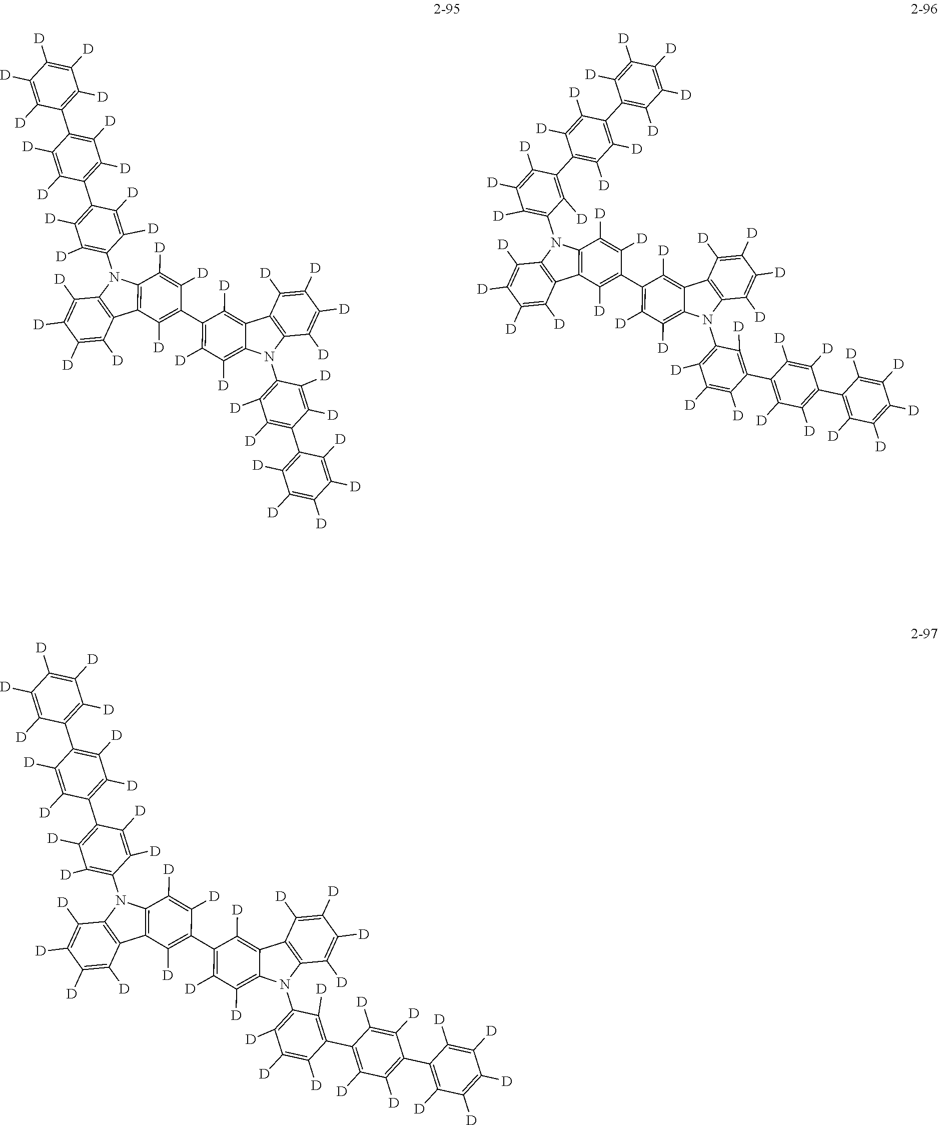

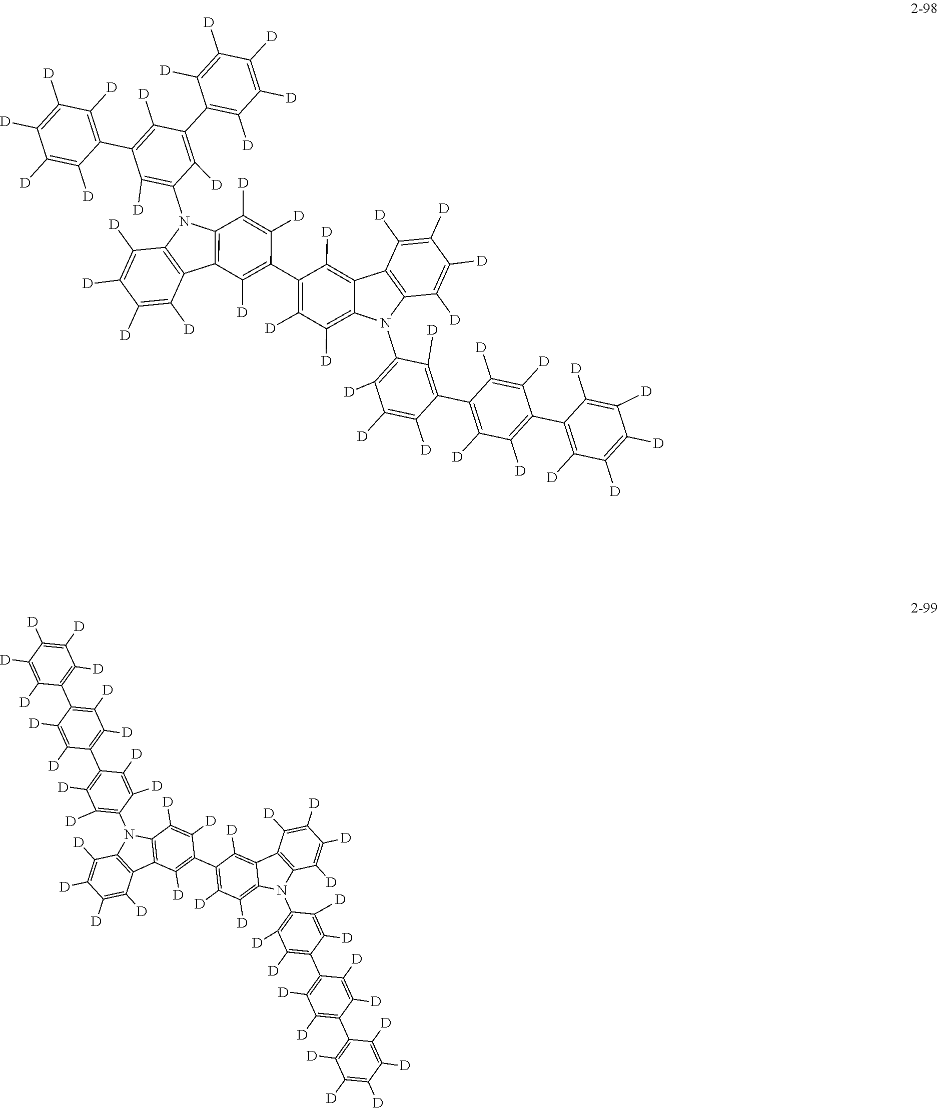

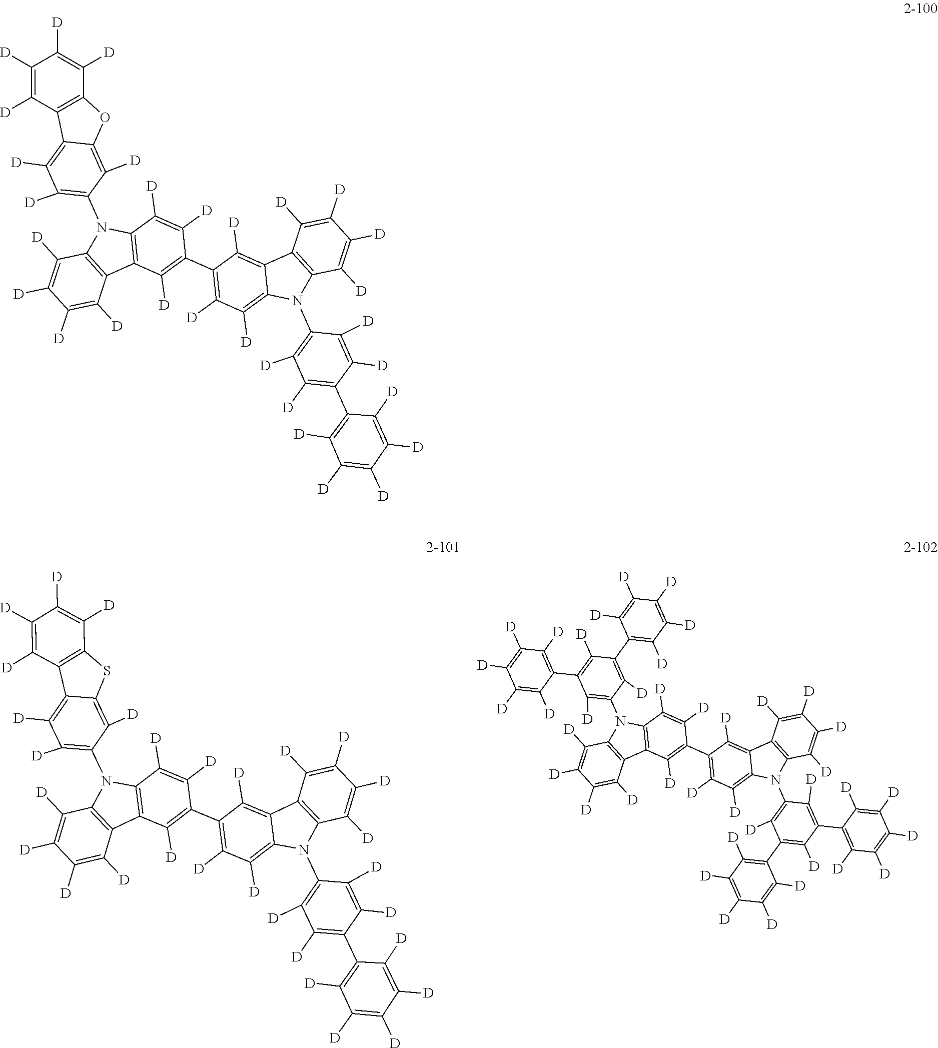

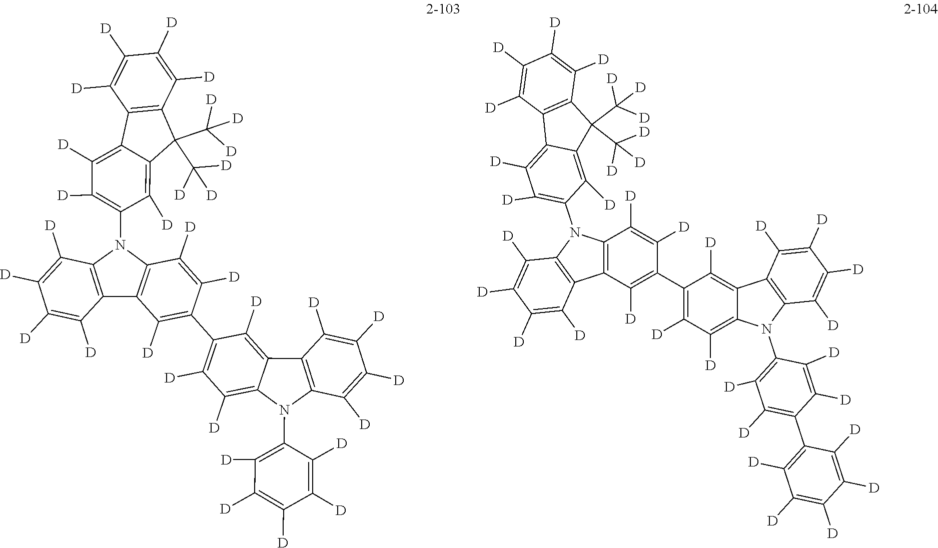

In an exemplary embodiment of the present specification, Chemical Formula 3 may be selected from the following compounds.

[chemical structure]

[chemical structure]

[chemical structure]

[chemical structure]

[chemical structure]

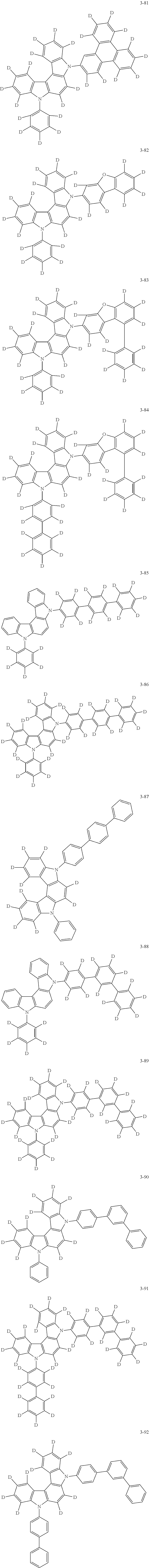

[chemical structure]

[chemical structure]

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present specification, the first electrode may be a positive electrode, and the second electrode may be a negative electrode.

In another exemplary embodiment of the present specification, the first electrode may be a negative electrode, and the second electrode may be a positive electrode.

The organic light emitting device according to an exemplary embodiment of the present specification may be manufactured by typical methods and materials for manufacturing an organic light emitting device, except that an organic material layer having one or more layers is formed by using the heterocyclic compound of the above-described Chemical Formula 1.

The heterocyclic compound of Chemical Formula 1 may be formed as an organic material layer by not only a vacuum deposition method, but also a solution application method when an organic light emitting device is manufactured. Here, the solution coating method means spin coating, dip coating, inkjet printing, screen printing, a spray method, roll coating, and the like, but is not limited thereto.

In an exemplary embodiment of the present specification, the organic light emitting device may be a blue organic light emitting device, and the heterocyclic compound of Chemical Formula 1 may be used as a material for the blue organic light emitting device. For example, the heterocyclic compound of Chemical Formula 1 may be included in a light emitting layer of a blue organic light emitting device.

In another exemplary embodiment of the present specification, the organic light emitting device may be a green organic light emitting device, and the heterocyclic compound of Chemical Formula 1 may be used as a material for the green organic light emitting device. For example, the heterocyclic compound of Chemical Formula 1 may be included in a light emitting layer of a green organic light emitting device.

In still another exemplary embodiment of the present specification, the organic light emitting device may be a red organic light emitting device, and the heterocyclic compound of Chemical Formula 1 may be used as a material for the red organic light emitting device. For example, the heterocyclic compound of Chemical Formula 1 may be included in a light emitting layer of a red organic light emitting device.

The organic light emitting device of the present invention may further include one or more layers selected from the group consisting of a light emitting layer, a hole injection layer, a hole transport layer, an electron injection layer, an electron transport layer, an electron blocking layer, and a hole blocking layer.

FIGS. 1 to 3 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

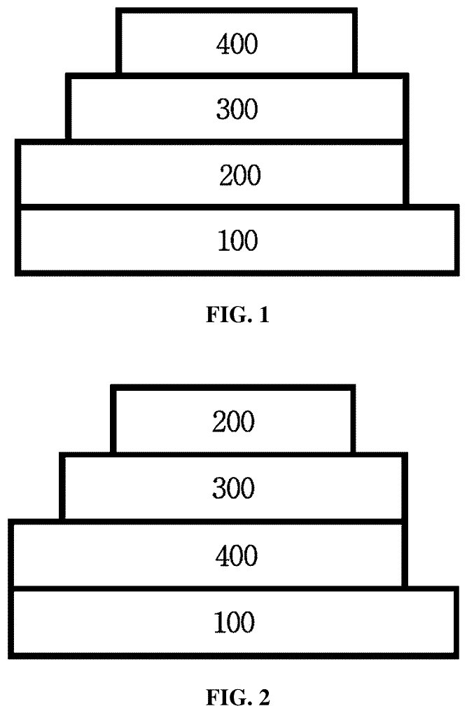

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as illustrated in FIG. 2, an organic light emitting device in which a negative electrode, an organic material layer, and a positive electrode are sequentially stacked on a substrate may also be implemented.

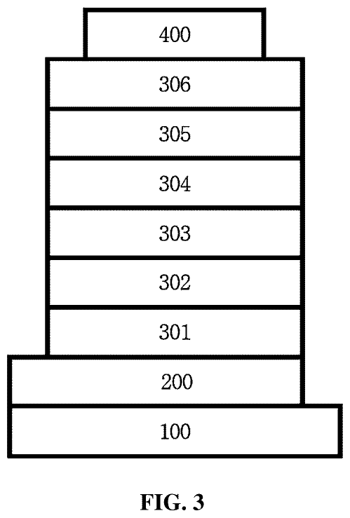

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

An organic material layer including the heterocyclic compound of Chemical Formula 1 may additionally include other materials, if necessary.

In the organic light emitting device according to an exemplary embodiment of the present specification, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrenesulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, any two or more materials from N-type host materials or P-type host materials may be selected and used as a host material for a light emitting layer.

The organic light emitting device according to an exemplary embodiment of the present specification may be a top emission type, a bottom emission type, or a dual emission type according to the material to be used.

The heterocyclic compound according to an exemplary embodiment of the present specification may act even in organic electronic devices including organic solar cells, organic photoconductors, organic transistors, and the like, based on the principle similar to those applied to organic light emitting devices.

In addition, it is possible to finely adjust an energy band-gap by introducing various substituents into the structure of Chemical Formula 1, and meanwhile, it is possible to improve characteristics at the interface between organic materials and diversify the use of the material.

Another exemplary embodiment of the present specification provides a composition for an organic material layer, including the heterocyclic compound of Chemical Formula 1.

In an exemplary embodiment of the present specification, the composition for an organic material layer may further include the compound of Chemical Formula 2 or 3.

In an exemplary embodiment of the present specification, the composition for an organic material layer may include the heterocyclic compound (Chemical Formula 1) and the compound (Chemical Formula 2 or 3) at a weight ratio of 1:10 to 10:1.

In an exemplary embodiment of the present specification, the composition for an organic material layer may include the heterocyclic compound and the compound at a weight ratio of 1:8 to 8:1, 1:5 to 5:1, or 1:3 to 3:1.

In an exemplary embodiment of the present specification, the composition for an organic material layer may include the heterocyclic compound and the compound at a weight ratio of 1:1 to 5:1, or 1:1 to 3:1.

Still another exemplary embodiment of the present specification provides a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the above-described composition for an organic material layer.

In an exemplary embodiment of the present specification, the forming of the organic material layer may include pre-mixing the composition for an organic material layer of the organic light emitting device to deposit the pre-mixed composition onto a single supply source.

The pre-mixing means that before the heterocyclic compound of Chemical Formula 1 and the compound of Chemical Formula 2 or 3 are deposited onto an organic material layer, the materials are first mixed and the mixture is contained in one common container and mixed. Since one deposition source is used instead of using two or more deposition sources during the pre-mixing, there is an advantage in that the process is more simplified.

When the composition for an organic material layer is pre-mixed, the deposition conditions, such as the deposition rate, may be significantly affected by the inherent thermal characteristics of the material during the deposition of the pre-mixed material, so that the inherent thermal characteristics of each pre-mixed material need to be confirmed. When the thermal properties of the materials are not similar, the deposition process cannot be repeated or reproduced, and a uniform OLED device cannot be manufactured.

In order to overcome this problem, the electrical characteristics of the material may be controlled by utilizing the appropriate combination of the basic structure of each material and the substituent, and simultaneously, the thermal characteristics may also be adjusted according to the form of the molecular structure. The thermal characteristics of each material may be adjusted to secure the diversity of various pre-mixing deposition processes between a host and a host. Through this, it is possible to secure the diversity of the pre-mixing deposition process utilizing not only two compounds as hosts but also 3 or more types of host materials.

In an exemplary embodiment of the present specification, the composition for an organic material layer may include other hosts in addition to the compound of Chemical Formula 2 or 3.

In an exemplary embodiment of the present specification, the composition for an organic material layer includes the compound of Chemical Formula 2 or 3, and may further include another host.

Hereinafter, the present specification will be described in more detail through Examples, but these Examples are provided only for exemplifying the present application, and are not intended to limit the scope of the present application.

### PREPARATION EXAMPLES

### <Preparation Example 1-1> Preparation of Compound 1-1

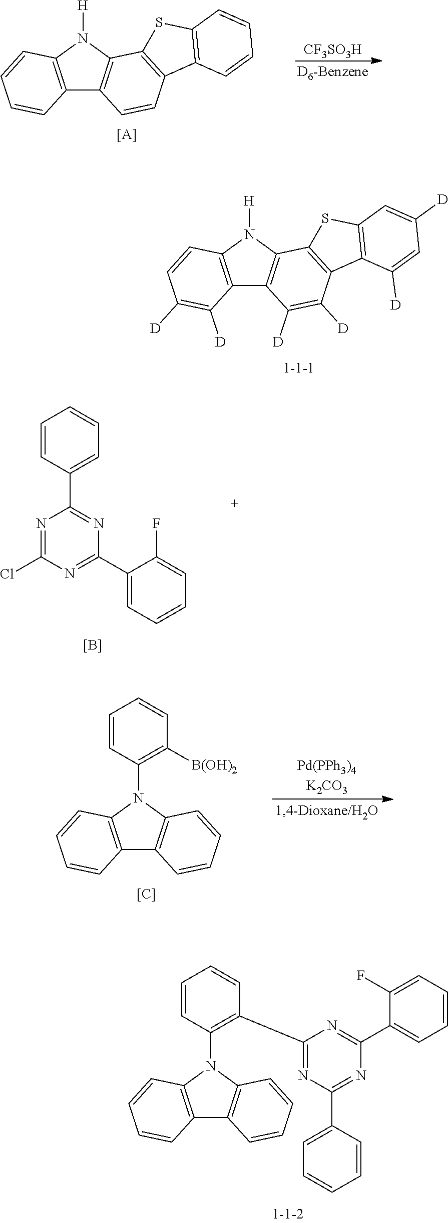

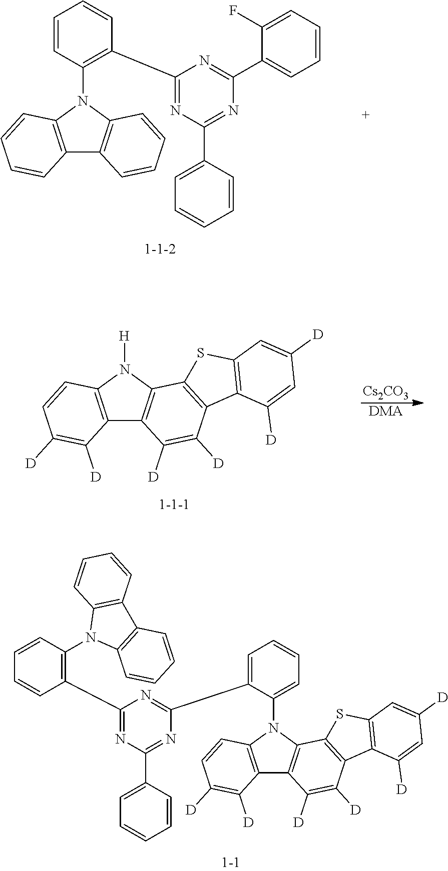

### 1) Preparation of Intermediate 1-1-1

12H-benzo[4,5]thieno[2,3-a]carbazole [A](10 g, 0.037 mol), triflic acid (CF₃SO₃H) (78.04 g, 0.52 mol), and D₆-benzene (200 mL) were put into a one-neck round-bottom flask, and the mixture was refluxed at 100° C. for 5 hours. After the reaction was completed, H₂O (100 mL) was added thereto for neutralization. A precipitated solid was filtered to obtain Intermediate 1-1-1. (8.48 g, yield 82%)

### 2) Preparation of Intermediate 1-1-2

2-Chloro-4-(2-fluorophenyl)-6-phenyl-1,3,5-triazine [B](5 g, 0.018 mol), (2-(9H-carbazol-9-yl)phenyl)boronic acid [C](5.69 g, 0.020 mol), Pd(PPh₃)₄ (0.62 g, 0.00054 mol), K₂CO₃ (4.98 g, 0.036 mol), and 1,4-dioxane (50 mL)/H₂O (10 mL) were put into a one-neck round-bottom flask, and the mixture was refluxed at 120° C. After the mixture was cooled, extraction was performed, and the organic layer was silica gel-filtered to obtain Intermediate 1-1-2. (8.16 g, yield 92%)

### 3) Preparation of Compound 1-1

Intermediate 1-1-2 (8.16 g, 0.017 mol), Intermediate 1-1-1 (4.75 g, 0.017 mol), Cs₂CO₃ (11.08 g, 0.034 mol), and DMA (80 mL) were put into a one-neck round-bottom flask, and the mixture was refluxed at 185° C. After the reaction was completed, a precipitated solid was filtered, and then the solid was dissolved in 1,2-dichlorobenzene, silica gel-filtered, and then concentrated to obtain Compound 1-1. (11.12 g, yield 87%)

### <Preparation Example 1-2> Preparation of Target Compound D of Following Table 1

The target compound D in the following Table 1 was obtained by performing synthesis in the same manner as in the above Preparation Example, except that the reaction products [A], [B], and [C] in the above Preparation Example were changed to the reaction products A, B, and C in the following Table 1.

TABLE 1

| Com- | | | |
|---|---|---|---|
| pound | Reaction | Reaction | |
| No. | Product A | Product B | Reaction Product C |
| 1-3 | 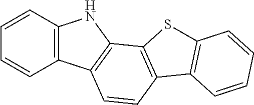 [SMILES: c1ccc2c(c1)[nH]c1c2ccc2c3ccccc3sc21] | 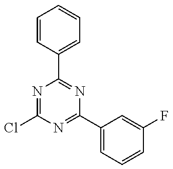 [SMILES: Fc1cccc(-c2nc(Cl)nc(-c3ccccc3)n2)c1] | 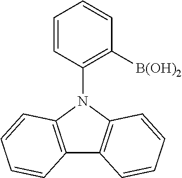 [SMILES: OB(O)c1ccccc1-n1c2ccccc2c2ccccc21] |
| 1-5 | 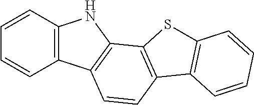 [SMILES: c1ccc2c(c1)[nH]c1c2ccc2c3ccccc3sc21] | 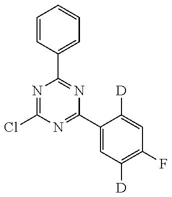 [SMILES: [2H]c1cc(-c2nc(Cl)nc(-c3ccccc3)n2)c([2H])cc1F] | 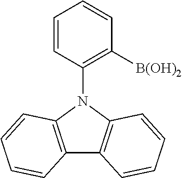 [SMILES: OB(O)c1ccccc1-n1c2ccccc2c2ccccc21] |
| 1-12 | 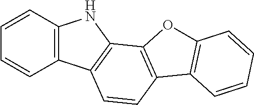 [SMILES: c1ccc2c(c1)[nH]c1c2ccc2c3ccccc3oc21] | 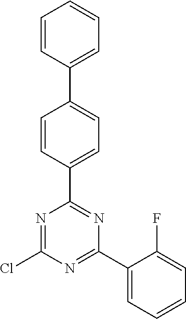 [SMILES: Fc1ccccc1-c1nc(Cl)nc(-c2ccc(-c3ccccc3)cc2)n1] | 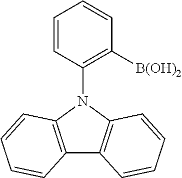 [SMILES: OB(O)c1ccccc1-n1c2ccccc2c2ccccc21] |
| 1-19 | 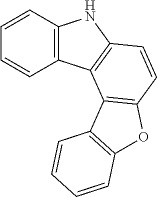 [SMILES: c1ccc2c(c1)[nH]c1ccc3oc4ccccc4c3c12] | 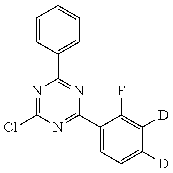 [SMILES: [2H]c1ccc(-c2nc(Cl)nc(-c3ccccc3)n2)c(F)c1[2H]] | 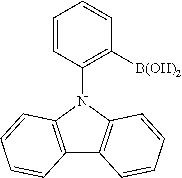 [SMILES: OB(O)c1ccccc1-n1c2ccccc2c2ccccc21] |
| 1-21 | 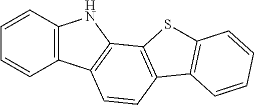 [SMILES: c1ccc2c(c1)[nH]c1c2ccc2c3ccccc3sc21] | 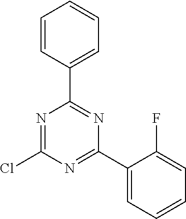 [SMILES: Fc1ccccc1-c1nc(Cl)nc(-c2ccccc2)n1] | 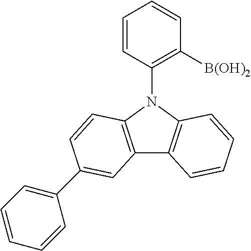 [SMILES: OB(O)c1ccccc1-n1c2ccccc2c2cc(-c3ccccc3)ccc21] |
| 1-22 | 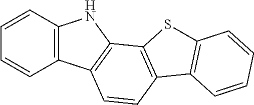 [SMILES: c1ccc2c(c1)[nH]c1c2ccc2c3ccccc3sc21] | 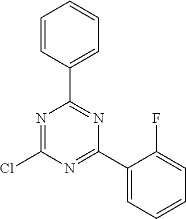 [SMILES: Fc1ccccc1-c1nc(Cl)nc(-c2ccccc2)n1] | 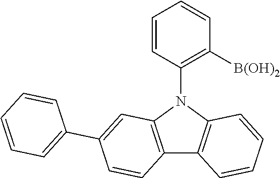 [SMILES: OB(O)c1ccccc1-n1c2ccccc2c2ccc(-c3ccccc3)cc21] |
| 1-23 | 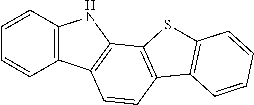 [SMILES: c1ccc2c(c1)[nH]c1c2ccc2c3ccccc3sc21] | 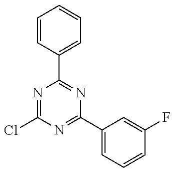 [SMILES: Fc1cccc(-c2nc(Cl)nc(-c3ccccc3)n2)c1] | 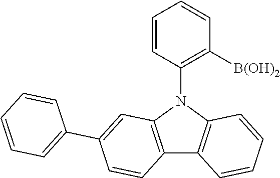 [SMILES: OB(O)c1ccccc1-n1c2ccccc2c2ccc(-c3ccccc3)cc21] |
| 1-25 | 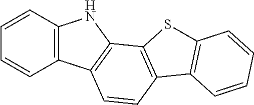 [SMILES: c1ccc2c(c1)[nH]c1c2ccc2c3ccccc3sc21] | 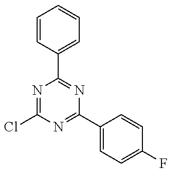 [SMILES: Fc1ccc(-c2nc(Cl)nc(-c3ccccc3)n2)cc1] | 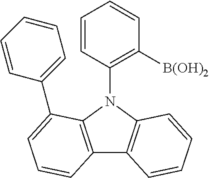 [SMILES: OB(O)c1ccccc1-n1c2ccccc2c2cccc(-c3ccccc3)c21] |
| 1-29 | 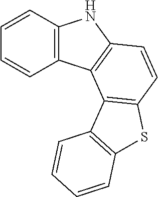 [SMILES: c1ccc2c(c1)[nH]c1ccc3sc4ccccc4c3c12] | 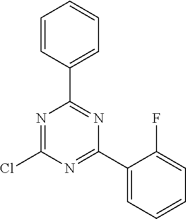 [SMILES: Fc1ccccc1-c1nc(Cl)nc(-c2ccccc2)n1] | 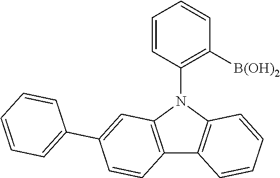 [SMILES: OB(O)c1ccccc1-n1c2ccccc2c2ccc(-c3ccccc3)cc21] |
| 1-34 | 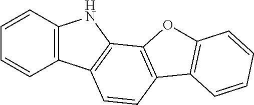 [SMILES: c1ccc2c(c1)[nH]c1c2ccc2c3ccccc3oc21] | 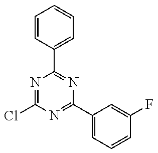 [SMILES: Fc1cccc(-c2nc(Cl)nc(-c3ccccc3)n2)c1] | 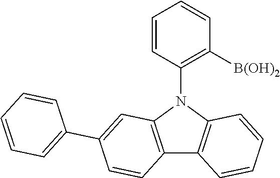 [SMILES: OB(O)c1ccccc1-n1c2ccccc2c2ccc(-c3ccccc3)cc21] |
| 1-38 | 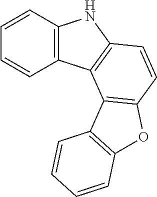 [SMILES: c1ccc2c(c1)[nH]c1ccc3oc4ccccc4c3c12] | 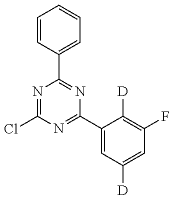 [SMILES: [2H]c1cc(F)c([2H])c(-c2nc(Cl)nc(-c3ccccc3)n2)c1] | 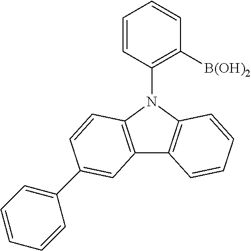 [SMILES: OB(O)c1ccccc1-n1c2ccccc2c2cc(-c3ccccc3)ccc21] |
| 1-42 | 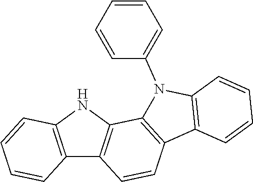 [SMILES: c1ccc(-n2c3ccccc3c3ccc4c5ccccc5[nH]c4c32)cc1] | 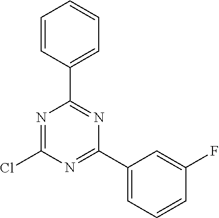 [SMILES: Fc1cccc(-c2nc(Cl)nc(-c3ccccc3)n2)c1] | 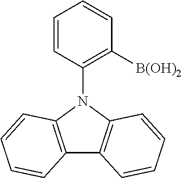 [SMILES: OB(O)c1ccccc1-n1c2ccccc2c2ccccc21] |
| 1-44 | 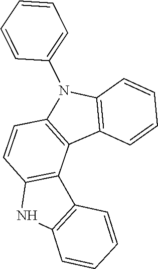 [SMILES: c1ccc(-n2c3ccccc3c3c4c(ccc32)[nH]c2ccccc24)cc1] | 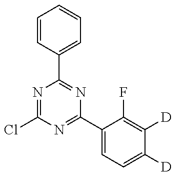 [SMILES: [2H]c1ccc(-c2nc(Cl)nc(-c3ccccc3)n2)c(F)c1[2H]] | 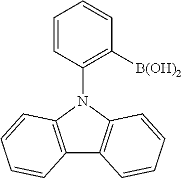 [SMILES: OB(O)c1ccccc1-n1c2ccccc2c2ccccc21] |
| 1-48 | 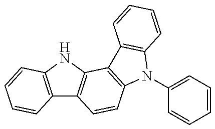 [SMILES: c1ccc(-n2c3ccccc3c3c4[nH]c5ccccc5c4ccc32)cc1] | 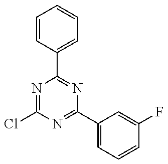 [SMILES: Fc1cccc(-c2nc(Cl)nc(-c3ccccc3)n2)c1] | 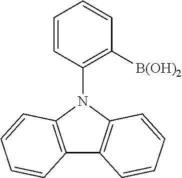 [SMILES: OB(O)c1ccccc1-n1c2ccccc2c2ccccc21] |
| 1-52 | 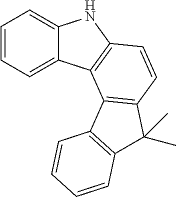 [SMILES: CC1(C)c2ccccc2-c2c1ccc1[nH]c3ccccc3c21] | 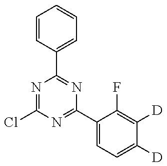 [SMILES: [2H]c1ccc(-c2nc(Cl)nc(-c3ccccc3)n2)c(F)c1[2H]] | 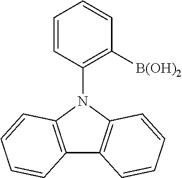 [SMILES: OB(O)c1ccccc1-n1c2ccccc2c2ccccc21] |
| 1-57 | 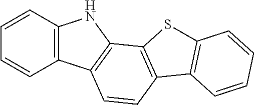 [SMILES: c1ccc2c(c1)[nH]c1c2ccc2c3ccccc3sc21] | 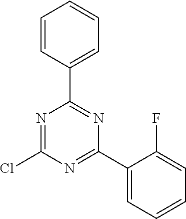 [SMILES: Fc1ccccc1-c1nc(Cl)nc(-c2ccccc2)n1] | 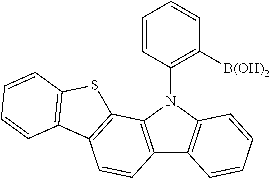 [SMILES: OB(O)c1ccccc1-n1c2ccccc2c2ccc3c4ccccc4sc3c21] |
| 1-58 | 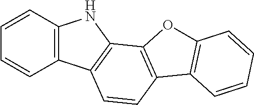 [SMILES: c1ccc2c(c1)[nH]c1c2ccc2c3ccccc3oc21] | 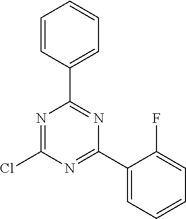 [SMILES: Fc1ccccc1-c1nc(Cl)nc(-c2ccccc2)n1] | 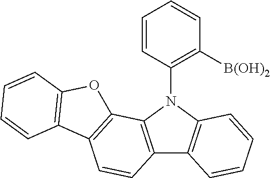 [SMILES: OB(O)c1ccccc1-n1c2ccccc2c2ccc3c4ccccc4oc3c21] |
| 1-59 | 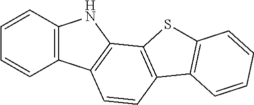 [SMILES: c1ccc2c(c1)[nH]c1c2ccc2c3ccccc3sc21] | 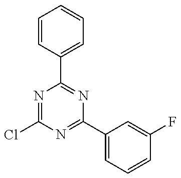 [SMILES: Fc1cccc(-c2nc(Cl)nc(-c3ccccc3)n2)c1] | 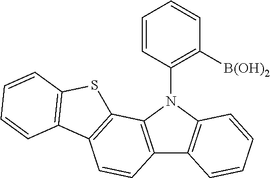 [SMILES: OB(O)c1ccccc1-n1c2ccccc2c2ccc3c4ccccc4sc3c21] |
| 1-64 | 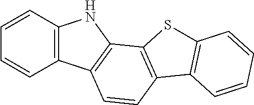 [SMILES: c1ccc2c(c1)[nH]c1c2ccc2c3ccccc3sc21] | 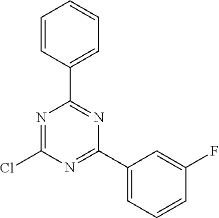 [SMILES: Fc1cccc(-c2nc(Cl)nc(-c3ccccc3)n2)c1] | 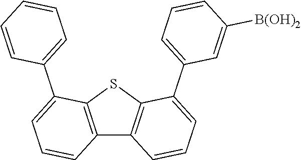 [SMILES: OB(O)c1cccc(-c2cccc3c2sc2c(-c4ccccc4)cccc23)c1] |
| 1-67 | 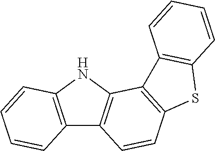 [SMILES: c1ccc2c(c1)[nH]c1c2ccc2sc3ccccc3c21] | 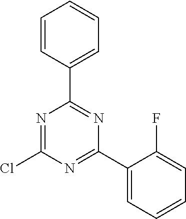 [SMILES: Fc1ccccc1-c1nc(Cl)nc(-c2ccccc2)n1] | 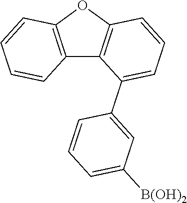 [SMILES: OB(O)c1cccc(-c2cccc3oc4ccccc4c23)c1] |
| 1-69 | 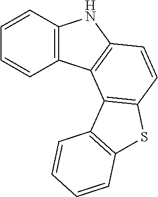 [SMILES: c1ccc2c(c1)[nH]c1ccc3sc4ccccc4c3c12] | 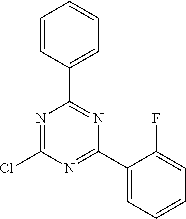 [SMILES: Fc1ccccc1-c1nc(Cl)nc(-c2ccccc2)n1] | 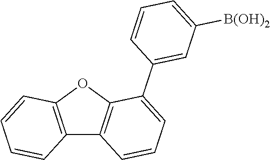 [SMILES: OB(O)c1cccc(-c2cccc3c2oc2ccccc23)c1] |
| 1-72 | 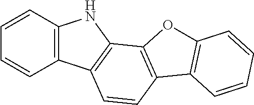 [SMILES: c1ccc2c(c1)[nH]c1c2ccc2c3ccccc3oc21] | 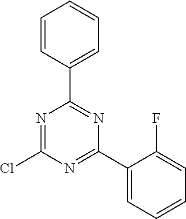 [SMILES: Fc1ccccc1-c1nc(Cl)nc(-c2ccccc2)n1] | 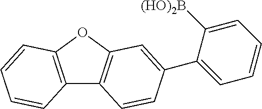 [SMILES: OB(O)c1ccccc1-c1ccc2c(c1)oc1ccccc12] |
| 1-80 | 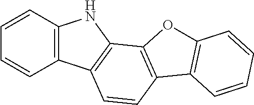 [SMILES: c1ccc2c(c1)[nH]c1c2ccc2c3ccccc3oc21] | 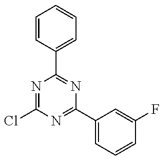 [SMILES: Fc1cccc(-c2nc(Cl)nc(-c3ccccc3)n2)c1] | 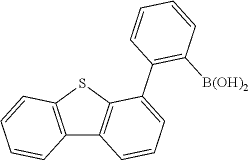 [SMILES: OB(O)c1ccccc1-c1cccc2c1sc1ccccc12] |
| 1-82 | 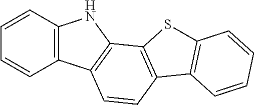 [SMILES: c1ccc2c(c1)[nH]c1c2ccc2c3ccccc3sc21] | 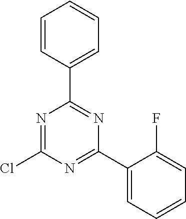 [SMILES: Fc1ccccc1-c1nc(Cl)nc(-c2ccccc2)n1] | 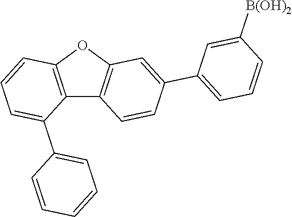 [SMILES: OB(O)c1cccc(-c2ccc3c(c2)oc2cccc(-c4ccccc4)c23)c1] |
| 1-85 | 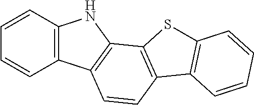 [SMILES: c1ccc2c(c1)[nH]c1c2ccc2c3ccccc3sc21] | 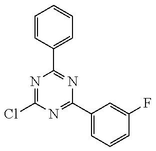 [SMILES: Fc1cccc(-c2nc(Cl)nc(-c3ccccc3)n2)c1] | 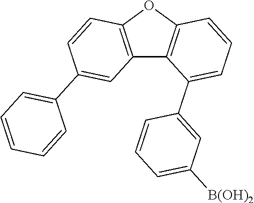 [SMILES: OB(O)c1cccc(-c2cccc3oc4ccc(-c5ccccc5)cc4c23)c1] |
| 1-90 | 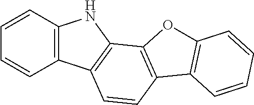 [SMILES: c1ccc2c(c1)[nH]c1c2ccc2c3ccccc3oc21] | 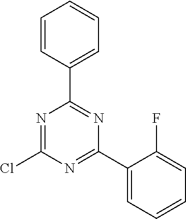 [SMILES: Fc1ccccc1-c1nc(Cl)nc(-c2ccccc2)n1] | 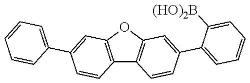 [SMILES: OB(O)c1ccccc1-c1ccc2c(c1)oc1cc(-c3ccccc3)ccc12] |
| 1-93 | 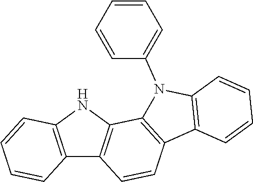 [SMILES: c1ccc(-n2c3ccccc3c3ccc4c5ccccc5[nH]c4c32)cc1] | 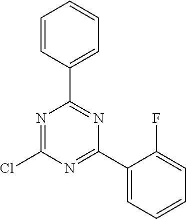 [SMILES: Fc1ccccc1-c1nc(Cl)nc(-c2ccccc2)n1] | 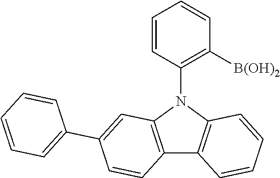 [SMILES: OB(O)c1ccccc1-n1c2ccccc2c2ccc(-c3ccccc3)cc21] |
| 1-94 | 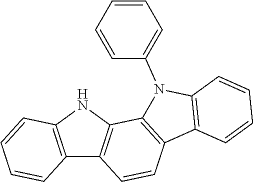 [SMILES: c1ccc(-n2c3ccccc3c3ccc4c5ccccc5[nH]c4c32)cc1] | 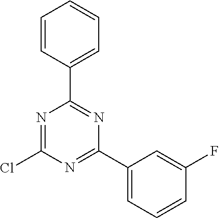 [SMILES: Fc1cccc(-c2nc(Cl)nc(-c3ccccc3)n2)c1] | 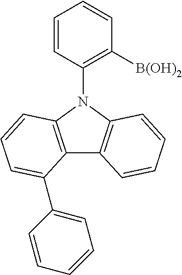 [SMILES: OB(O)c1ccccc1-n1c2ccccc2c2c(-c3ccccc3)cccc21] |
| 1-98 | 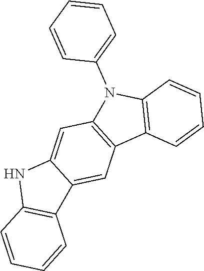 [SMILES: c1ccc(-n2c3ccccc3c3cc4c(cc32)[nH]c2ccccc24)cc1] | 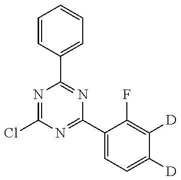 [SMILES: [2H]c1ccc(-c2nc(Cl)nc(-c3ccccc3)n2)c(F)c1[2H]] | 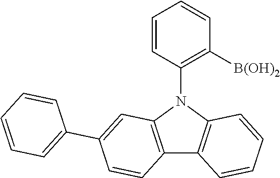 [SMILES: OB(O)c1ccccc1-n1c2ccccc2c2ccc(-c3ccccc3)cc21] |
| 1-100 | 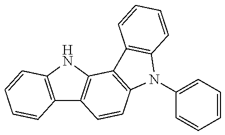 [SMILES: c1ccc(-n2c3ccccc3c3c4[nH]c5ccccc5c4ccc32)cc1] | 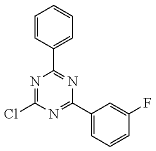 [SMILES: Fc1cccc(-c2nc(Cl)nc(-c3ccccc3)n2)c1] | 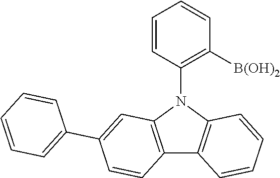 [SMILES: OB(O)c1ccccc1-n1c2ccccc2c2ccc(-c3ccccc3)cc21] |
| 1-103 | 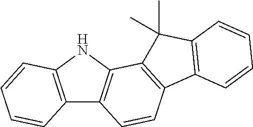 [SMILES: CC1(C)c2ccccc2-c2ccc3c([nH]c4ccccc43)c21] | 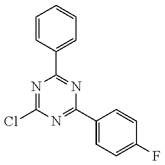 [SMILES: Fc1ccc(-c2nc(Cl)nc(-c3ccccc3)n2)cc1] | 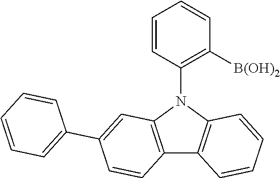 [SMILES: OB(O)c1ccccc1-n1c2ccccc2c2ccc(-c3ccccc3)cc21] |
| 1-112 | 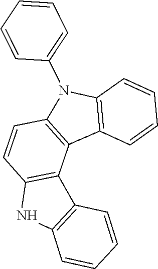 [SMILES: c1ccc(-n2c3ccccc3c3c4c(ccc32)[nH]c2ccccc24)cc1] | 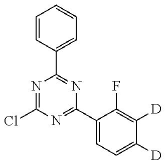 [SMILES: [2H]c1ccc(-c2nc(Cl)nc(-c3ccccc3)n2)c(F)c1[2H]] | 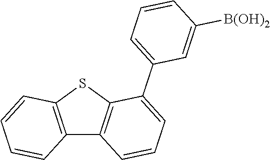 [SMILES: OB(O)c1cccc(-c2cccc3c2sc2ccccc23)c1] |
| 1-113 | 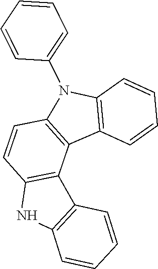 [SMILES: c1ccc(-n2c3ccccc3c3c4c(ccc32)[nH]c2ccccc24)cc1] | 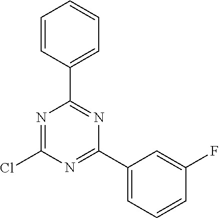 [SMILES: Fc1cccc(-c2nc(Cl)nc(-c3ccccc3)n2)c1] | 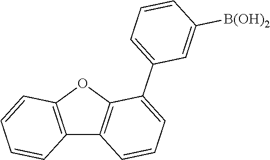 [SMILES: OB(O)c1cccc(-c2cccc3c2oc2ccccc23)c1] |
| 1-115 | 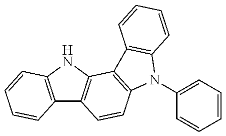 [SMILES: c1ccc(-n2c3ccccc3c3c4[nH]c5ccccc5c4ccc32)cc1] | 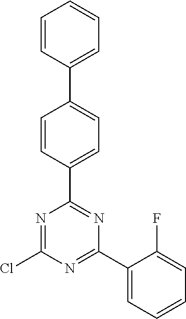 [SMILES: Fc1ccccc1-c1nc(Cl)nc(-c2ccc(-c3ccccc3)cc2)n1] | 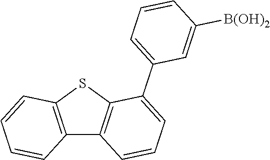 [SMILES: OB(O)c1cccc(-c2cccc3c2sc2ccccc23)c1] |
| 1-118 | 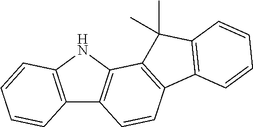 [SMILES: CC1(C)c2ccccc2-c2ccc3c([nH]c4ccccc43)c21] | 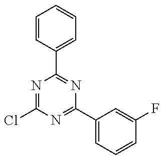 [SMILES: Fc1cccc(-c2nc(Cl)nc(-c3ccccc3)n2)c1] | 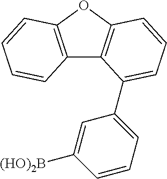 [SMILES: OB(O)c1cccc(-c2cccc3oc4ccccc4c23)c1] |
| 1-126 | 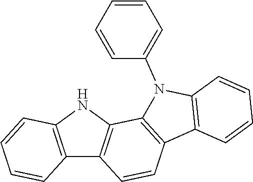 [SMILES: c1ccc(-n2c3ccccc3c3ccc4c5ccccc5[nH]c4c32)cc1] | 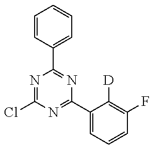 [SMILES: [2H]c1c(F)cccc1-c1nc(Cl)nc(-c2ccccc2)n1] | 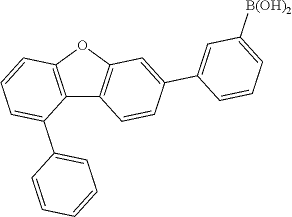 [SMILES: OB(O)c1cccc(-c2ccc3c(c2)oc2cccc(-c4ccccc4)c23)c1] |
| 1-131 | 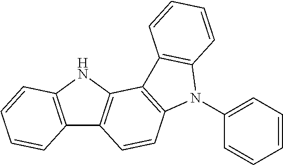 [SMILES: c1ccc(-n2c3ccccc3c3c4[nH]c5ccccc5c4ccc32)cc1] | 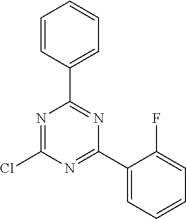 [SMILES: Fc1ccccc1-c1nc(Cl)nc(-c2ccccc2)n1] | 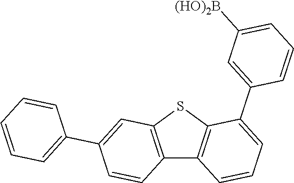 [SMILES: OB(O)c1cccc(-c2cccc3c2sc2cc(-c4ccccc4)ccc23)c1] |
| 1-132 | 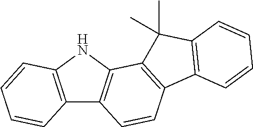 [SMILES: CC1(C)c2ccccc2-c2ccc3c([nH]c4ccccc43)c21] | 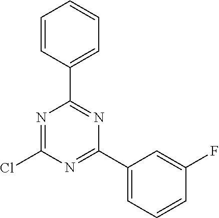 [SMILES: Fc1cccc(-c2nc(Cl)nc(-c3ccccc3)n2)c1] | 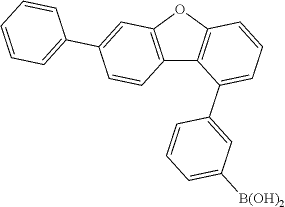 [SMILES: OB(O)c1cccc(-c2cccc3oc4cc(-c5ccccc5)ccc4c23)c1] |

| Compound | Target Compound | |
|---|---|---|
| No. | D | Yield |
| 1-3 | 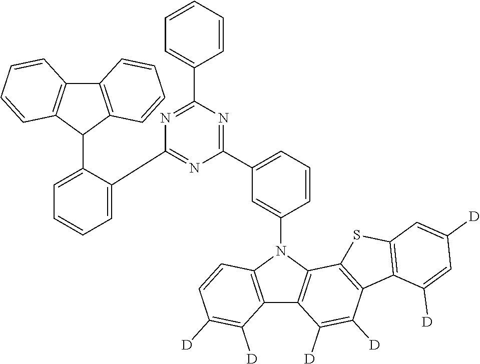 | 88% |
| 1-5 | 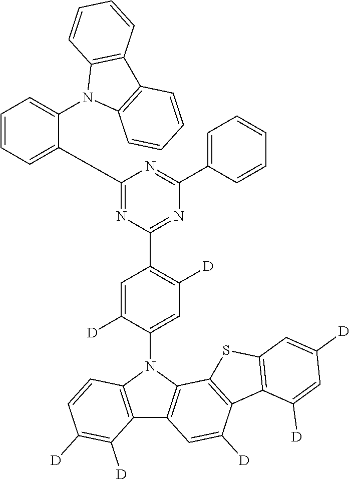 | 79% |
| 1-12 | 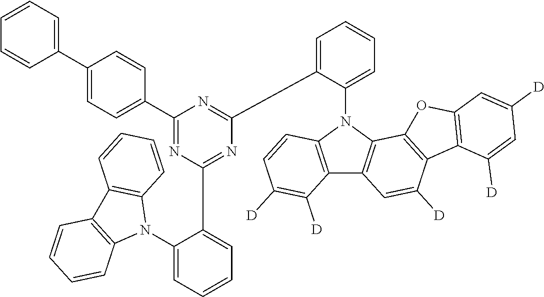 | 85% |
| 1-19 | 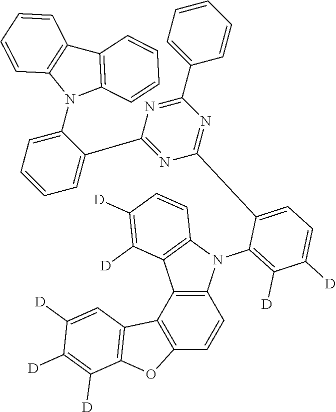 | 71% |
| 1-21 | 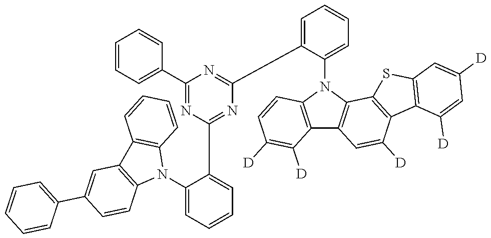 | 70% |
| 1-22 | 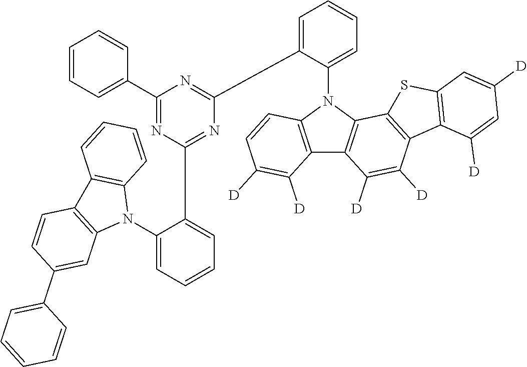 | 68% |
| 1-23 | 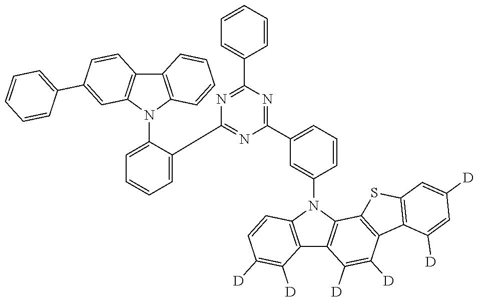 | 62% |
| 1-25 | 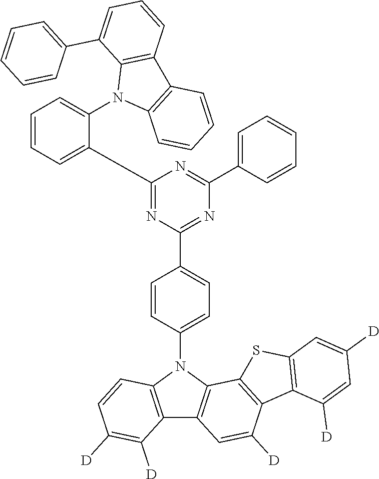 | 89% |
| 1-29 | 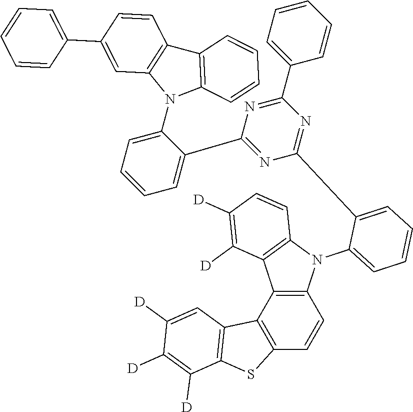 | 94% |
| 1-34 | 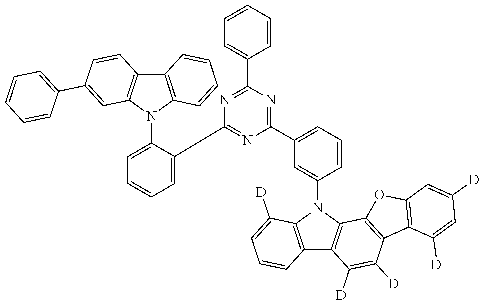 | 91% |
| 1-38 | 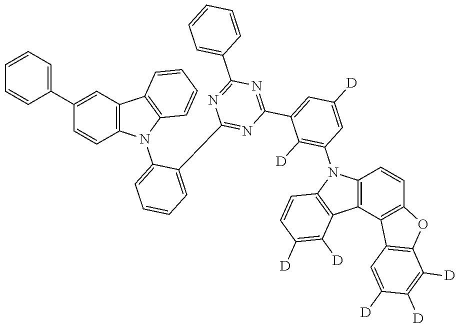 | 77% |
| 1-42 | 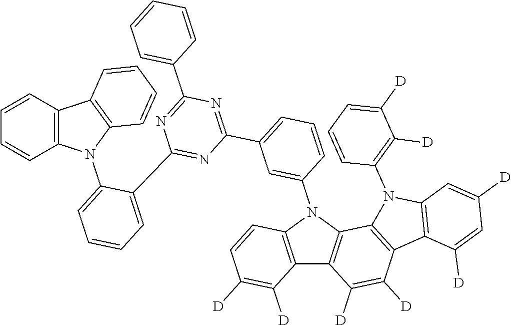 | 74% |
| 1-44 | 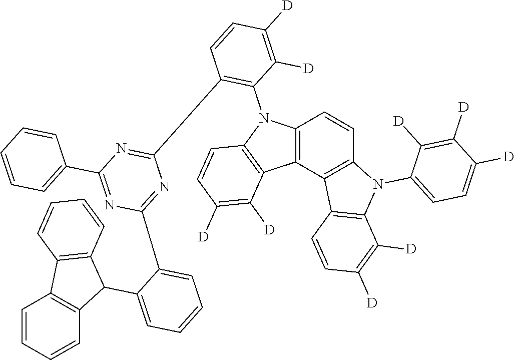 | 70% |
| 1-48 | 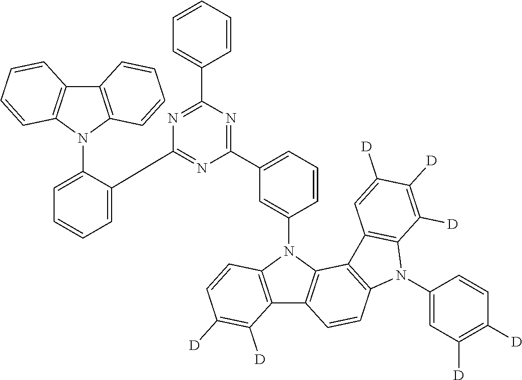 | 61% |
| 1-52 | 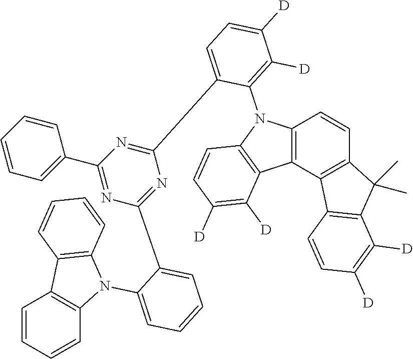 | 63% |
| 1-57 | 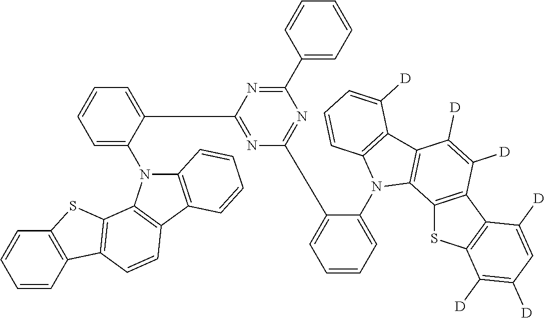 | 67% |
| 1-58 | 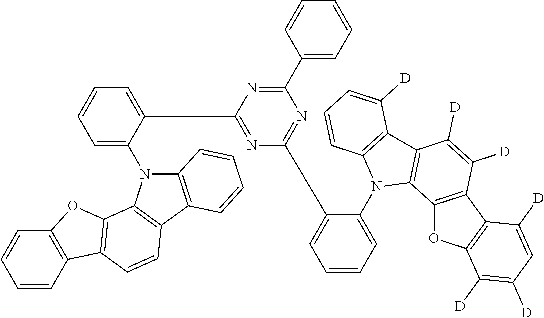 | 84% |
| 1-59 | 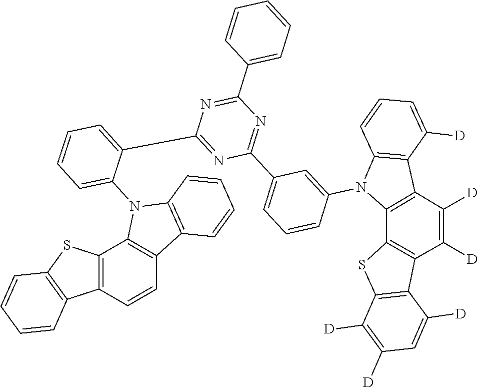 | 89% |
| 1-64 | 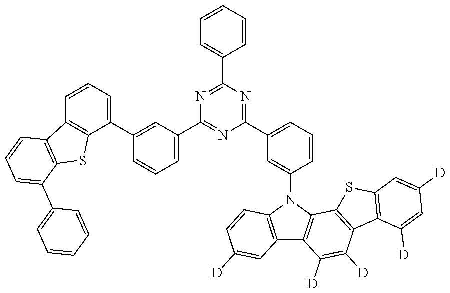 | 93% |
| 1-67 | 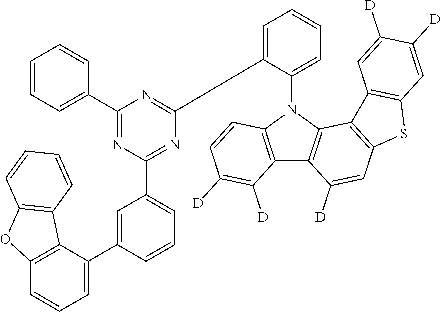 | 91% |
| 1-69 | 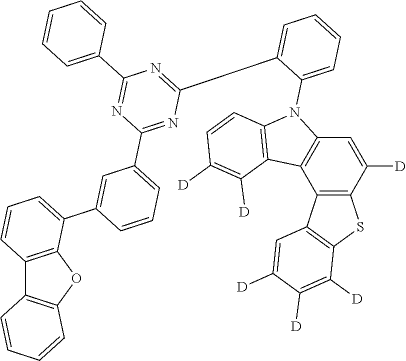 | 90% |
| 1-72 | 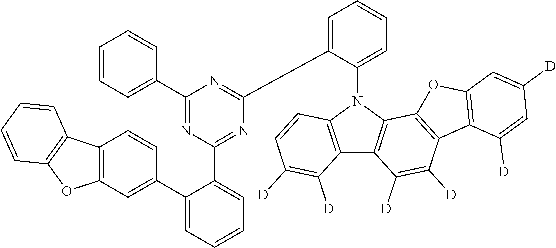 | 75% |
| 1-80 | 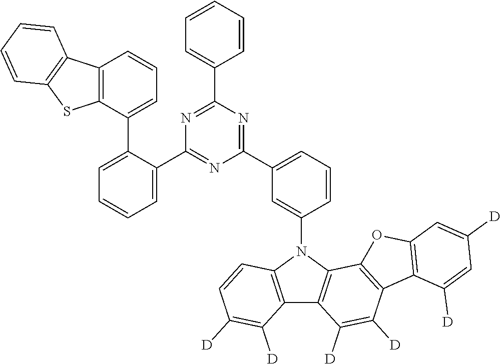 | 73% |
| 1-82 | 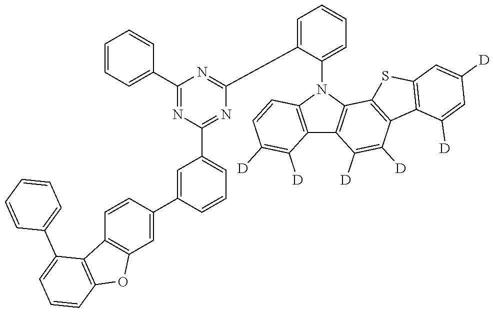 | 80% |
| 1-85 | 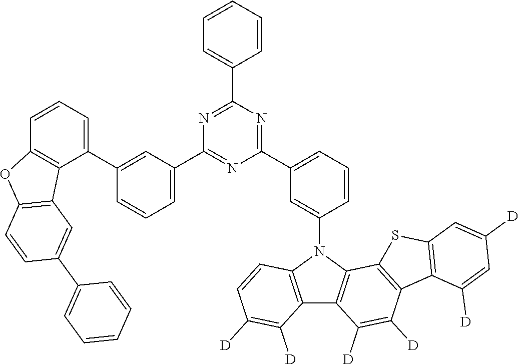 | 83% |
| 1-90 | 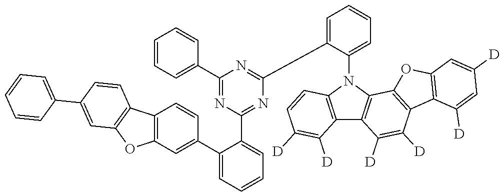 | 74% |
| 1-93 | 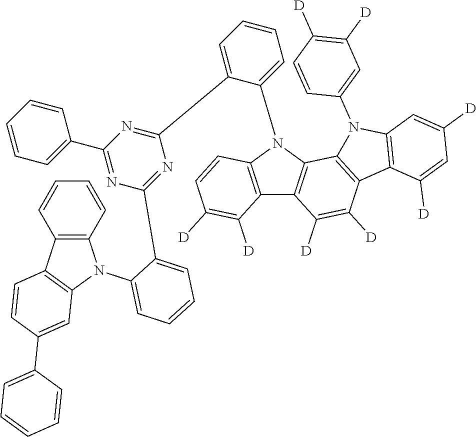 | 70% |
| 1-94 | 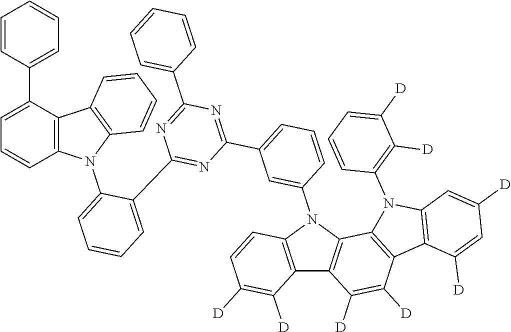 | 65% |
| 1-98 | 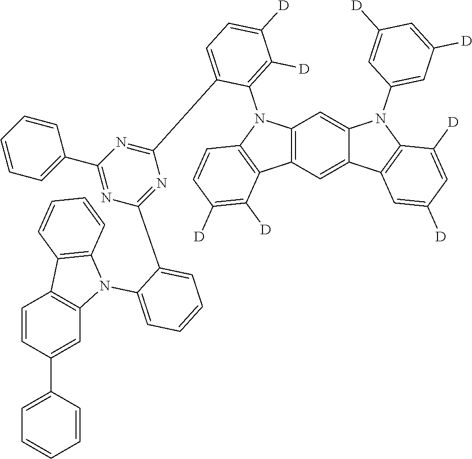 | 61% |
| 1-100 | 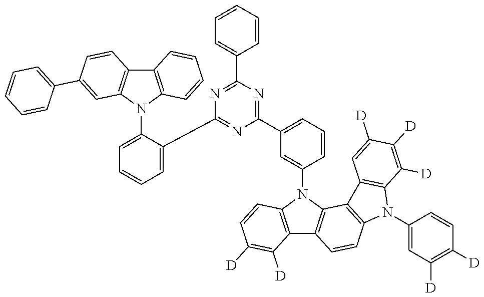 | 85% |
| 1-103 | 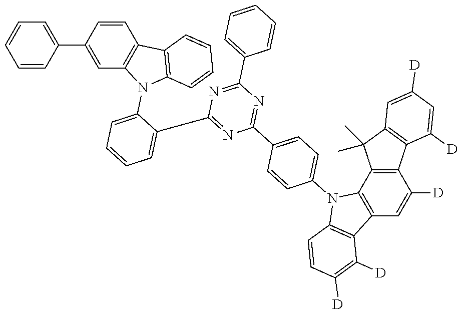 | 87% |
| 1-112 | 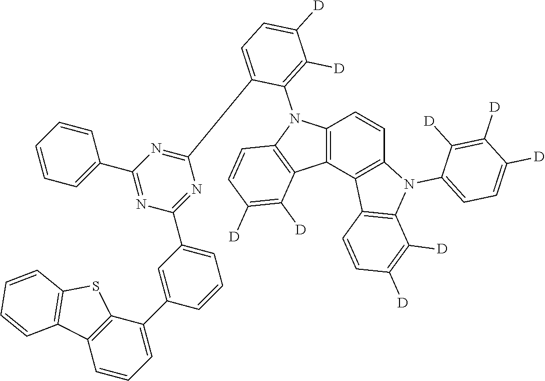 | 70% |
| 1-113 | 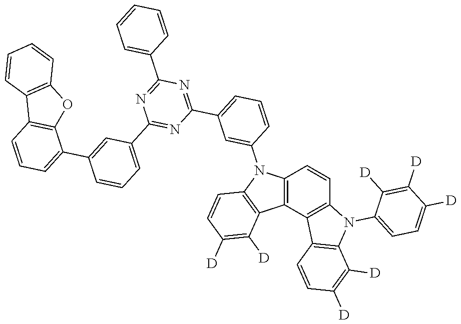 | 62% |
| 1-115 | 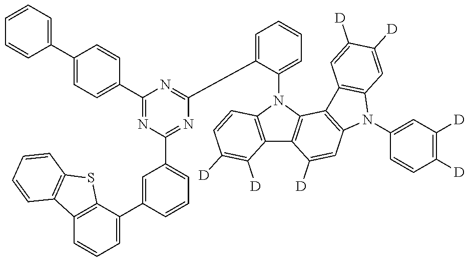 | 58% |
| 1-118 | 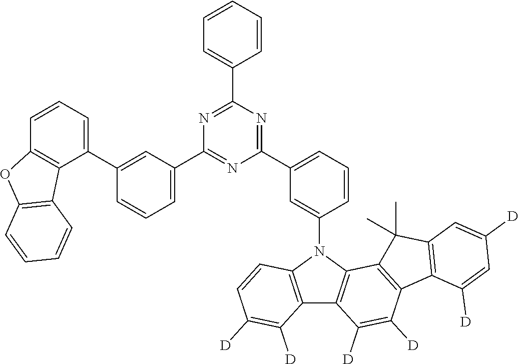 | 67% |
| 1-126 | 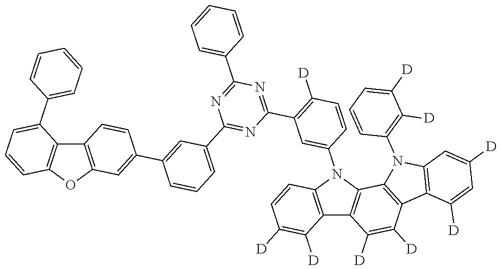 | 61% |
| 1-131 | 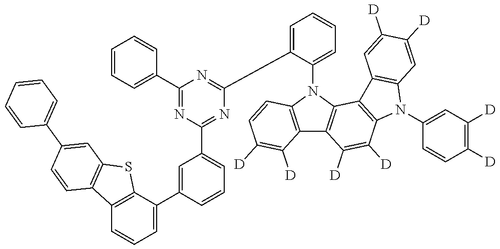 | 52% |
| 1-132 | 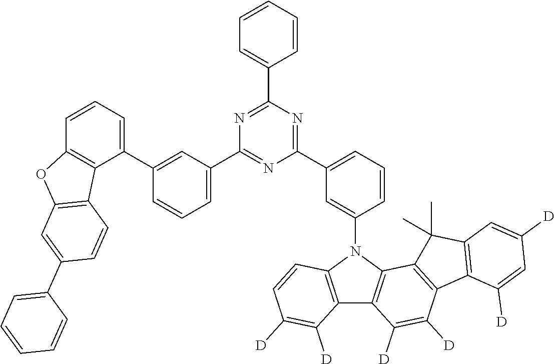 | 66% |

### <Preparation Example 2-1> Preparation of Compound 1-141

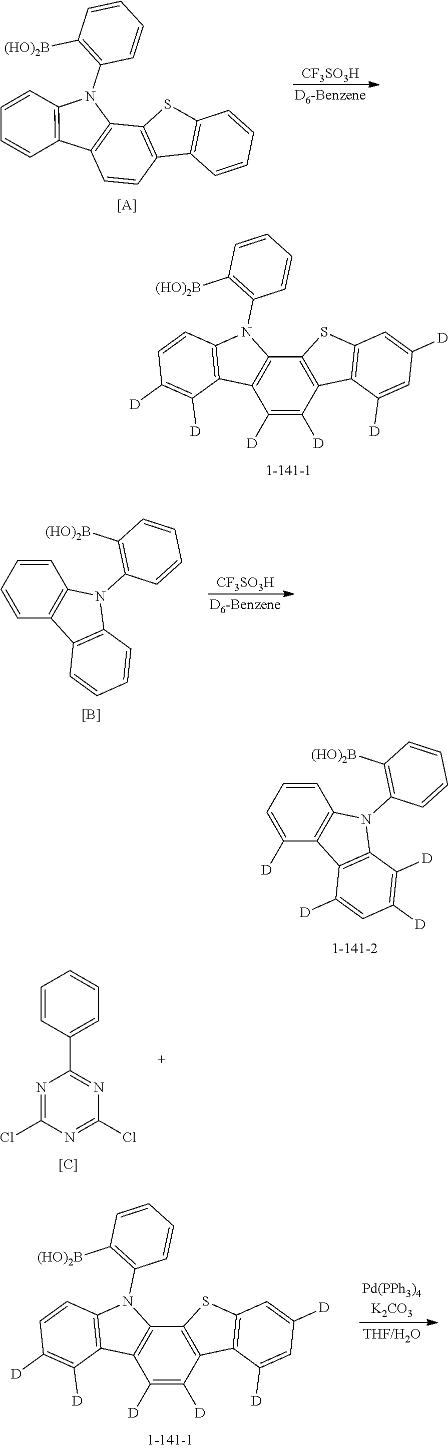

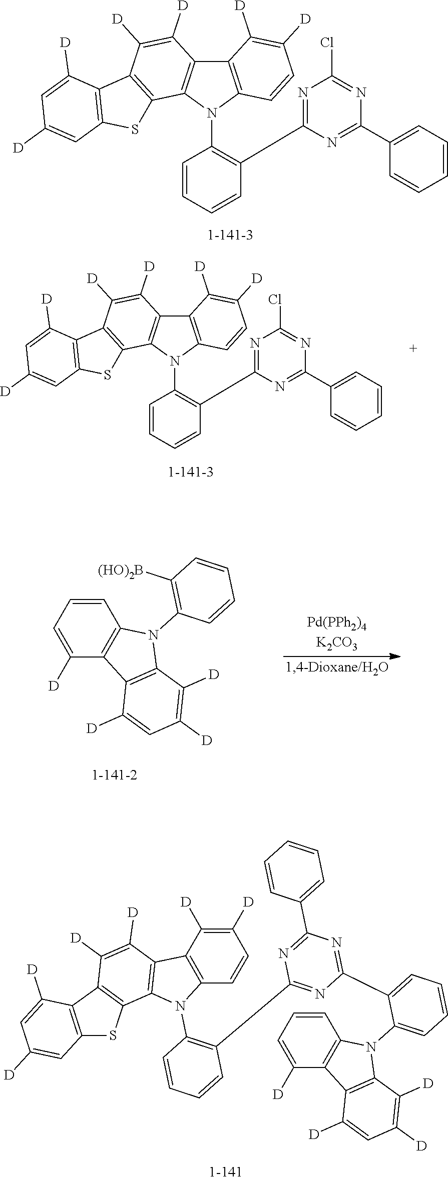 [SMILES: [2H]c1cc([2H])c2c(c1)sc1c2c([2H])c([2H])c2c3c([2H])c([2H])ccc3n(-c3ccccc3-c3nc(Cl)nc(-c4ccccc4)n3)c12 ; [2H]c1cc([2H])c2c(c1)sc1c2c([2H])c([2H])c2c3c([2H])c([2H])ccc3n(-c3ccccc3-c3nc(Cl)nc(-c4ccccc4)n3)c12 ; [2H]c1cc([2H])c2c3c([2H])cccc3n(-c3ccccc3B(O)O)c2c1[2H] ; [2H]c1cc([2H])c2c(c1)sc1c2c([2H])c([2H])c2c3c([2H])c([2H])ccc3n(-c3ccccc3-c3nc(-c4ccccc4)nc(-c4ccccc4-n4c5cccc([2H])c5c5c([2H])cc([2H])c([2H])c54)n3)c12]

### 1) Preparation of Intermediate 1-141-1

(2-(12H-benzo[4,5]thieno[2,3-a]carbazol-12-yl)phenyl)boronic acid [A](10 g, 0.025 mol), triflic acid (52.53 g, 0.35 mol), and D₆-benzene (200 mL) were put into a one-neck round-bottom flask, and the mixture was refluxed at 100° C. for 5 hours. After the reaction was completed, H₂O (100 mL) was added thereto for neutralization. A precipitated solid was filtered to obtain Intermediate 1-141-1. (8.78 g, yield 88%)

### 2) Preparation of Intermediate 1-141-2

(2-(9H-carbazol-9-yl)phenyl)boronic acid [B](10 g, 0.035 mol), triflic acid (53.54 g, 0.49 mol), and D₆-benzene (200 mL) were put into a one-neck round-bottom flask, and the mixture was refluxed at 100° C. for 5 hours. After the reaction was completed, H₂O (100 mL) was added thereto for neutralization. A precipitated solid was filtered to obtain Intermediate 1-141-2. (13.27 g, yield 93%)

### 3) Preparation of Intermediate 1-141-3

2,4-Dichloro-6-phenyl-1,3,5-triazine [C](5 g, 0.022 mol), Intermediate 1-141-1 (8.78 g, 0.022 mol), Pd(PPh₃)₄(0.76 g, 0.00066 mol), K₂CO₃ (6.08 g, 0.044 mol), and THF (50 mL)/H₂O (20 mL) were put into a one-neck round-bottom flask, and the mixture was refluxed at 100° C. After the mixture was cooled, extraction was performed, and the organic layer was silica gel-filtered to obtain Intermediate 1-141-3. (6.72 g, yield 56%)

### 4) Preparation of Compound 1-141

Intermediate 1-141-3 (6.72 g, 0.012 mol), Intermediate 1-141-2 (3.49 g, 0.012 mol), Pd(PPh₃)₄(0.42 g, 0.00036 mol), K₂CO₃ (3.32 g, 0.024 mol), and 1,4-dioxane (60 mL)/H₂O (30 mL) were put into a one-neck round-bottom flask, and the mixture was refluxed at 120° C. After the mixture was cooled, extraction was performed, and the organic layer was silica gel-filtered to obtain Compound 1-141. (8.07 g, yield 89%)

### <Preparation Example 2-2> Preparation of Target Compound D of Following Table 2

The target compound D in the following Table 2 was obtained by performing synthesis in the same manner as in the above Preparation Example, except that the reaction products [A], [B], and [C] in the above Preparation Example were changed to the reaction products A, B, and C in the following Table 2.

TABLE 2

| Compound | Reaction | Reaction | |
|---|---|---|---|
| No. | Product A | Product B | Reaction Product C |
| 1-137 | 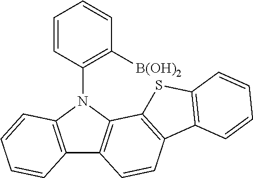 [SMILES: OB(O)c1ccccc1-n1c2ccccc2c2ccc3c4ccccc4sc3c21] | 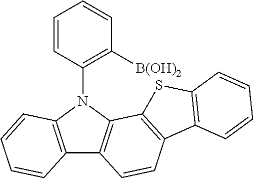 [SMILES: OB(O)c1ccccc1-n1c2ccccc2c2ccc3c4ccccc4sc3c21] | 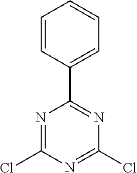 [SMILES: Clc1nc(Cl)nc(-c2ccccc2)n1] |
| 1-138 | 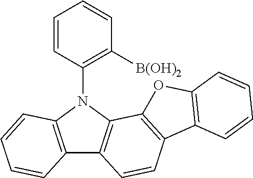 [SMILES: OB(O)c1ccccc1-n1c2ccccc2c2ccc3c4ccccc4oc3c21] | 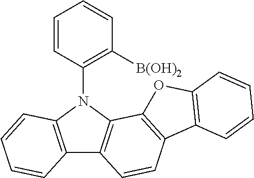 [SMILES: OB(O)c1ccccc1-n1c2ccccc2c2ccc3c4ccccc4oc3c21] | 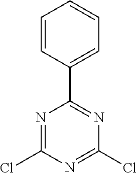 [SMILES: Clc1nc(Cl)nc(-c2ccccc2)n1] |
| 1-140 | 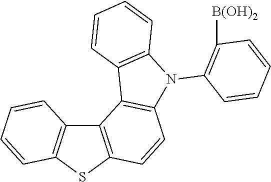 [SMILES: OB(O)c1ccccc1-n1c2ccccc2c2c3c(ccc21)sc1ccccc13] | 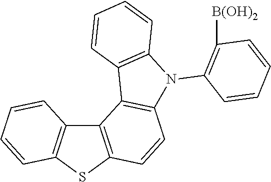 [SMILES: OB(O)c1ccccc1-n1c2ccccc2c2c3c(ccc21)sc1ccccc13] | 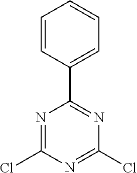 [SMILES: Clc1nc(Cl)nc(-c2ccccc2)n1] |
| 1-143 | 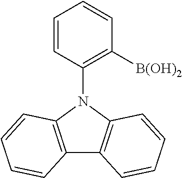 [SMILES: OB(O)c1ccccc1-n1c2ccccc2c2ccccc21] | 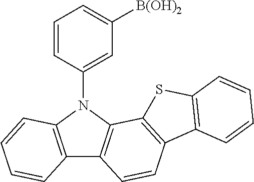 [SMILES: OB(O)c1cccc(-n2c3ccccc3c3ccc4c5ccccc5sc4c32)c1] | 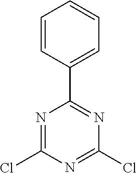 [SMILES: Clc1nc(Cl)nc(-c2ccccc2)n1] |
| 1-146 | 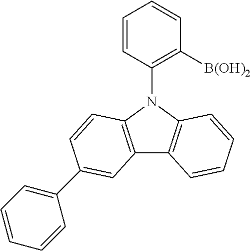 [SMILES: OB(O)c1ccccc1-n1c2ccccc2c2cc(-c3ccccc3)ccc21] | 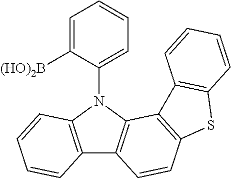 [SMILES: OB(O)c1ccccc1-n1c2ccccc2c2ccc3sc4ccccc4c3c21] | 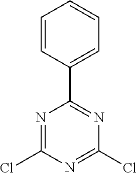 [SMILES: Clc1nc(Cl)nc(-c2ccccc2)n1] |
| 1-148 | 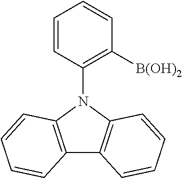 [SMILES: OB(O)c1ccccc1-n1c2ccccc2c2ccccc21] | 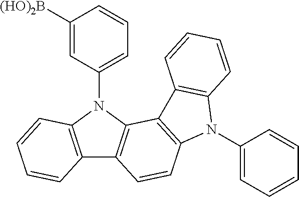 [SMILES: OB(O)c1cccc(-n2c3ccccc3c3ccc4c(c5ccccc5n4-c4ccccc4)c32)c1] | 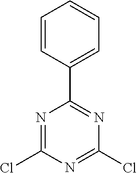 [SMILES: Clc1nc(Cl)nc(-c2ccccc2)n1] |
| 1-149 | 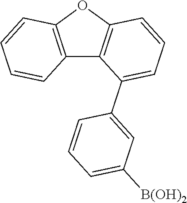 [SMILES: OB(O)c1cccc(-c2cccc3oc4ccccc4c23)c1] | 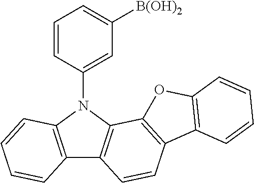 [SMILES: OB(O)c1cccc(-n2c3ccccc3c3ccc4c5ccccc5oc4c32)c1] | 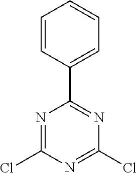 [SMILES: Clc1nc(Cl)nc(-c2ccccc2)n1] |
| 1-152 | 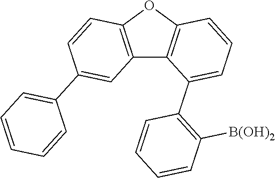 [SMILES: OB(O)c1ccccc1-c1cccc2oc3ccc(-c4ccccc4)cc3c12] | 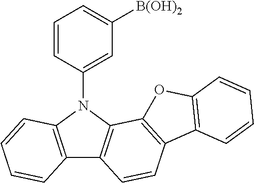 [SMILES: OB(O)c1cccc(-n2c3ccccc3c3ccc4c5ccccc5oc4c32)c1] | 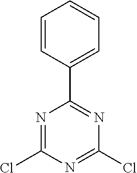 [SMILES: Clc1nc(Cl)nc(-c2ccccc2)n1] |

| Compound | Target Compound | |
|---|---|---|
| No. | D | Yield |
| 1-137 | 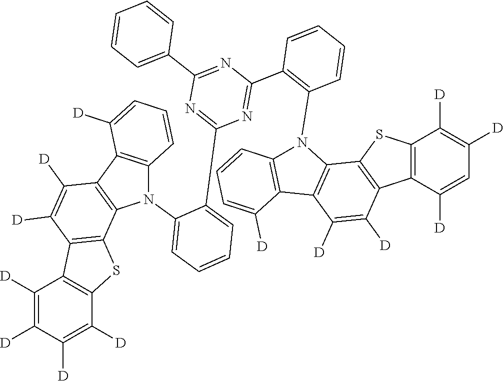 | 88% |
| 1-138 | 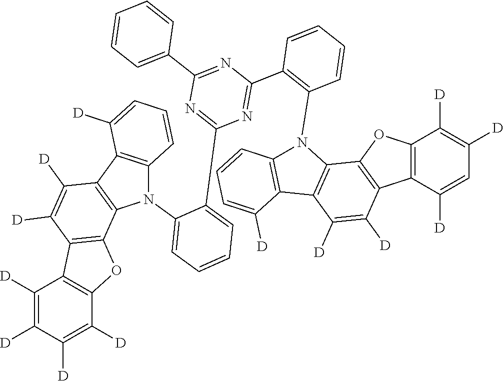 | 72% |
| 1-140 | 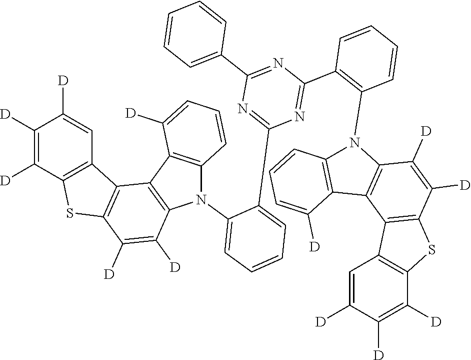 | 75% |
| 1-143 | 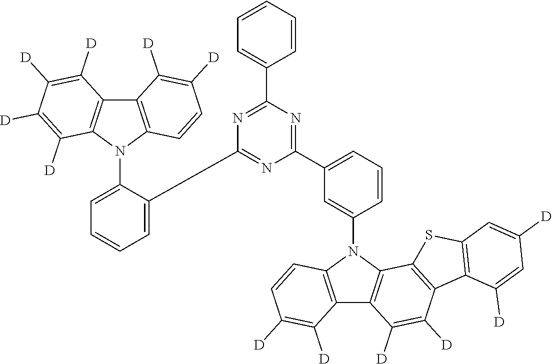 | 83% |
| 1-146 | 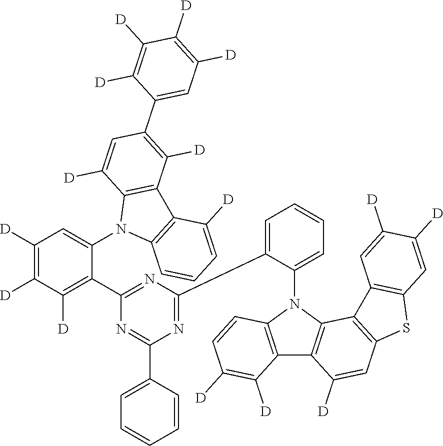 | 94% |
| 1-148 | 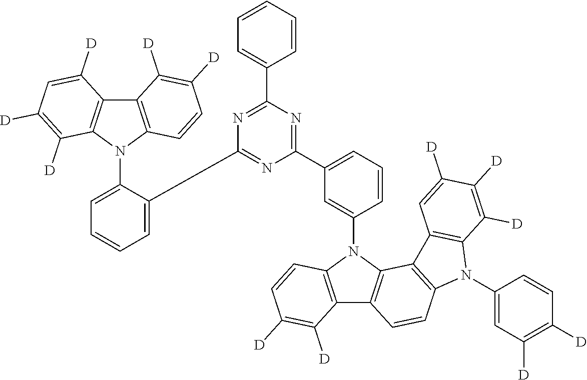 | 67% |
| 1-149 | 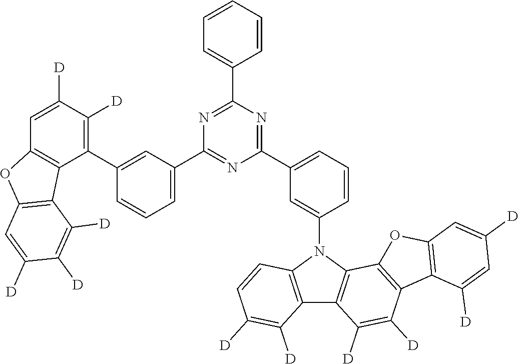 | 65% |
| 1-152 | 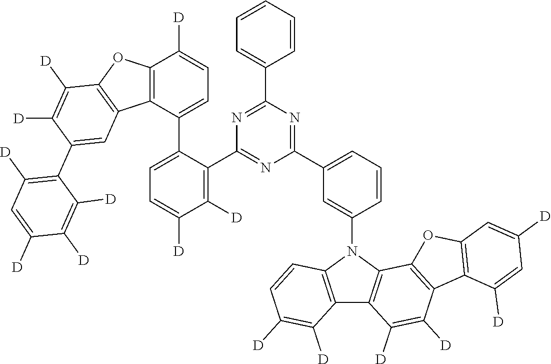 | 83% |

### <Preparation Example 3-1> Preparation of Compound 2-1

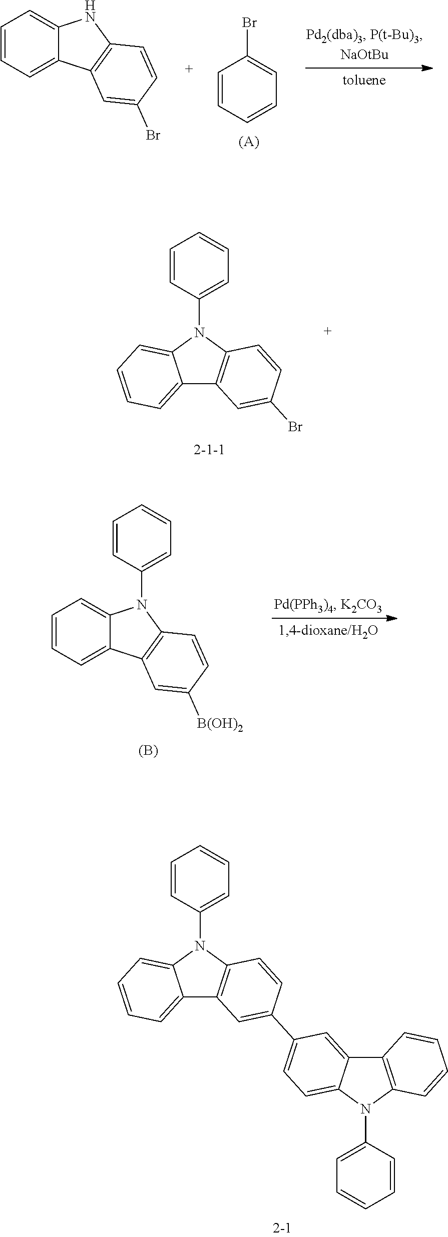

### 1) Preparation of Intermediate 2-1-1

3-Bromo-9H-carbazole (10 g, 0.041 mol), bromobenzene [A](6.41 g, 0.041 mol), Pd₂(dba)₃ (3.75 g, 0.0041 mol), P(t-Bu)₃ (1.66 g, 0.0082 mmol), NaOtBu (7.88 g, 0.082 mol), and toluene (100 mL) were put into a one-neck round-bottom flask, and the mixture was refluxed at 100° C. After the reaction was completed and the mixture was cooled, extraction was performed, and then the organic layer was silica gel-filtered to obtain Intermediate 2-1-1. (9.51 g, yield 72%)

### 2) Preparation of Compound 2-1

Intermediate 2-1-1 (9.51 g, 0.030 mol), (9-phenyl-9H-carbazol-3-yl)boronic acid [B](8.61 g, 0.030 mol), Pd(PPh₃)₄(1.73 g, 0.0015 mol), K₂CO₃ (8.29 g, 0.06 mol), and 1,4-dioxane (90 mL)/H₂O (30 mL) were put into a one-neck round-bottom flask, and the mixture was refluxed at 125° C. After the mixture was cooled, extraction was performed, and then the organic layer was column-purified to obtain Compound 2-1. (9.89 g, yield 68%)

### <Preparation Example 3-2> Preparation of Target Compound C of Following Table 3

The target compound C in the following Table 3 was obtained by performing synthesis in the same manner as in the above Preparation Example, except that the reaction products [A] and [B] in the above Preparation Example were changed to the reaction products A and B in the following Table 3.

TABLE 3

| Com- | | | | |
|---|---|---|---|---|
| pound | Reaction | Reaction | Target | |
| No. | Product A | Product B | Compound C | Yield |
| 2-3 | 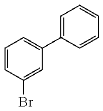 [SMILES: Brc1cccc(-c2ccccc2)c1] | 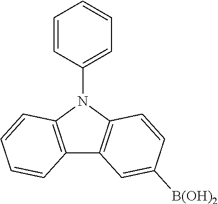 [SMILES: OB(O)c1ccc2c(c1)c1ccccc1n2-c1ccccc1] | 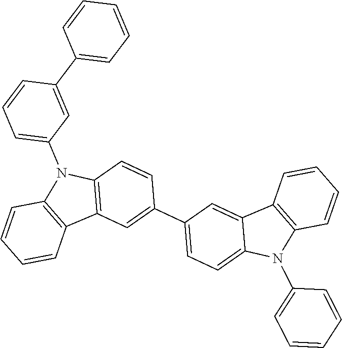 [SMILES: c1ccc(-c2cccc(-n3c4ccccc4c4cc(-c5ccc6c(c5)c5ccccc5n6-c5ccccc5)ccc43)c2)cc1] | 88% |
| 2-4 | 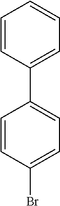 [SMILES: Brc1ccc(-c2ccccc2)cc1] | 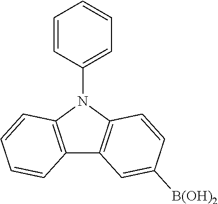 [SMILES: OB(O)c1ccc2c(c1)c1ccccc1n2-c1ccccc1] | 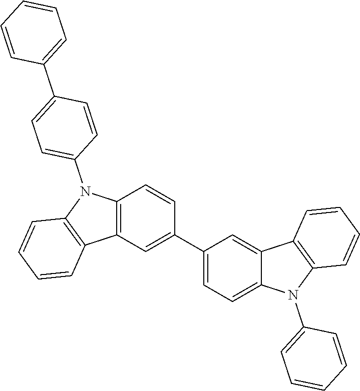 [SMILES: c1ccc(-c2ccc(-n3c4ccccc4c4cc(-c5ccc6c(c5)c5ccccc5n6-c5ccccc5)ccc43)cc2)cc1] | 94% |
| 2-16 | 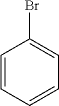 | 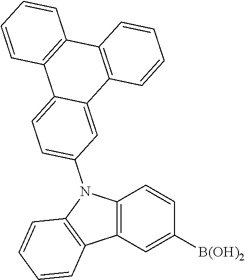 [SMILES: OB(O)c1ccc2c(c1)c1ccccc1n2-c1ccc2c3ccccc3c3ccccc3c2c1] | 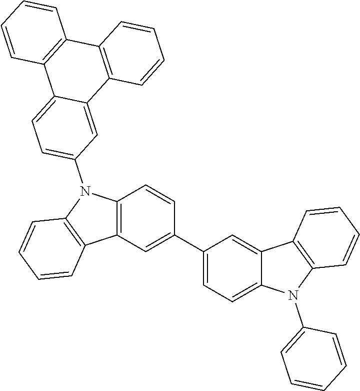 [SMILES: c1ccc(-n2c3ccccc3c3cc(-c4ccc5c(c4)c4ccccc4n5-c4ccc5c6ccccc6c6ccccc6c5c4)ccc32)cc1] | 87% |
| 2-27 | 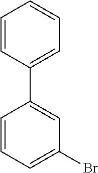 [SMILES: Brc1cccc(-c2ccccc2)c1] | 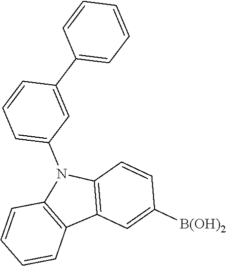 [SMILES: OB(O)c1ccc2c(c1)c1ccccc1n2-c1cccc(-c2ccccc2)c1] | 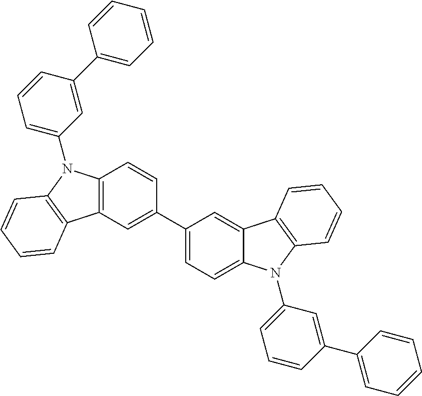 [SMILES: c1ccc(-c2cccc(-n3c4ccccc4c4cc(-c5ccc6c(c5)c5ccccc5n6-c5cccc(-c6ccccc6)c5)ccc43)c2)cc1] | 66% |
| 2-28 | 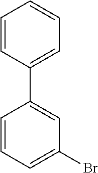 [SMILES: Brc1cccc(-c2ccccc2)c1] | 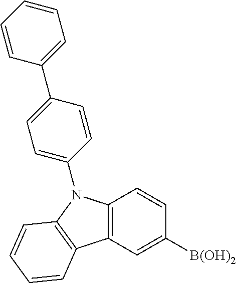 [SMILES: OB(O)c1ccc2c(c1)c1ccccc1n2-c1ccc(-c2ccccc2)cc1] | 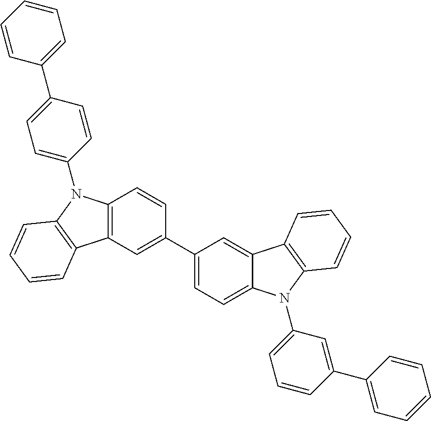 [SMILES: c1ccc(-c2ccc(-n3c4ccccc4c4cc(-c5ccc6c(c5)c5ccccc5n6-c5cccc(-c6ccccc6)c5)ccc43)cc2)cc1] | 54% |
| 2-32 | 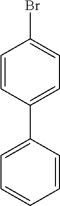 [SMILES: Brc1ccc(-c2ccccc2)cc1] | 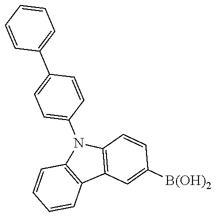 [SMILES: OB(O)c1ccc2c(c1)c1ccccc1n2-c1ccc(-c2ccccc2)cc1] | 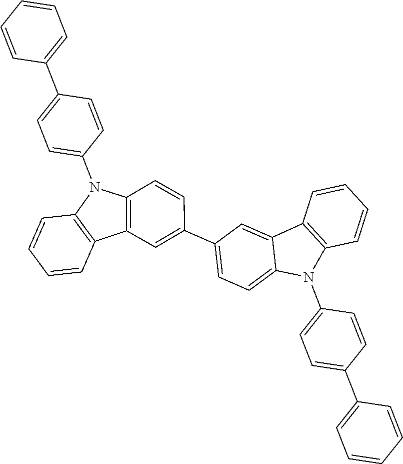 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 73% |

### <Preparation Example 4-1> Preparation of Compound 2-82

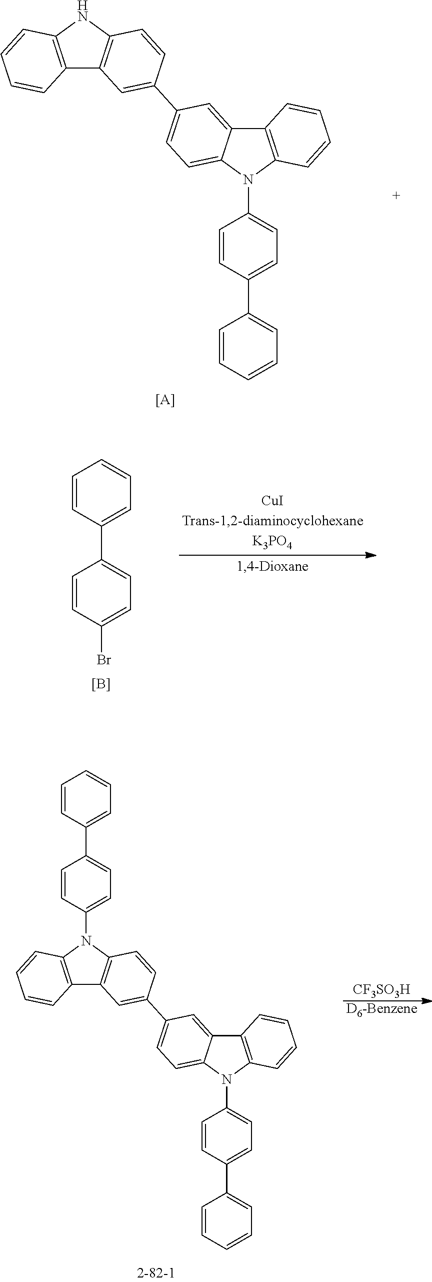

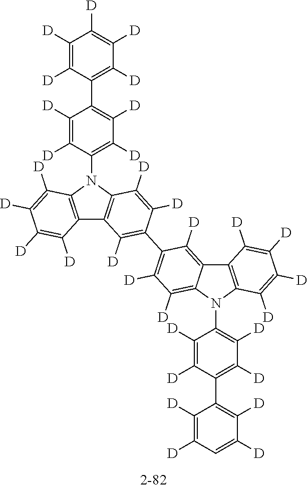 [SMILES: [2H]c1cc([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c1[2H]]

### 1) Preparation of Intermediate 2-82-1

9-([1,1′-biphenyl]-4-yl)-9H,9′H-3,3′-bicarbazole [A](10 g, 0.021 mol), 4-bromo-1,1′-biphenyl [B](5.38 g, 0.023 mol), CuI (0.40 g, 0.0021 mol), trans-1,4-diaminocyclohexane (0.024 g, 0.0021 mol), K₃PO₄ (8.92 g, 0.042 mol), and 1,4-dioxane (100 mL) were put into a one-neck round-bottom flask, and the mixture was refluxed at 125° C. for 8 hours. After the reaction was completed, distilled water and dichloromethane (DCM) were added thereto at room temperature, extraction was performed, the organic layer was dried over MgSO₄, and then the solvent was removed by a rotary evaporator. The reaction product was purified by column chromatography (DCM:hexane=1:3) and recrystallized with methanol to obtain Intermediate 2-82-1. (12.17 g, 91%) 2) Preparation of Compound 2-82 Intermediate 2-82-1 (9.89 g, 0.016 mol), CF₃SO₃H (33.02 g, 0.22 mol), and D₆-benzene (200 mL) were put into a one-neck round-bottom flask, and the mixture was refluxed at 90° C. for 24 hours. After the reaction was completed, H₂O (1 L) was slowly added for quenching, and a precipitated solid was filtered to obtain Compound 2-82. (8.88 g, yield 83%)

### <Preparation Example 4-2> Preparation of Target Compound C of Following Table 4

The target compound C in the following Table 4 was obtained by performing synthesis in the same manner as in the above Preparation Example, except that the reaction products [A] and [B] in the above Preparation Example were changed to the reaction products A and B in the following Table 4.

TABLE 4

| Compound | Reaction | Reaction | Target | |
|---|---|---|---|---|
| No. | Product A | Product B | Compound C | Yield |
| 2-85 | 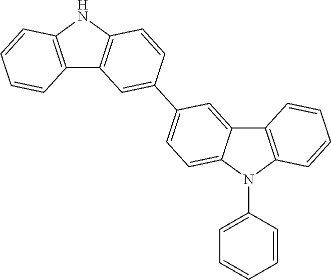 [SMILES: c1ccc(-n2c3ccccc3c3cc(-c4ccc5[nH]c6ccccc6c5c4)ccc32)cc1] | 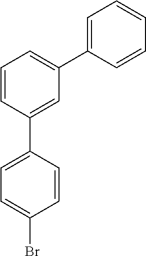 [SMILES: Brc1ccc(-c2cccc(-c3ccccc3)c2)cc1] | 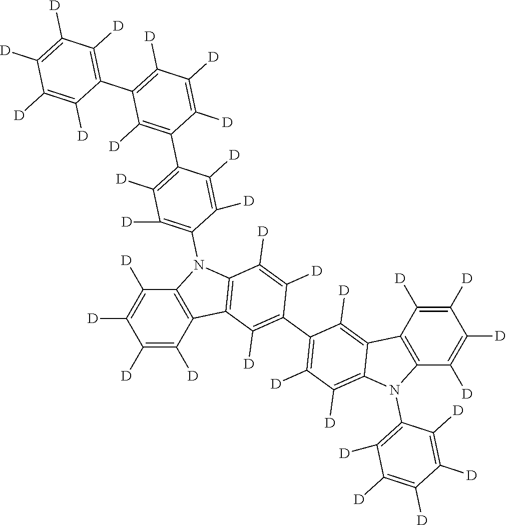 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c2[2H])c([2H])c1[2H]] | 74% |
| 2-88 | 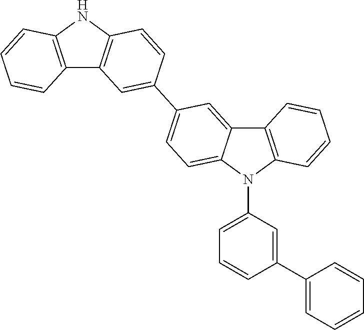 [SMILES: c1ccc(-c2cccc(-n3c4ccccc4c4cc(-c5ccc6[nH]c7ccccc7c6c5)ccc43)c2)cc1] | 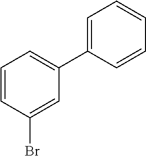 [SMILES: Brc1cccc(-c2ccccc2)c1] | 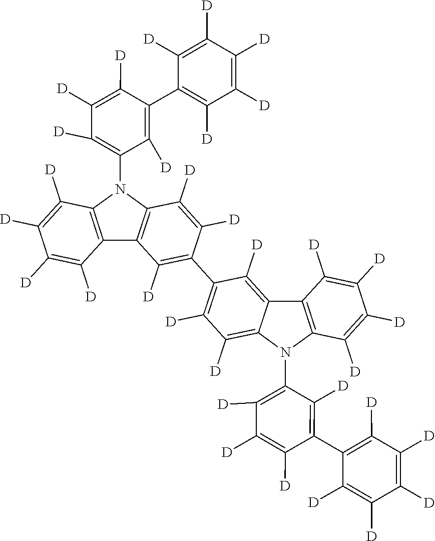 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c2[2H])c([2H])c1[2H]] | 76% |
| 2-89 | 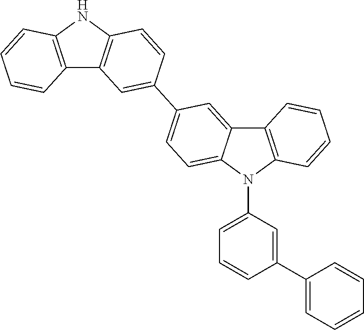 [SMILES: c1ccc(-c2cccc(-n3c4ccccc4c4cc(-c5ccc6[nH]c7ccccc7c6c5)ccc43)c2)cc1] | 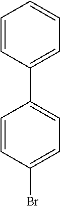 [SMILES: Brc1ccc(-c2ccccc2)cc1] | 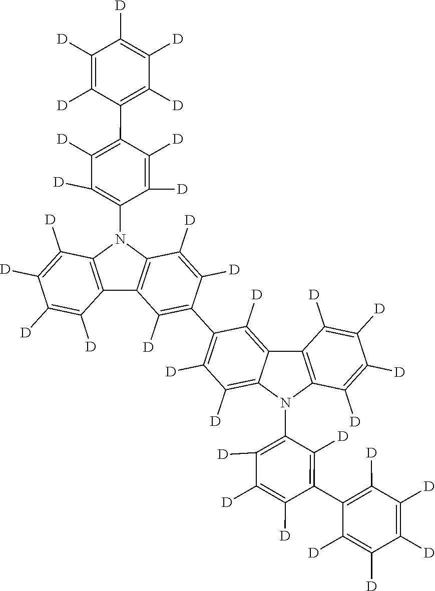 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H]] | 62% |

### <Preparation Example 5-1> Preparation of Compound 3-4

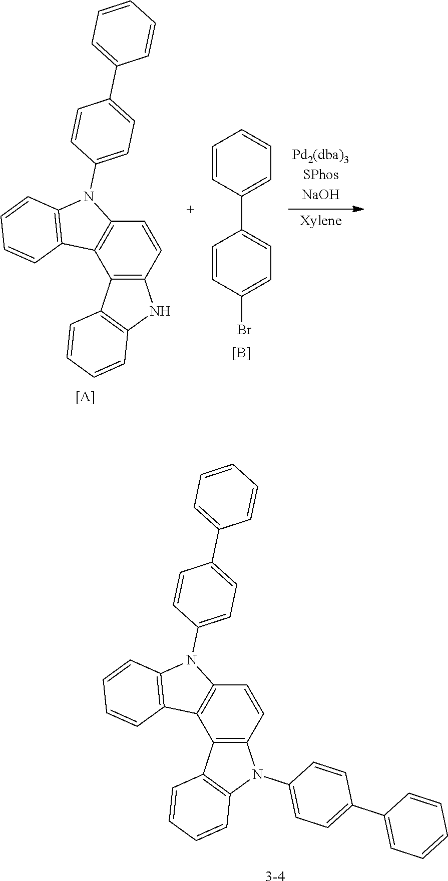

5-([1,1′-biphenyl]-4-yl)-5,8-dihydroindolo[2,3-c]carbazole [A](10 g, 0.024 mol), 4-bromo-1,1′-biphenyl [B](5.71 g, 0.024 mol), Pd₂(dba)₃ (1.10 g, 0.0012 mol), SPhos (1.97 g, 0.0048 mmol), NaOH (1.92 g, 0.048 mol), and xylene (100 mL) were put into a one-neck round-bottom flask, and the mixture was refluxed at 153° C. After the reaction was completed, the mixture was cooled, and then extraction was performed, and the organic layer was silica gel-filtered to obtain Compound 3-4. (8.48 g, yield 63%)

### <Preparation Example 5-2> Preparation of Target Compound C of Following Table 5

The target compound C in the following Table 5 was obtained by performing synthesis in the same manner as in the above Preparation Example, except that the reaction products [A] and [B] in the above Preparation Example were changed to the reaction products A and B in the following Table 5.

TABLE 5

| Com- | | | | |
|---|---|---|---|---|
| pound | Reaction | Reaction | Target | |
| No. | Product A | Product B | Compound C | Yield |
| 3-11 | 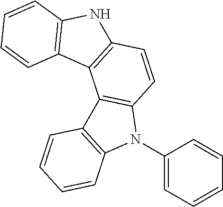 [SMILES: c1ccc(-n2c3ccccc3c3c4c(ccc32)[nH]c2ccccc24)cc1] | 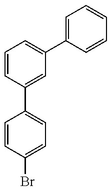 [SMILES: Brc1ccc(-c2cccc(-c3ccccc3)c2)cc1] | 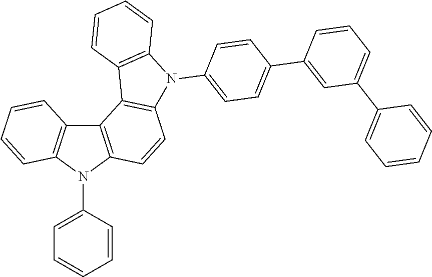 [SMILES: c1ccc(-c2cccc(-c3ccc(-n4c5ccccc5c5c6c7ccccc7n(-c7ccccc7)c6ccc54)cc3)c2)cc1] | 79% |
| 3-12 | 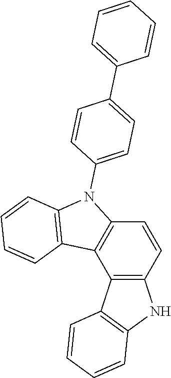 [SMILES: c1ccc(-c2ccc(-n3c4ccccc4c4c5c(ccc43)[nH]c3ccccc35)cc2)cc1] | 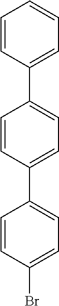 [SMILES: Brc1ccc(-c2ccc(-c3ccccc3)cc2)cc1] | 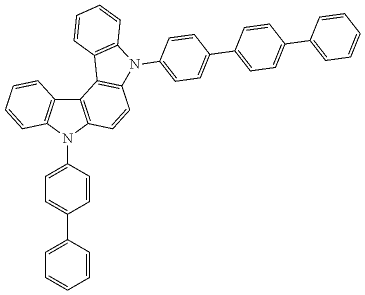 [SMILES: c1ccc(-c2ccc(-c3ccc(-n4c5ccccc5c5c6c7ccccc7n(-c7ccc(-c8ccccc8)cc7)c6ccc54)cc3)cc2)cc1] | 67% |
| 3-32 | 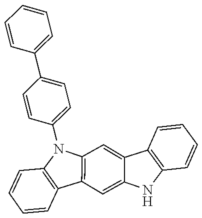 [SMILES: c1ccc(-c2ccc(-n3c4ccccc4c4cc5[nH]c6ccccc6c5cc43)cc2)cc1] | 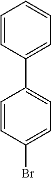 [SMILES: Brc1ccc(-c2ccccc2)cc1] | 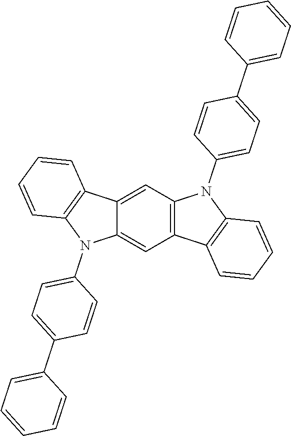 [SMILES: c1ccc(-c2ccc(-n3c4ccccc4c4cc5c(cc43)c3ccccc3n5-c3ccc(-c4ccccc4)cc3)cc2)cc1] | 84% |
| 3-41 | 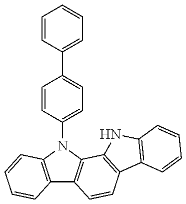 [SMILES: c1ccc(-c2ccc(-n3c4ccccc4c4ccc5c6ccccc6[nH]c5c43)cc2)cc1] | 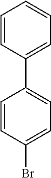 [SMILES: Brc1ccc(-c2ccccc2)cc1] | 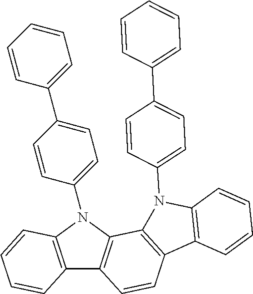 [SMILES: c1ccc(-c2ccc(-n3c4ccccc4c4ccc5c6ccccc6n(-c6ccc(-c7ccccc7)cc6)c5c43)cc2)cc1] | 60% |

### <Preparation Example 6-1> Preparation of Compound 3-80

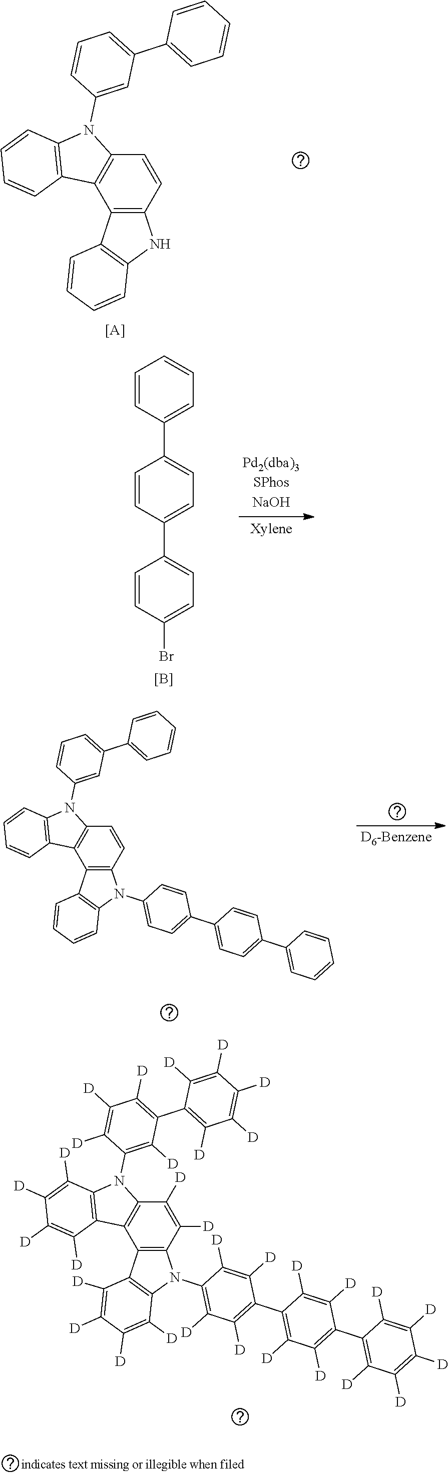

### 1) Preparation of Intermediate 3-80-1

5-([1,1′-biphenyl]-3-yl)-5,8-dihydroindolo[2,3-c]carbazole [A](10 g, 0.024 mol), 4-bromo-1,1′:4′,1″-terphenyl [B](7.42 g, 0.024 mol), Pd₂(dba)₃ (1.10 g, 0.0012 mol), SPhos (1.97 g, 0.0048 mmol), NaOH (1.92 g, 0.048 mol), and xylene (100 mL) were put into a one-neck round-bottom flask, and the mixture was refluxed at 153° C. After the mixture was cooled, extraction was performed, and the organic layer was silica gel-filtered to obtain Intermediate 3-80-1. (9.93 g, yield 65%)

### 2) Preparation of Compound 3-80

Intermediate 3-80-1 (9.93 g, 0.016 mol), triflic acid (40.8 g, 0.27 mol), and D₆-benzene (120 mL) were put into a one-neck round-bottom flask, and the mixture was refluxed at 70° C. The resulting product was quenched and extracted with DCM and H₂O and concentrated, and then silica gel-filtered. The filtered product was concentrated, and then treated with methanol to obtain Compound 3-80. (6.74 g, 63%)

### <Preparation Example 6-2> Preparation of Target Compound C of Following Table 6

The target compound C in the following Table 6 was obtained by performing synthesis in the same manner as in the above Preparation Example, except that the reaction products [A] and [B] in the above Preparation Example were changed to the reaction products A and B in the following Table 6.

TABLE 6

| Compound | Reaction | Reaction | Target | |
|---|---|---|---|---|
| No. | Product A | Product B | Compound C | Yield |
| 3-86 | 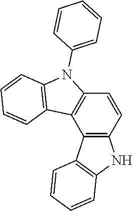 [SMILES: c1ccc(-n2c3ccccc3c3c4c(ccc32)[nH]c2ccccc24)cc1] | 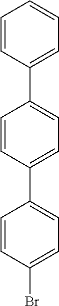 [SMILES: Brc1ccc(-c2ccc(-c3ccccc3)cc2)cc1] | 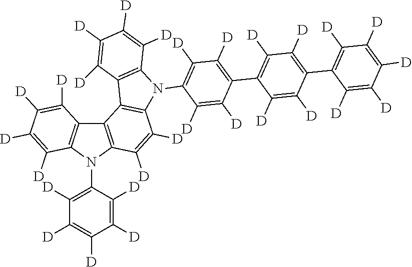 [SMILES: [2H]c1c([2H])c([2H])c(-c2c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c([2H])c3[2H])c([2H])c2[2H])c([2H])c1[2H]] | 67% |
| 3-98 | 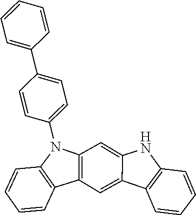 [SMILES: c1ccc(-c2ccc(-n3c4ccccc4c4cc5c(cc43)[nH]c3ccccc35)cc2)cc1] | 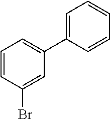 [SMILES: Brc1cccc(-c2ccccc2)c1] | 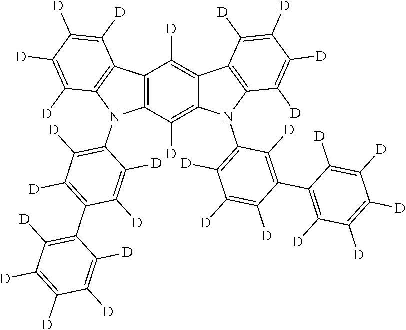 [SMILES: [2H]c1c([2H])c([2H])c(-c2c([2H])c([2H])c(-n3c4c([2H])c([2H])c([2H])c([2H])c4c4c([2H])c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c43)c([2H])c2[2H])c([2H])c1[2H]] | 79% |
| 3-112 | 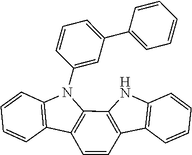 [SMILES: c1ccc(-c2cccc(-n3c4ccccc4c4ccc5c6ccccc6[nH]c5c43)c2)cc1] | 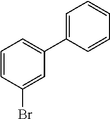 [SMILES: Brc1cccc(-c2ccccc2)c1] | 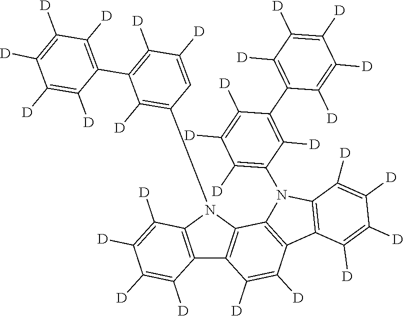 [SMILES: [2H]c1cc(-n2c3c([2H])c([2H])c([2H])c([2H])c3c3c([2H])c([2H])c4c5c([2H])c([2H])c([2H])c([2H])c5n(-c5c([2H])c([2H])c([2H])c(-c6c([2H])c([2H])c([2H])c([2H])c6[2H])c5[2H])c4c32)c([2H])c(-c2c([2H])c([2H])c([2H])c([2H])c2[2H])c1[2H]] | 84% |

It was confirmed by FD-mass spectrometry and ¹H-NMR that the compounds synthesized in the above Preparation Examples were synthesized as the desired compounds. The measured values by field desorption mass spectrometry (FD-Mass) are shown in the following Table 7, and the measured values by ¹HNMR (DMSO, 300 MHz) are shown in the following Table 8.

TABLE 7

| Compound | FD-Mass | Compound | FD-Mass |
|---|---|---|---|
| 1-1 | m/z = 751.27 | 1-3 | m/z = 751.27 |
| | (C51H25D6N5S = 751.94) | | (C51H25D6N5S = 751.94) |
| 1-5 | m/z = 752.27 | 1-12 | m/z = 810.32 |
| | (C51H24D7N5S = 752.95) | | (C57H30D5N5O = 810.97) |
| 1-19 | m/z = 736.30 | 1-21 | m/z = 827.30 |
| | (C51H24D7N5O = 736.89) | | (C57H29D6N5S = 828.04) |
| 1-22 | m/z = 827.30 | 1-23 | m/z = 827.30 |
| | (C57H29D6N5S = 828.04) | | (C57H29D6N5S = 828.04) |
| 1-25 | m/z = 826.29 | 1-29 | m/z = 826.29 |
| | (C57H30D5N5S = 827.03) | | (C57H30D5N5S = 827.03) |
| 1-34 | m/z = 810.32 | 1-38 | m/z = 812.33 |
| | (C57H30D5N5O = 810.97) | | (C57H28D7N5O = 812.98) |
| 1-42 | m/z = 812.35 | 1-44 | m/z = 813.36 |
| | (C57H28D8N6 = 813.01) | | (C57H27D9N6 = 813.36) |
| 1-48 | m/z = 811.34 | 1-52 | m/z = 761.34 |
| | (C57H29D7N6 = 812.00) | | (C54H31D6N5 = 761.34) |
| 1-57 | m/z = 857.26 | 1-58 | m/z = 825.30 |
| | (C57H27D6N5S2 = 858.08) | | (C57H27D6N5O2 = 825.96) |
| 1-59 | m/z = 857.26 | 1-64 | m/z = 843.25 |
| | (C57H27D6N5S2 = 858.08) | | (C57H29D5N4S2 = 844.08) |
| 1-67 | m/z = 751.25 | 1-69 | m/z = 725.25 |
| | (C51H25D5N4OS = 751.92) | | (C51H24D6N4OS = 752.92) |
| 1-72 | m/z = 736.27 | 1-80 | m/z = 752.25 |
| | (C51H24D6N4O2 = 736.86) | | (C51H24D6N4OS = 752.92) |
| 1-82 | m/z = 828.28 | 1-85 | m/z = 828.28 |
| | (C57H27D6N4OS = 829.02) | | (C57H27D6N4OS = 829.02) |
| 1-90 | m/z = 821.31 | 1-93 | m/z = 888.38 |
| | (C57H26D6N4O2 = 812.96) | | (C63H32D8N6 = 889.10) |
| 1-94 | m/z = 888.38 | 1-98 | m/z = 888.38 |
| | (C63H32D8N6 = 889.10) | | (C63H32D8N6 = 889.10) |
| 1-100 | m/z = 887.38 | 1-103 | m/z = 886.37 |
| | (C63H33D7N5 = 888.10) | | (C60H36D5N5 = 837.05) |
| 1-112 | m/z = 830.32 | 1-113 | m/z = 812.33 |
| | (C57H26D9N5S = 831.06) | | (C57H28D7N5O = 812.98) |
| 1-115 | m/z = 904.34 | 1-118 | m/z = 762.33 |
| | (C63H32D7N5S = 905.14) | | (C54H30D6N4O = 762.95) |
| 1-126 | m/z = 889.37 | 1-131 | m/z = 904.34 |
| | (C63H31D8N5O = 890.09) | | (C63H32D7N5S = 905.14) |
| 1-132 | m/z = 838.36 | 1-137 | m/z = 864.30 |
| | (C60H34D6N4O = 839.04) | | (C57H20D13N5S2 = 865.13) |
| 1-138 | m/z = 832.35 | 1-140 | m/z = 863.29 |
| | (C57H20D13N5O2 = 833.00) | | (C57H21D12N5S2 = 864.12) |
| 1-141 | m/z = 758.31 | 1-143 | m/z = 575.31 |
| | (C51H18D13N5S = 758.98) | | (C51H19D12N5S = 757.98) |
| 1-146 | m/z = 836.36 | 1-148 | m/z = 816.38 |
| | (C57H20D15N5S = 837.09) | | (C57H24D12N6 = 817.03) |
| 1-149 | m/z = 741.31 | 1-152 | m/z = 821.36 |
| | (C51H19D11N4O2 = 741.89) | | (C57H19D15N4O2 = 822.02) |
| 2-1 | m/z = 484.59(C36H24N2 = | 2-3 | m/z = 560.23 |
| | 484.19) | | (C42H28N2 = 560.70) |
| 2-4 | m/z = 560.23 | 2-16 | m/z = 634.24 |
| | (C42H28N2 = 560.70) | | (C48H30N2 = 634.78) |
| 2-27 | m/z = 636.26 | 2-28 | m/z = 636.26 |
| | (C48H32N2 = 636.80) | | (C48H32N2 = 636.80) |
| 2-32 | m/z = 636.26 | 2-82 | m/z = 668.46 |
| | (C48H32N2 = 636.80) | | (C48D32N2 = 668.99) |
| 2-85 | m/z = 668.46 | 2-88 | m/z = 668.46 |
| | (C48D32N2 = 668.99) | | (C48D32N2 = 668.99) |
| 2-89 | m/z = 668.46 | 3-4 | m/z = 560.23 |
| | (C48D32N2 = 668.99) | | (C42H28N2 = 560.70) |
| 3-11 | m/z = 574.20 | 3-12 | m/z = 560.23 |
| | (C42H26N2O = 574.68) | | (C42H28N2 = 560.70) |
| 3-32 | m/z = 560.23 | 3-41 | m/z = 560.23 |
| | (C42H28N2 = 560.70) | | (C42H28N2 = 560.70) |
| 3-80 | m/z = 668.46 | 3-86 | m/z = 588.40 |
| | (C48D32N2 = 668.99) | | (C42D28N2 = 588.87) |
| 3-98 | m/z = 588.40 | 3-112 | m/z = 587.39 |
| | (C42D28N2 = 588.87) | | (C42HD27N2 = 587.86) |

TABLE 8

| Compound | ¹H NMR(DMSO, 300 Mz) |
|---|---|
| 1-1 | δ = 8.55 (1H, d), 8.36 (2H, d), 8.19 (1H d), 7.91~7.99 (7H, m), 7.80 (2H, t), 7.46~7.58 |
| | (9H, m), 7.35 (1H, t), 7.16~7.20 (2H, t) |
| 1-3 | δ = 8.55 (1H, d), 8.36 (2H, d), 8.19~8.24 (3H, m), 7.91~7.94 (4H, m), 7.80 (1H, t), |
| | 7.46~7.68 (9H, m), 7.35 (1H, t), 7.16~7.20 (2H, t) |
| 1-5 | δ = 8.55 (1H, d), 8.36 (2H, d), 8.19 (1H, d), 7.92~7.99 (4H, m), 7.80~7.81 (2H, m), |
| | 7.46~7.58 (10H, m), 7.35 (1H, t), 7.16~7.20 (2H, t) |
| 1-12 | δ = 8.55 (1H, d), 8.19 (1H, d), 7.91~7.96 (8H, m), 7.75~7.80 (3H, m), 7.65 (1H, s), |
| | 7.35~7.58 (10H, m), 7.16~7.25 (4H, m) |
| 1-19 | δ = 8.55 (1H, d), 8.36 (2H, m), 8.19 (1H, d), 7.91~7.94 (5H, m), 7.80 (1H, t), 7.72 (1H, |
| | s), 7.46~7.60 (9H, m), 7.35 (1H, t), 7.16~7.20 (3H, m) |
| 1-21 | δ = 8.55 (1H, d), 8.36 (2H, m), 7.89~7.99 (8H, m), 7.75~7.80 (5H, m), 7.41~7.50 |
| | (10H, m), 8.36 (1H, t) |
| 1-22 | δ = 8.55 (1H, d), 8.31~8.36 (3H, m), 7.91~7.99 (8H, m), 7.74~7.80 (5H, m), 7.35~7.50 |
| | (10H, m), 7.16 (1H, t) |
| 1-23 | δ = 8.55 (1H, d), 8.21~8.36 (5H, m), 7.91~7.99 (6H, m), 7.60~7.80 (6H, m), 7.41~7.50 |
| | (9H, m), 7.16 (1H, t) |
| 1-25 | δ = 8.55 (1H, d), 8.29~8.36 (3H, m), 8.06 (1H, d), 7.19~7.99 (9H, m), 7.80 (1H, t), |
| | 7.41~7.50 (10H, m), 7.16~7.19 (5H, m) |
| 1-29 | δ = 8.55 (1H, d), 8.31~8.36 (3H, m), 7.91~7.96 (9H, m), 7.74~7.80 (5H, m), 7.31~7.50 |
| | (11H, m), 7.16 (1H, t) |
| 1-34 | δ = 8.55 (2H, d), 8.21~8.36 (5H, m), 7.91~7.94 (4H, m), 7.60~7.80 (7H, m), 7.36~7.50 |
| | (10H, m), 7.16~7.25 (2H, m) |
| 1-38 | δ = 8.55 (1H, d), 8.36 (2H, d), 7.89~7.94 (6H, m), 7.72~7.80 (5H, m), 7.35~7.63 (12H, |
| | m), 7.16~7.19 (2H, m) |
| 1-42 | δ = 8.55 (1H, d), 8.36 (2H, m), 8.18~8.24 (3H, m), 7.91~7.94 (4H, m), 7.29~7.81 |
| | (16H, m), 7.16~7.20 (2H, m) |
| 1-44 | δ = 8.55 (1H, d), 8.36~8.43 (3H, m), 8.19 (1H, d), 7.91~7.94 (5H, m), 7.73~7.80 (2H, |
| | m), 7.41~7.58 (12H, m), 7.16~7.26 (3H, m) |
| 1-48 | δ = 8.55 (1H, d), 8.36 (2H, m), 8.12~8.21 (4H, m), 7.92~7.96 (5H, m), 7.45~7.68 |
| | (12H, m), 7.35 (1H, t), 7.16~7.20 (3H, m) |
| 1-52 | δ = 8.55 (1H, d), 8.36 (2H, m), 8.19~8.24 (2H, m), 7.91~7.94 (5H, m), 7.80 (1H, t), |
| | 7.48~7.67 (10H, m), 7.35 (2H, m), 7.16~7.20 (2H, m), 1.69 (6H, s) |
| 1-57 | δ = 8.55 (1H, d), 8.45 (1H, d), 8.36 (2H, d), 8.05 (1H, d), 7.91~7.94 (7H, m), 7.80 (1H, |
| | t), 7.35~7.60 (11H, m), 7.16 (1H, t) |
| 1-58 | δ = 8.55 (1H, d), 8.36 (2H, d), 7.80~7.98 (10H, m), 7.31~7.54 (12H, m), 7.16 (1H, t) |
| 1-59 | δ = 8.55 (1H, d), 8.45 (1H, d), 8.36 (2H, m), 8.21~8.24 (2H, m), 8.05 (1H, d), |
| | 7.91~7.94 (5H, m), 7.80 (1H, t), 7.33~7.66 (13H, m), 7.16 (1H, t) |
| 1-64 | δ = 8.55 (2H, d), 8.32~8.38 (4H, m), 8.21~8.24 (2H, m), 7.94~7.99 (4H, m), 7.60~7.73 |
| | (6H, m), 7.41~7.51 (10H, m) |
| 1-67 | δ = 8.36~8.38 (3H, m), 7.91~7.99 (8H, m), 7.69~7.80 (4H, m), 7.31~7.57 (10H, m) |
| 1-69 | δ = 8.36~8.38 (3H, m), 7.91~8.08 (8H, m), 7.73~7.80 (2H, m), 7.31~7.61 (10H, m) |
| 1-72 | δ = 8.36 (2H, m), 7.91~8.03 (7H, m), 7.76~7.82 (3H, m), 7.31~7.65 (12H, m) |
| 1-80 | δ = 8.55 (1H, d), 8.45 (1H, d), 8.32~8.36 (3H, m), 8.21~8.24 (2H, m), 7.93~7.96 (4H, |
| | m), 7.49~7.70 (12H, m), 7.36 (1H, s) |
| 1-82 | δ = 8.36~8.38 (31h, m), 7.91~8.03 (6H, m), 7.69~7.82 (8H, m), 7.41~7.61 (11H, m) |
| 1-85 | δ = 8.36~8.38 (3H, m), 8.21~8.24 (2H, m), 7.46~7.99 (23H, m) |
| 1-90 | δ = 8.36 (2H, m), 7.91~8.03 (7H, m), 7.75~7.82 (7H, m), 7.60~7.65 (3H, m), |
| | 7.36~7.50 (9H, m) |
| 1-93 | δ = 8.55 (1H, d), 8.31~8.36 (3H, m), 7.91~7.94 (7H, m), 7.73~7.81 (7H, m), 7.41~7.50 |
| | (13H, m), 7.16 (1H, t) |
| 1-94 | δ = 8.55 (1H, d), 8.36 (2H, m), 8.21~8.24 (2H, m), 7.91~7.94 (4H, m), 7.29~7.68 |
| | (21H, m), 7.16 (1H, t) |
| 1-98 | δ = 8.55 (1H, d), 8.31~8.36 (3H, m), 7.91~7.97 (7H, m), 7.29~7.55 (15H, m), 7.16 |
| | (1H, t) |
| 1-100 | δ = 8.55 (1H, d), 8.21~8.36 (5H, m), 8.12 (1H, d), 7.91~7.96 (5H, m), 7.60~7.80 (6H, |
| | m), 7.35~7.50 (11H, m), 7.16~7.18 (2H, m) |
| 1-103 | δ = 8.55 (1H, d), 8.31~8.36 (3H, m), 8.06 (1H, s), 7.91~7.94 (9H, m), 7.74~7.80 (4H, |
| | m), 7.35~7.52 (10H, m), 7.16 (1H, t), 1.69 (6H, s) |
| 1-112 | δ = 8.55 (1H, d), 8.32~8.45 (6H, m), 7.91~7.94 (4H, m), 7.70~7.73 (3H, m), 7.41~7.61 |
| | (11H, m) |
| 1-113 | δ = 8.36~8.43 (4H, m), 8.21~8.24 (2H, m), 7.94~8.08 (5H, m), 7.26~7.73 (17H, m) |
| 1-115 | δ = 8.55 (1H, d), 8.32~8.45 (3H, m), 7.91~7.96 (8H, m), 7.70~7.81 (6H, m), 7.41~7.61 |
| | (11H, m), 7.25 (2H, d) |
| 1-118 | δ = 8.36~8.38 (3H, m), 8.21~8.24 (2H, m), 7.94~7.98 (3H, m), 7.82 (1H, d), 7.50~7.73 |
| | (11H, m), 7.31~7.39 (3H, m) |
| 1-126 | δ = 8.36~8.38 (3H, m), 8.21 (1H, s), 8.03 (1H, d), 7.94 (2H, m), 7.41~7.83 (22H, m), |
| | 7.29 (1H, d) |
| 1-131 | δ = 8.55 (1H, d), 8.32~8.36 (3H, m), 8.17~8.24 (3H, m), 7.91~7.94 (5H, m), 7.73~7.81 |
| | (7H, m), 7.61 (1H, s), 7.41~7.50 (11H, m) |
| 1-132 | δ = 8.36~8.38 (3H, m), 8.21~8.24 (2H, m), 8.03 (2H, d), 7.94 (2H, m), 7.35~7.82 |
| | (20H, m), 1.69 (6H, s) |
| 1-137 | δ = 8.36 (2H, m), 7.91~7.94 (6H, m), 7.80 (2H, t), 7.33~7.50 (9H, m) |
| 1-138 | δ = 8.36 (2H, m), 7.91~7.94 (6H, m), 7.80 (2H, t), 7.33~7.50 (10H, m) |
| 1-140 | δ = 8.36 (2H, d), 7.91~7.94 (8H, m), 7.80 (2H, t), 7.33~7.50 (9H, m) |
| 1-141 | δ = 8.36 (2H, m), 7.91~7.99 (5H, m), 7.80 (1H, t), 7.64 (1H, s), 7.41~7.50 (9H, m) |
| 1-143 | δ = 8.36 (2H, m), 8.21~8.24 (2H, m), 7.91~7.99 (5H, m), 7.80 (1H, t), 7.60~7.68 (2H, |
| | m), 7.46~7.50 (6H, m) |
| 1-146 | δ = 8.36 (2H, m), 7.91~7.99 (7H, m), 7.80~7.84 (2H, m), 7.64~7.67 (2H, d), 7.33~7.50 |
| | (7H, m) |
| 1-148 | δ = 8.36 (2H, m), 8.21~8.24 (2H, m), 8.12 (1H, d), 7.91~7.96 (5H, m), 7.60~7.80 (4H, |
| | m), 7.42~7.50 (9H, m), 7.18 (1H, d) |
| 1-149 | δ = 8.36~8.38 (3H, m), 8.21~8.24 (2H, m), 7.94 (2H, m), 7.60~7.75 (7H, m), |
| | 7.49~7.50 (4H, m), 7.36 (1H, s) |
| 1-152 | δ = 8.36 (2H, m), 8.21~8.24 (2H, m), 8.06 (1H, s), 7.94~7.96 (2H, m), 7.82 (2H, d), |
| | 7.60~7.68 (4H, m), 7.49~7.52 (5H, m), 7.36 (1H, s) |
| 2-1 | δ = 8.55 (1H, d), 8.30 (1H, d), 8.13~8.19 (2H, m), 7.89~7.99 (4H, m), 7.77 (1H, d), |
| | 7.50~7.62 (12H, m), 7.35 (1H, t), 7.16~7.20 (2H, t) |
| 2-3 | δ = 8.55 (1H, d), 8.30 (1H, d), 8.13~8.21 (3H, m), 7.89~7.99 (4H, m), 7.35~7.77 (17H, |
| | m), 7.16~7.20 (2H, m) |
| 2-4 | δ = 8.55 (1H, d), 8.30 (1H, d), 8.13~8.19 (2H, m), 7.89~7.99 (8H, m), 7.75~7.77 (3H, |
| | m), 7.35~7.62 (11H, m), 7.16~7.20 (2H, m) |
| 2-16 | δ = 9.05 (1H, s), 8.55 (1H, d), 8.13~8.33 (7H, m), 7.89~7.99 (5H, m), 7.50~7.77 (13H, |
| | m), 7.35 (1H, t), 7.16~7.20 (2H, t) |
| 2-27 | δ = 8.55 (1H, d), 8.30 (1H, d), 8.13~8.21 (3H, m), 7.89~7.99 (8H, m), 7.35~7.77 (17H, |
| | m), 7.16~7.20 (2H, m) |
| 2-28 | δ = 8.55 (1H, d), 8.30 (1H, d), 8.13~8.21 (3H, m), 7.89~7.99 (8H, m), |
| 2-32 | δ = 8.55 (1H, d), 8.30 (1H, d), 8.13~8.19 (2H, m), 7.89~7.99 (12H, m), 7.75~7.77 (5H, |
| | m), 7.58 (1H, d), 7.35~7.50 (8H, m), 7.16~7.20 (2H, m) |
| 2-82 | δ = deuterium content of 100% with no ¹H NMR peak |
| 2-85 | δ = deuterium content of 100% with no ¹H NMR peak |
| 2-88 | δ = deuterium content of 100% with no ¹H NMR peak |
| 2-89 | δ = deuterium content of 100% with no ¹H NMR peak |
| 3-4 | δ = 8.55 (2H, d), 7.91~7.94 (10H, m), 7.75 (4H, d), 7.35~7.49 (10H, m), 7.16 (2H, t) |
| 3-11 | δ = 8.55 (2H, d), 7.91~7.94 (7H, m), 7.73~7.75 (3H, m), 7.35~7.62 (18H, m), 7.26 |
| | (1H, d), 7.16 (2H, t) |
| 3-12 | δ = 8.55 (2H, d), 7.91~7.94 (10H, m), 7.75 (4H, d), 7.58 (1H, d), 7.35~7.49 (8H, m), |
| | 7.25 (4H, d), 7.16 (1H, t) |
| 3-32 | δ = 8.55 (1H, d), 8.19 (1H, d), 7.91~7.94 (9H, m), 7.75 (4H, d), 7.35~7.58 (11H, m), |
| | 7.16~7.20 (2H, m) |
| 3-41 | δ = 8.55 (2H, d), 7.91~7.94 (10H, m), 7.75 (4H, d), 7.35~7.49 (8H, m), 7.16 (2H, t) |
| 3-80 | δ = deuterium content of 100% with no ¹H NMR peak |
| 3-86 | δ = deuterium content of 100% with no ¹H NMR peak |
| 3-98 | δ = deuterium content of 100% with no ¹H NMR peak |
| 3-112 | δ = deuterium content of 100% with no ¹H NMR peak |

### Experimental Example 1

### 1) Manufacture of Organic Light Emitting Device

A glass substrate, in which ITO was thinly coated to have a thickness of 1,500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to have a thickness of 400 Å by using a compound described in the following Table 9 as a host and tris(2-phenylpyridine)iridium (Ir(ppy)₃) as a green phosphorescent dopant to dope the host with Ir(ppy)₃ in an amount of 7%. Thereafter, bathocuproine (BCP) as a hole blocking layer was deposited to have a thickness of 60 Å, and Alq₃ as an electron transport layer was deposited to have a thickness of 200 Å thereon. Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1200 Å on the electron injection layer to form a negative electrode.

Meanwhile, all the organic compounds required for manufacturing an OLED device were subjected to vacuum sublimed purification under 10⁻⁸ to 10⁻⁶ torr for each material, and used for the manufacture of the OLED.

### 2) Driving Voltage and Light Emitting Efficiency of Organic Light Emitting Device

For the organic light emitting device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m².

The results of measuring the driving voltage, light emitting efficiency, color coordinate (CIE) and service life of the organic light emitting device manufactured according to the present invention are shown in the following Table 9.

The structures of Comparative Compounds A to M are as follows.

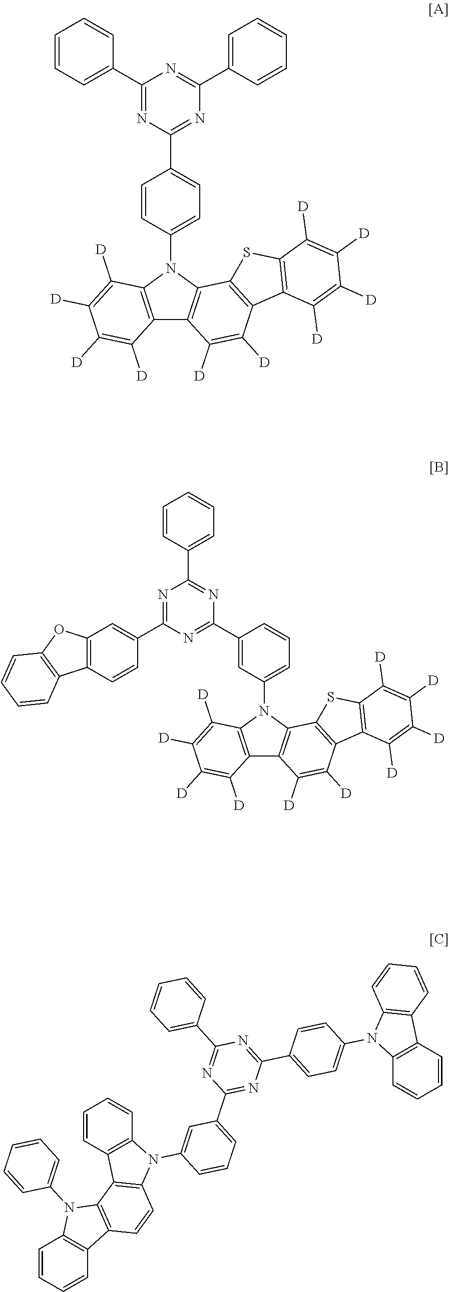 [SMILES: [2H]c1c([2H])c([2H])c2c(sc3c2c([2H])c([2H])c2c4c([2H])c([2H])c([2H])c([2H])c4n(-c4ccc(-c5nc(-c6ccccc6)nc(-c6ccccc6)n5)cc4)c32)c1[2H] ; [2H]c1c([2H])c([2H])c2c(sc3c2c([2H])c([2H])c2c4c([2H])c([2H])c([2H])c([2H])c4n(-c4cccc(-c5nc(-c6ccccc6)nc(-c6ccc7c(c6)oc6ccccc67)n5)c4)c32)c1[2H] ; c1ccc(-c2nc(-c3ccc(-n4c5ccccc5c5ccccc54)cc3)nc(-c3cccc(-n4c5ccccc5c5c4ccc4c6ccccc6n(-c6ccccc6)c45)c3)n2)cc1]

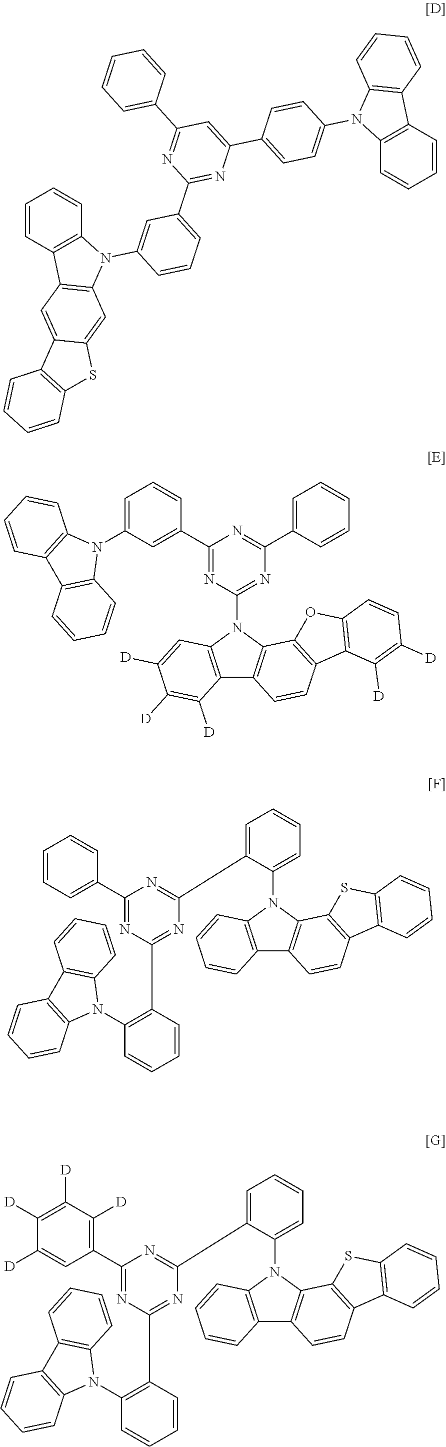

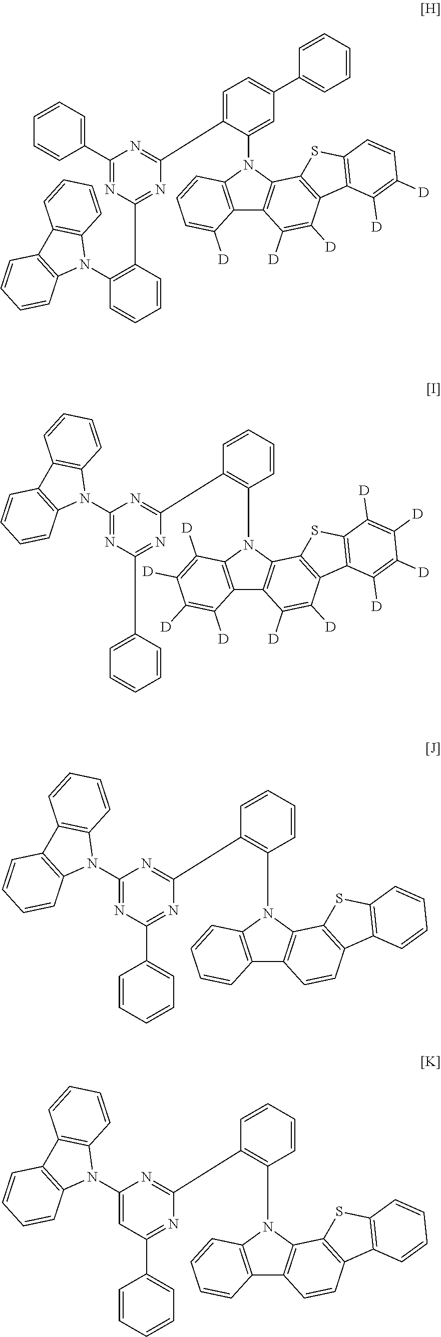

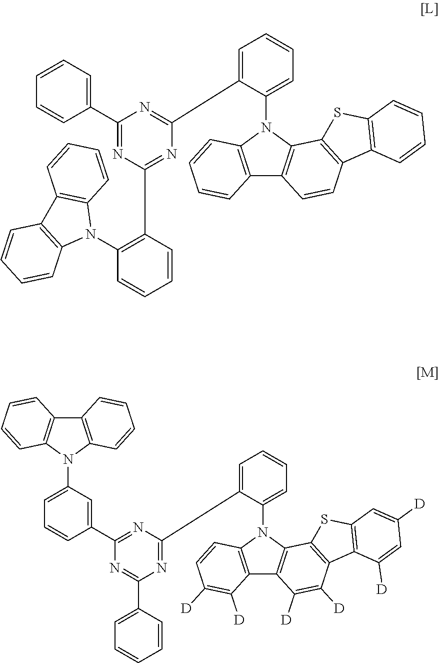

TABLE 9

| | | Driving | | | Service |
|---|---|---|---|---|---|
| | | voltage | Efficiency | | life |
| | Compound | (V) | (cd/A) | Color | (T₉₀) |
| Comparative | A | 6.56 | 55.6 | Green | 75 |
| Example 1 | | | | | |
| Comparative | B | 6.66 | 56.2 | Green | 77 |
| Example 2 | | | | | |
| Comparative | C | 6.52 | 54.3 | Green | 85 |
| Example 3 | | | | | |
| Comparative | D | 6.70 | 53.2 | Green | 86 |
| Example 4 | | | | | |
| Comparative | E | 6.65 | 52.2 | Green | 83 |
| Example 5 | | | | | |
| Comparative | F | 6.53 | 55.6 | Green | 80 |
| Example 6 | | | | | |
| Comparative | G | 6.42 | 58.7 | Green | 85 |
| Example 7 | | | | | |
| Comparative | H | 6.44 | 60.2 | Green | 84 |
| Example 8 | | | | | |
| Comparative | I | 6.65 | 50.3 | Green | 70 |
| Example 9 | | | | | |
| Comparative | J | 6.64 | 50.8 | Green | 73 |
| Example 10 | | | | | |
| Comparative | K | 6.69 | 51.2 | Green | 71 |
| Example 11 | | | | | |
| Comparative | L | 6.68 | 50.1 | Green | 72 |
| Example 12 | | | | | |
| Comparative | M | 6.71 | 48.7 | Green | 68 |
| Example 13 | | | | | |
| Example 1 | 1-1 | 4.56 | 155.3 | Green | 250 |
| Example 2 | 1-3 | 4.63 | 154.2 | Green | 241 |
| Example 3 | 1-5 | 4.66 | 150.8 | Green | 244 |
| Example 4 | 1-12 | 4.32 | 160.2 | Green | 263 |
| Example 5 | 1-19 | 4.38 | 159.5 | Green | 258 |
| Example 6 | 1-21 | 4.46 | 164.2 | Green | 252 |
| Example 7 | 1-22 | 4.32 | 163.8 | Green | 244 |
| Example 8 | 1-23 | 4.35 | 161.9 | Green | 256 |
| Example 9 | 1-25 | 4.48 | 160.6 | Green | 258 |
| Example 10 | 1-29 | 4.29 | 123.8 | Green | 267 |
| Example 11 | 1-34 | 4.25 | 132.6 | Green | 264 |
| Example 12 | 1-38 | 4.37 | 133.8 | Green | 261 |
| Example 13 | 1-42 | 4.52 | 123.9 | Green | 260 |
| Example 14 | 1-44 | 4.55 | 130.7 | Green | 268 |
| Example 15 | 1-48 | 4.62 | 133.9 | Green | 270 |
| Example 16 | 1-52 | 4.43 | 134.2 | Green | 274 |
| Example 17 | 1-57 | 4.38 | 152.9 | Green | 300 |
| Example 18 | 1-58 | 4.28 | 155.3 | Green | 294 |
| Example 19 | 1-59 | 4.23 | 165.2 | Green | 298 |
| Example 20 | 1-64 | 5.64 | 85.5 | Green | 180 |
| Example 21 | 1-67 | 5.52 | 84.6 | Green | 174 |
| Example 22 | 1-69 | 5.48 | 82.7 | Green | 177 |
| Example 23 | 1-72 | 5.55 | 80.5 | Green | 172 |
| Example 24 | 1-80 | 5.49 | 91.8 | Green | 173 |
| Example 25 | 1-82 | 5.42 | 92.5 | Green | 168 |
| Example 26 | 1-85 | 5.44 | 88.3 | Green | 165 |
| Example 27 | 1-90 | 5.62 | 87.2 | Green | 179 |
| Example 28 | 1-93 | 4.66 | 164.2 | Green | 250 |
| Example 29 | 1-94 | 4.69 | 165.5 | Green | 255 |
| Example 30 | 1-98 | 4.75 | 152.8 | Green | 262 |
| Example 31 | 1-100 | 4.72 | 150.3 | Green | 274 |
| Example 32 | 1-103 | 4.88 | 151.7 | Green | 270 |
| Example 33 | 1-112 | 5.77 | 92.4 | Green | 180 |
| Example 34 | 1-113 | 5.82 | 91.5 | Green | 174 |
| Example 35 | 1-115 | 5.74 | 90.3 | Green | 156 |
| Example 36 | 1-118 | 5.70 | 87.2 | Green | 163 |
| Example 37 | 1-126 | 5.68 | 88.5 | Green | 166 |
| Example 38 | 1-131 | 5.72 | 84.3 | Green | 152 |
| Example 39 | 1-132 | 5.66 | 85.9 | Green | 150 |
| Example 40 | 1-137 | 3.62 | 170.5 | Green | 342 |
| Example 41 | 1-138 | 3.69 | 173.2 | Green | 350 |
| Example 42 | 1-140 | 3.70 | 175.1 | Green | 310 |
| Example 43 | 1-141 | 3.74 | 180.6 | Green | 325 |
| Example 44 | 1-143 | 3.87 | 177.4 | Green | 348 |
| Example 45 | 1-146 | 3.59 | 172.3 | Green | 344 |
| Example 46 | 1-148 | 3.56 | 171.9 | Green | 312 |
| Example 47 | 1-149 | 5.12 | 97.3 | Green | 230 |
| Example 48 | 1-152 | 5.23 | 98.2 | Green | 229 |

From the results in the above table, it can be confirmed that the results of Examples 1 to 48, in which the heterocyclic compound of the present invention was used, are better than those of Comparative Examples 1 to 13.

Specifically, the heterocyclic compound of the present invention includes, as the characteristic structures thereof, disubstituted benzene (Feature 1), a substituent Ar2 (Feature 2), a deuterium-containing carbazole derivative (Feature 3), and triazine (Feature 4).

In Examples 1 to 48, the heterocyclic compound of the present invention, which satisfies all the characteristics, was used as a material for an organic light emitting device, and it can be seen that the driving voltage of the device was lowered and the efficiency and service life were remarkably improved.

In contrast, the compounds used in each of the comparative examples do not satisfy some of the characteristics of the present invention, as shown in the following Table 10.

TABLE 10

| | | | Feature 3 | |
|---|---|---|---|---|
| | | | (Deuterium- | |
| | Feature 1 | Feature 2 | containing | |
| | (Disubstituted | (Substituent | carbazole | Feature 4 |
| | benzene) | Ar2) | derivative) | (Triazine) |
| Comparative | ◯ | X | ◯ | ◯ |
| Compound A | | | | |
| Comparative | ◯ | X | ◯ | ◯ |
| Compound B | | | | |
| Comparative | ◯ | X | X | ◯ |
| Compound C | | | | |
| Comparative | ◯ | X | X | X |
| Compound D | | | | |
| Comparative | X | X | ◯ | ◯ |
| Compound E | | | | |
| Comparative | ◯ | ◯ | X | ◯ |
| Compound F | | | | |
| Comparative | ◯ | ◯ | X | ◯ |
| Compound G | | | | |
| Comparative | X | ◯ | ◯ | ◯ |
| Compound H | | | | |
| Comparative | ◯ | X | ◯ | ◯ |
| Compound I | | | | |
| Comparative | ◯ | X | X | ◯ |
| Compound J | | | | |
| Comparative | ◯ | X | X | X |
| Compound K | | | | |
| Comparative | ◯ | ◯ | X | X |
| Compound L | | | | |
| Comparative | ◯ | X | ◯ | ◯ |
| Compound M | | | | |

In particular, Comparative Compound G (Comparative Example 7) includes deuterium in a phenyl group, which is a substituent of triazine, and thus includes deuterium based on the overall structure, but does not include deuterium in the carbazole derivative as in the present invention. Comparative Compound G is different from Compound 1-1 (Example 1) of the present invention in terms of deuterium substitution position, but when comparing each of the evaluation results, it was confirmed that the driving voltage of Example 1 was reduced by 30%, the efficiency was about 2.6-fold increased, and the service life was nearly 3-fold increased, compared to Comparative Example 7. From this, it can be seen that the performance improvement effect is not shown even though deuterium is included in the entire compound, but a more improved effect is exhibited when deuterium is included at a specific position, that is, in the carbazole derivative of the present invention.

Furthermore, Comparative Compound H (Comparative Example 8) includes a trisubstituted benzene structure and thus does not satisfy Feature 1 of the present invention. Comparative Compound H is also different from Compound 1-1 (Example 1) of the present invention in that there is a difference in the configuration of the additional substituent (a phenyl group) on the benzene, and the number of deuterium atoms in the carbazole derivative in Comparative Compound H is smaller by 1 than that in Example 1. Therefore, it can be determined that there is no significant difference in structure between Comparative Compound H and Compound 1-1, but from the above evaluation results, it can be confirmed that the above difference results in remarkable improvement effects in terms of all of the driving voltage, efficiency, and service life.

Further, Comparative Compound I (Comparative Example 9) does not satisfy Feature 2 of the present invention in that there is no linker between the triazine and carbazole groups. Likewise, although only a linker structure was omitted based on Compound 1-1 (Example 1) of the present invention, the evaluation results confirmed that the data of Comparative Example 9 had a 1.45-fold higher driving voltage, an efficiency at a level of 30%, and a service life at a level of 28%, compared to Example 1.

Finally, Comparative Compound M (Comparative Example 13) does not satisfy Feature 2 of the present invention in that the carbazole group and triazine group of the benzene ring are substituted at the meta position in the substituent Ar2 of triazine. Comparative Compound M (Comparative Example 13) is different from Compound 1-1 (Example 1) of the present invention only in the substitution position of the reference substituent Ar2, but it was confirmed that the driving voltage was 1.47-fold higher, the efficiency was reduced by 67%, and the service life was less than 30%, compared to the evaluation results of Example 1.

Therefore, it can be seen that heterocyclic compounds satisfying all the characteristics of the present invention provide remarkably excellent performance as materials for an organic light emitting device compared to compounds with similar structures.

### Experimental Example 2

### 1) Manufacture of Organic Light Emitting Device

A glass substrate, in which ITO was thinly coated to have a thickness of 1,500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to 400 Å by including one type of the heterocyclic compounds having a structure of Chemical Formula 1 or the comparative compounds described in the following Table 11 and one type of the compounds having a structure of Chemical Formula 2 or 3 as hosts at the ratio (weight ratio) described in the following Table 11, using tris(2-phenylpyridine)iridium (Ir(ppy)₃) as a green phosphorescent dopant, and doping the host with Ir(ppy)₃ in an amount of 7%. Thereafter, bathocuproine (BCP) as a hole blocking layer was deposited to have a thickness of 60 Å, and Alq₃ as an electron transport layer was deposited to have a thickness of 200 Å thereon. Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1200 Å on the electron injection layer to form a negative electrode.

Meanwhile, all the organic compounds required for manufacturing an OLED device were subjected to vacuum sublimed purification under 10⁻⁸ to 10⁻⁶ torr for each material, and used for the manufacture of the OLED.

### 2) Driving Voltage and Light Emitting Efficiency of Organic Light Emitting Device

For the organic light emitting device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/in².

The results of measuring the driving voltage, light emitting efficiency, color coordinate (CIE), and service life of the organic light emitting device manufactured according to the present invention are shown in the following Table 11.

TABLE 11

| | | | Driving | Effi- | Color | Service |
|---|---|---|---|---|---|---|
| | | | voltage | ciency | coor- | life |
| | Compound | Ratio | (V) | (cd/A) | dinate | (T₉₀) |
| Comparative | [A]:2-82 | 1:1 | 6.11 | 76.5 | Green | 100 |
| Example 14 | | | | | | |
| Comparative | | 1:2 | 6.13 | 75.1 | Green | 106 |
| Example 15 | | | | | | |
| Comparative | | 1:3 | 6.18 | 74.8 | Green | 115 |
| Example 16 | | | | | | |
| Comparative | [B]:2-89 | 1:1 | 6.15 | 76.2 | Green | 103 |
| Example 17 | | | | | | |
| Comparative | | 1:2 | 6.19 | 75.8 | Green | 108 |
| Example 18 | | | | | | |
| Comparative | | 1:3 | 6.21 | 74.3 | Green | 111 |
| Example 19 | | | | | | |
| Comparative | [C]:2-88 | 1:1 | 6.15 | 72.3 | Green | 105 |
| Example 20 | | | | | | |
| Comparative | | 1:2 | 6.17 | 71.6 | Green | 109 |
| Example 21 | | | | | | |
| Comparative | | 1:3 | 6.19 | 70.4 | Green | 114 |
| Example 22 | | | | | | |
| Comparative | [D]:3-98 | 1:1 | 6.17 | 73.2 | Green | 98 |
| Example 23 | | | | | | |
| Comparative | | 1:2 | 6.18 | 72.5 | Green | 102 |
| Example 24 | | | | | | |
| Comparative | | 1:3 | 6.20 | 71.4 | Green | 109 |
| Example 25 | | | | | | |
| Comparative | [E]:3-80 | 1:1 | 6.13 | 65.2 | Green | 95 |
| Example 26 | | | | | | |
| Comparative | | 1:2 | 6.15 | 64.8 | Green | 97 |
| Example 27 | | | | | | |
| Comparative | | 1:3 | 6.19 | 63.2 | Green | 99 |
| Example 28 | | | | | | |
| Comparative | [F]:3-86 | 1:1 | 6.02 | 68.7 | Green | 94 |
| Example 29 | | | | | | |
| Comparative | | 1:2 | 6.08 | 67.2 | Green | 96 |
| Example 30 | | | | | | |
| Comparative | | 1:3 | 6.11 | 66.3 | Green | 99 |
| Example 31 | | | | | | |
| Comparative | [G]:3-112 | 1:1 | 6.05 | 70.3 | Green | 100 |
| Example 32 | | | | | | |
| Comparative | | 1:2 | 6.07 | 69.7 | Green | 109 |
| Example 33 | | | | | | |
| Comparative | | 1:3 | 6.09 | 68.5 | Green | 116 |
| Example 34 | | | | | | |
| Comparative | [H]:2-89 | 1:1 | 6.01 | 74.2 | Green | 118 |
| Example 35 | | | | | | |
| Comparative | | 1:2 | 6.03 | 73.5 | Green | 120 |
| Example 36 | | | | | | |
| Comparative | | 1:3 | 6.08 | 72.1 | Green | 122 |
| Example 37 | | | | | | |
| Comparative | [I]:3-80 | 1:1 | 6.21 | 65.3 | Green | 93 |
| Example 38 | | | | | | |
| Comparative | | 1:2 | 6.23 | 65.0 | Green | 96 |
| Example 39 | | | | | | |
| Comparative | | 1:3 | 6.25 | 64.2 | Green | 98 |
| Example 40 | | | | | | |
| Comparative | [J]:3-98 | 1:1 | 6.20 | 65.8 | Green | 94 |
| Example 41 | | | | | | |
| Comparative | | 1:2 | 6.22 | 65.5 | Green | 96 |
| Example 42 | | | | | | |
| Comparative | | 1:3 | 6.24 | 64.9 | Green | 98 |
| Example 43 | | | | | | |
| Comparative | [K]:2-89 | 1:1 | 6.19 | 63.8 | Green | 90 |
| Example 44 | | | | | | |
| Comparative | | 1:2 | 6.21 | 63.2 | Green | 93 |
| Example 45 | | | | | | |
| Comparative | | 1:3 | 6.23 | 63.0 | Green | 95 |
| Example 46 | | | | | | |
| Comparative | [L]:3-98 | 1:1 | 6.18 | 63.5 | Green | 91 |
| Example 47 | | | | | | |
| Comparative | | 1:2 | 6.19 | 63.1 | Green | 95 |
| Example 48 | | | | | | |
| Comparative | | 1:3 | 6.21 | 62.9 | Green | 97 |
| Example 49 | | | | | | |
| Comparative | [M]:3-98 | 1:1 | 6.25 | 62.2 | Green | 87 |
| Example 50 | | | | | | |
| Comparative | | 1:2 | 6.28 | 61.8 | Green | 90 |
| Example 51 | | | | | | |
| Comparative | | 1:3 | 6.32 | 61.5 | Green | 92 |
| Example 52 | | | | | | |
| Example 49 | 1-1:2-89 | 1:1 | 2.99 | 185.2 | Green | 350 |
| Example 50 | | 1:2 | 3.02 | 184.3 | Green | 355 |
| Example 51 | | 1:3 | 3.13 | 182.0 | Green | 359 |
| Example 52 | 1-3:2-88 | 1:1 | 2.87 | 180.5 | Green | 362 |
| Example 53 | | 1:2 | 2.94 | 179.3 | Green | 366 |
| Example 54 | | 1:3 | 2.97 | 177.2 | Green | 368 |
| Example 55 | 1-5:2-88 | 1:1 | 3.12 | 170.3 | Green | 320 |
| Example 56 | | 1:2 | 3.24 | 168.7 | Green | 329 |
| Example 57 | | 1:3 | 3.29 | 165.2 | Green | 333 |
| Example 58 | 1-12:3-98 | 1:1 | 3.05 | 183.2 | Green | 342 |
| Example 59 | | 1:2 | 3.14 | 182.0 | Green | 349 |
| Example 60 | | 1:3 | 3.23 | 181.2 | Green | 354 |
| Example 61 | 1-19:3-112 | 1:1 | 2.97 | 172.6 | Green | 355 |
| Example 62 | | 1:2 | 2.99 | 171.2 | Green | 362 |
| Example 63 | | 1:3 | 3.04 | 170.3 | Green | 366 |
| Example 64 | 1-21:3-98 | 1:1 | 3.24 | 177.3 | Green | 374 |
| Example 65 | | 1:2 | 3.35 | 175.2 | Green | 382 |
| Example 66 | | 1:3 | 3.42 | 174.0 | Green | 385 |
| Example 67 | 1-22:3-86 | 1:1 | 3.21 | 182.6 | Green | 377 |
| Example 68 | | 1:2 | 3.26 | 180.3 | Green | 385 |
| Example 69 | | 1:3 | 3.30 | 178.2 | Green | 393 |
| Example 70 | 1-23:2-89 | 1:1 | 3.23 | 177.3 | Green | 372 |
| Example 71 | | 1:2 | 3.25 | 173.5 | Green | 376 |
| Example 72 | | 1:3 | 3.29 | 170.2 | Green | 380 |
| Example 73 | 1-25:2-89 | 1:1 | 3.41 | 174.5 | Green | 326 |
| Example 74 | | 1:2 | 3.45 | 172.6 | Green | 330 |
| Example 75 | | 1:3 | 3.49 | 171.3 | Green | 331 |
| Example 76 | 1-29:3-112 | 1:1 | 3.11 | 183.2 | Green | 352 |
| Example 77 | | 1:2 | 3.16 | 182.5 | Green | 355 |
| Example 78 | | 1:3 | 3.20 | 181.3 | Green | 364 |
| Example 79 | 1-34:3-98 | 1:1 | 2.97 | 177.6 | Green | 359 |
| Example 80 | | 1:2 | 3.02 | 175.3 | Green | 368 |
| Example 81 | | 1:3 | 3.14 | 173.2 | Green | 372 |
| Example 82 | 1-38:3-80 | 1:1 | 2.94 | 184.3 | Green | 355 |
| Example 83 | | 1:2 | 2.96 | 182.6 | Green | 364 |
| Example 84 | | 1:3 | 3.09 | 181.2 | Green | 370 |
| Example 85 | 1-42:2-85 | 1:1 | 3.12 | 177.5 | Green | 380 |
| Example 86 | | 1:2 | 3.26 | 174.2 | Green | 388 |
| Example 87 | | 1:3 | 3.29 | 173.0 | Green | 391 |
| Example 88 | 1-44:2-85 | 1:1 | 3.42 | 183.2 | Green | 374 |
| Example 89 | | 1:2 | 3.44 | 182.5 | Green | 377 |
| Example 90 | | 1:3 | 3.52 | 181.3 | Green | 380 |
| Example 91 | 1-48:3-98 | 1:1 | 3.15 | 175.2 | Green | 345 |
| Example 92 | | 1:2 | 3.21 | 174.6 | Green | 352 |
| Example 93 | | 1:3 | 3.26 | 173.2 | Green | 364 |
| Example 94 | 1-52:3-80 | 1:1 | 3.11 | 179.5 | Green | 355 |
| Example 95 | | 1:2 | 3.14 | 178.4 | Green | 364 |
| Example 96 | | 1:3 | 3.22 | 177.0 | Green | 370 |
| Example 97 | 1-57:3-80 | 1:1 | 3.24 | 169.2 | Green | 359 |
| Example 98 | | 1:2 | 3.29 | 168.5 | Green | 361 |
| Example 99 | | 1:3 | 3.32 | 162.8 | Green | 366 |
| Example 100 | 1-58:3-86 | 1:1 | 2.97 | 174.2 | Green | 345 |
| Example 101 | | 1:2 | 3.12 | 173.5 | Green | 352 |
| Example 102 | | 1:3 | 3.25 | 172.6 | Green | 355 |
| Example 103 | 1-59:2-89 | 1:1 | 2.87 | 180.6 | Green | 356 |
| Example 104 | | 1:2 | 2.93 | 179.3 | Green | 364 |
| Example 105 | | 1:3 | 2.95 | 178.2 | Green | 369 |
| Example 106 | 1-64:3-98 | 1:1 | 5.12 | 150.2 | Green | 250 |
| Example 107 | | 1:2 | 5.20 | 149.7 | Green | 252 |
| Example 108 | | 1:3 | 5.22 | 148.3 | Green | 255 |
| Example 109 | 1-67:2-88 | 1:1 | 5.06 | 149.5 | Green | 254 |
| Example 110 | | 1:2 | 5.13 | 147.2 | Green | 263 |
| Example 111 | | 1:3 | 5.15 | 146.2 | Green | 266 |
| Example 112 | 1-69:3-98 | 1:1 | 5.11 | 145.3 | Green | 249 |
| Example 113 | | 1:2 | 5.16 | 142.6 | Green | 253 |
| Example 114 | | 1:3 | 5.19 | 141.8 | Green | 261 |
| Example 115 | 1-72:2-85 | 1:1 | 4.92 | 132.6 | Green | 203 |
| Example 116 | | 1:2 | 4.99 | 131.2 | Green | 212 |
| Example 117 | | 1:3 | 5.02 | 130.8 | Green | 223 |
| Example 118 | 1-80:2-82 | 1:1 | 4.87 | 133.6 | Green | 215 |
| Example 119 | | 1:2 | 4.93 | 132.7 | Green | 226 |
| Example 120 | | 1:3 | 4.95 | 130.5 | Green | 230 |
| Example 121 | 1-82:3-98 | 1:1 | 5.13 | 128.7 | Green | 223 |
| Example 122 | | 1:2 | 5.20 | 127.5 | Green | 235 |
| Example 123 | | 1:3 | 5.23 | 126.3 | Green | 241 |
| Example 124 | 1-85:3-80 | 1:1 | 5.15 | 125.6 | Green | 217 |
| Example 125 | | 1:2 | 5.19 | 124.9 | Green | 225 |
| Example 126 | | 1:3 | 5.23 | 123.2 | Green | 231 |
| Example 127 | 1-90:2-82 | 1:1 | 4.82 | 116.5 | Green | 209 |
| Example 128 | | 1:2 | 4.88 | 114.7 | Green | 213 |
| Example 129 | | 1:3 | 4.92 | 110.6 | Green | 219 |
| Example 130 | 1-93:2-85 | 1:1 | 3.87 | 185.2 | Green | 320 |
| Example 131 | | 1:2 | 3.89 | 183.5 | Green | 329 |
| Example 132 | | 1:3 | 3.94 | 182.9 | Green | 331 |
| Example 133 | 1-94:2-88 | 1:1 | 3.72 | 180.6 | Green | 326 |
| Example 134 | | 1:2 | 3.75 | 179.4 | Green | 333 |
| Example 135 | | 1:3 | 3.84 | 178.2 | Green | 339 |
| Example 136 | 1-98:3-80 | 1:1 | 3.77 | 177.9 | Green | 352 |
| Example 137 | | 1:2 | 3.84 | 175.3 | Green | 359 |
| Example 138 | | 1:3 | 3.85 | 174.6 | Green | 364 |
| Example 139 | 1-100:3-98 | 1:1 | 3.66 | 180.3 | Green | 354 |
| Example 140 | | 1:2 | 3.71 | 175.2 | Green | 357 |
| Example 141 | | 1:3 | 3.75 | 173.9 | Green | 361 |
| Example 142 | 1-103:2-85 | 1:1 | 3.65 | 176.3 | Green | 364 |
| Example 143 | | 1:2 | 3.72 | 174.2 | Green | 371 |
| Example 144 | | 1:3 | 3.77 | 173.9 | Green | 379 |
| Example 145 | 1-112:3-86 | 1:1 | 5.02 | 150.3 | Green | 200 |
| Example 146 | | 1:2 | 5.13 | 148.7 | Green | 213 |
| Example 147 | | 1:3 | 5.19 | 146.5 | Green | 225 |
| Example 148 | 1-113:3-86 | 1:1 | 5.23 | 147.3 | Green | 204 |
| Example 149 | | 1:2 | 5.25 | 145.2 | Green | 212 |
| Example 150 | | 1:3 | 5.30 | 143.5 | Green | 219 |
| Example 151 | 1-115:2-82 | 1:1 | 5.16 | 148.7 | Green | 219 |
| Example 152 | | 1:2 | 5.20 | 147.5 | Green | 222 |
| Example 153 | | 1:3 | 5.22 | 146.3 | Green | 226 |
| Example 154 | 1-118:2-88 | 1:1 | 5.12 | 138.7 | Green | 225 |
| Example 155 | | 1:2 | 5.15 | 135.2 | Green | 234 |
| Example 156 | | 1:3 | 5.23 | 133.9 | Green | 239 |
| Example 157 | 1-126:3-86 | 1:1 | 5.26 | 129.7 | Green | 259 |
| Example 158 | | 1:2 | 5.31 | 128.5 | Green | 264 |
| Example 159 | | 1:3 | 5.33 | 127.4 | Green | 269 |
| Example 160 | 1-131:3-80 | 1:1 | 5.19 | 132.6 | Green | 248 |
| Example 161 | | 1:2 | 5.24 | 130.1 | Green | 253 |
| Example 162 | | 1:3 | 5.36 | 128.7 | Green | 267 |
| Example 163 | 1-132:2-85 | 1:1 | 5.34 | 129.5 | Green | 266 |
| Example 164 | | 1:2 | 5.41 | 127.4 | Green | 270 |
| Example 165 | | 1:3 | 5.44 | 126.5 | Green | 274 |
| Example 166 | 1-137:2-89 | 1:1 | 1.23 | 200.4 | Green | 415 |
| Example 167 | | 1:2 | 1.32 | 198.7 | Green | 423 |
| Example 168 | | 1:3 | 1.39 | 197.4 | Green | 433 |
| Example 169 | 1-138:3-86 | 1:1 | 2.13 | 197.5 | Green | 429 |
| Example 170 | | 1:2 | 2.25 | 195.3 | Green | 435 |
| Example 171 | | 1:3 | 2.31 | 193.6 | Green | 441 |
| Example 172 | 1-140:2-85 | 1:1 | 2.22 | 195.8 | Green | 419 |
| Example 173 | | 1:2 | 2.29 | 194.6 | Green | 426 |
| Example 174 | | 1:3 | 2.35 | 192.7 | Green | 430 |
| Example 175 | 1-141:3-98 | 1:1 | 2.41 | 193.2 | Green | 452 |
| Example 176 | | 1:2 | 2.44 | 191.0 | Green | 455 |
| Example 177 | | 1:3 | 2.56 | 190.8 | Green | 463 |
| Example 178 | 1-143:3-112 | 1:1 | 1.68 | 195.7 | Green | 447 |
| Example 179 | | 1:2 | 1.74 | 194.6 | Green | 453 |
| Example 180 | | 1:3 | 1.77 | 192.9 | Green | 459 |
| Example 181 | 1-146:3-112 | 1:1 | 1.56 | 194.5 | Green | 423 |
| Example 182 | | 1:2 | 1.67 | 193.4 | Green | 436 |
| Example 183 | | 1:3 | 1.78 | 192.0 | Green | 449 |
| Example 184 | 1-148:2-85 | 1:1 | 1.02 | 195.5 | Green | 452 |
| Example 185 | | 1:2 | 1.13 | 194.1 | Green | 456 |
| Example 186 | | 1:3 | 1.15 | 192.6 | Green | 464 |
| Example 187 | 1-149:3-86 | 1:1 | 4.35 | 160.3 | Green | 300 |
| Example 188 | | 1:2 | 4.42 | 158.7 | Green | 313 |
| Example 189 | | 1:3 | 4.53 | 156.2 | Green | 326 |
| Example 190 | 1-152:2-85 | 1:1 | 4.52 | 159.4 | Green | 295 |
| Example 191 | | 1:2 | 4.55 | 157.3 | Green | 308 |
| Example 192 | | 1:3 | 4.68 | 155.9 | Green | 310 |

Comparing the results of Table 11 above with the results of Table 9 above, it can be seen that when the heterocyclic compound of the present invention is used in combination with the compound of Chemical Formula 2 or 3 as a material for a light emitting layer, the performance of the device can be further improved.

In contrast, in the case of the comparative compounds, it was confirmed that the performance was improved to some extent when combined with the compound of Chemical Formula 2 or 3, but it was determined that the improvement effects of the comparative examples are insignificant compared to the present invention, and it was confirmed that even though the comparative compounds were used in combination with the compound of Chemical Formula 2 or 3, inferior performance was exhibited compared to when the heterocyclic compound of the present invention was used alone.

What is claimed is:

1. A heterocyclic compound of the following Chemical Formula 1: 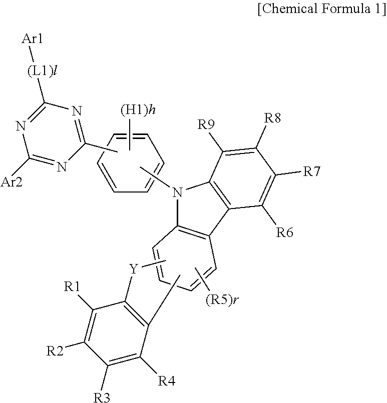
wherein, in Chemical Formula 1,
L1 is a direct bond; a substituted or unsubstituted C6 to C60 arylene group; or a substituted or unsubstituted C2 to C60 heteroarylene group,
1 is an integer from 1 to 3, and when 1 is 2 or greater, L1 is the same or different,
Y is O; S; C(R10)(R11) or N(R12),
H1 is hydrogen; or deuterium,
h is an integer from 1 to 4, and when h is 2 or greater, H1 is the same or different,
R1 to R12 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
at least one of R1 to R9 is deuterium,
r is 1 or 2, and when r is 2, R5 is the same or different,
Ar1 is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
Ar2 is a group represented by the following Chemical Formula A or B, 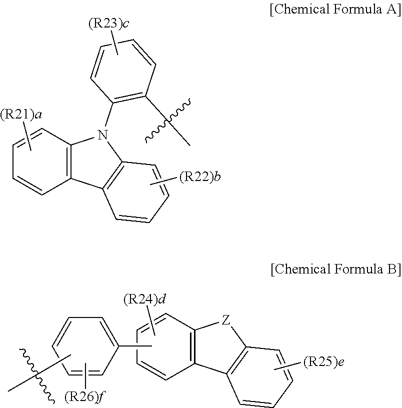
in Chemical Formulae A and B, [symbol]
is a moiety bonded to Chemical Formula 1,
Z is O; S; or C(R27)(R28),
R21 to R28 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or bonded to an adjacent group to form a substituted or unsubstituted aromatic hydrocarbon ring; or a substituted or unsubstituted aromatic hetero ring, and
a, b, c, e, and f are each an integer from 0 to 4, d is an integer from 0 to 3, and when a to f are each 2 or greater, substituents in the parenthesis are the same or different.

2. The heterocyclic compound of claim 1, wherein Chemical Formula 1 is represented by the following Chemical Formula 1-A or 1-B: 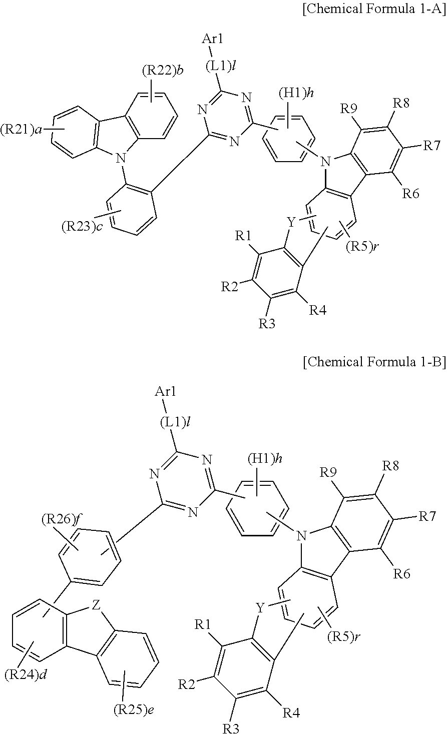
in Chemical Formulae 1-A and 1-B,
the definition of each substituent is the same as the definition in Chemical Formula 1.

3. The heterocyclic compound of claim 1, wherein Chemical Formula 1 is represented by any one of the following Chemical Formulae 1-1 to 1-6: 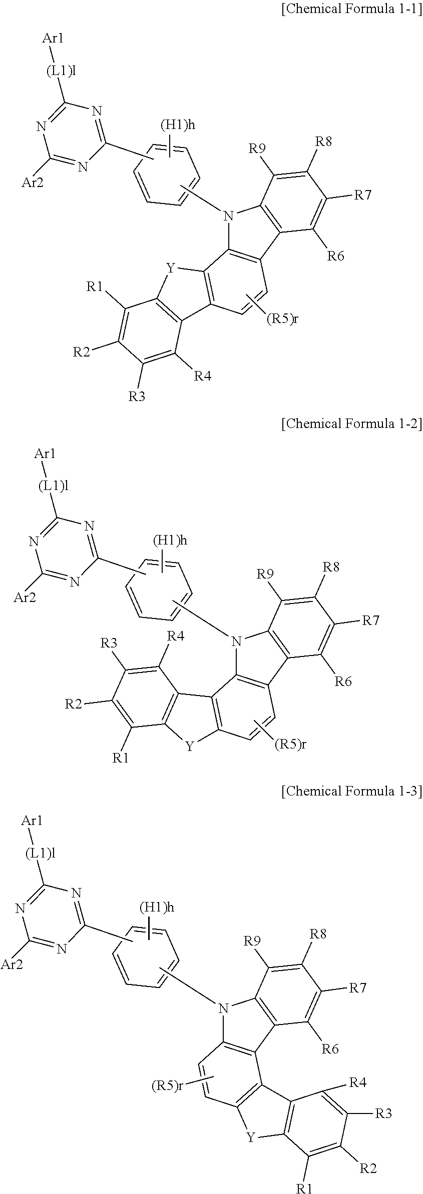 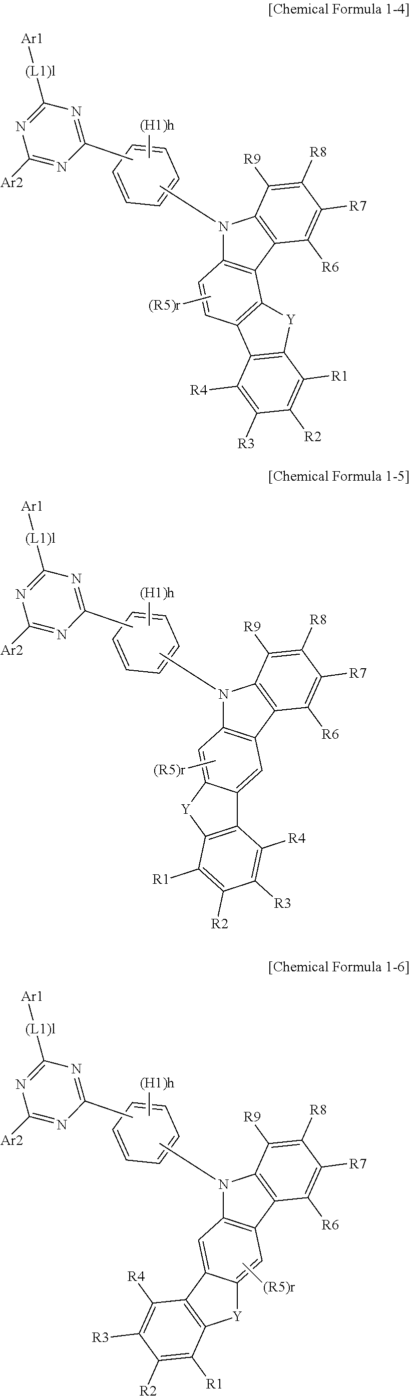
in Chemical Formulae 1-1 to 1-6,
the definition of each substituent is the same as the definition in Chemical Formula 1.

4. The heterocyclic compound of claim 1, wherein Chemical Formula 1 is represented by any one of the following Chemical Formulae 1-a to 1-d: 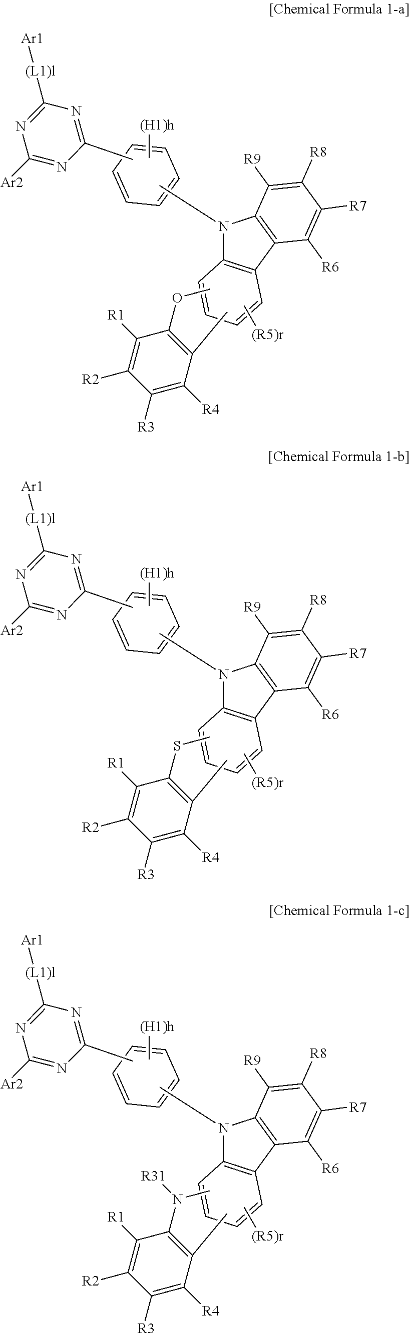 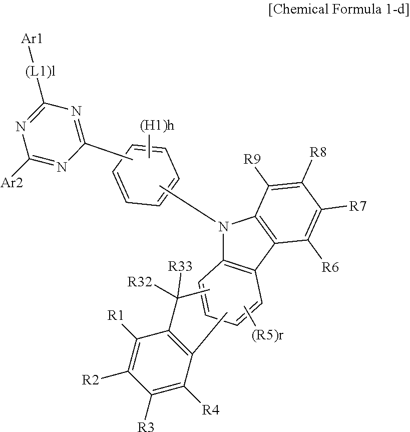
in Chemical Formulae 1-a to 1-d,
R31 is a substituted or unsubstituted C6 to C60 aryl group,
R32 and R33 are each independently a substituted or unsubstituted C1 to C60 alkyl group, and
the definitions of the other substituents are the same as the definitions in Chemical Formula 1.

5. The heterocyclic compound of claim 1, wherein Ar1 is a substituted or unsubstituted C6 to C60 aryl group.

6. The heterocyclic compound of claim 1, wherein Chemical Formula 1 is represented by the following Chemical Formula 1-D: 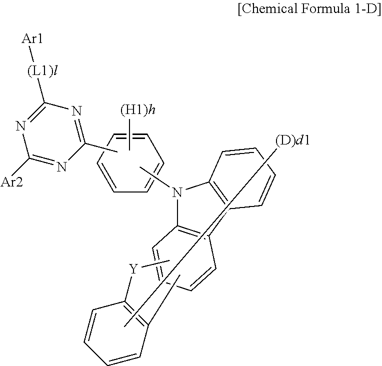
in Chemical Formula 1-D,
D is deuterium,
d1 is an integer from 1 to 10, and
the definitions of the other substituents are the same as the definitions in Chemical Formula 1.

7. The heterocyclic compound of claim 1, wherein a deuterium substitution rate of the 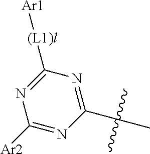
structure in Chemical Formula 1 is 0%, or 10% to 100%.

8. The heterocyclic compound of claim 1, wherein the deuterium substitution rate of Chemical Formula 1 is 10% to 100%.

9. The heterocyclic compound of claim 1, wherein Chemical Formula 1 is represented by any one of the following compounds:

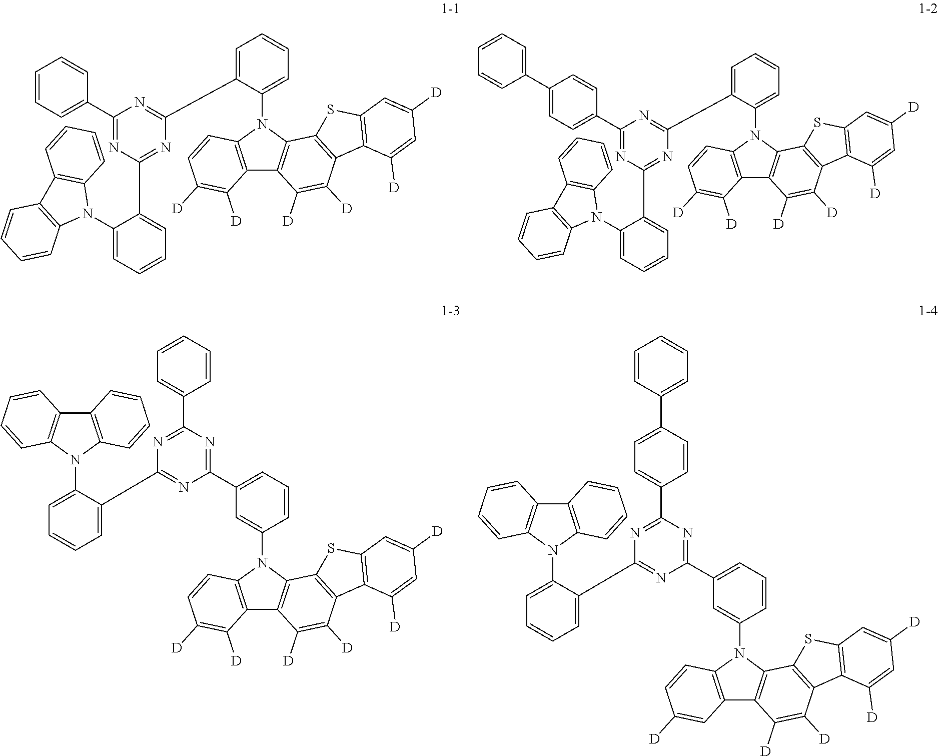

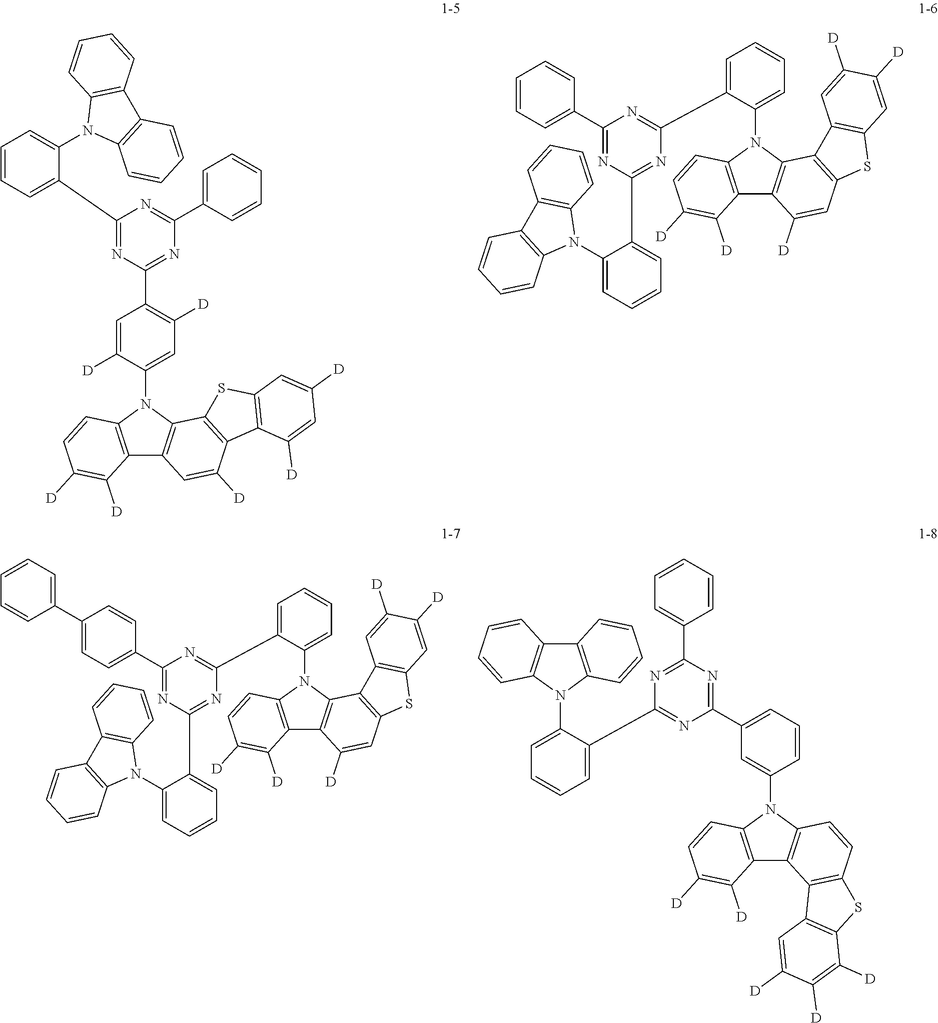

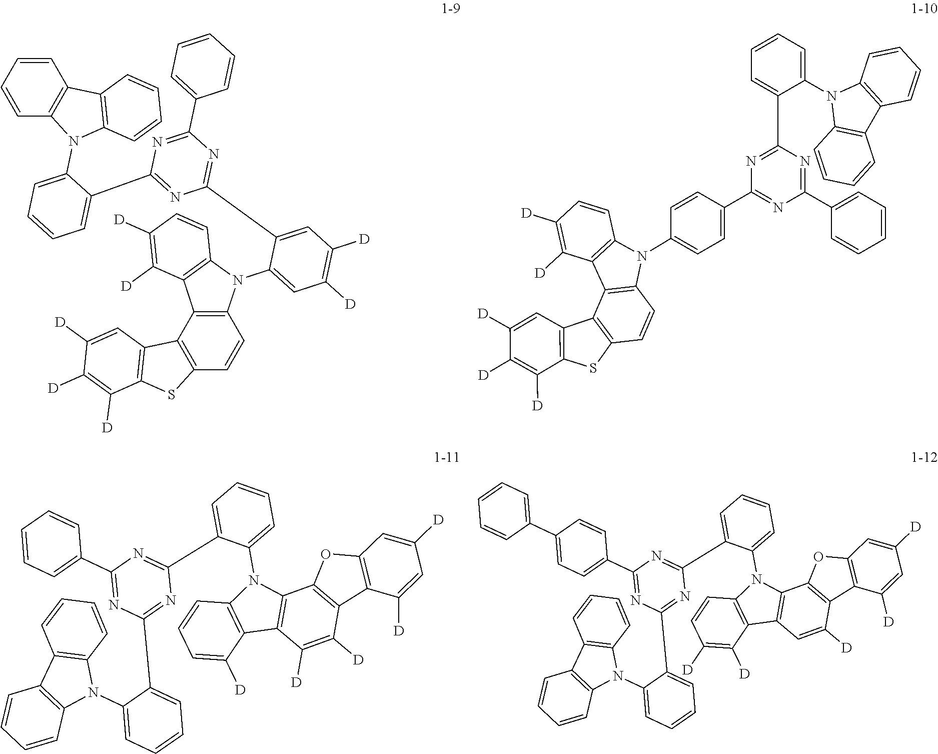

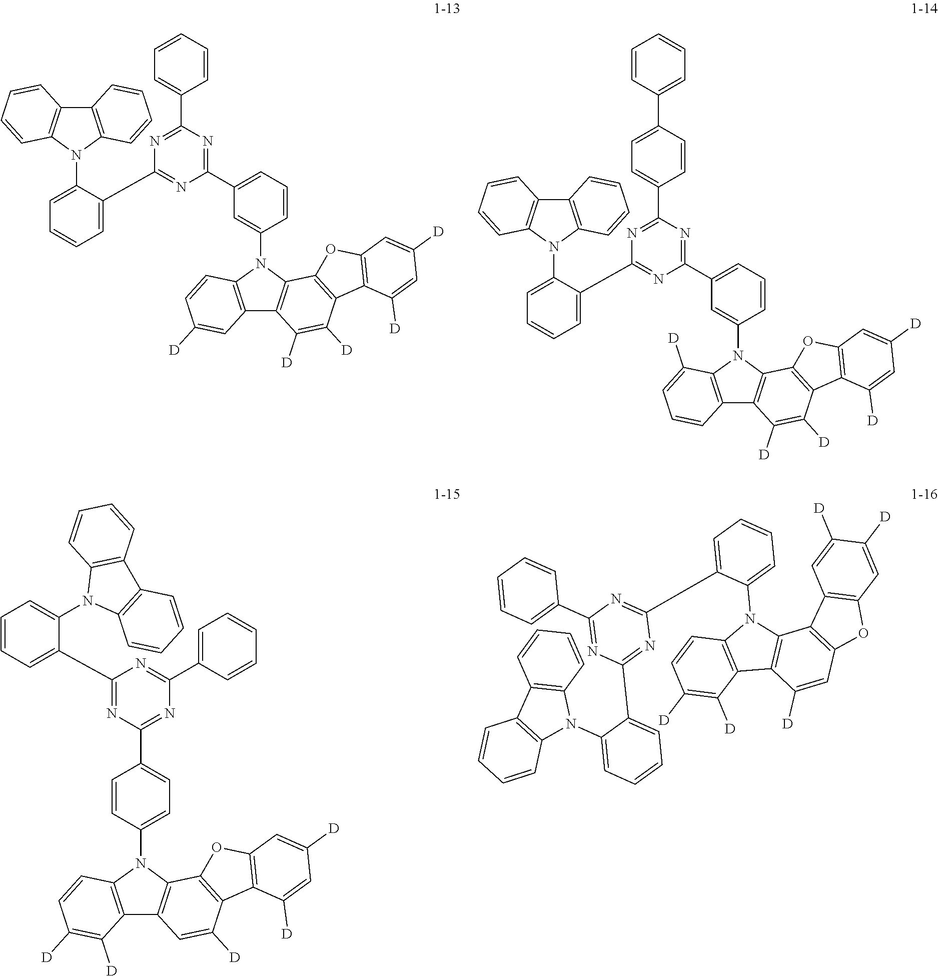

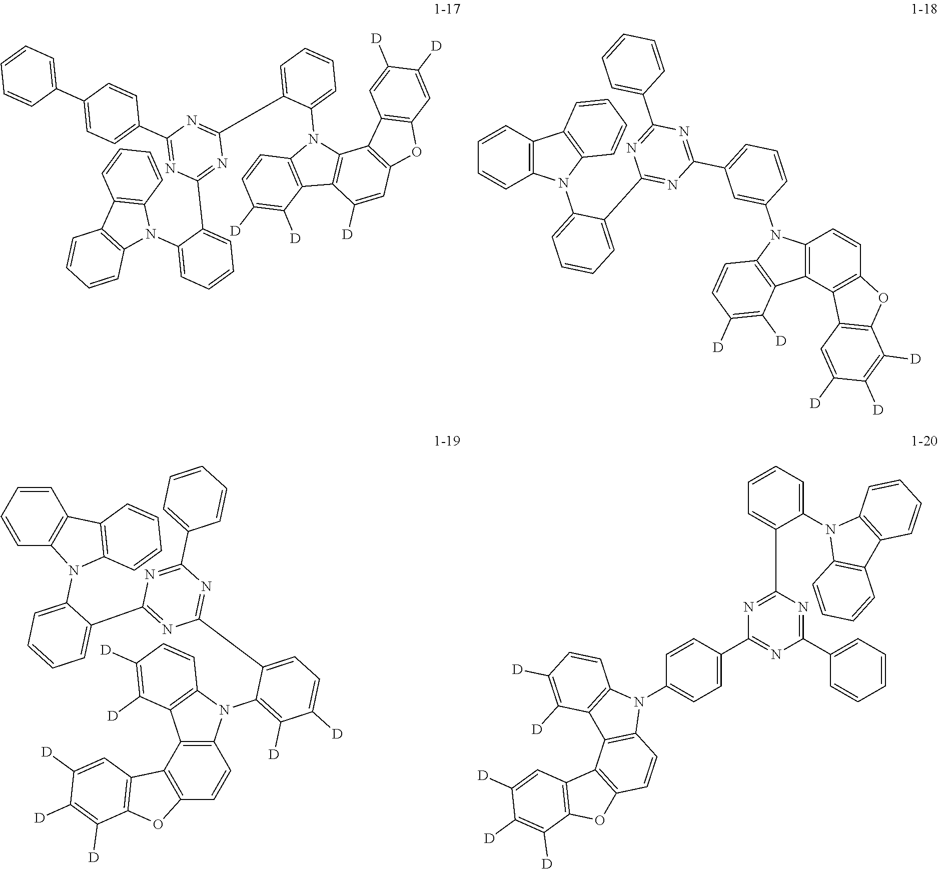

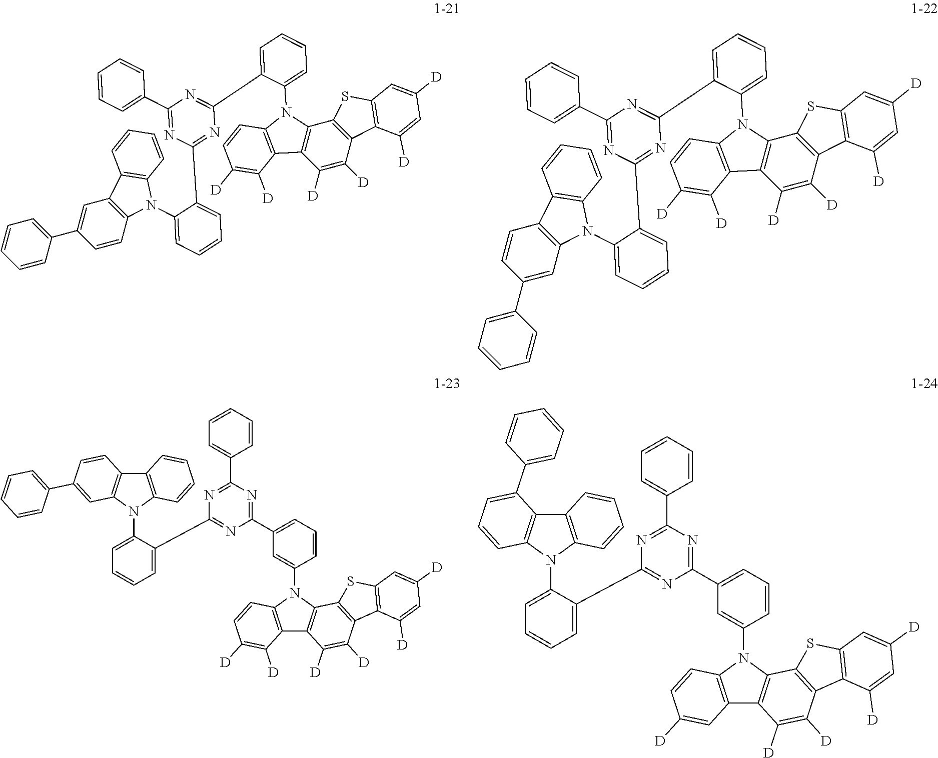

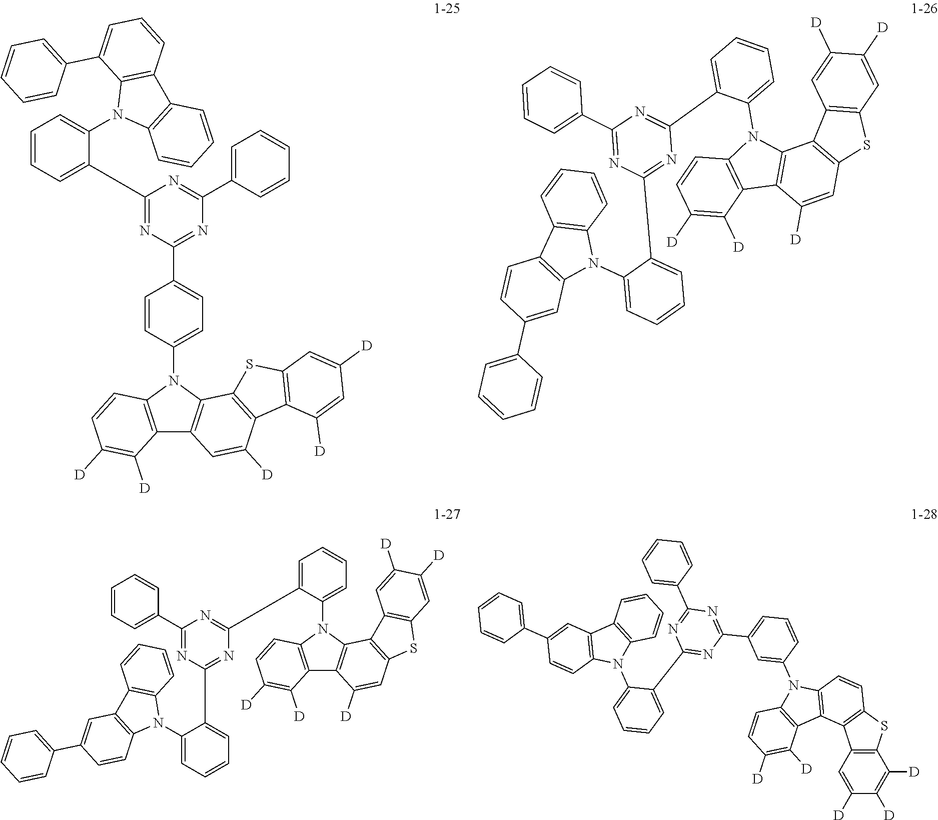

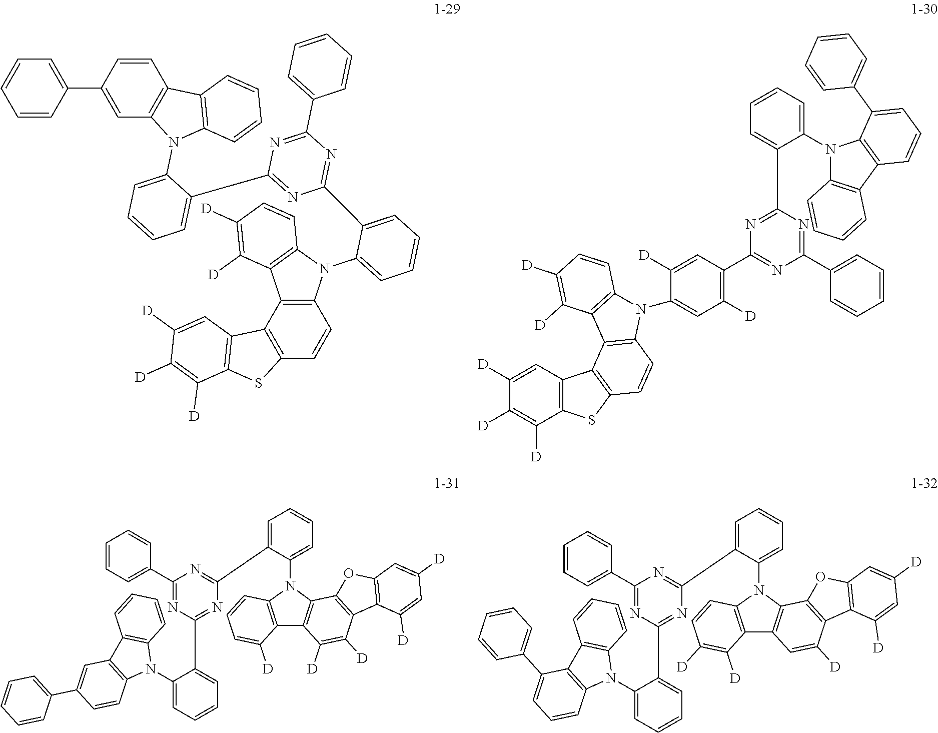

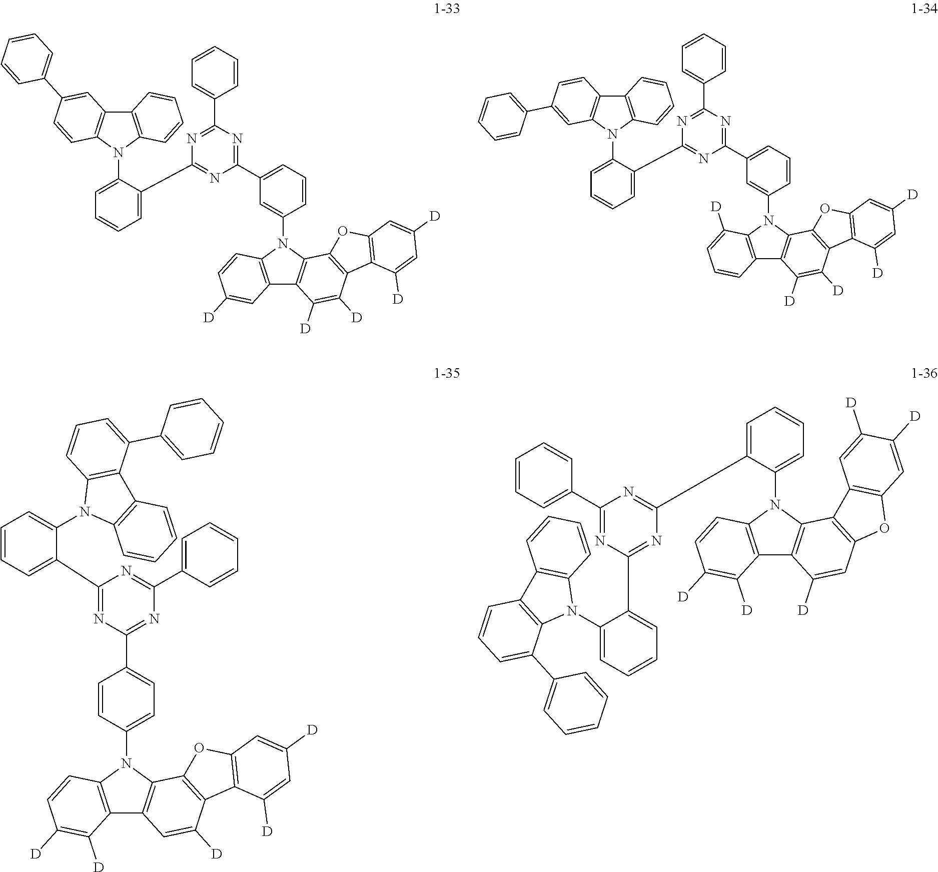

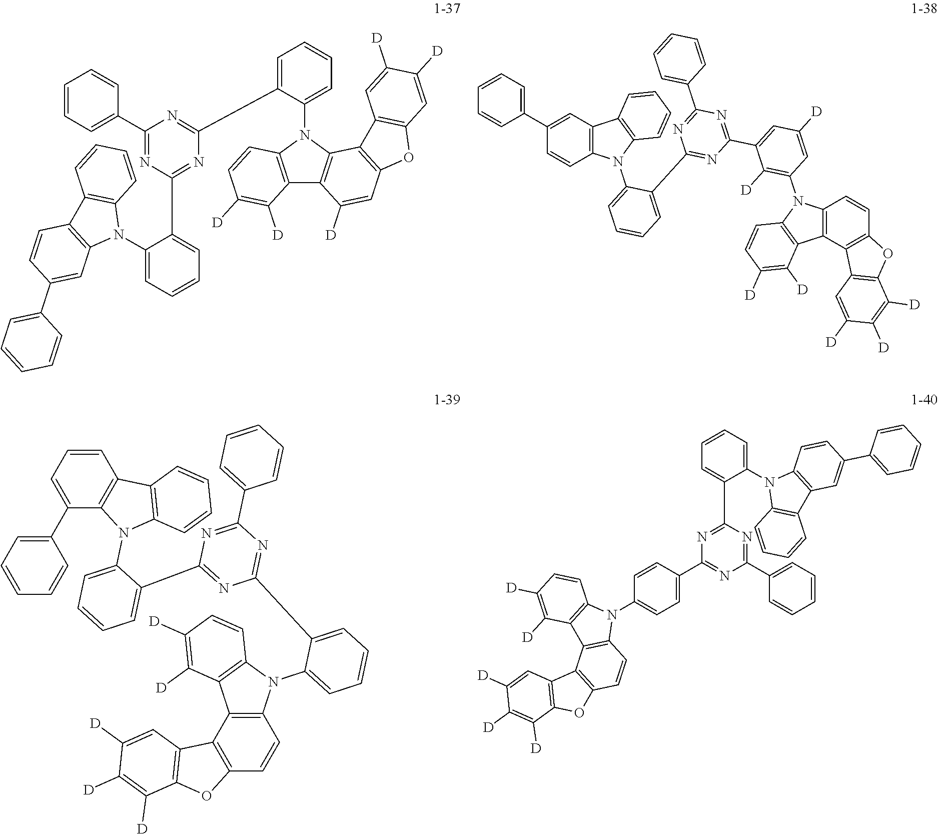

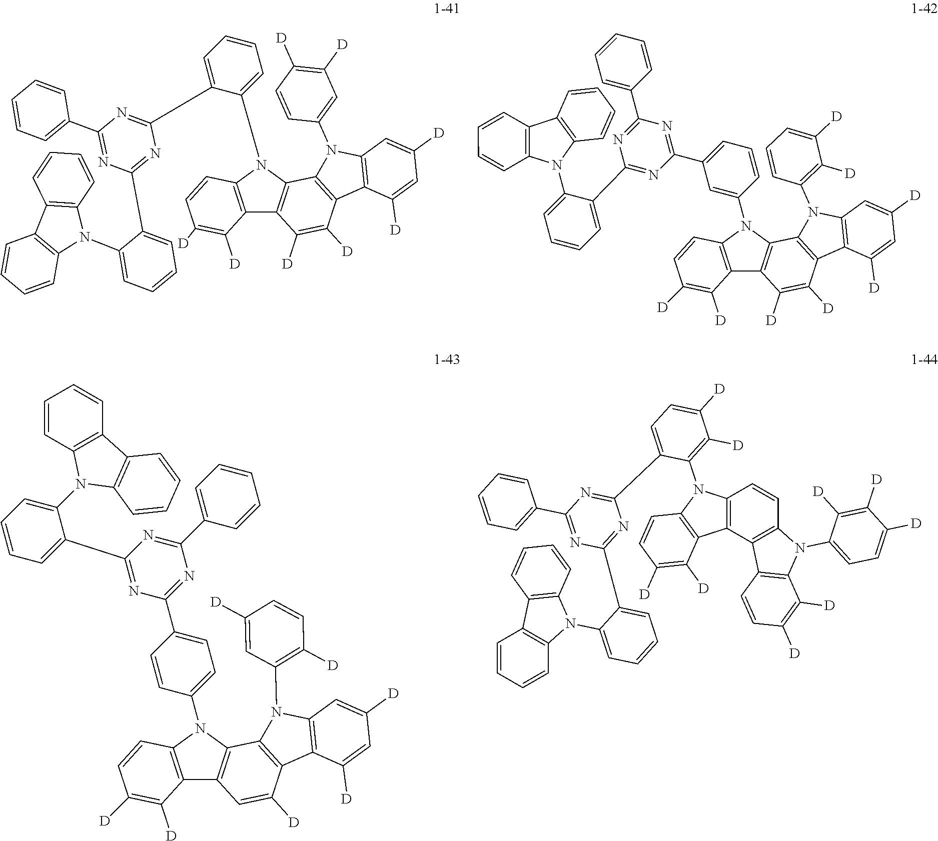

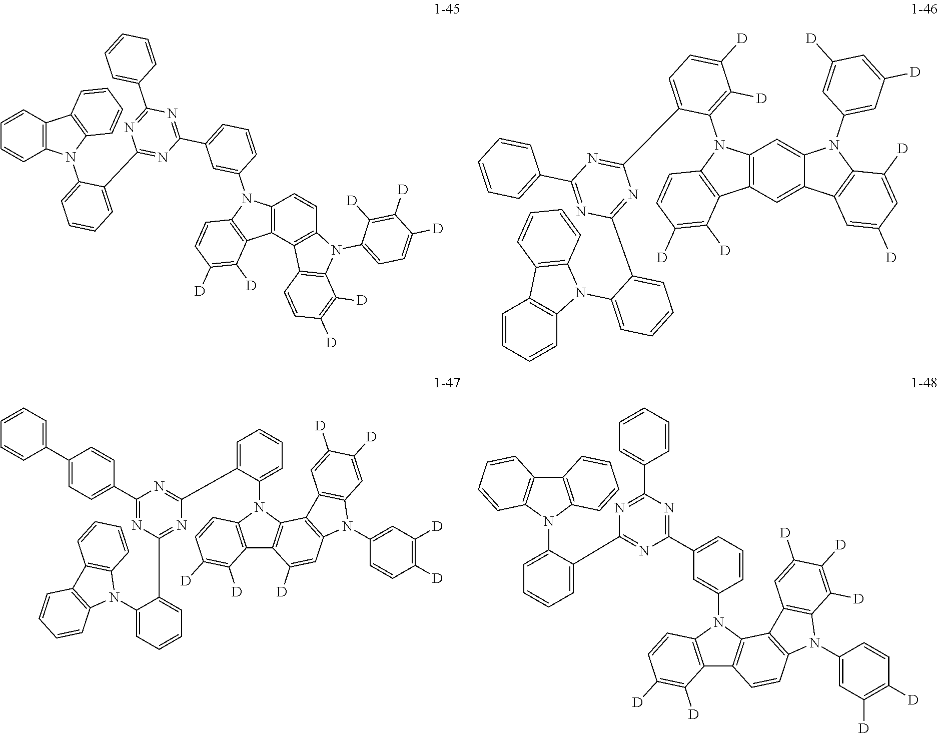

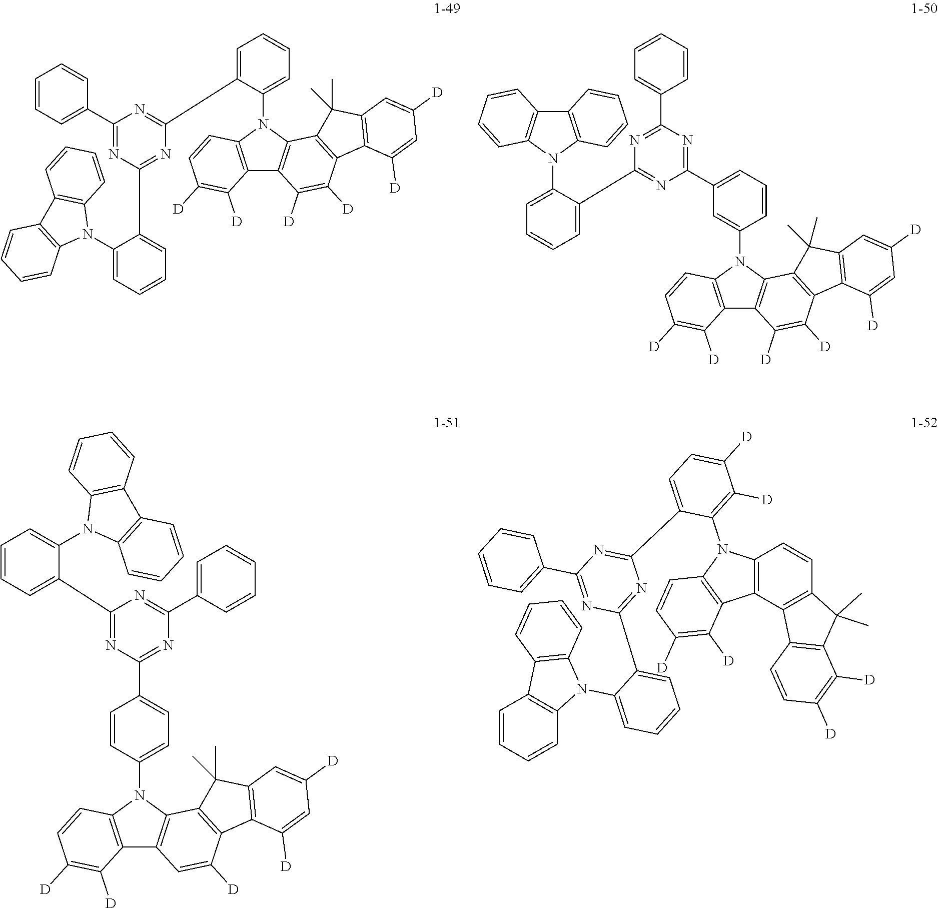

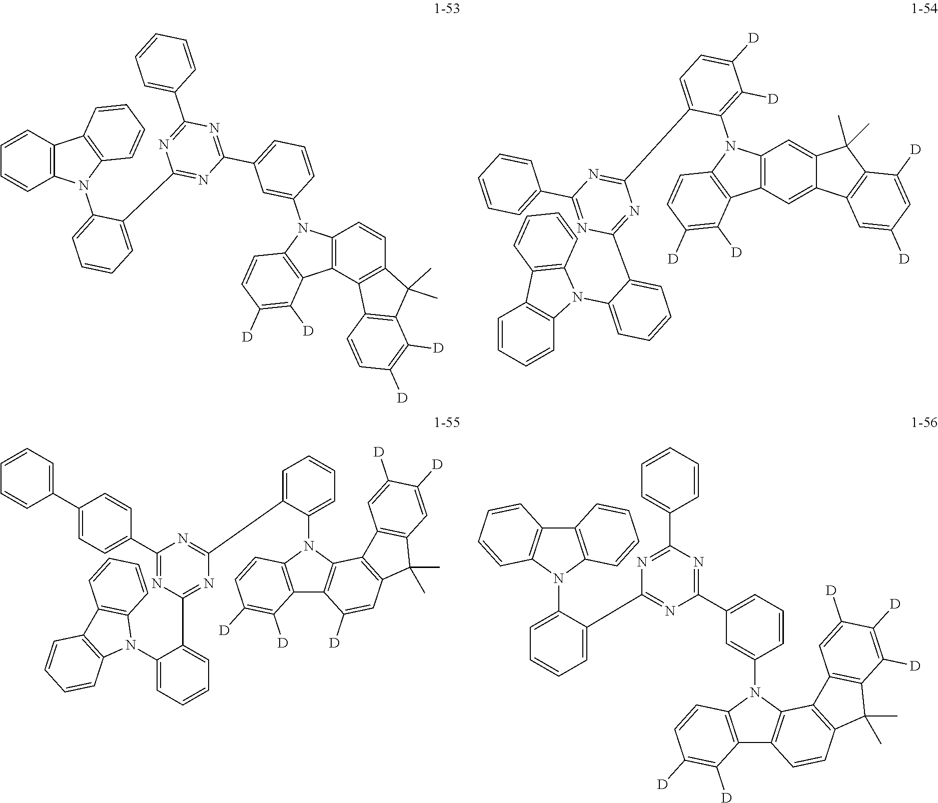

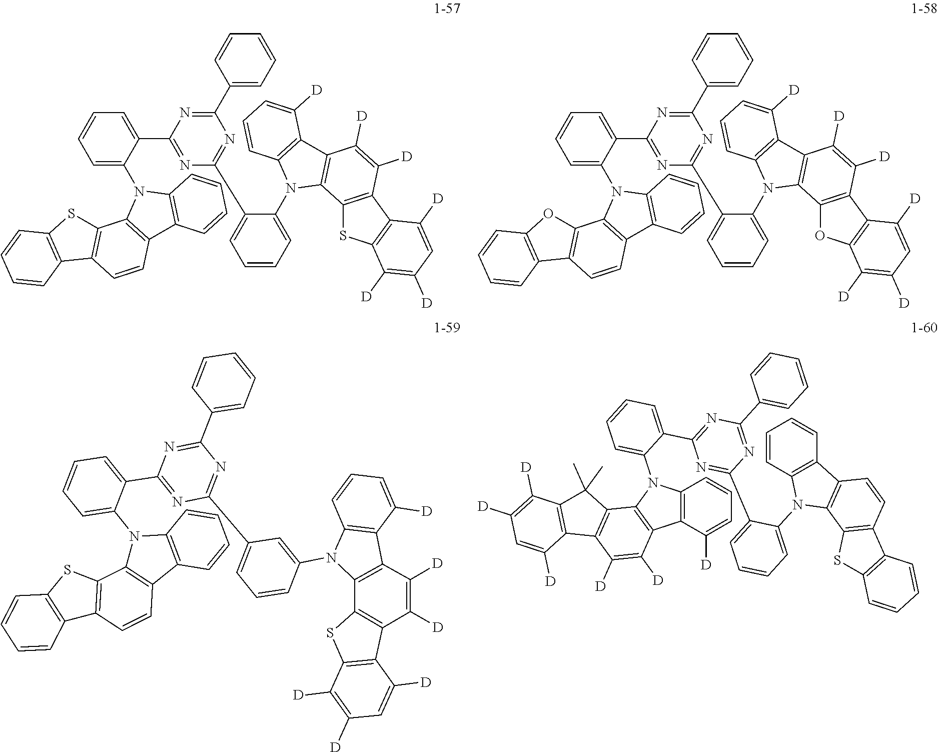

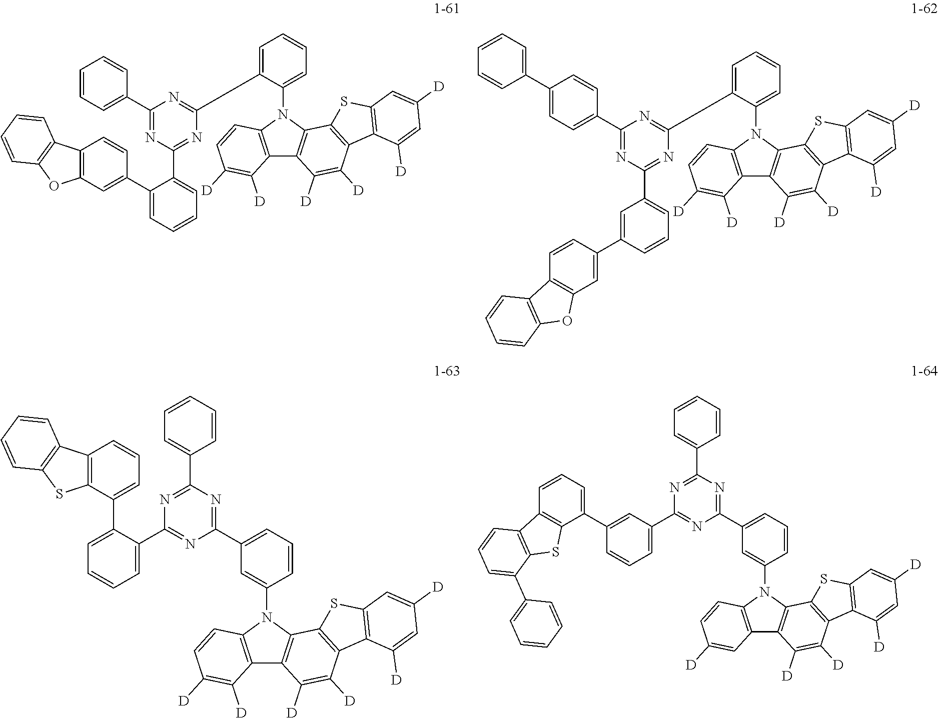

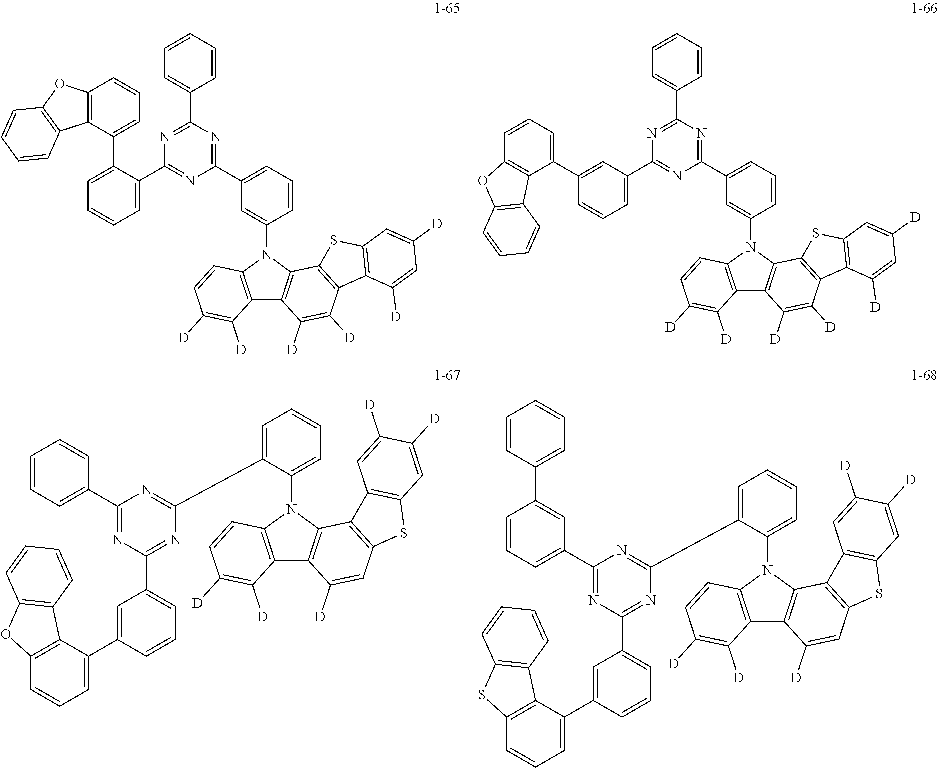

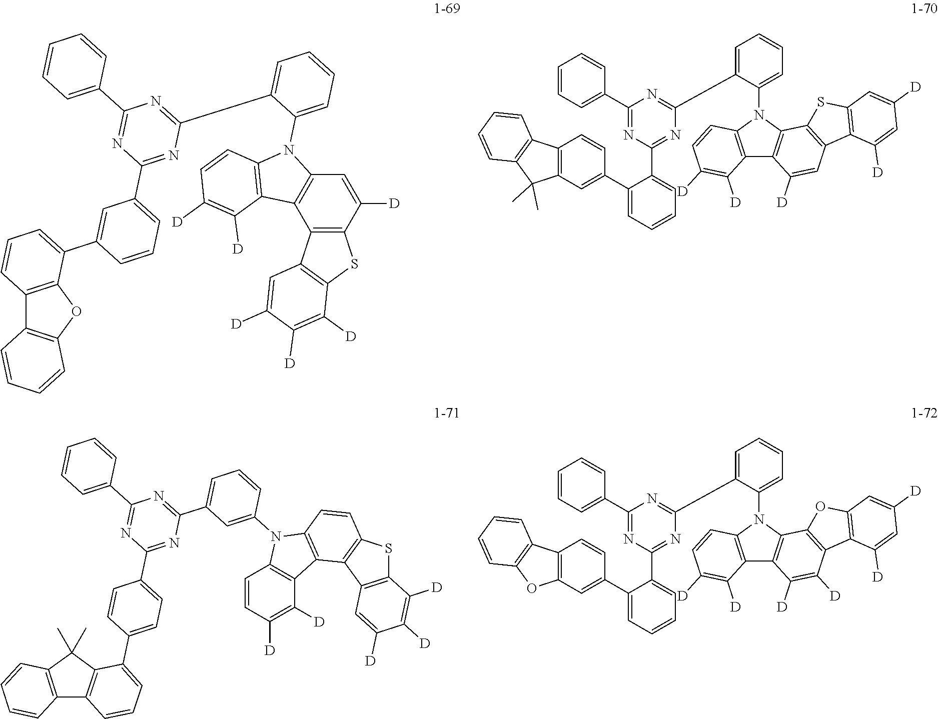

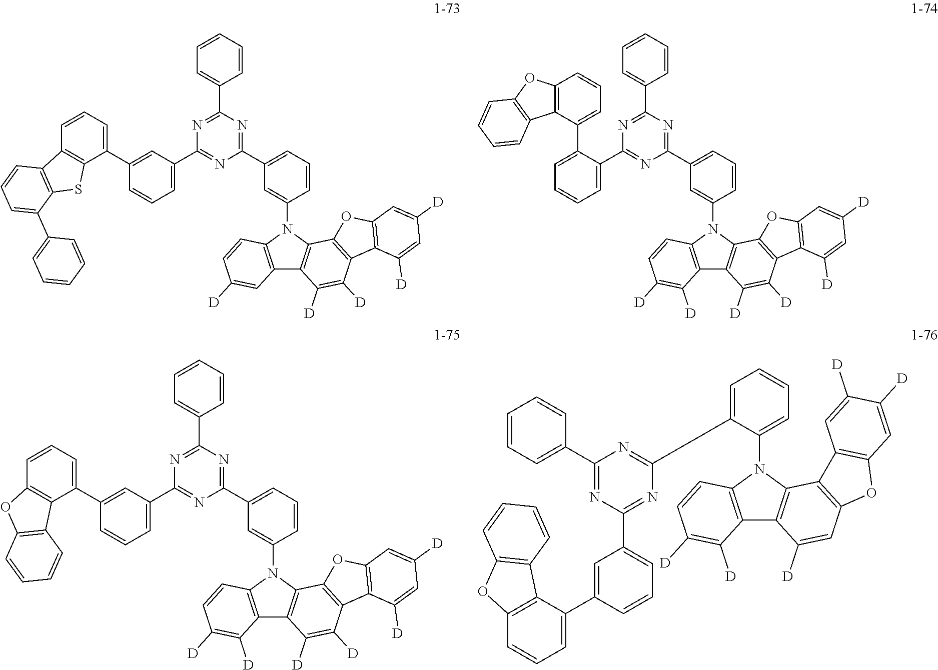

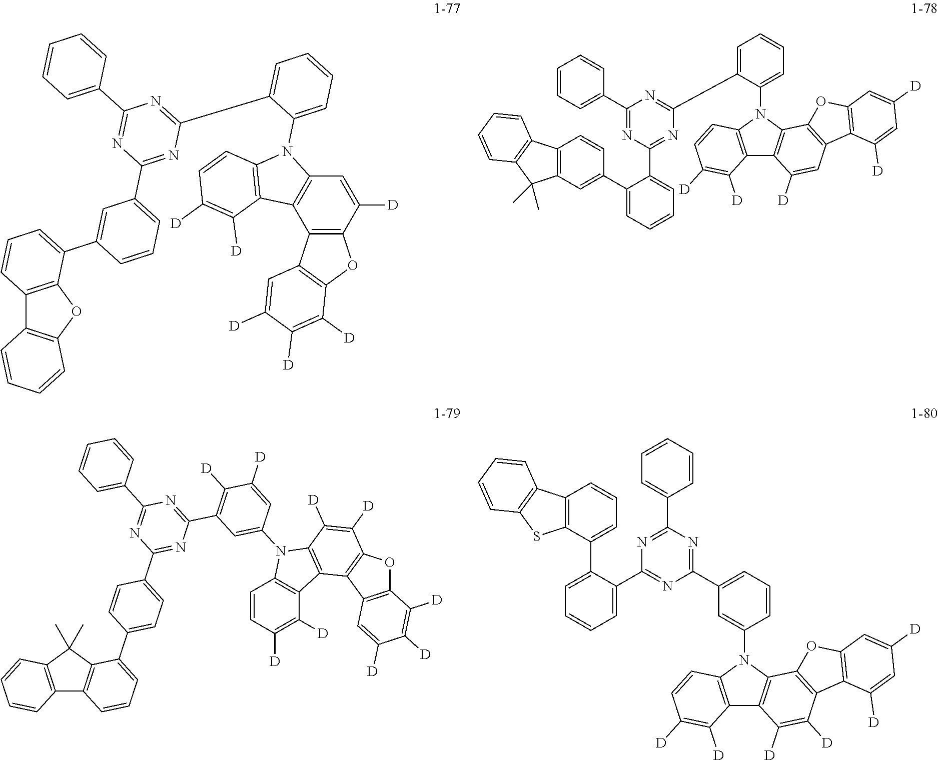

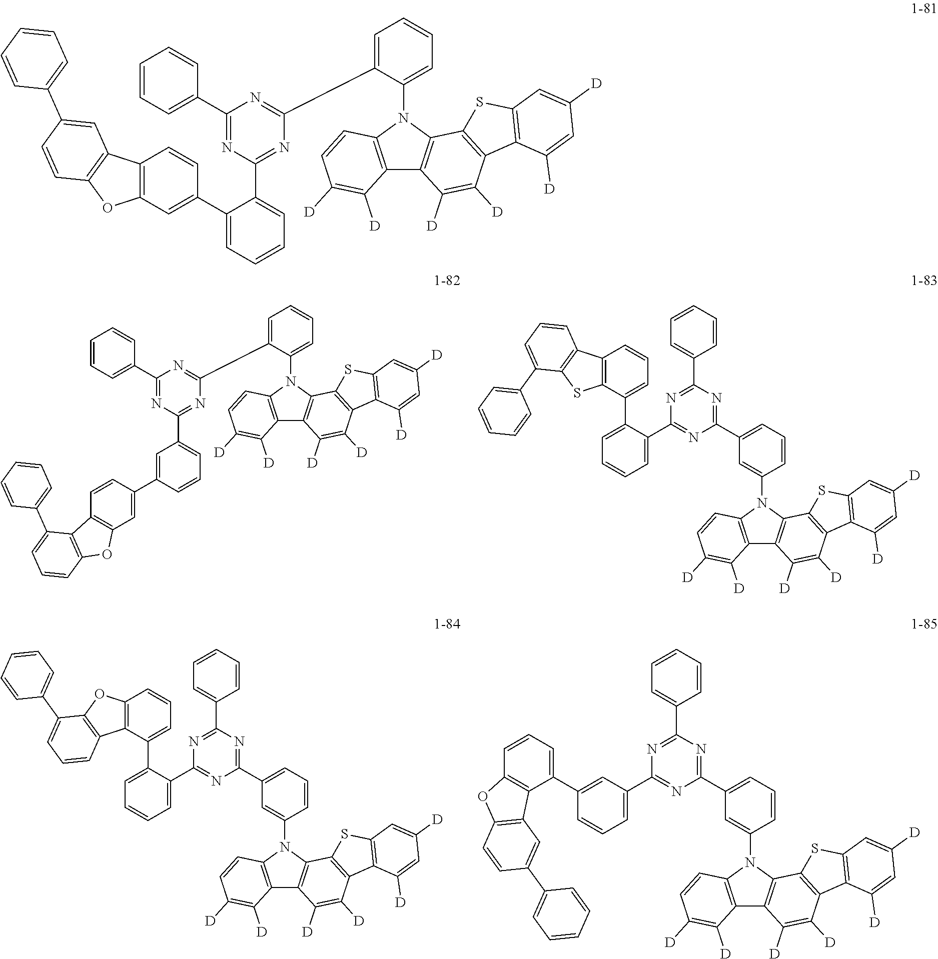

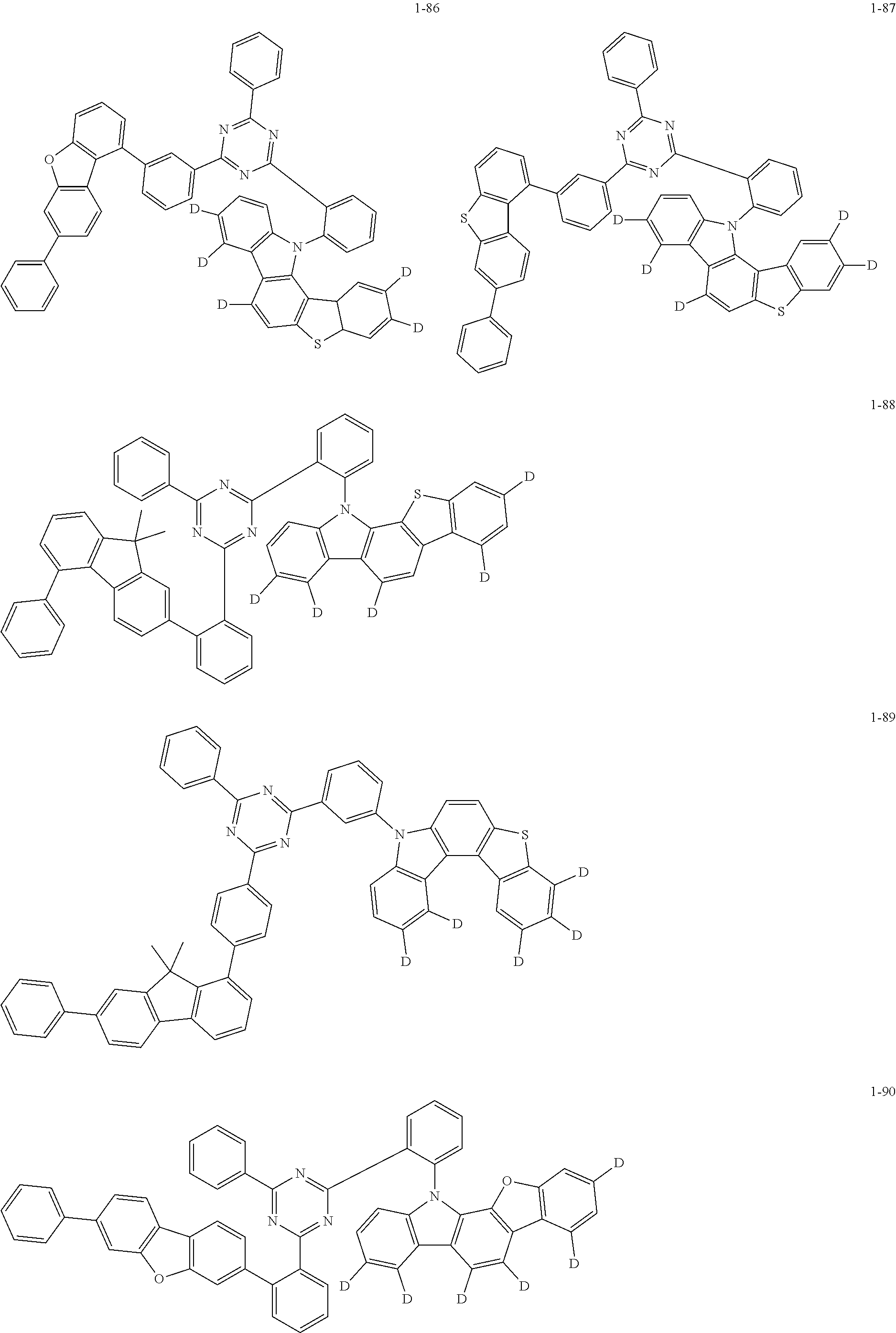

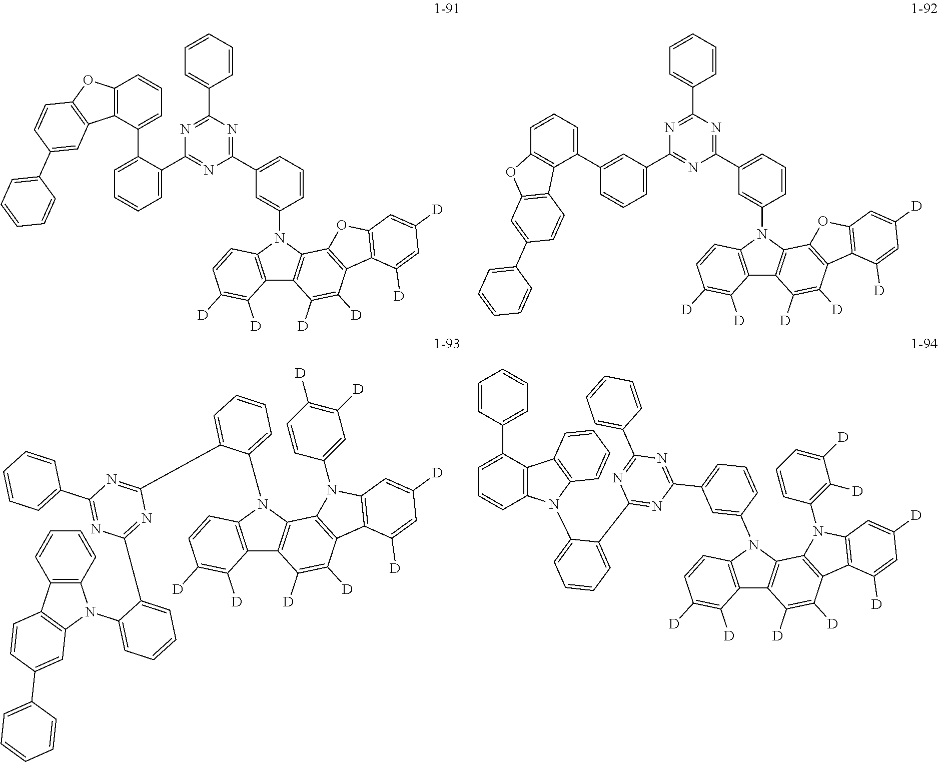

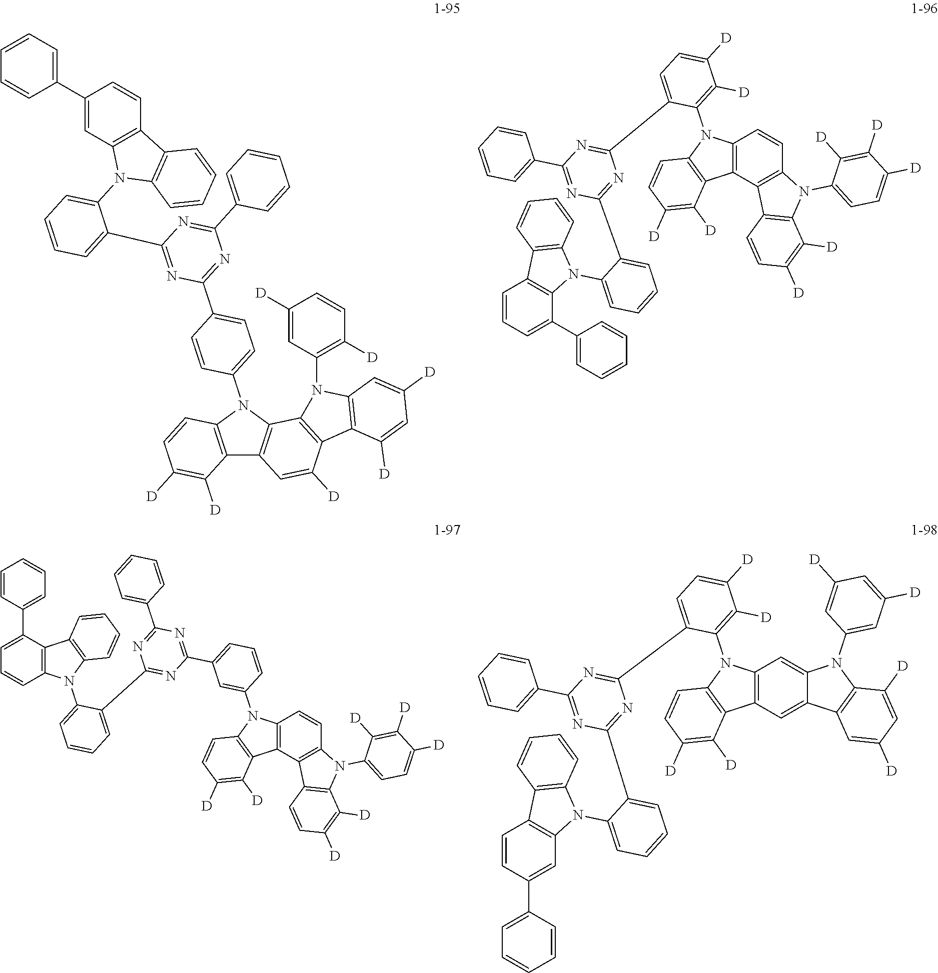

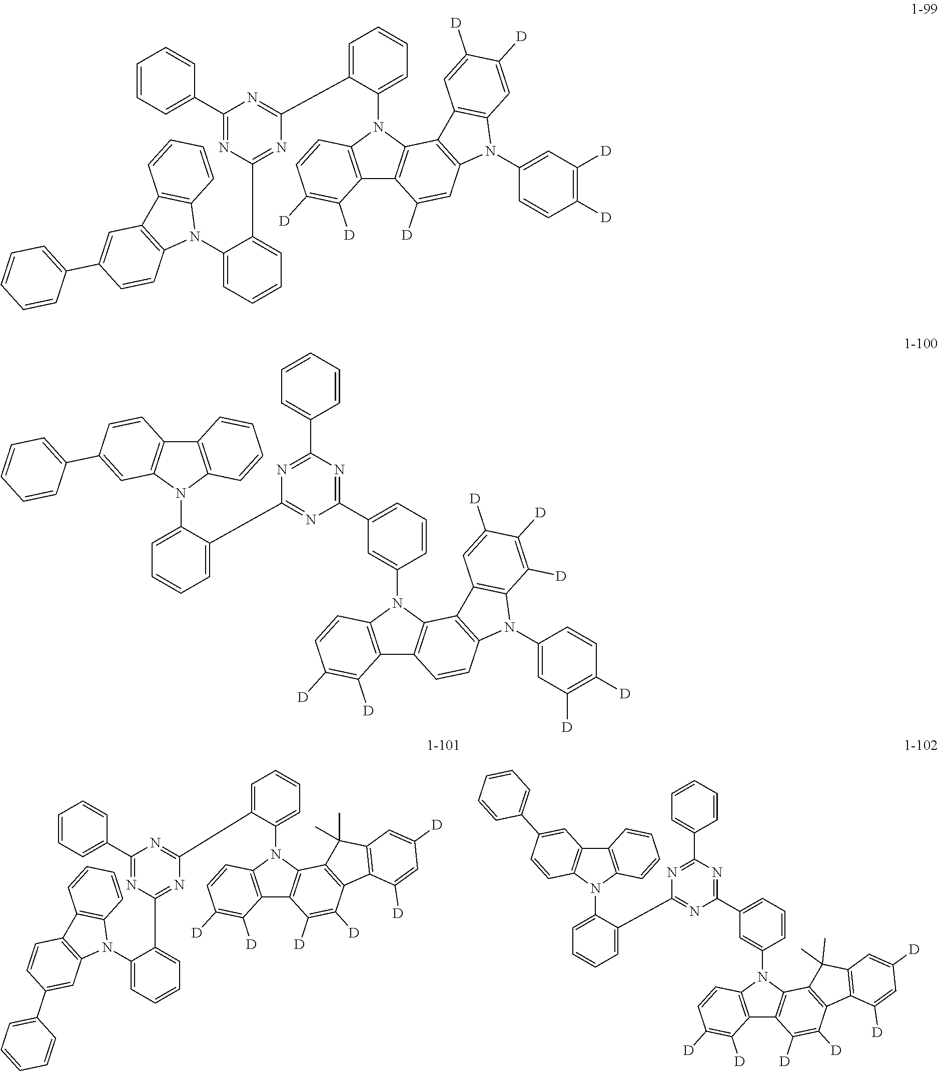

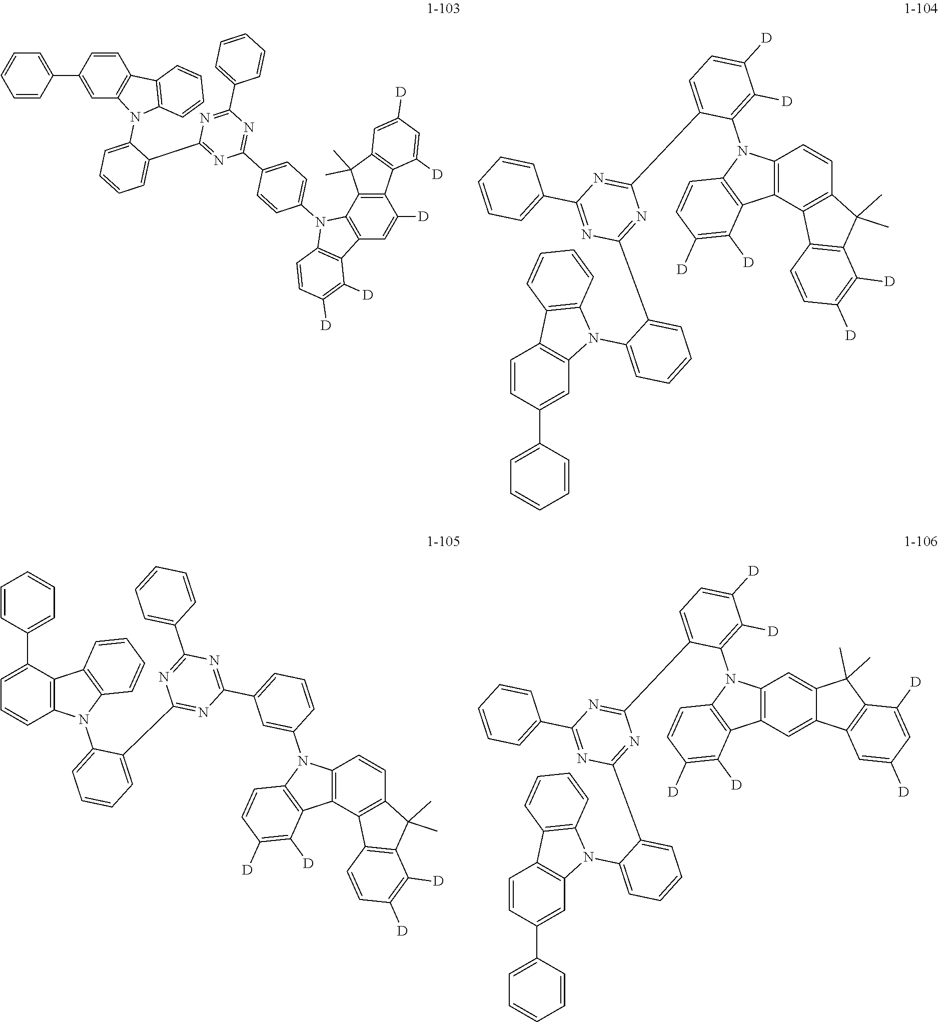

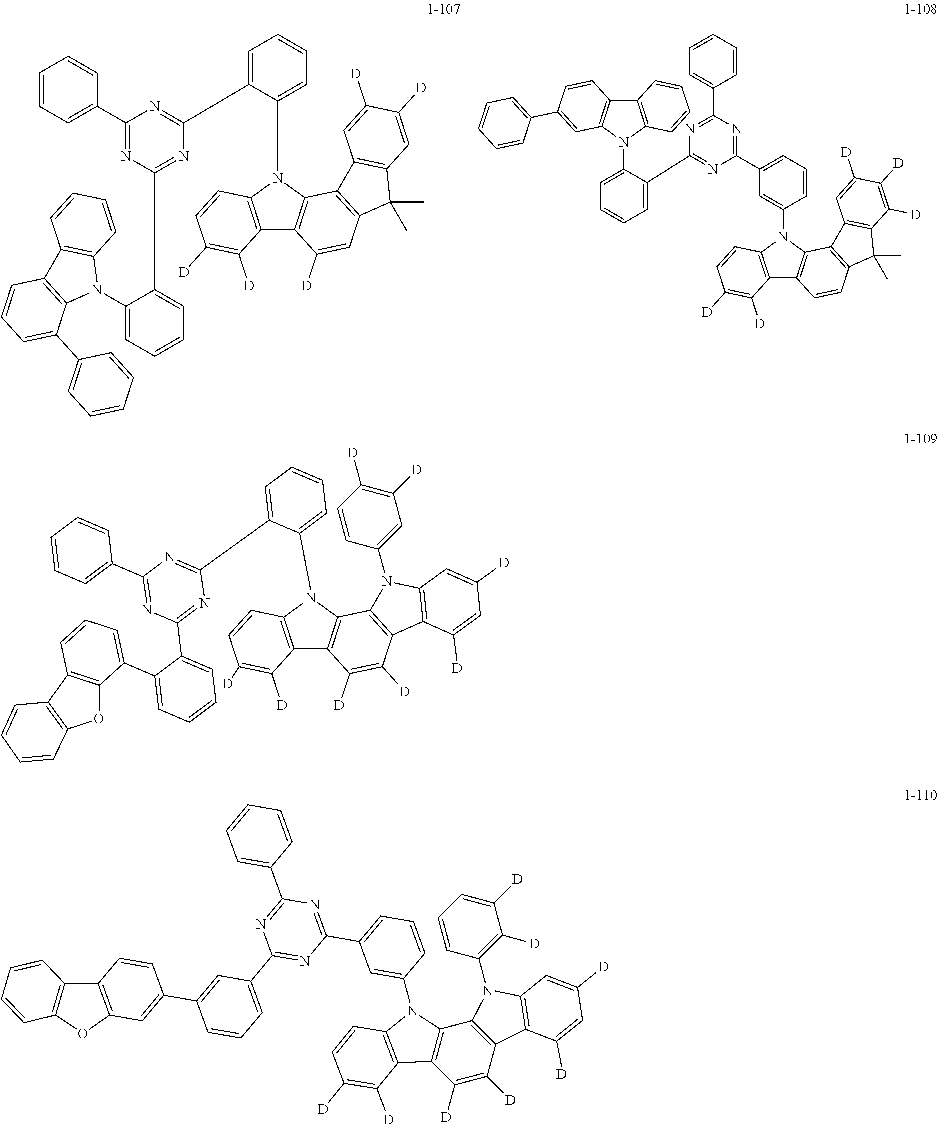

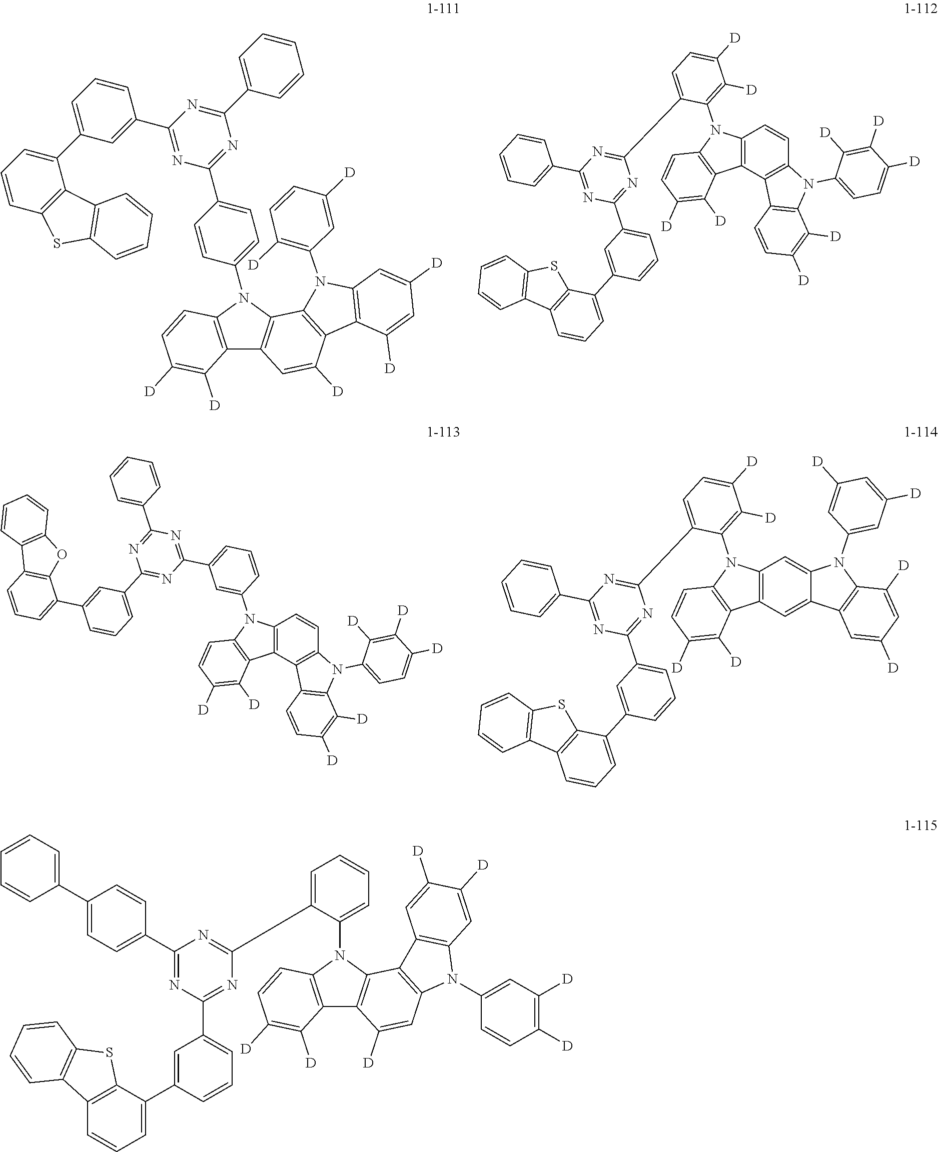

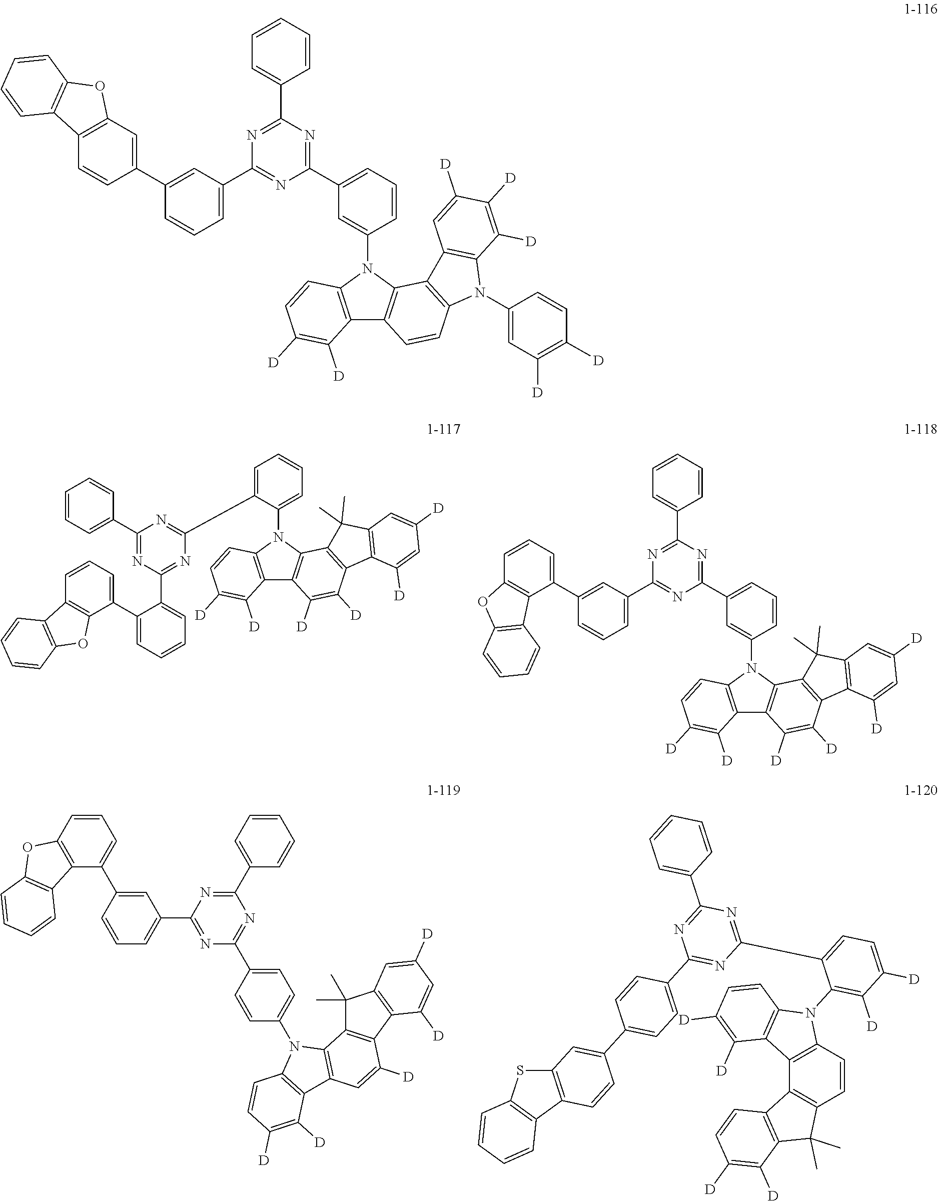

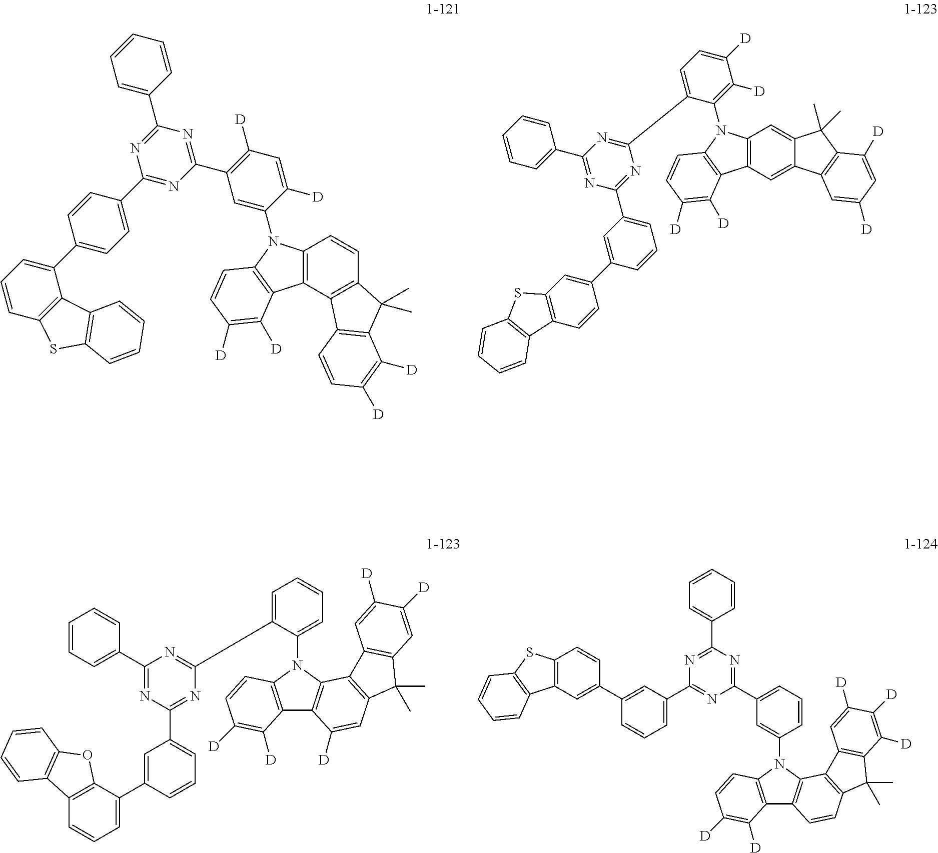

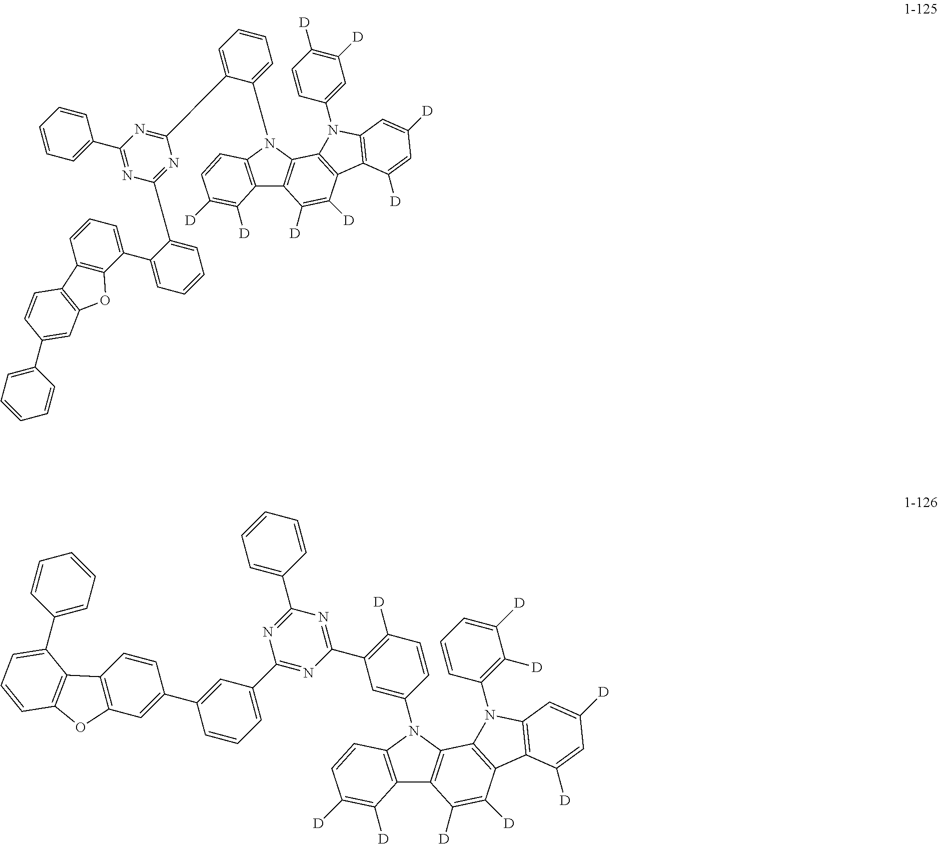 [SMILES: [2H]c1cc([2H])c2c3c([2H])c([2H])c4c5c([2H])c([2H])ccc5n(-c5ccccc5-c5nc(-c6ccccc6)nc(-c6ccccc6-c6cccc7c6oc6cc(-c8ccccc8)ccc67)n5)c4c3n(-c3ccc([2H])c([2H])c3)c2c1 ; [2H]c1cc([2H])c2c3c([2H])c([2H])c4c5c([2H])c([2H])ccc5n(-c5ccc([2H])c(-c6nc(-c7ccccc7)nc(-c7cccc(-c8ccc9c(c8)oc8cccc(-c%10ccccc%10)c89)c7)n6)c5)c4c3n(-c3cccc([2H])c3[2H])c2c1]

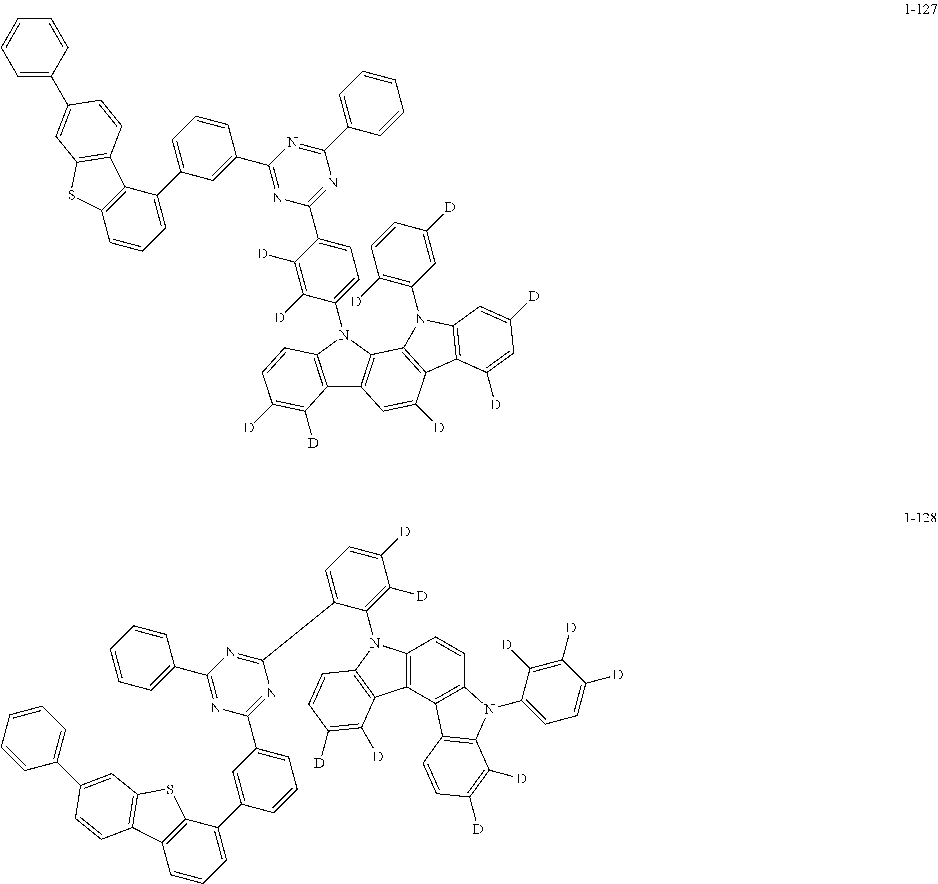 [SMILES: [2H]c1ccc([2H])c(-n2c3cc([2H])cc([2H])c3c3c([2H])cc4c5c([2H])c([2H])ccc5n(-c5ccc(-c6nc(-c7ccccc7)nc(-c7cccc(-c8cccc9sc%10cc(-c%11ccccc%11)ccc%10c89)c7)n6)c([2H])c5[2H])c4c32)c1 ; [2H]c1ccc(-n2c3ccc4c(c5c([2H])c([2H])ccc5n4-c4c(-c5nc(-c6ccccc6)nc(-c6cccc(-c7cccc8c7sc7cc(-c9ccccc9)ccc78)c6)n5)ccc([2H])c4[2H])c3c3ccc([2H])c([2H])c32)c([2H])c1[2H]]

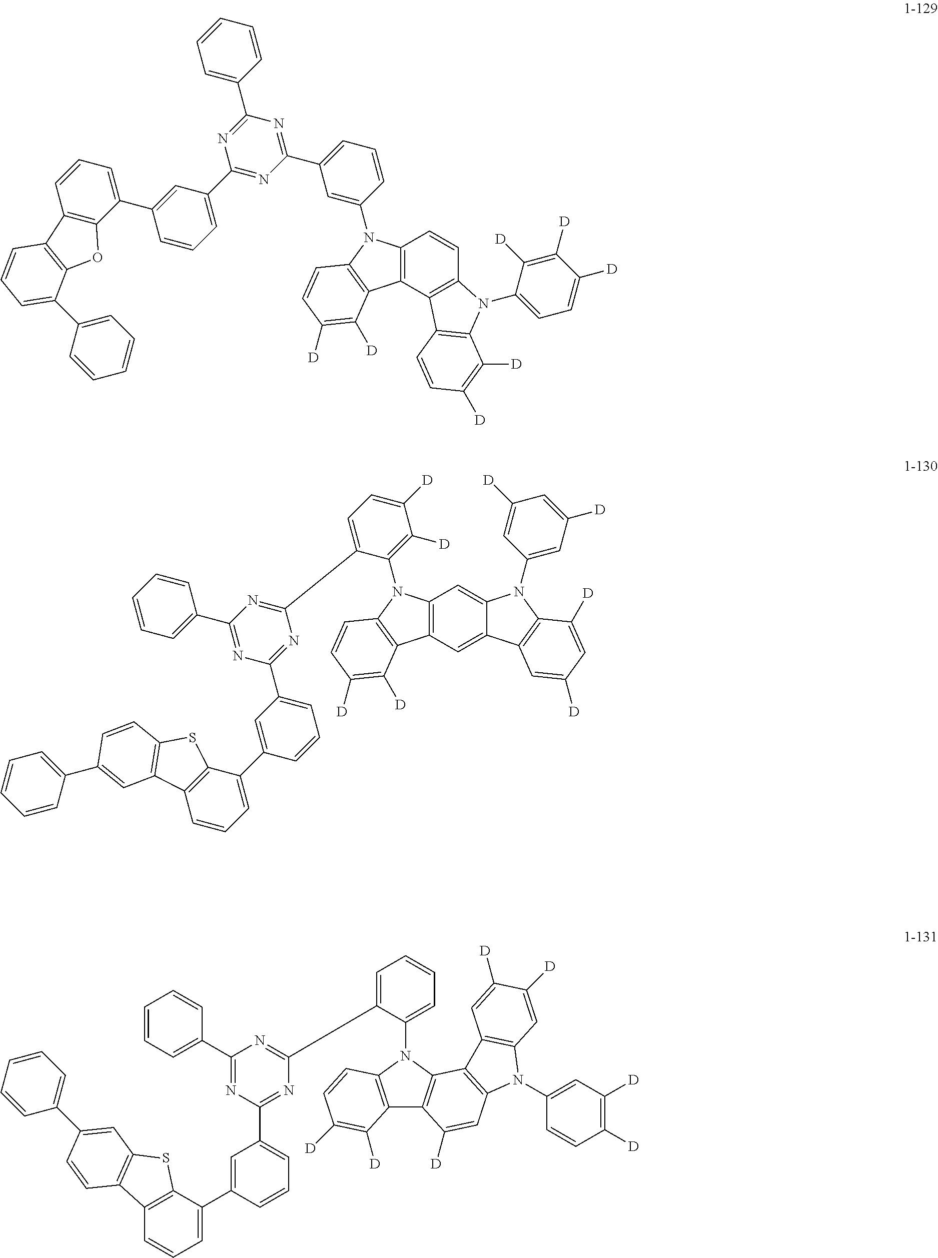 [SMILES: [2H]c1ccc(-n2c3ccc4c(c5c([2H])c([2H])ccc5n4-c4cccc(-c5nc(-c6ccccc6)nc(-c6cccc(-c7cccc8c7oc7c(-c9ccccc9)cccc78)c6)n5)c4)c3c3ccc([2H])c([2H])c32)c([2H])c1[2H] ; [2H]c1cc([2H])cc(-n2c3cc4c(cc3c3cc([2H])cc([2H])c32)c2c([2H])c([2H])ccc2n4-c2c(-c3nc(-c4ccccc4)nc(-c4cccc(-c5cccc6c5sc5ccc(-c7ccccc7)cc56)c4)n3)ccc([2H])c2[2H])c1 ; [2H]c1ccc(-n2c3cc([2H])c([2H])cc3c3c2cc([2H])c2c4c([2H])c([2H])ccc4n(-c4ccccc4-c4nc(-c5ccccc5)nc(-c5cccc(-c6cccc7c6sc6cc(-c8ccccc8)ccc67)c5)n4)c23)cc1[2H]]

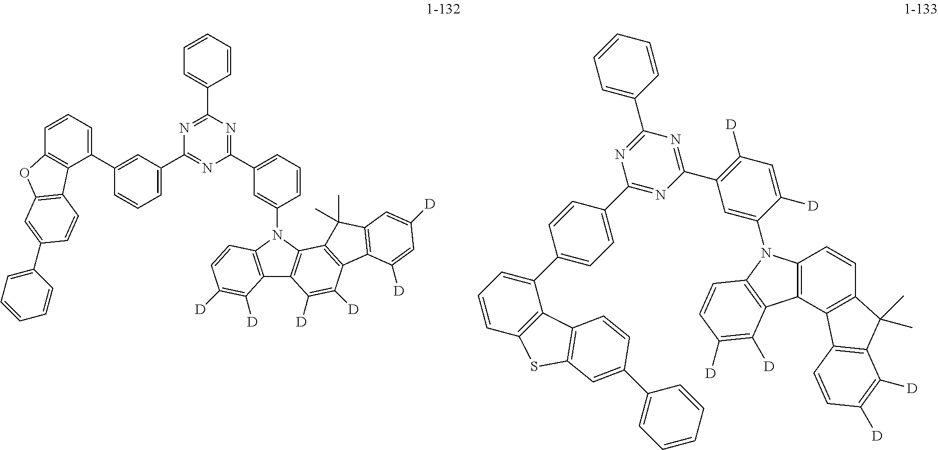 [SMILES: [2H]c1cc([2H])c2c(c1)C(C)(C)c1c-2c([2H])c([2H])c2c3c([2H])c([2H])ccc3n(-c3cccc(-c4nc(-c5ccccc5)nc(-c5cccc(-c6cccc7oc8cc(-c9ccccc9)ccc8c67)c5)n4)c3)c12 ; [2H]c1cc([2H])c(-n2c3ccc([2H])c([2H])c3c3c4c(ccc32)C(C)(C)c2c-4ccc([2H])c2[2H])cc1-c1nc(-c2ccccc2)nc(-c2ccc(-c3cccc4sc5cc(-c6ccccc6)ccc5c34)cc2)n1]

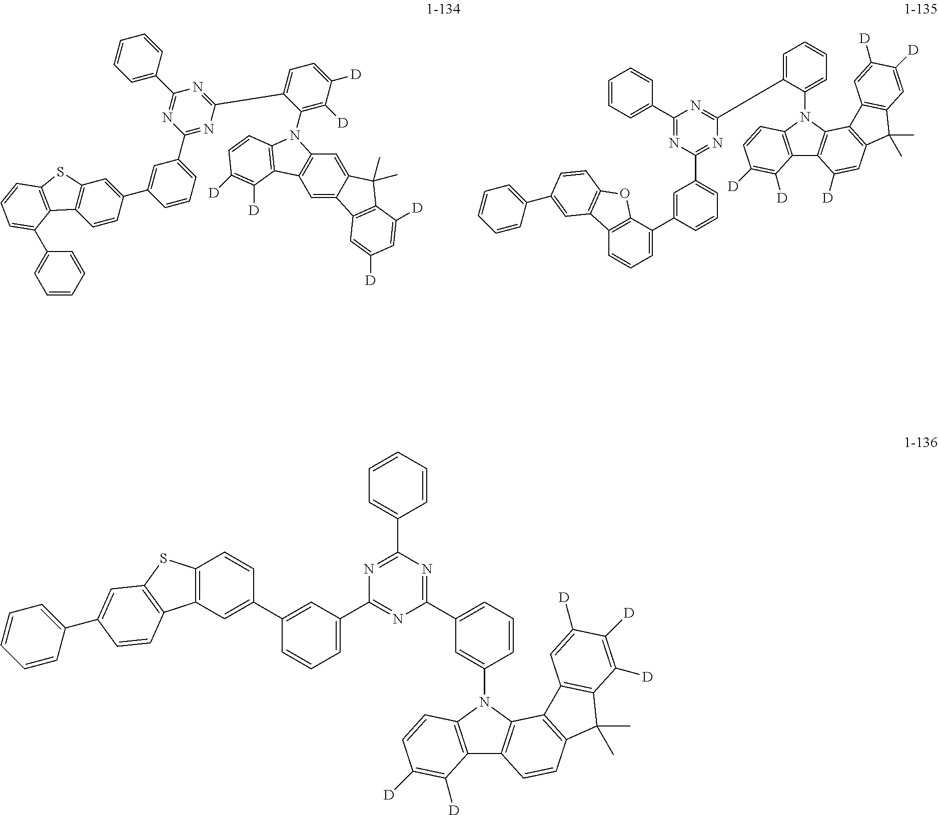 [SMILES: [2H]c1cc([2H])c2c(c1)-c1cc3c4c([2H])c([2H])ccc4n(-c4c(-c5nc(-c6ccccc6)nc(-c6cccc(-c7ccc8c(c7)sc7cccc(-c9ccccc9)c78)c6)n5)ccc([2H])c4[2H])c3cc1C2(C)C ; [2H]c1cc2c(cc1[2H])C(C)(C)c1cc([2H])c3c4c([2H])c([2H])ccc4n(-c4ccccc4-c4nc(-c5ccccc5)nc(-c5cccc(-c6cccc7c6oc6ccc(-c8ccccc8)cc67)c5)n4)c3c1-2 ; [2H]c1cc2c(c([2H])c1[2H])C(C)(C)c1ccc3c4c([2H])c([2H])ccc4n(-c4cccc(-c5nc(-c6ccccc6)nc(-c6cccc(-c7ccc8sc9cc(-c%10ccccc%10)ccc9c8c7)c6)n5)c4)c3c1-2]

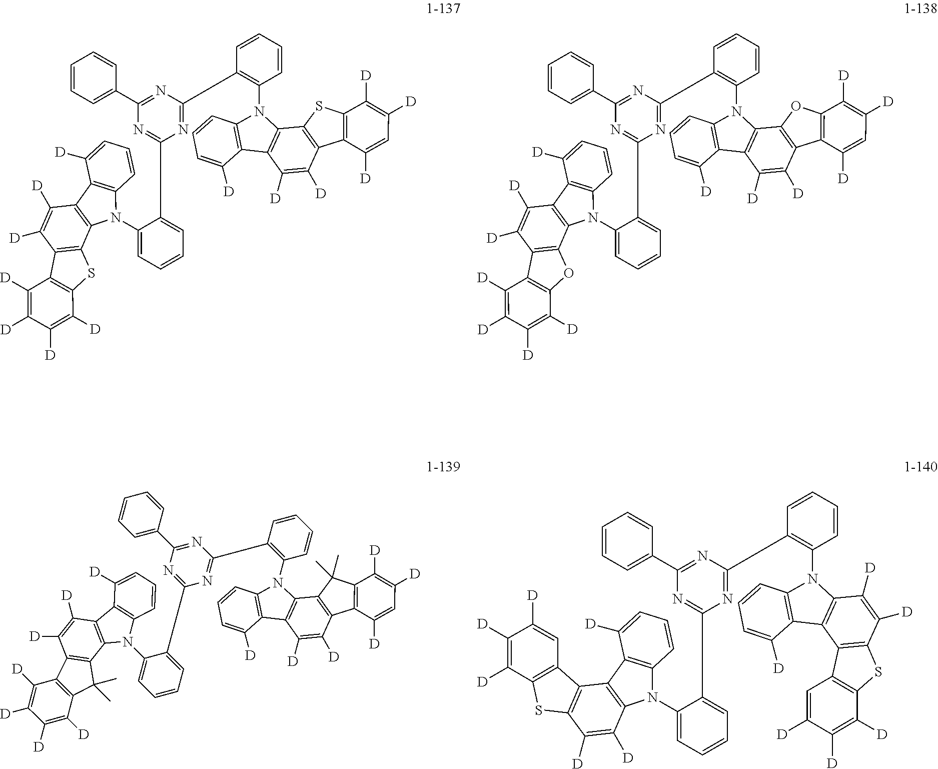

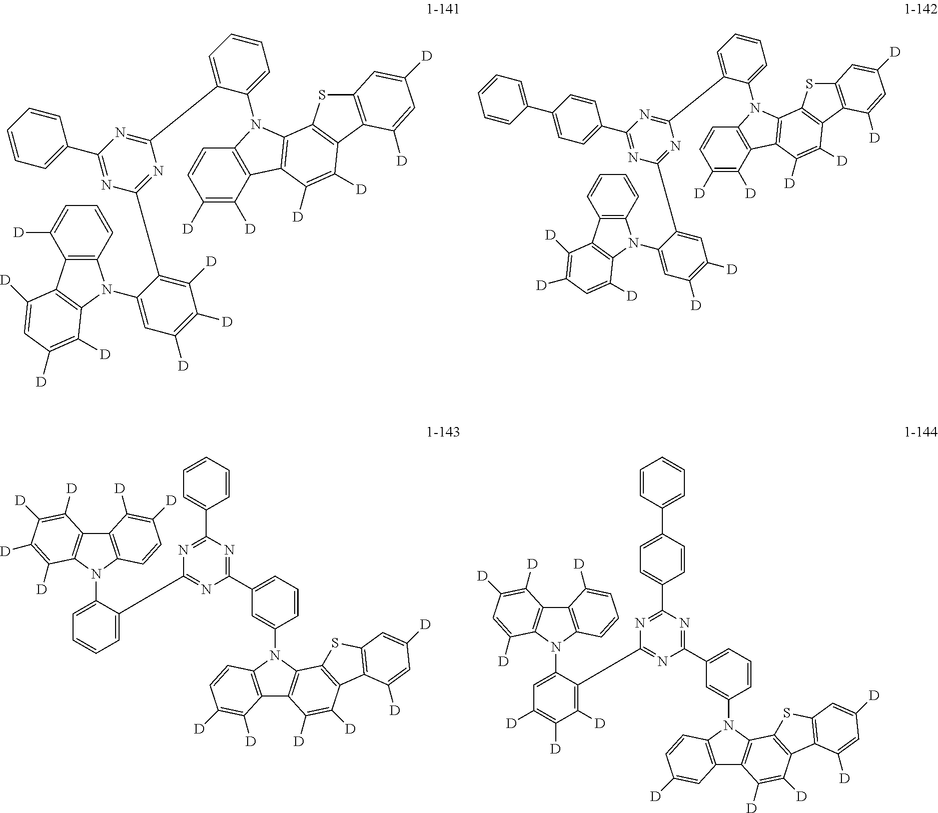

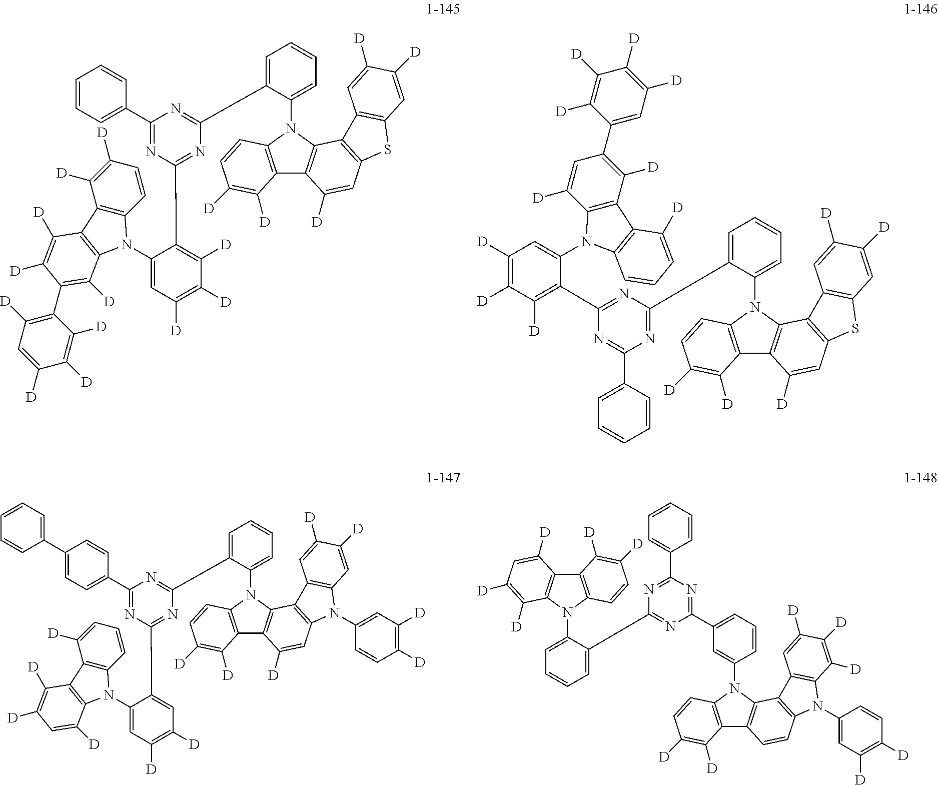

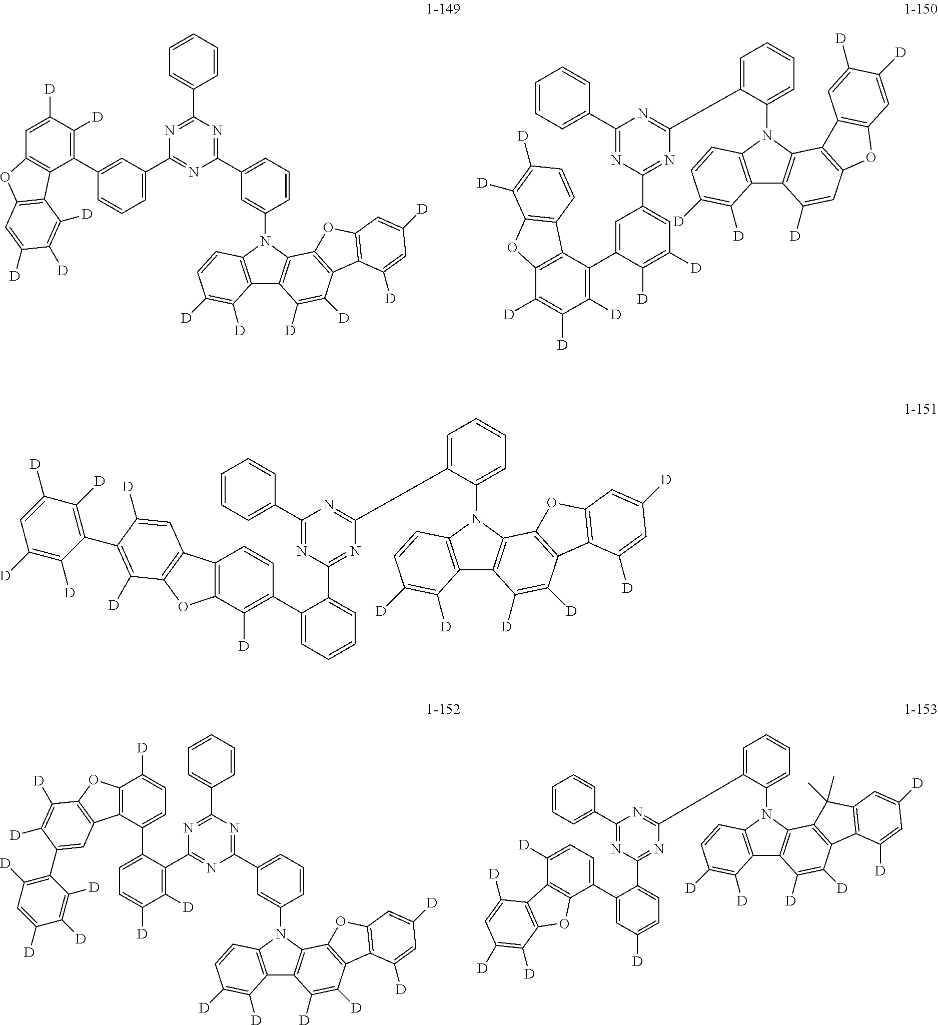

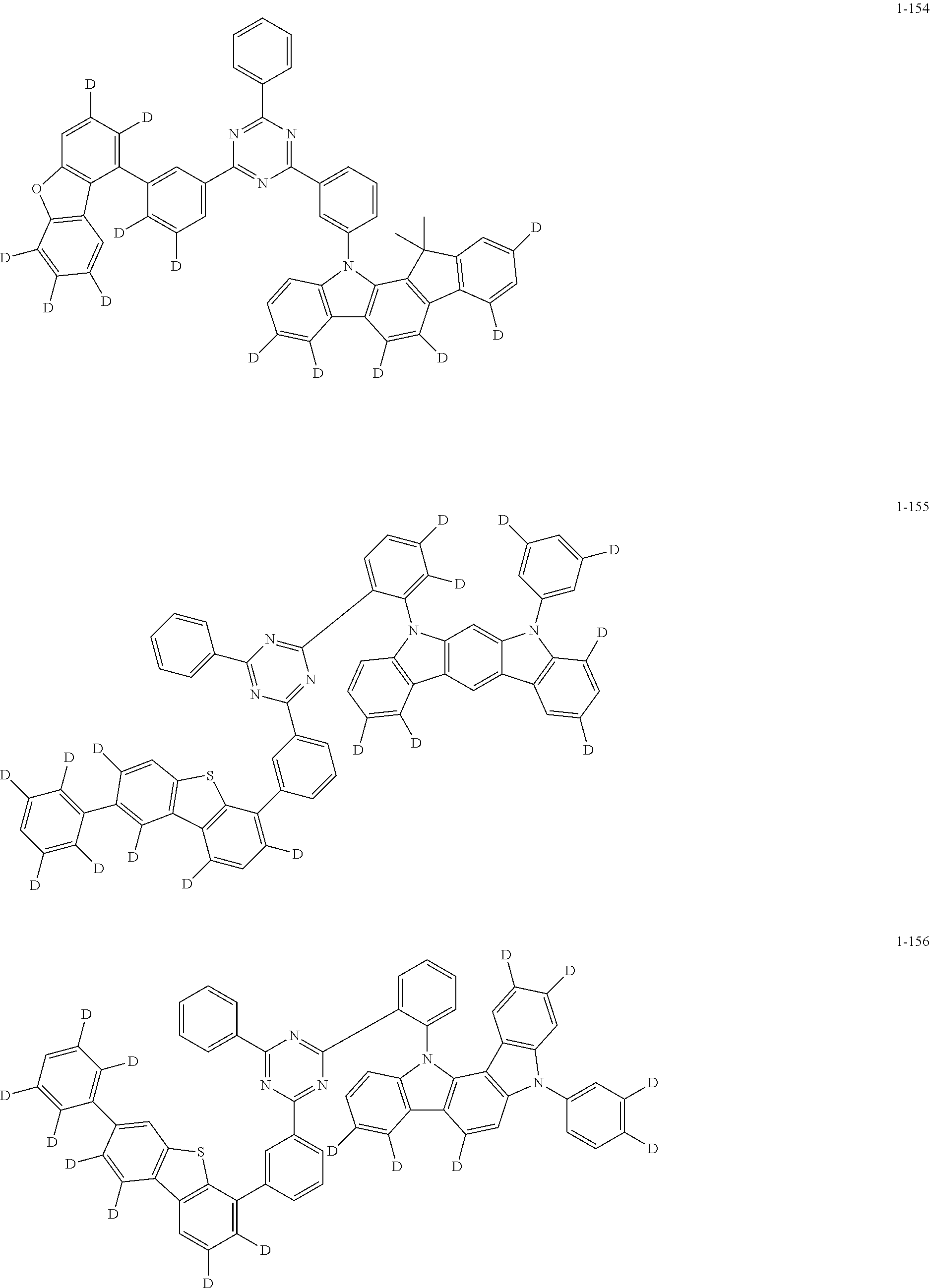

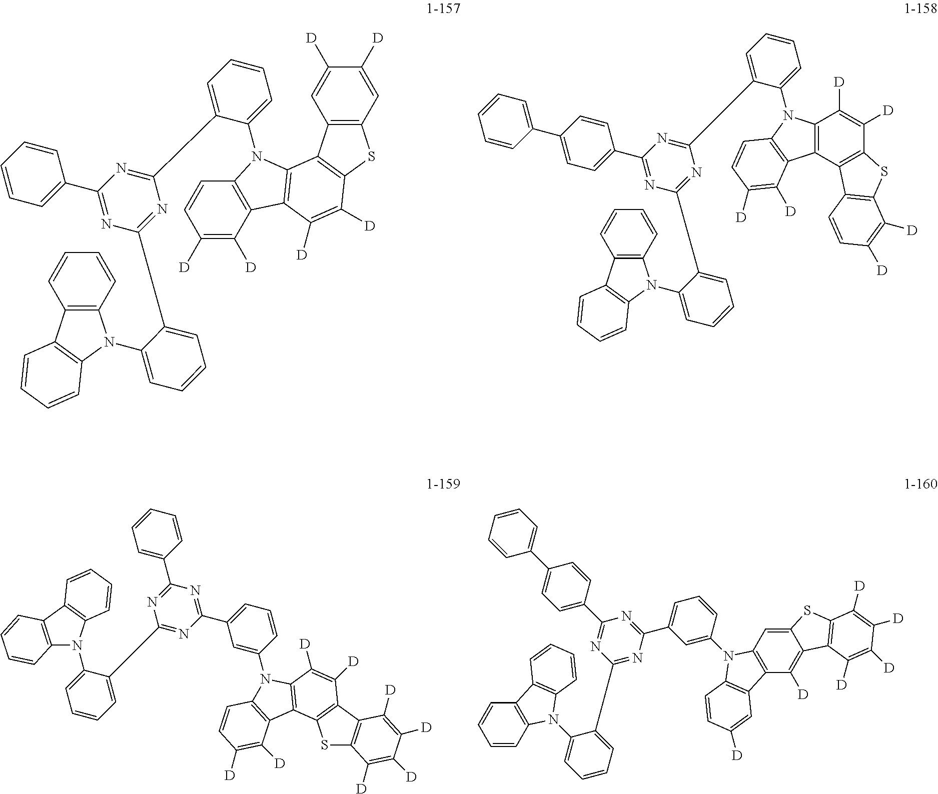

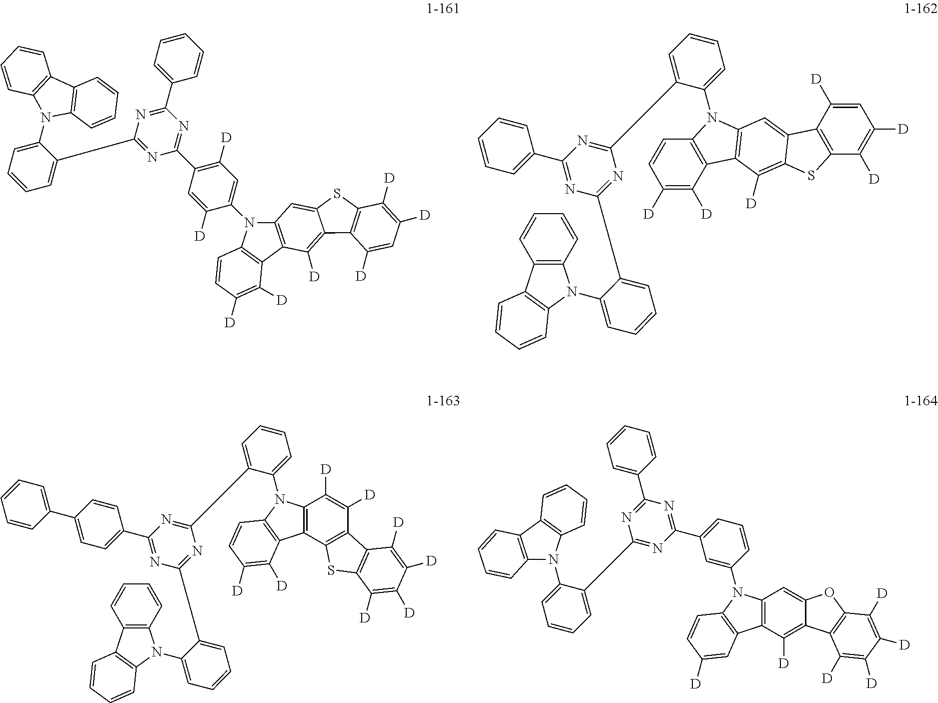

10. An organic light emitting device comprising:
a first electrode;
a second electrode; and
an organic material layer having one or more layers provided between the first electrode and the second electrode,
wherein one or more layers of the organic material layer comprise one or more of the heterocyclic compounds of claim 1.

11. The organic light emitting device of claim 10, wherein the organic material layer comprises a light emitting layer, and the light emitting layer comprises one or more of the heterocyclic compounds.

12. The organic light emitting device of claim 10, wherein the organic material layer comprising the heterocyclic compound further comprises a compound of the following Chemical Formula 2 or 3: 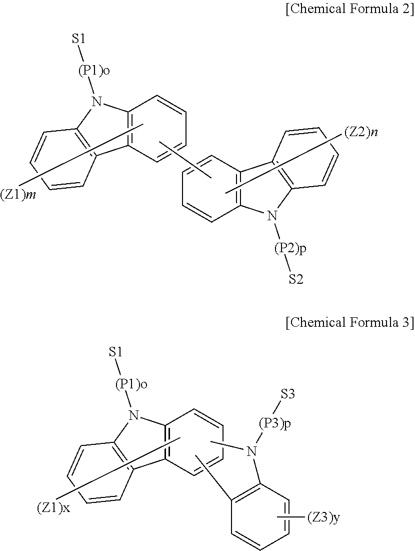
in Chemical Formulae 2 and 3,
P1 to P3 are each independently a direct bond; a substituted or unsubstituted C6 to C60 arylene group; or a substituted or unsubstituted C2 to C60 heteroarylene group,
S1 to S3 are each independently a cyano group; a substituted or unsubstituted triarylsilyl group; a substituted or unsubstituted C6 to C60 aryl group; or a substituted or unsubstituted C2 to C60 heteroaryl group,
Z1 to Z3 are each independently hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
m and n are each an integer of 1 to 7,
x is an integer from 1 to 6,
y is an integer from 1 to 4,
o, p, and q are each an integer from 1 to 3, and
when m, n, x, y, o, p, and g are each 2 or greater, substituents in the parenthesis are the same as or different from each other.

13. The organic light emitting device of claim 12, wherein Chemical Formula 2 is selected from the following compounds:

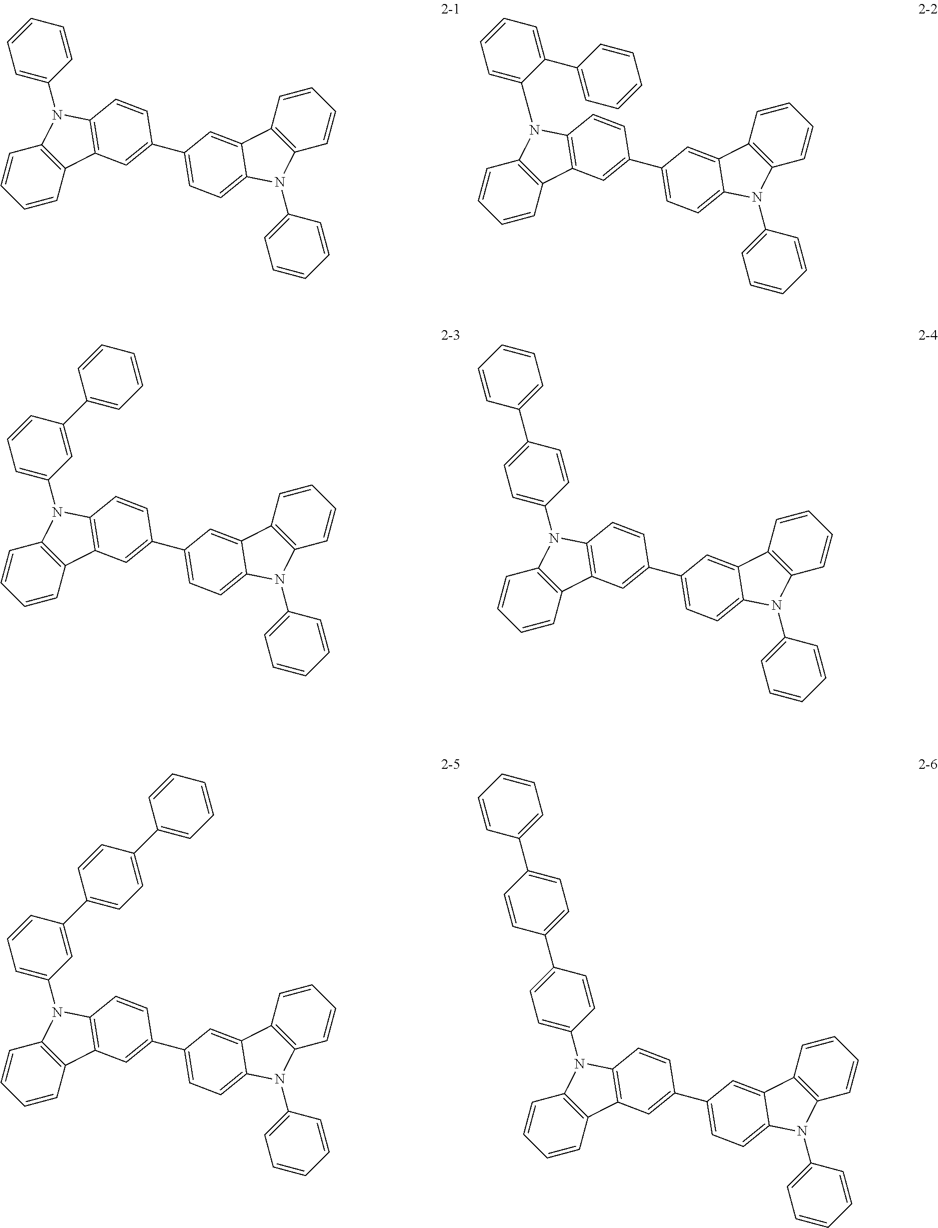 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

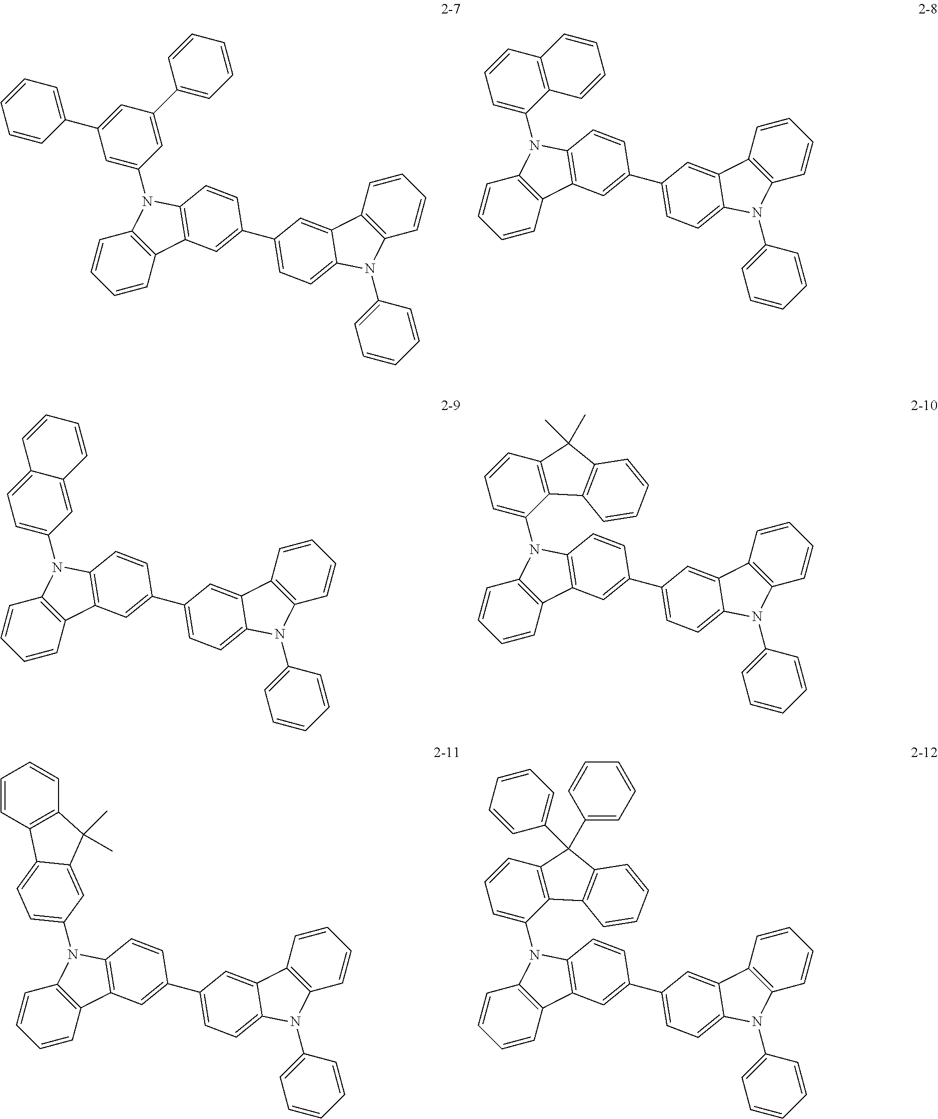 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

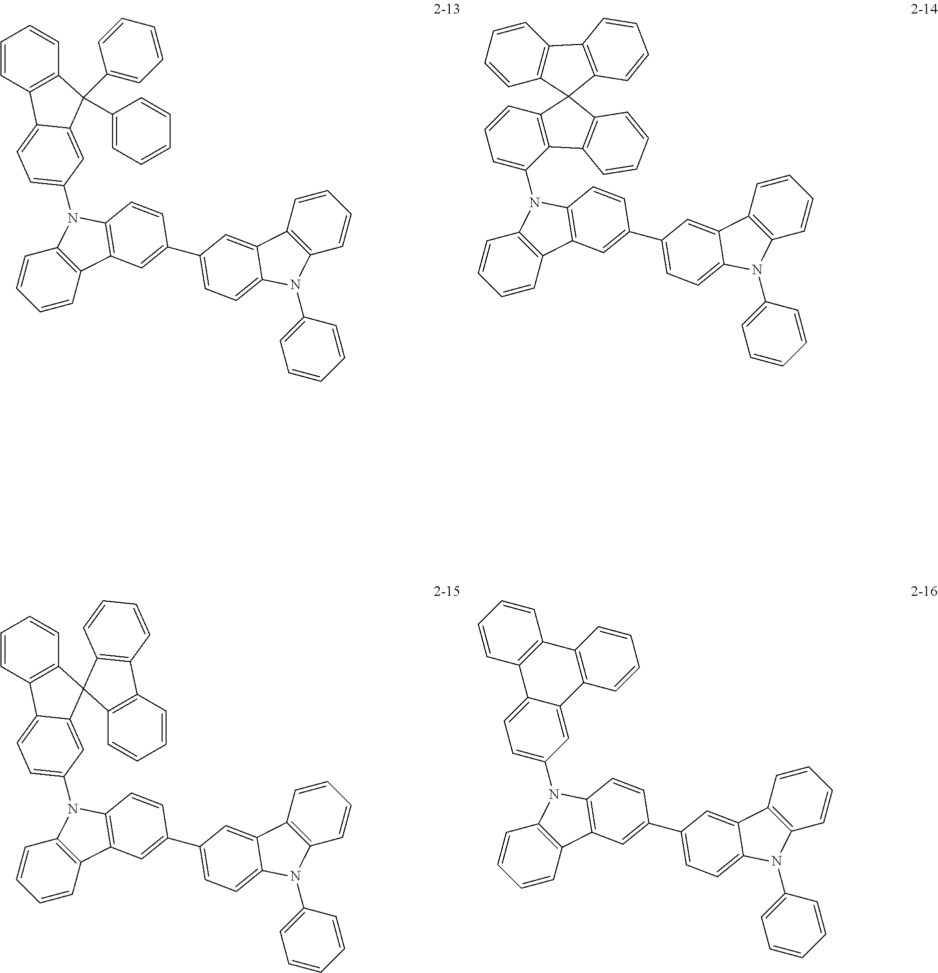 [SMILES: c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

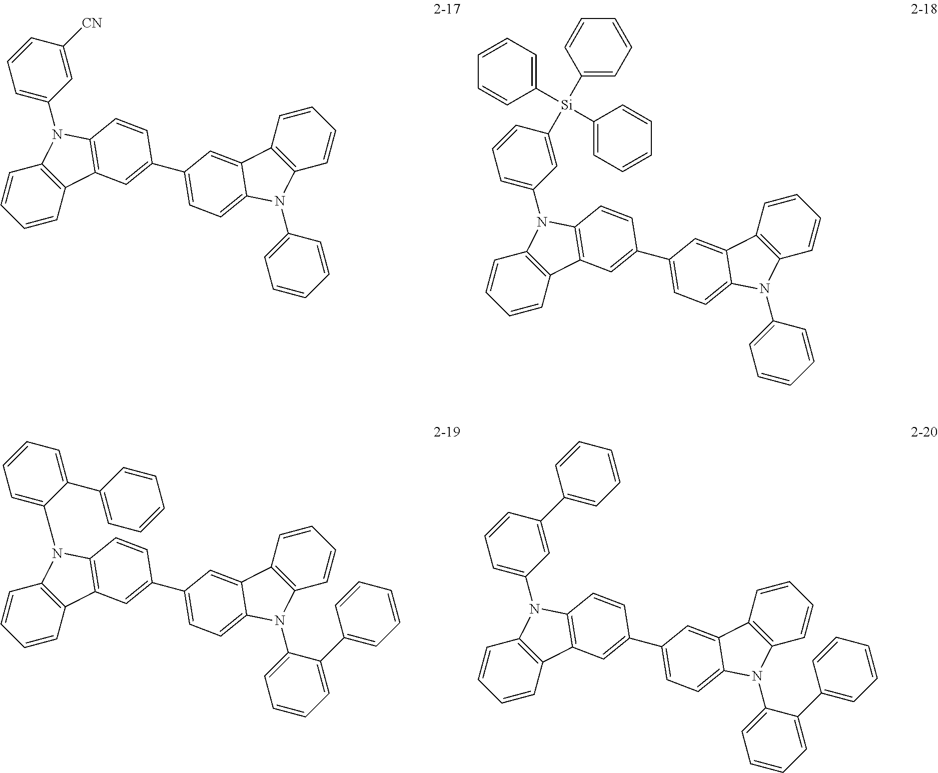 [SMILES: N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1]

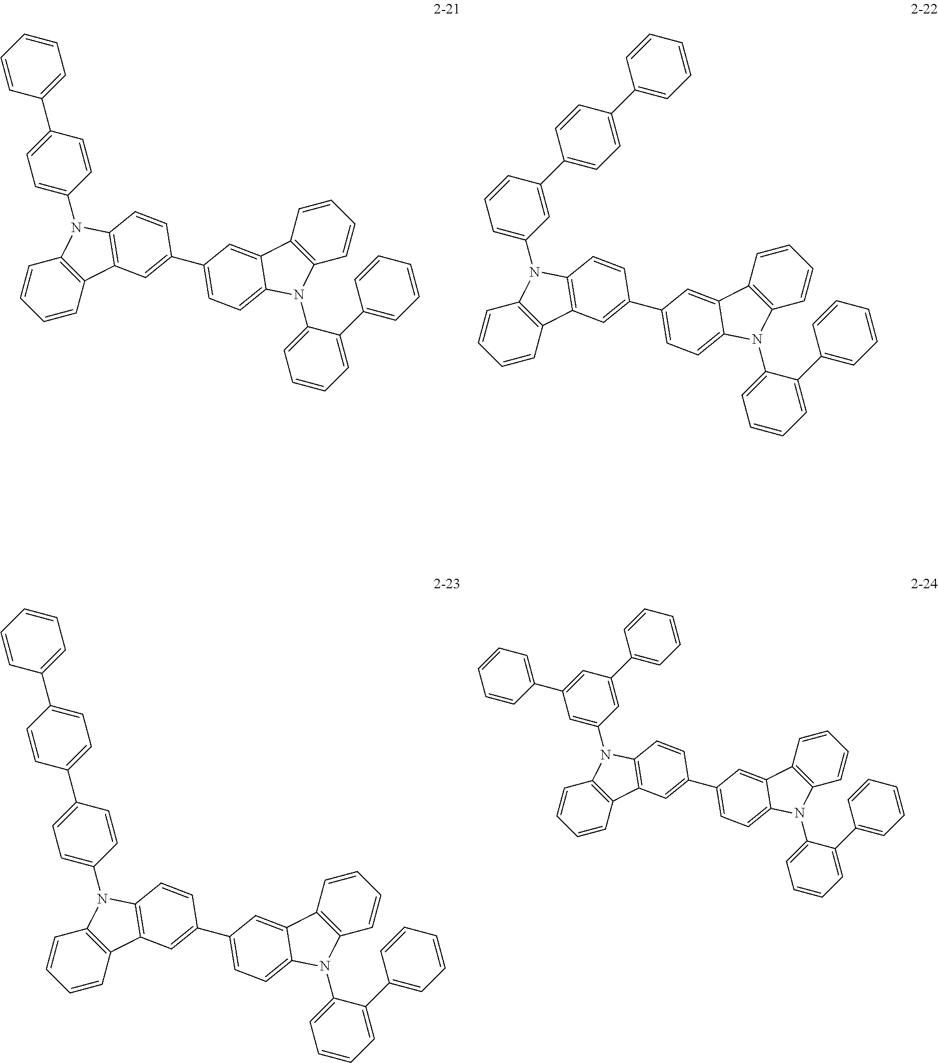 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1]

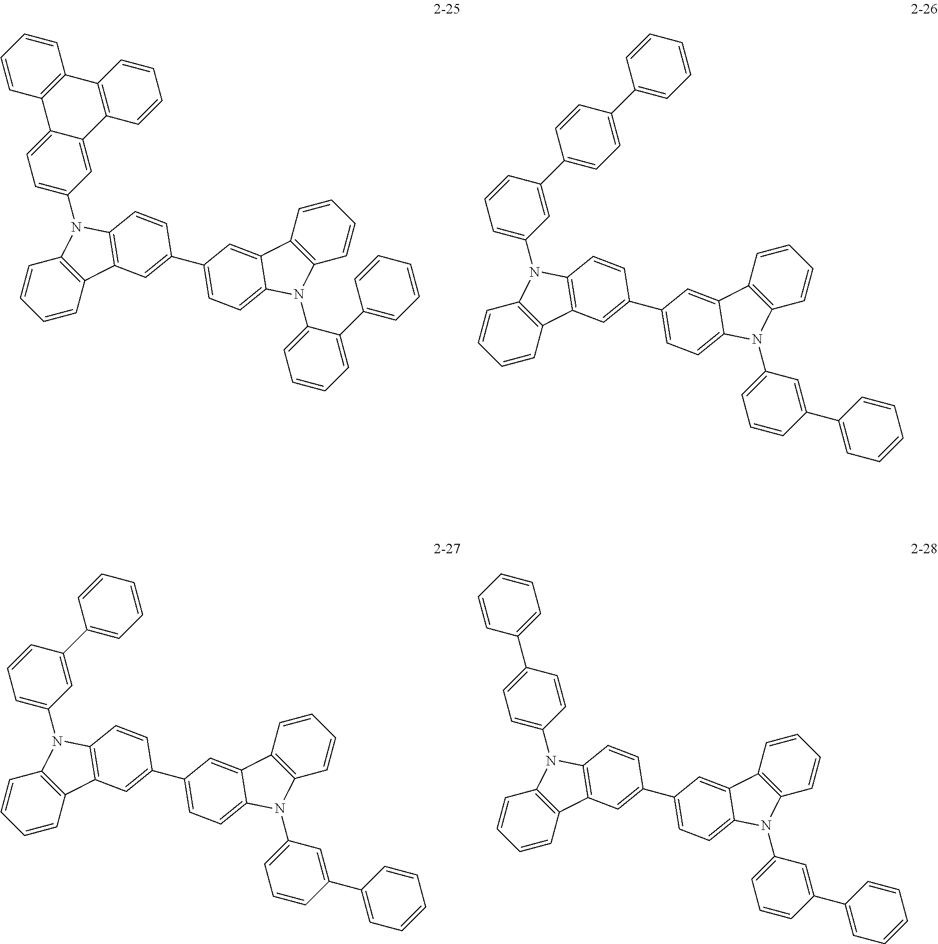 [SMILES: c1ccc(-c2ccccc2-n2c3ccccc3c3cc(-c4ccc5c(c4)c4ccccc4n5-c4ccc5c6ccccc6c6ccccc6c5c4)ccc32)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

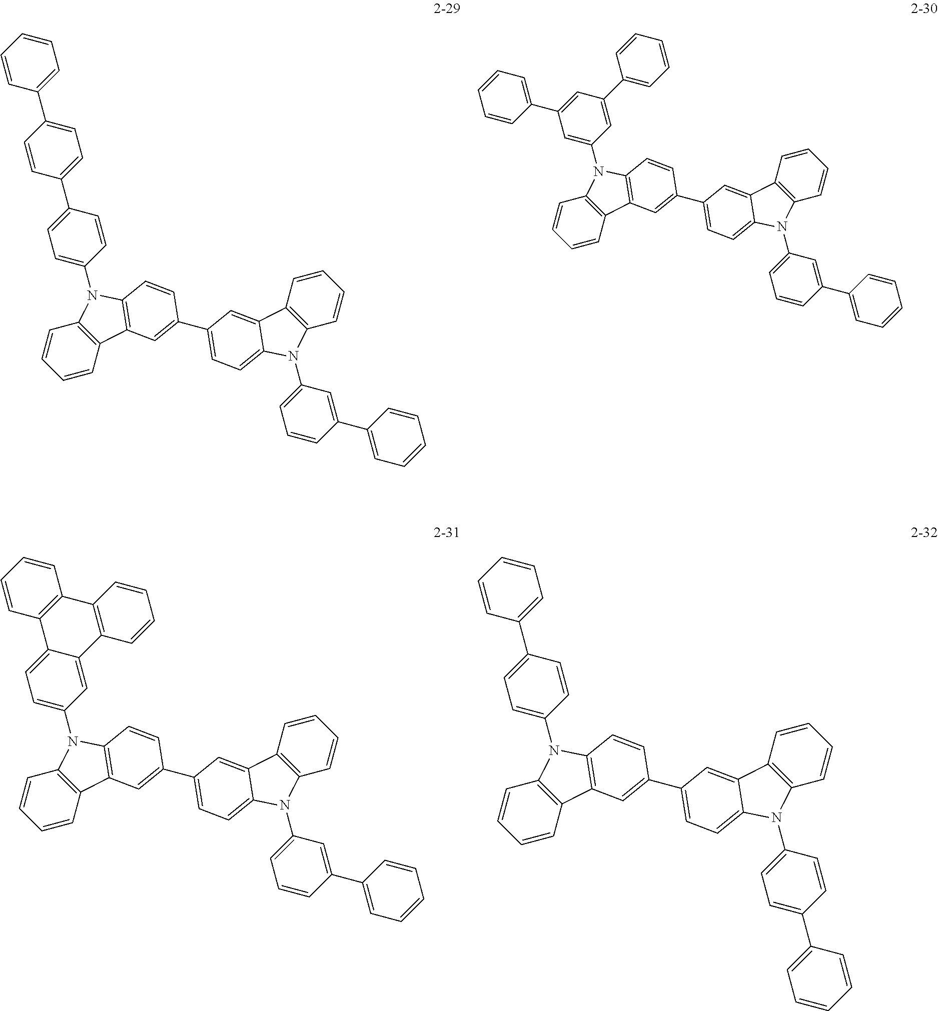 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

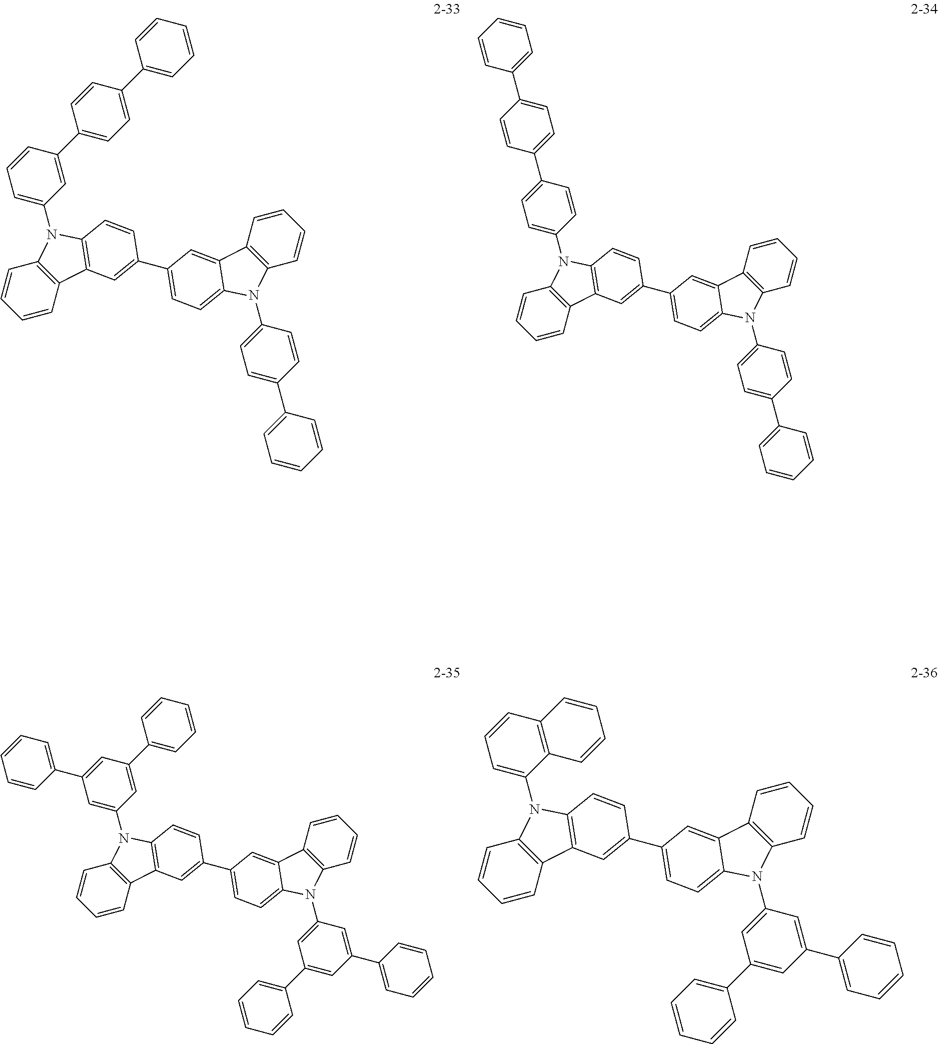 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

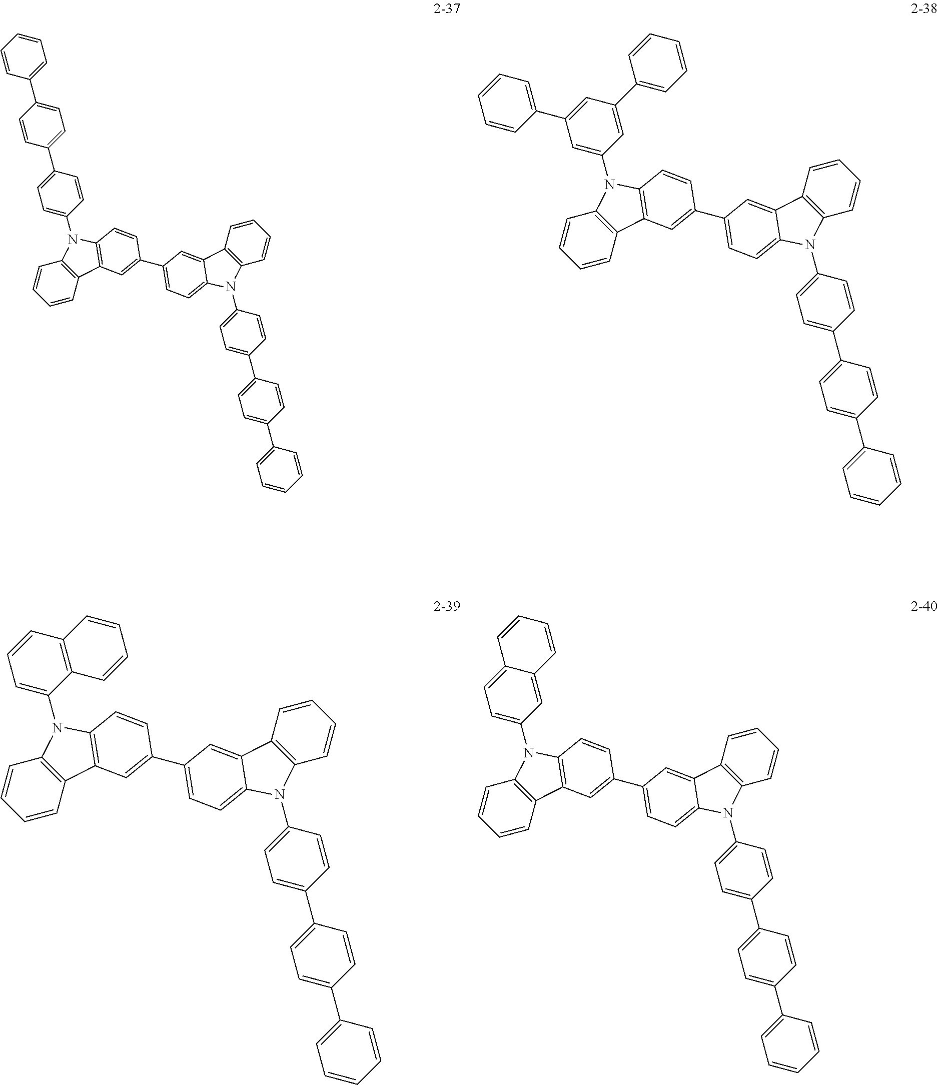 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

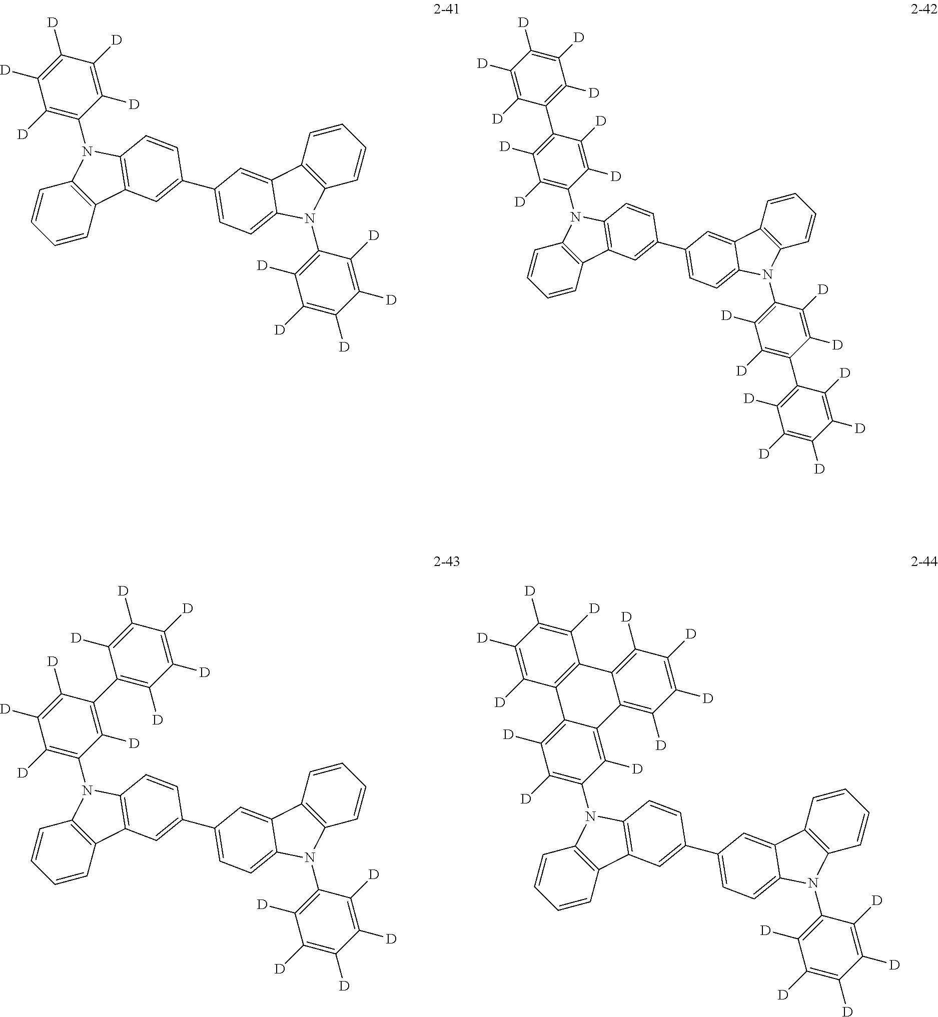

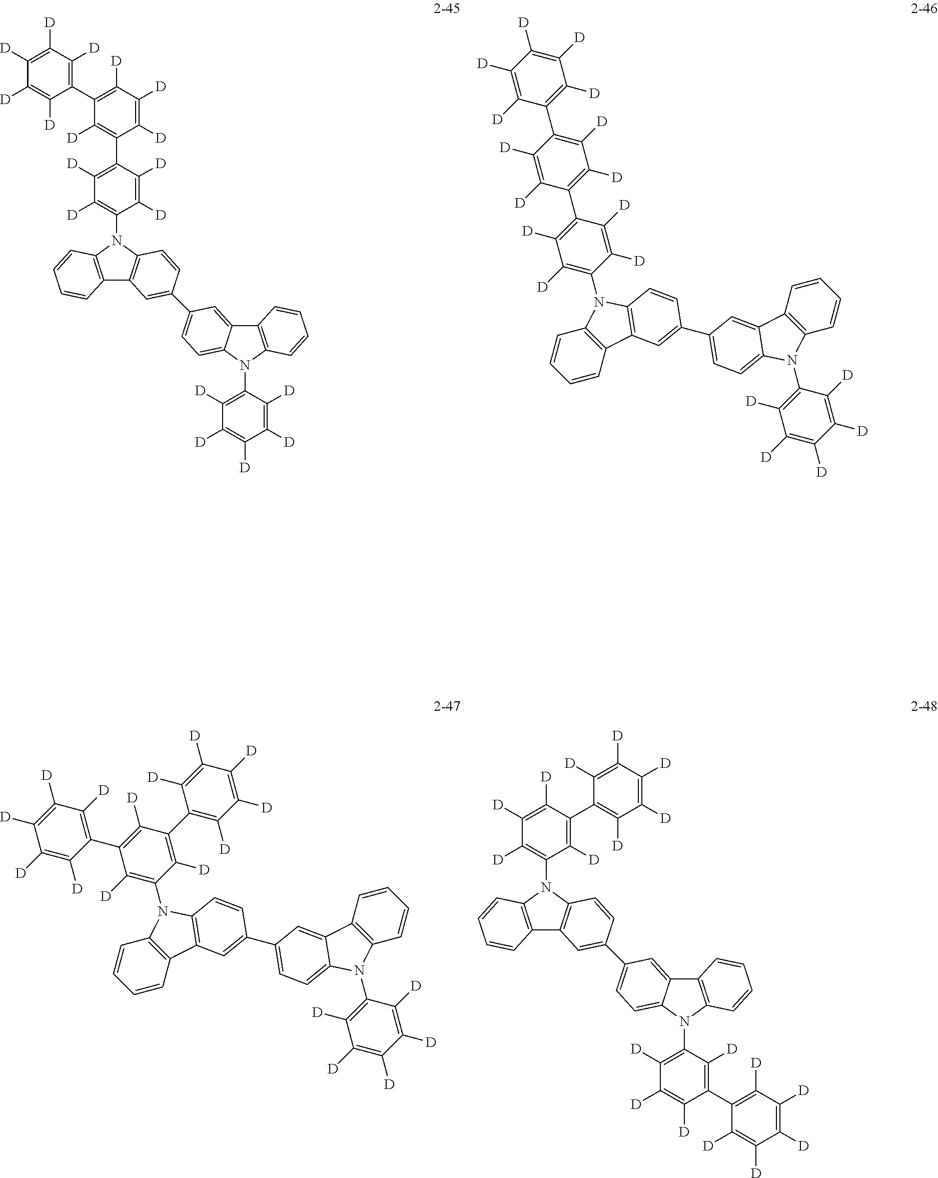

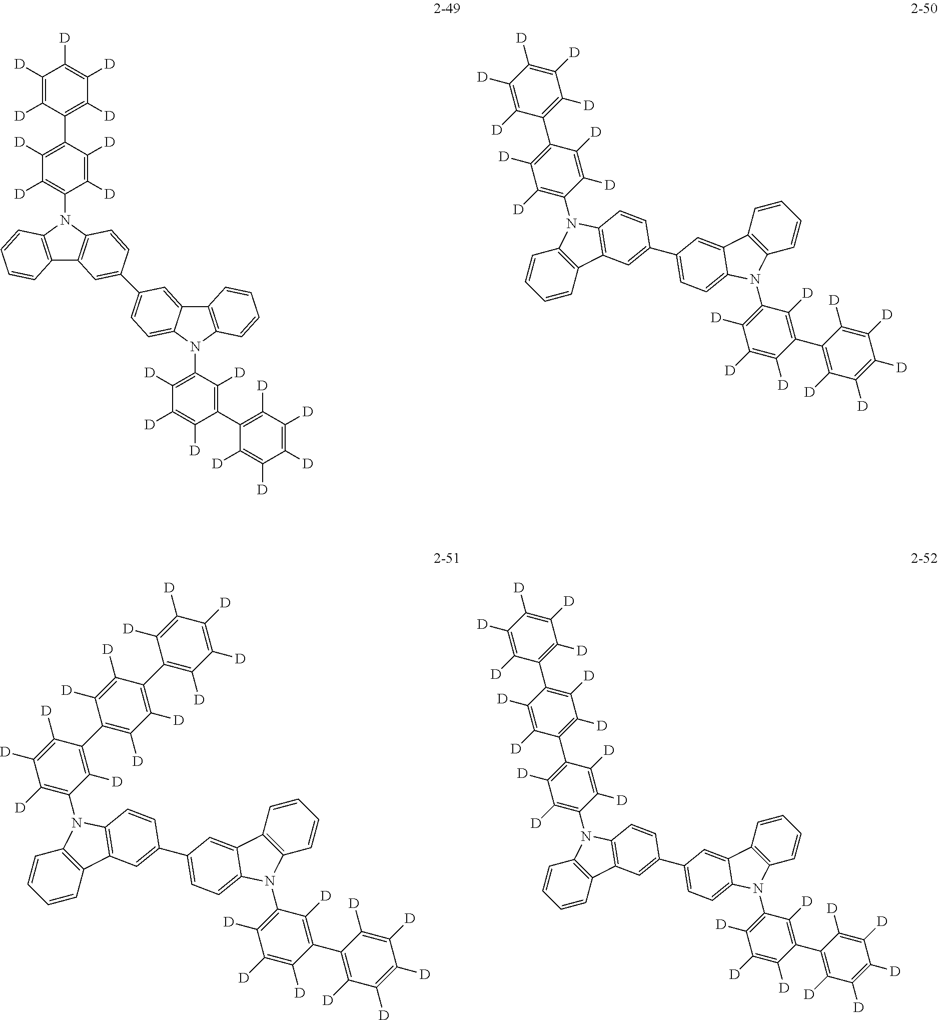

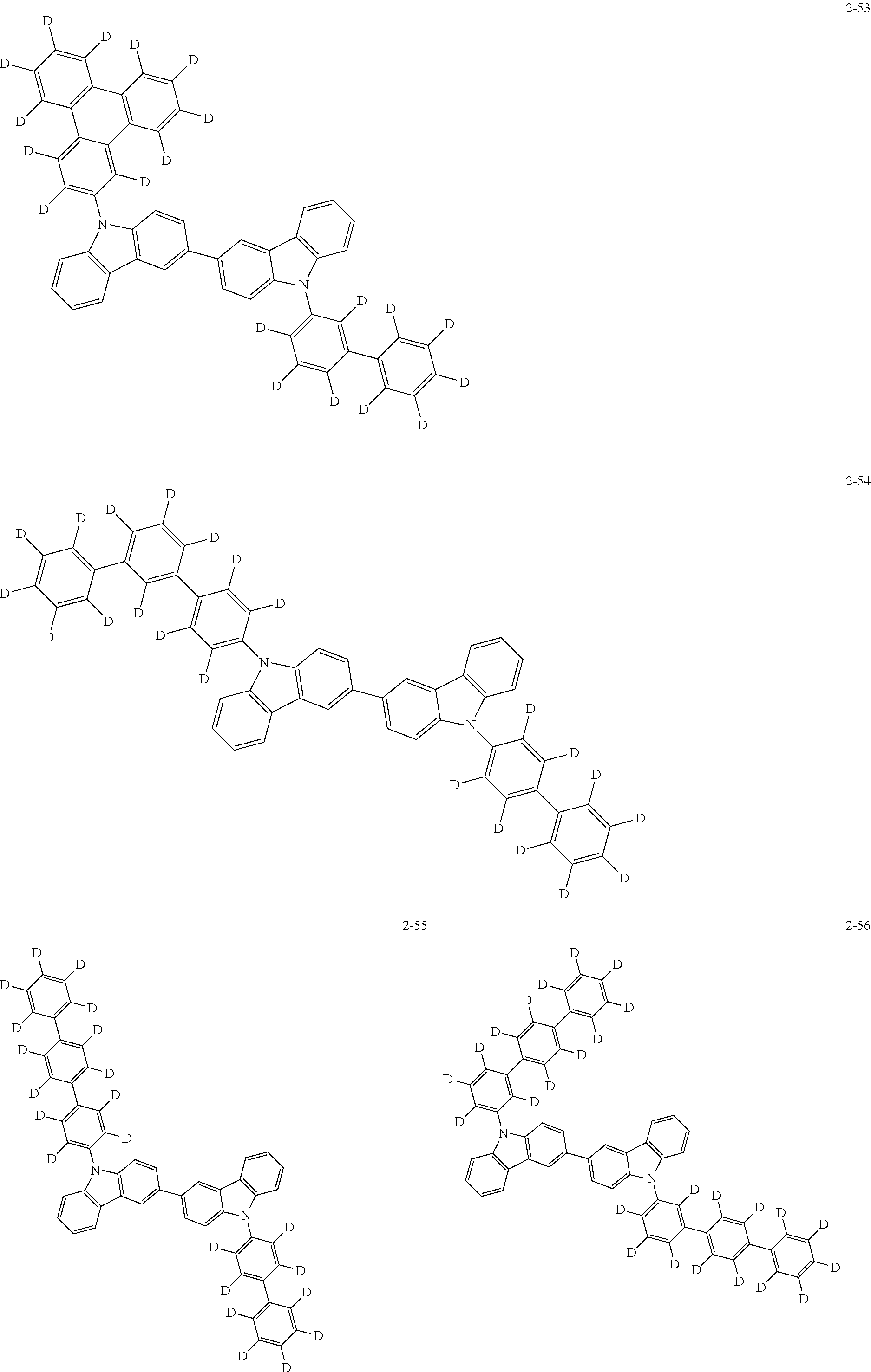

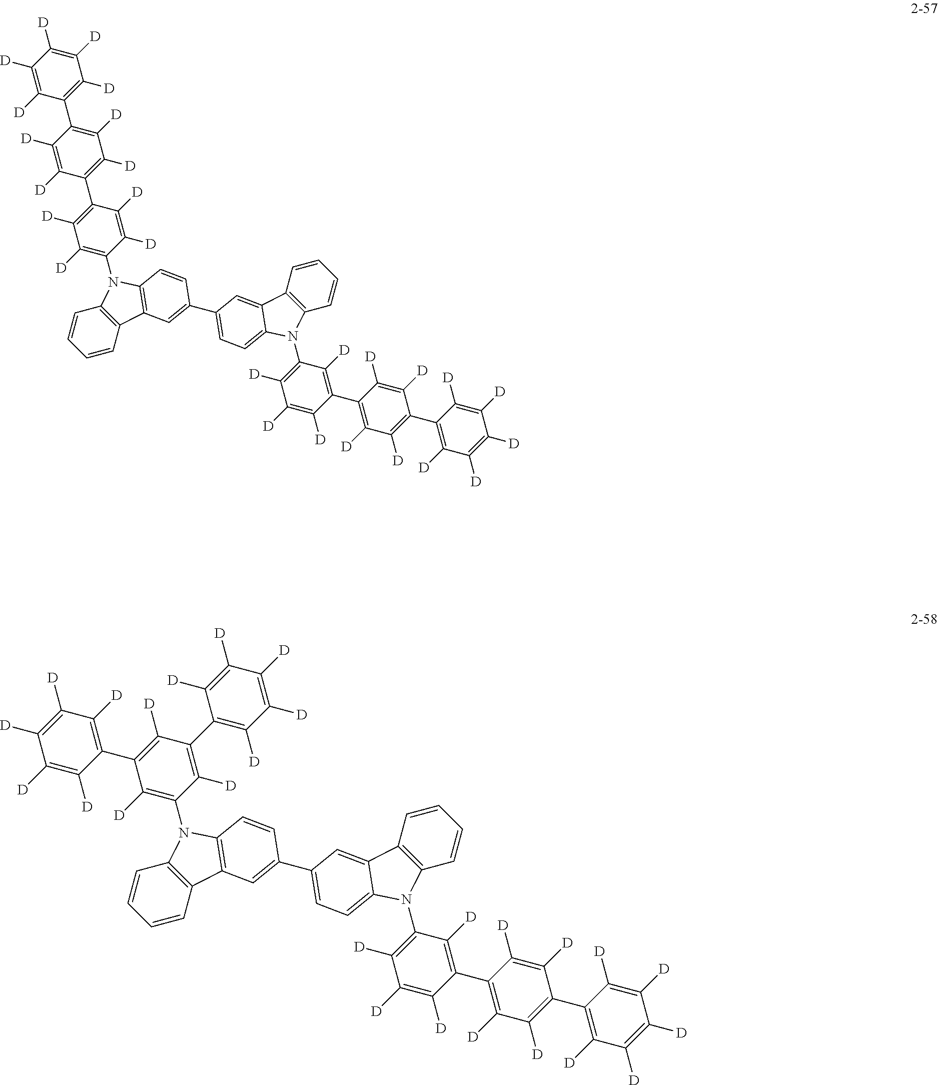

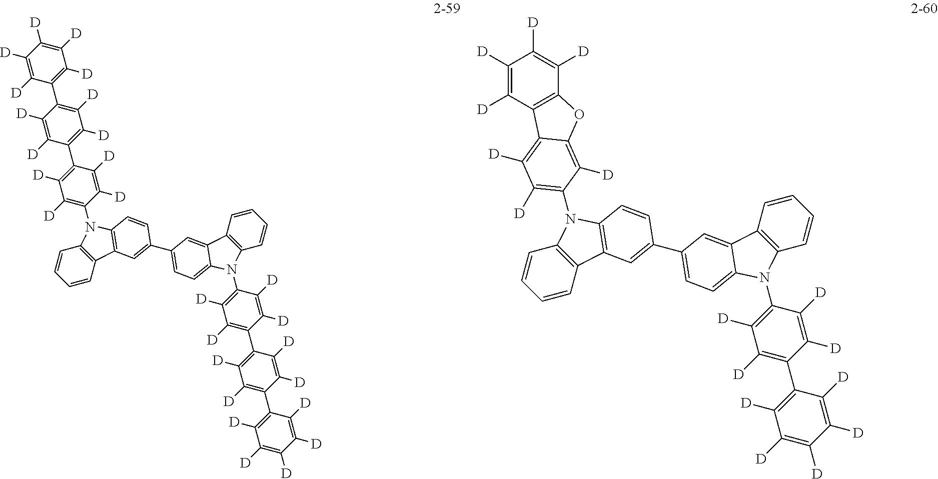 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c(-c8c([2H])c([2H])c([2H])c([2H])c8[2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H]]

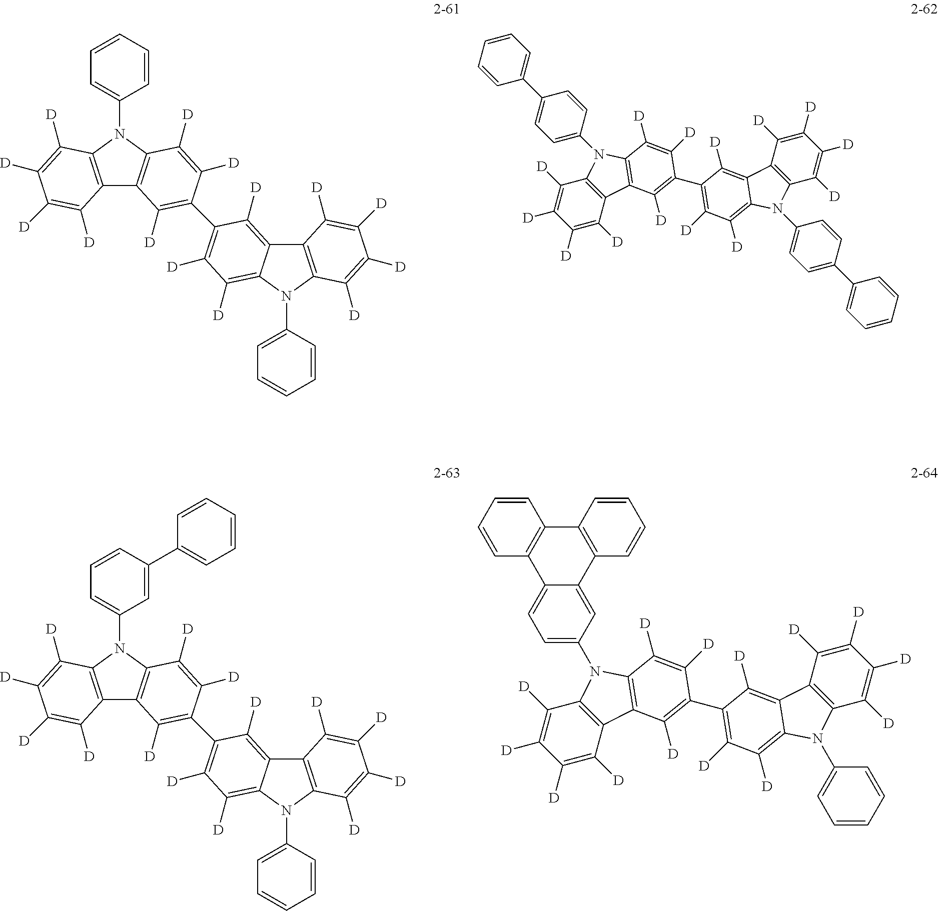

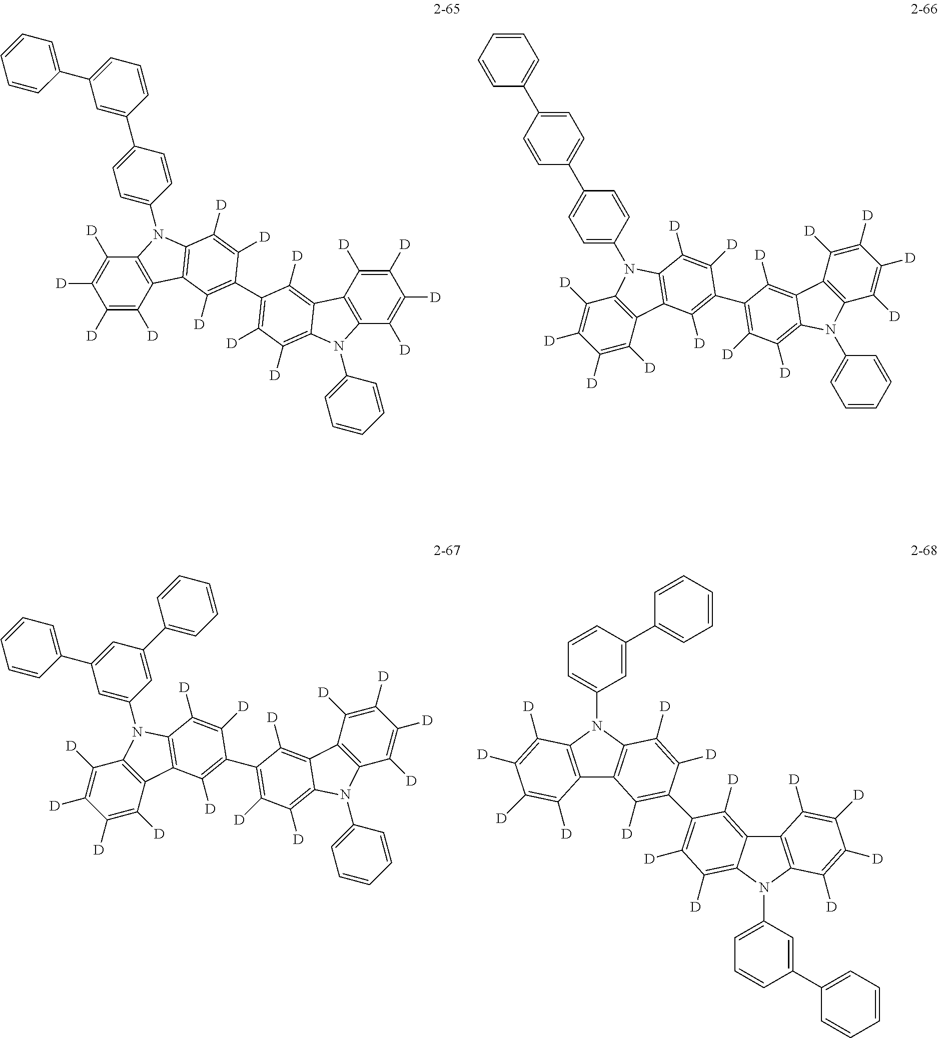

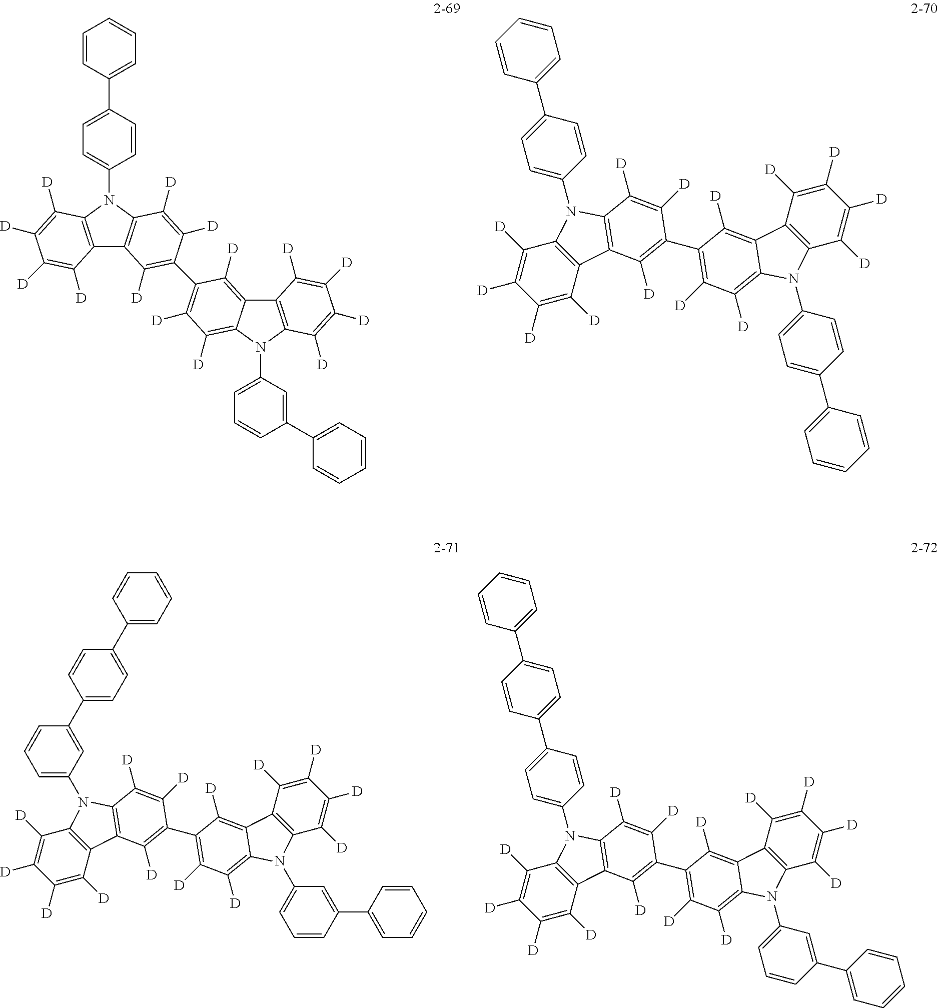

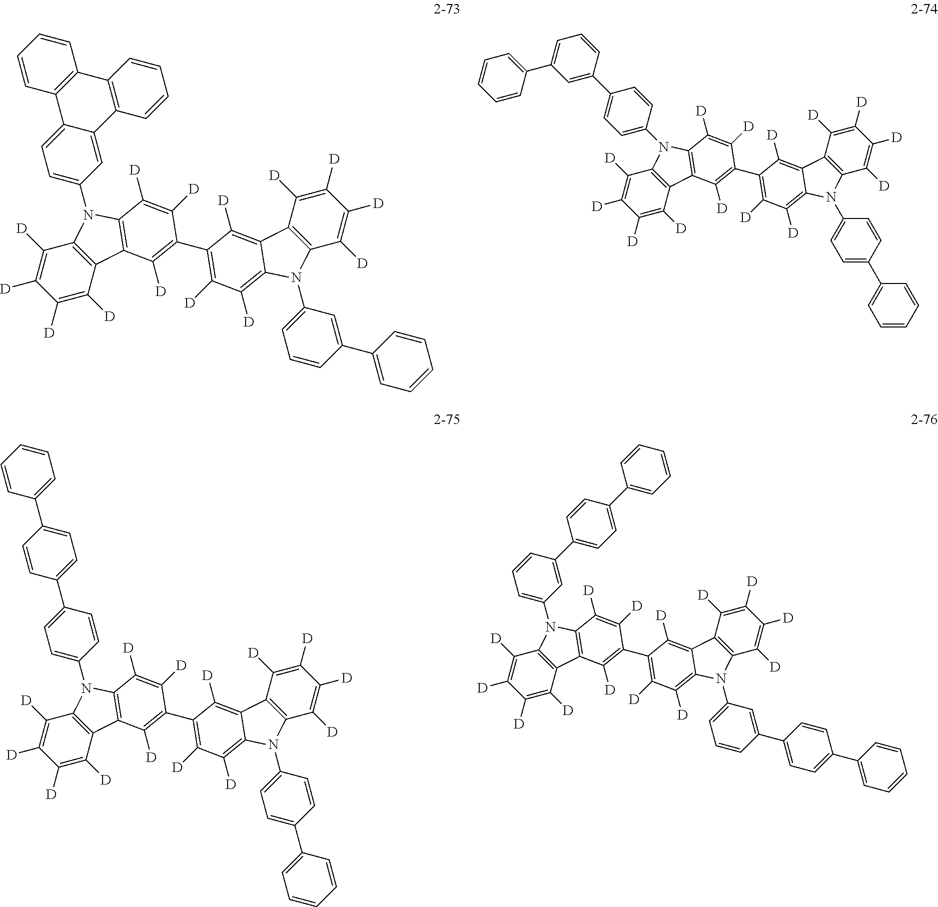

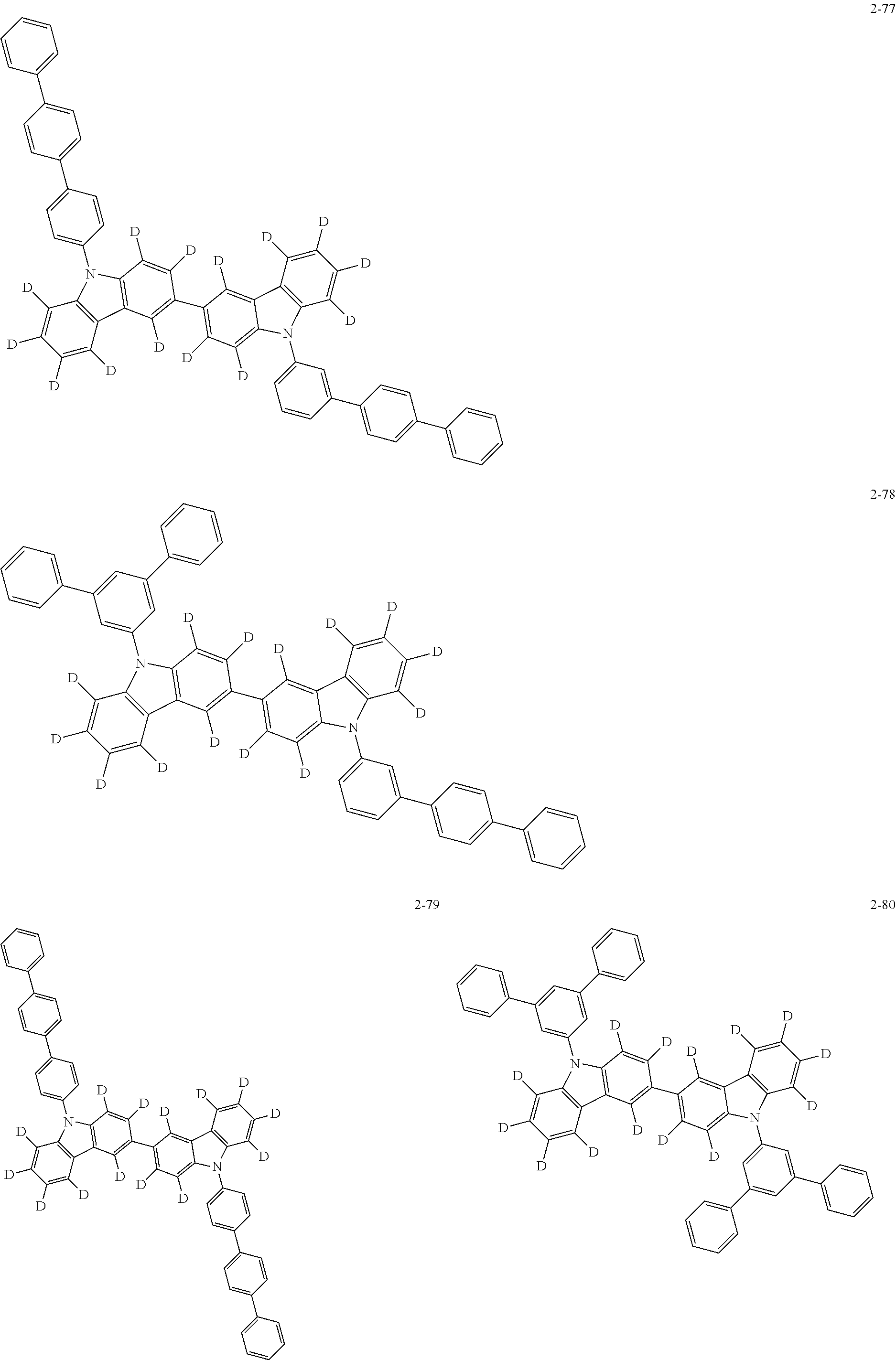

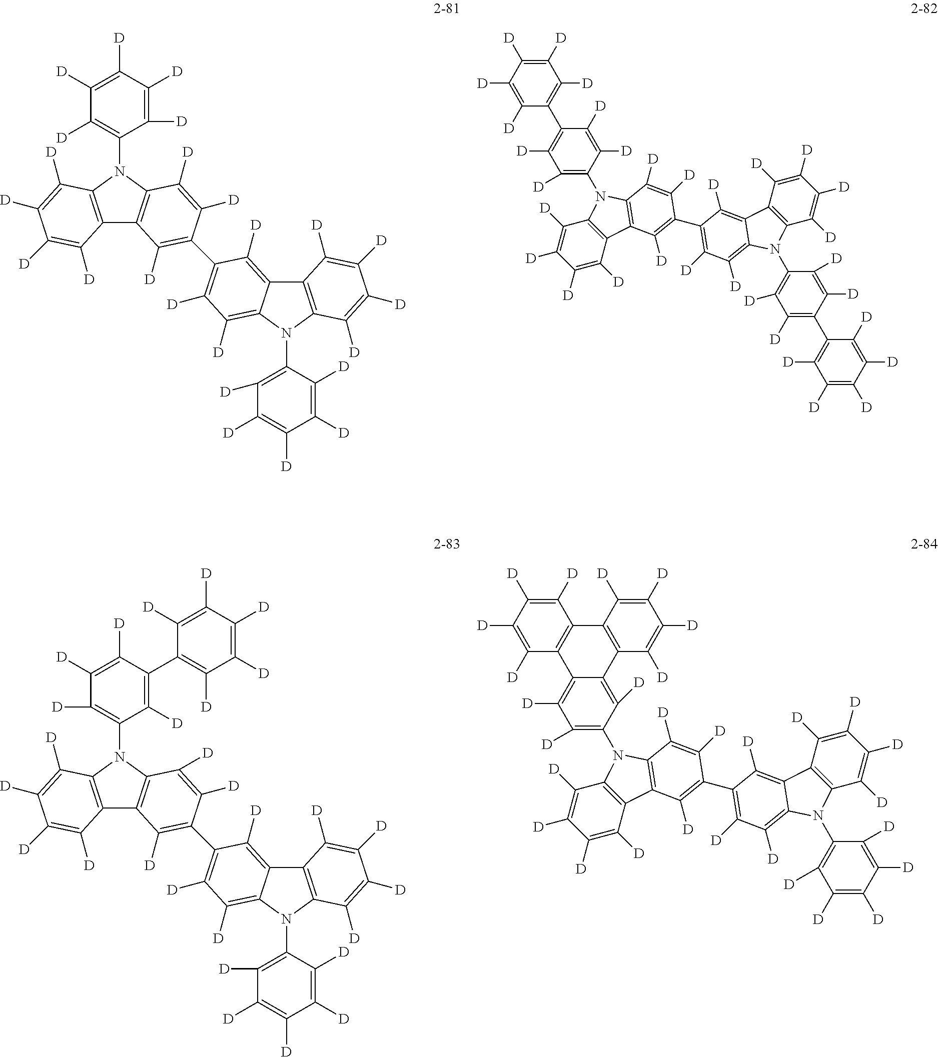

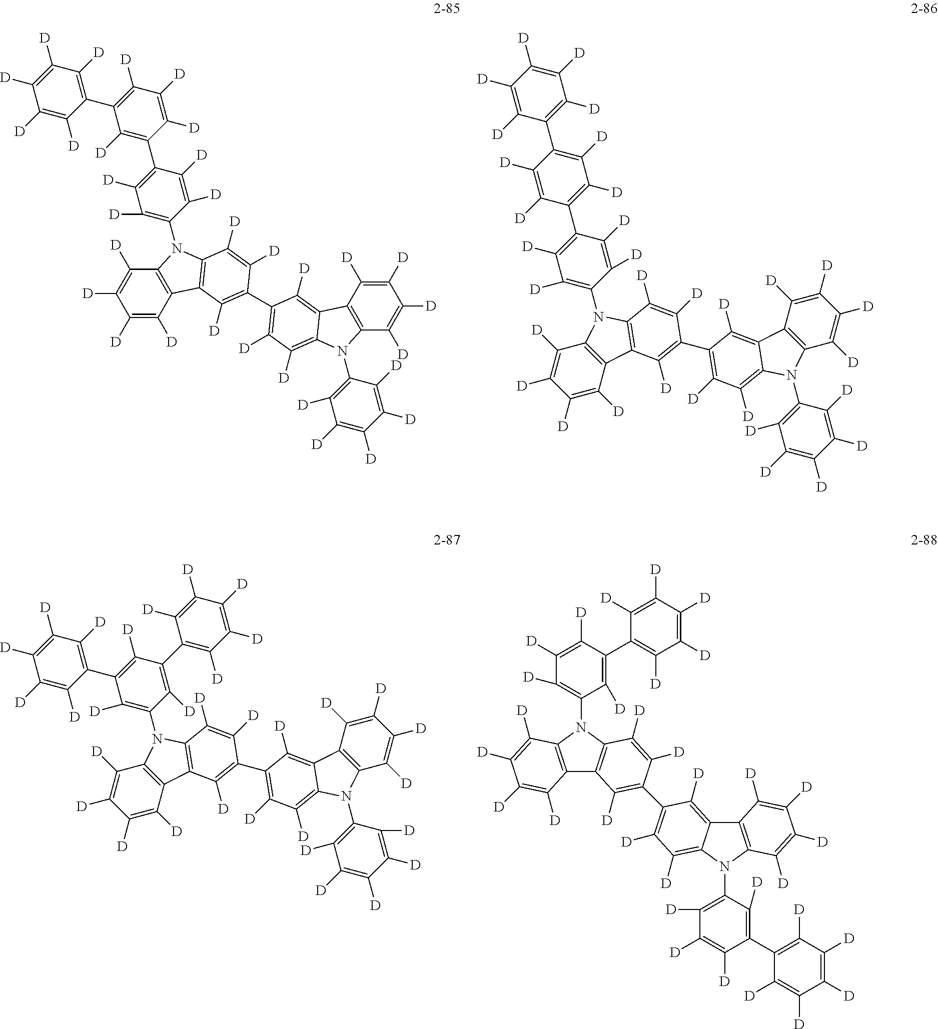

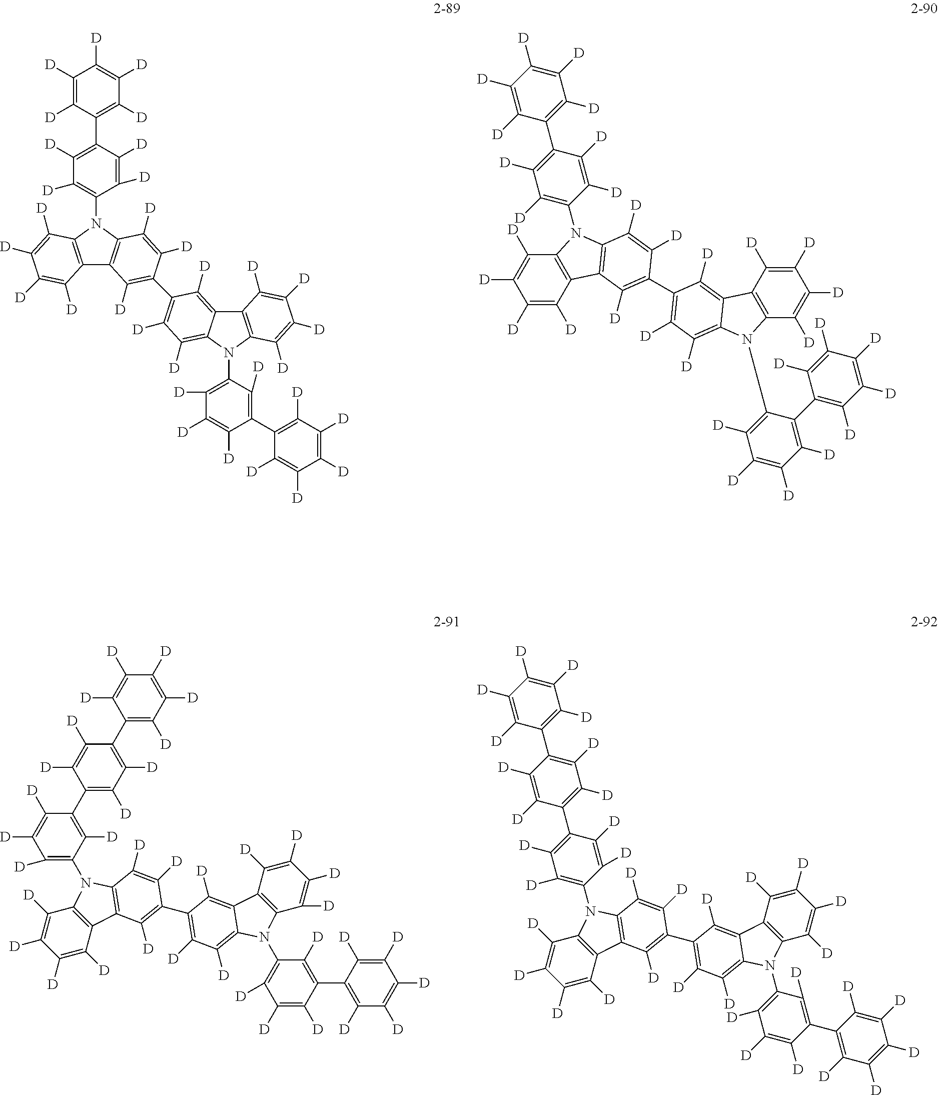

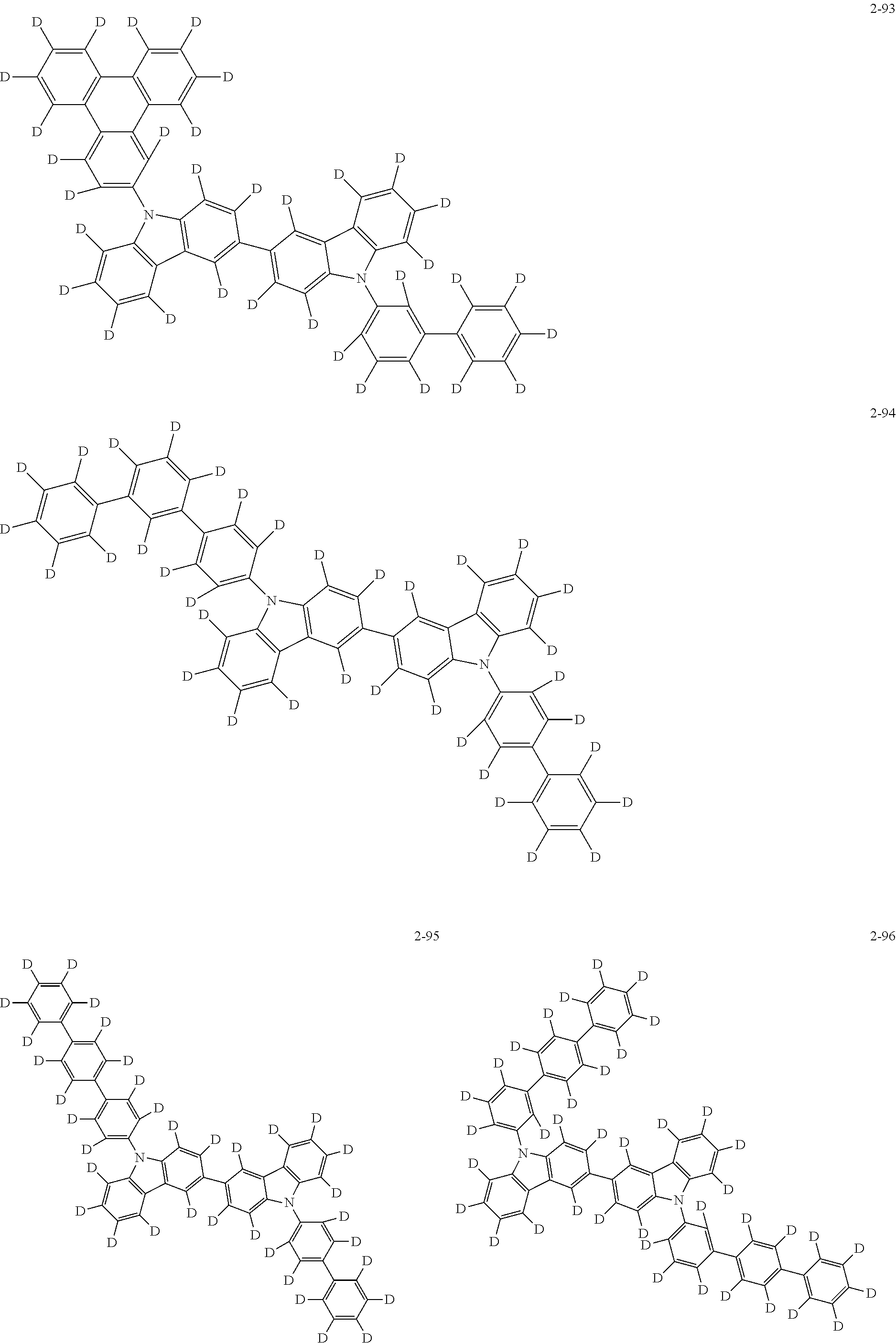

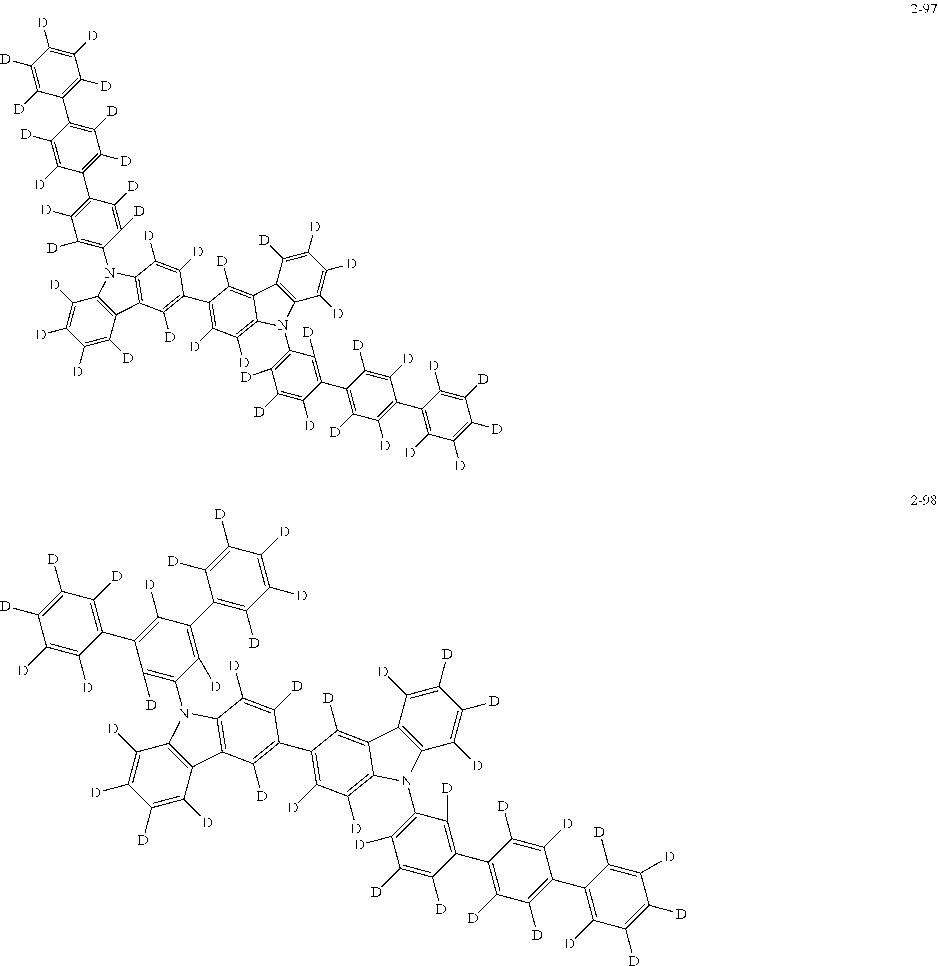

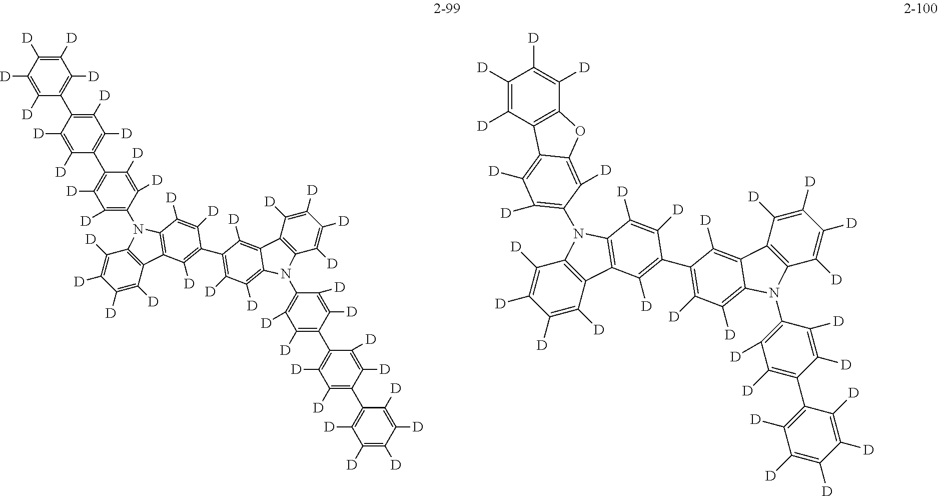

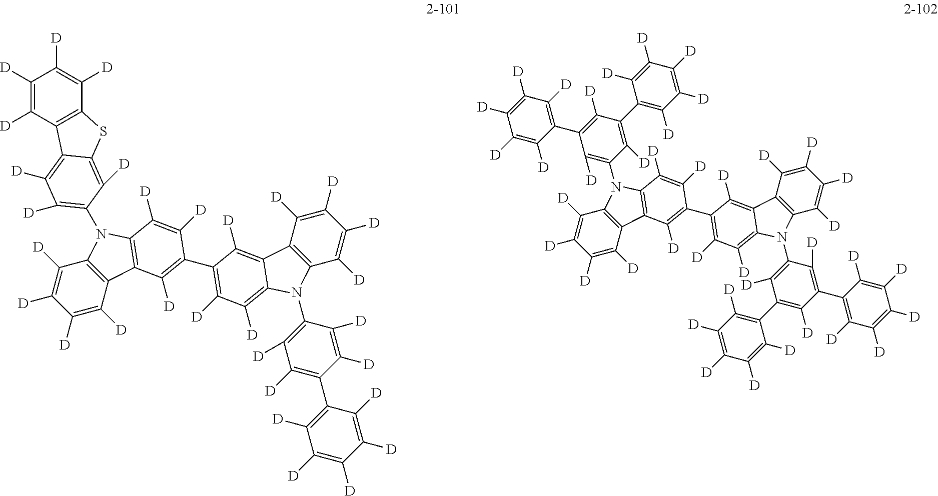

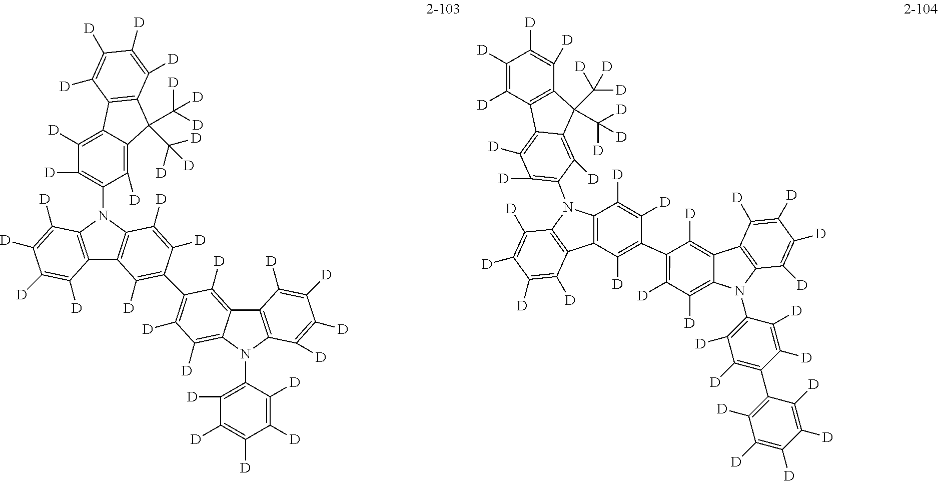

14. The organic light emitting device of claim 12, wherein Chemical Formula 3 is selected from the following compounds:

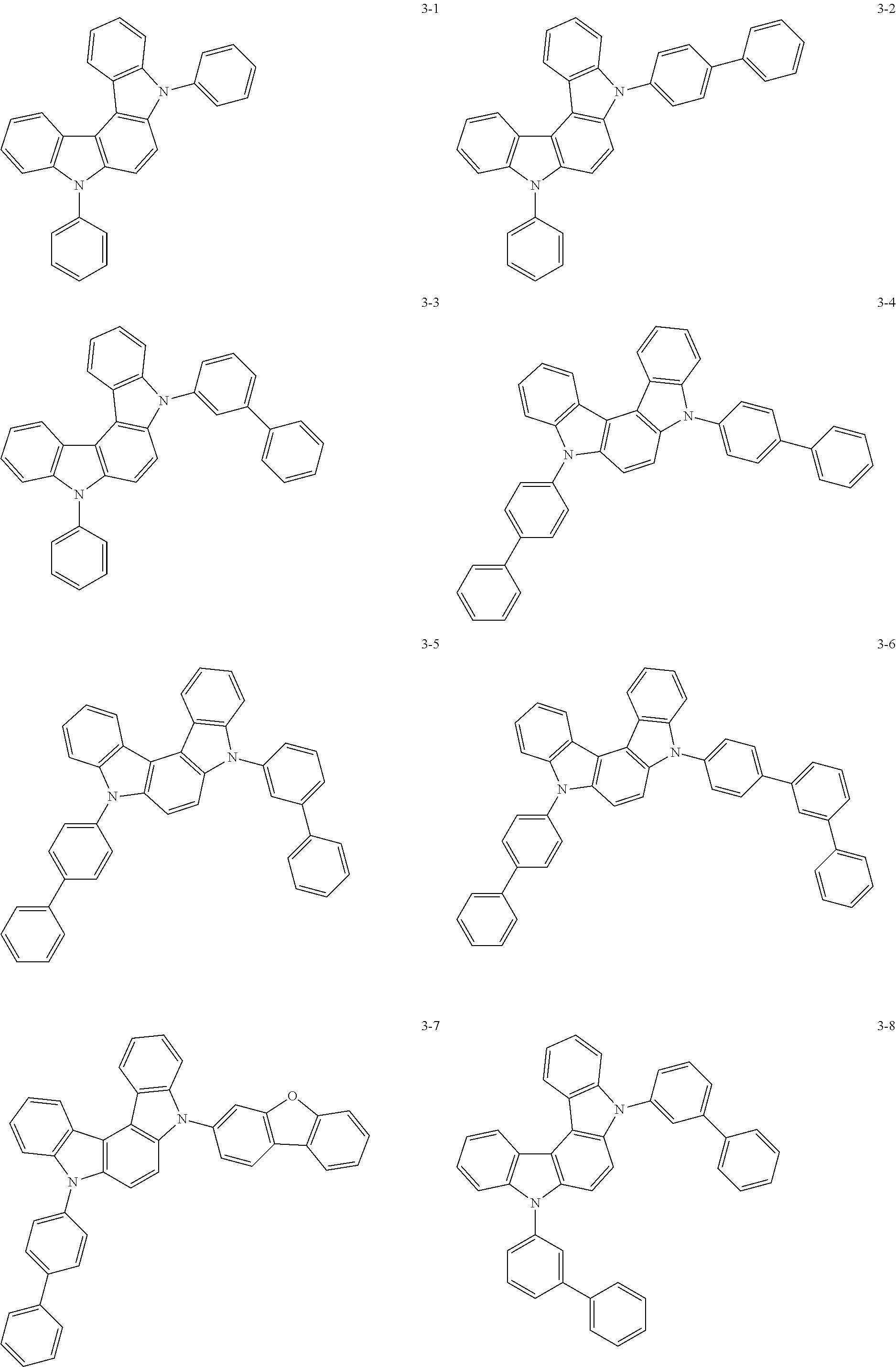

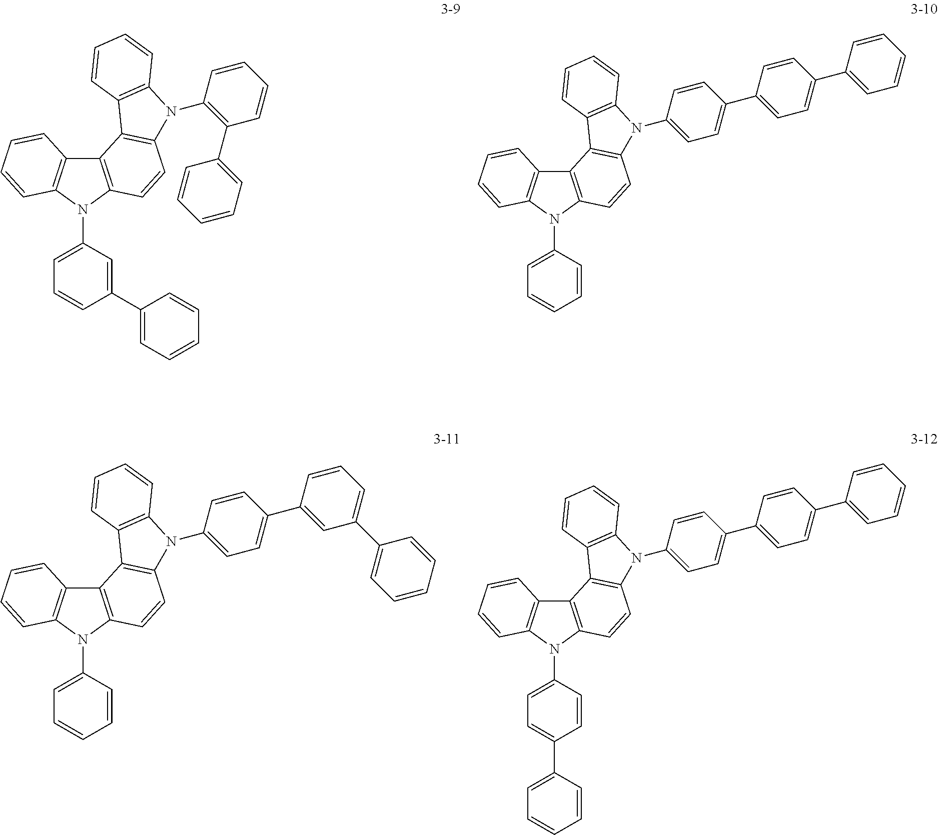 [SMILES: c1ccc(-c2cccc(-n3c4ccccc4c4c5c6ccccc6n(-c6ccccc6-c6ccccc6)c5ccc43)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1 ; c1ccc(-c2ccc(-c3ccc(-n4c5ccccc5c5c6c7ccccc7n(-c7ccc(-c8ccccc8)cc7)c6ccc54)cc3)cc2)cc1]

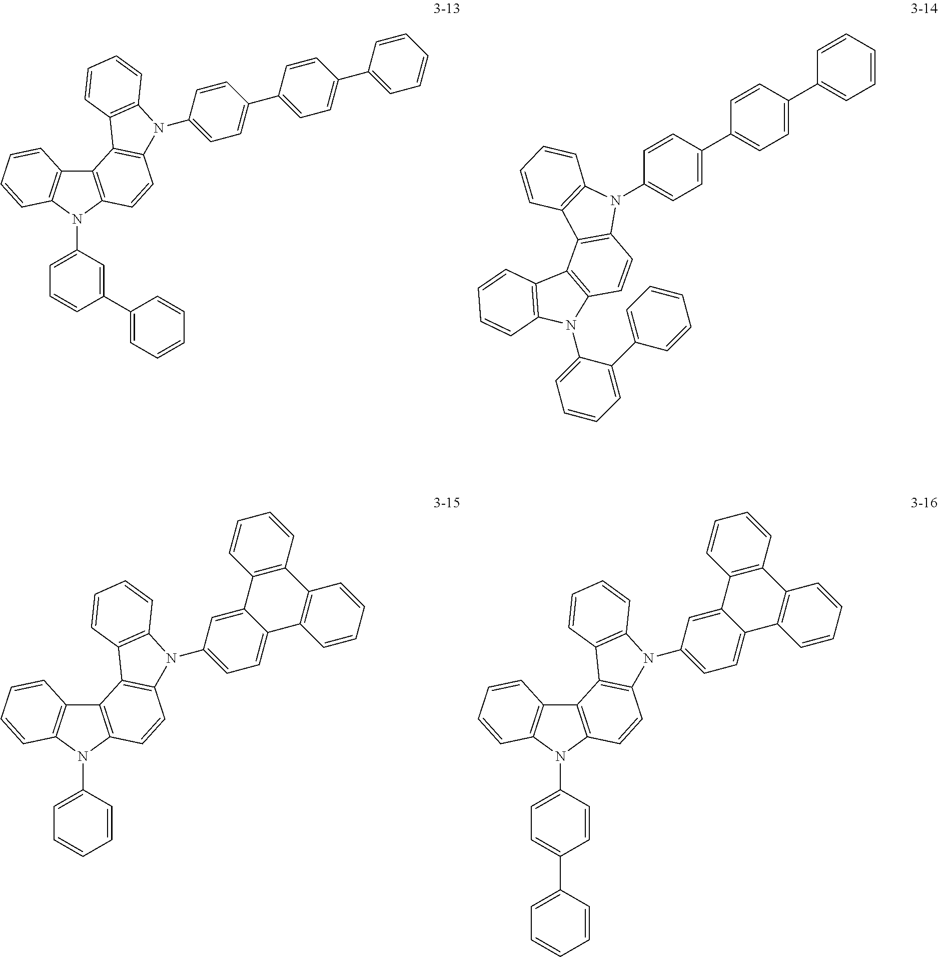 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2ccc(-c3ccc(-n4c5ccccc5c5c6c7ccccc7n(-c7ccccc7-c7ccccc7)c6ccc54)cc3)cc2)cc1 ; c1ccc(-n2c3ccccc3c3c4c5ccccc5n(-c5ccc6c7ccccc7c7ccccc7c6c5)c4ccc32)cc1 ; c1ccc(-c2ccc(-n3c4ccccc4c4c5c6ccccc6n(-c6ccc7c8ccccc8c8ccccc8c7c6)c5ccc43)cc2)cc1]

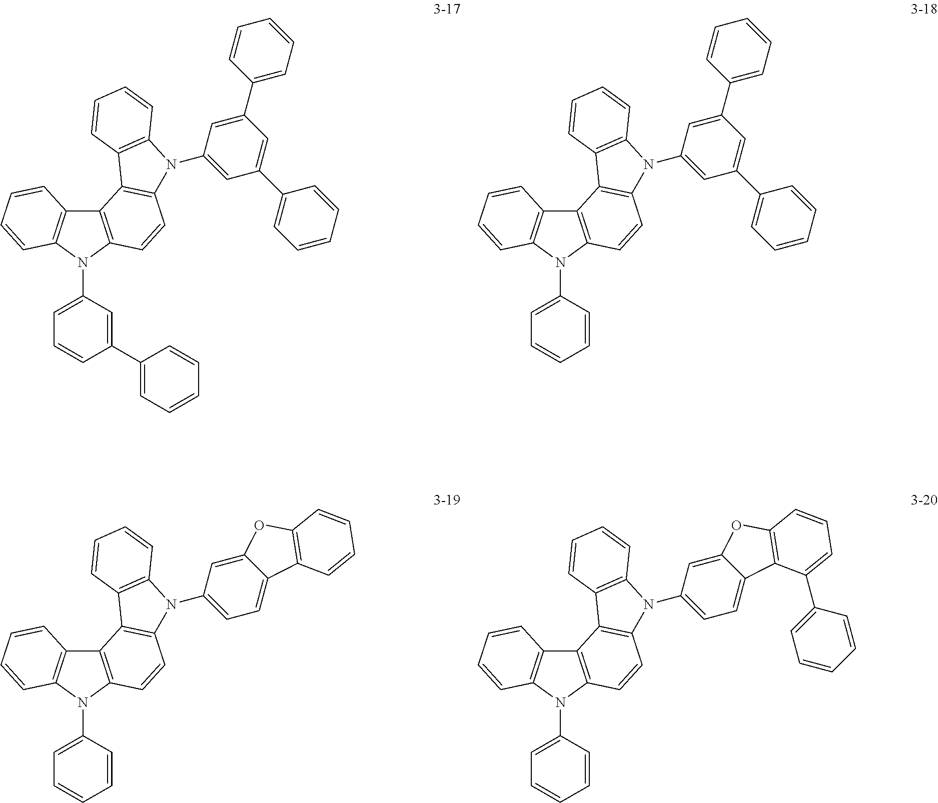 [SMILES: c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6ccccc6)c5ccc43)c2)cc1 ; c1ccc(-n2c3ccccc3c3c4c5ccccc5n(-c5ccc6c(c5)oc5ccccc56)c4ccc32)cc1 ; c1ccc(-c2cccc3oc4cc(-n5c6ccccc6c6c7c8ccccc8n(-c8ccccc8)c7ccc65)ccc4c23)cc1]

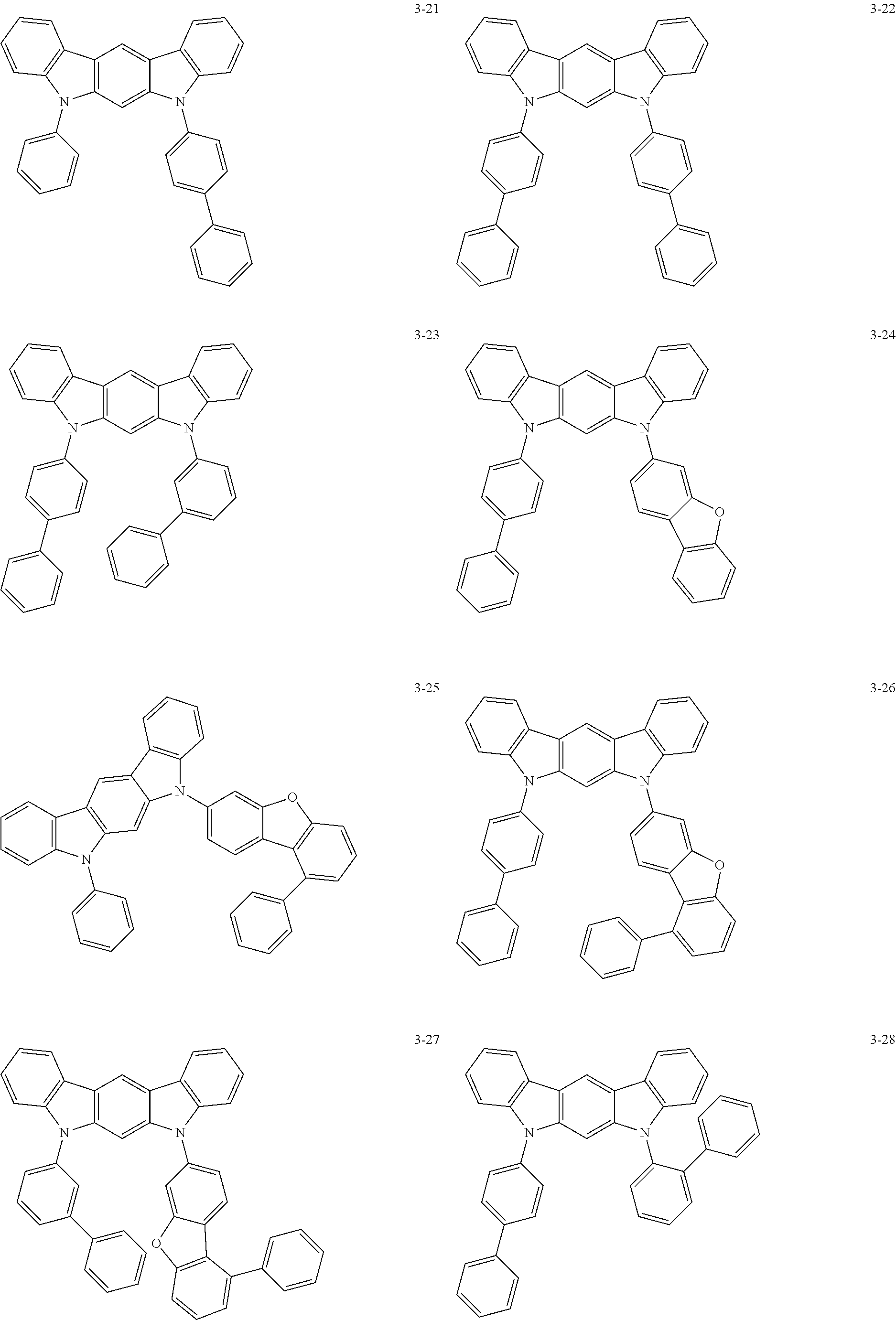

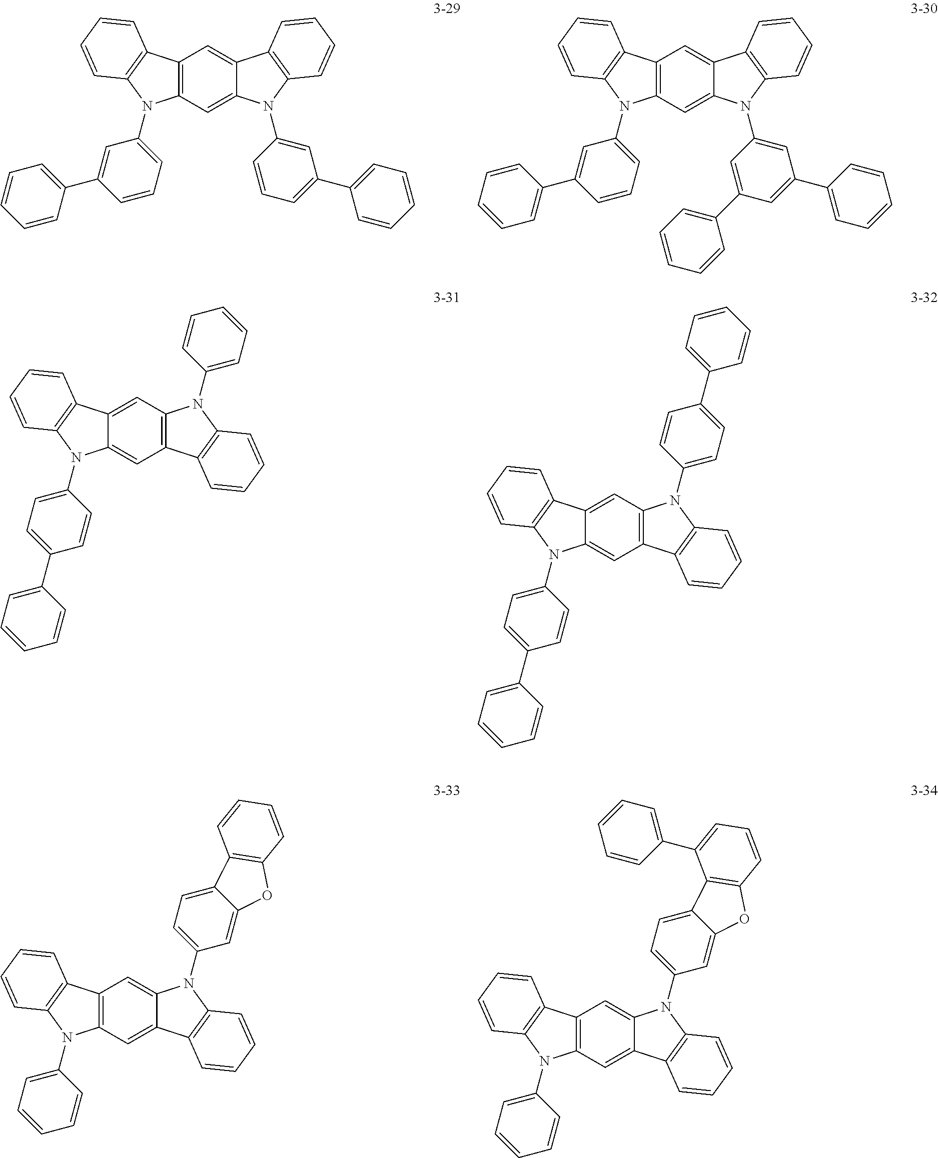

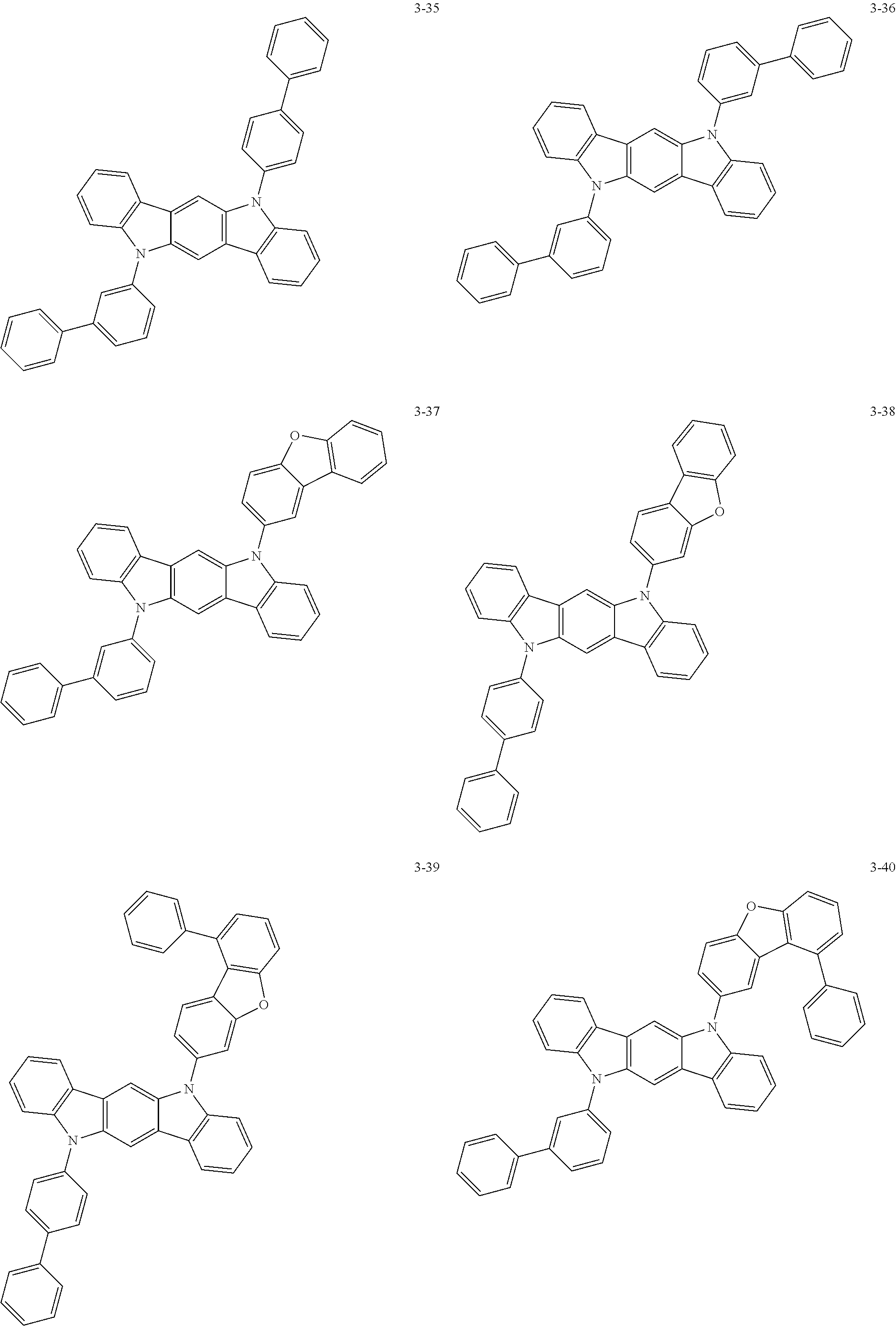 [SMILES: c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1 ; c1ccc(-c2cccc(-n3c4ccccc4c4cc5c(cc43)c3ccccc3n5-c3ccc4oc5ccccc5c4c3)c2)cc1 ; c1ccc(-c2ccc(-n3c4ccccc4c4cc5c(cc43)c3ccccc3n5-c3ccc4c(c3)oc3ccccc34)cc2)cc1 ; c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

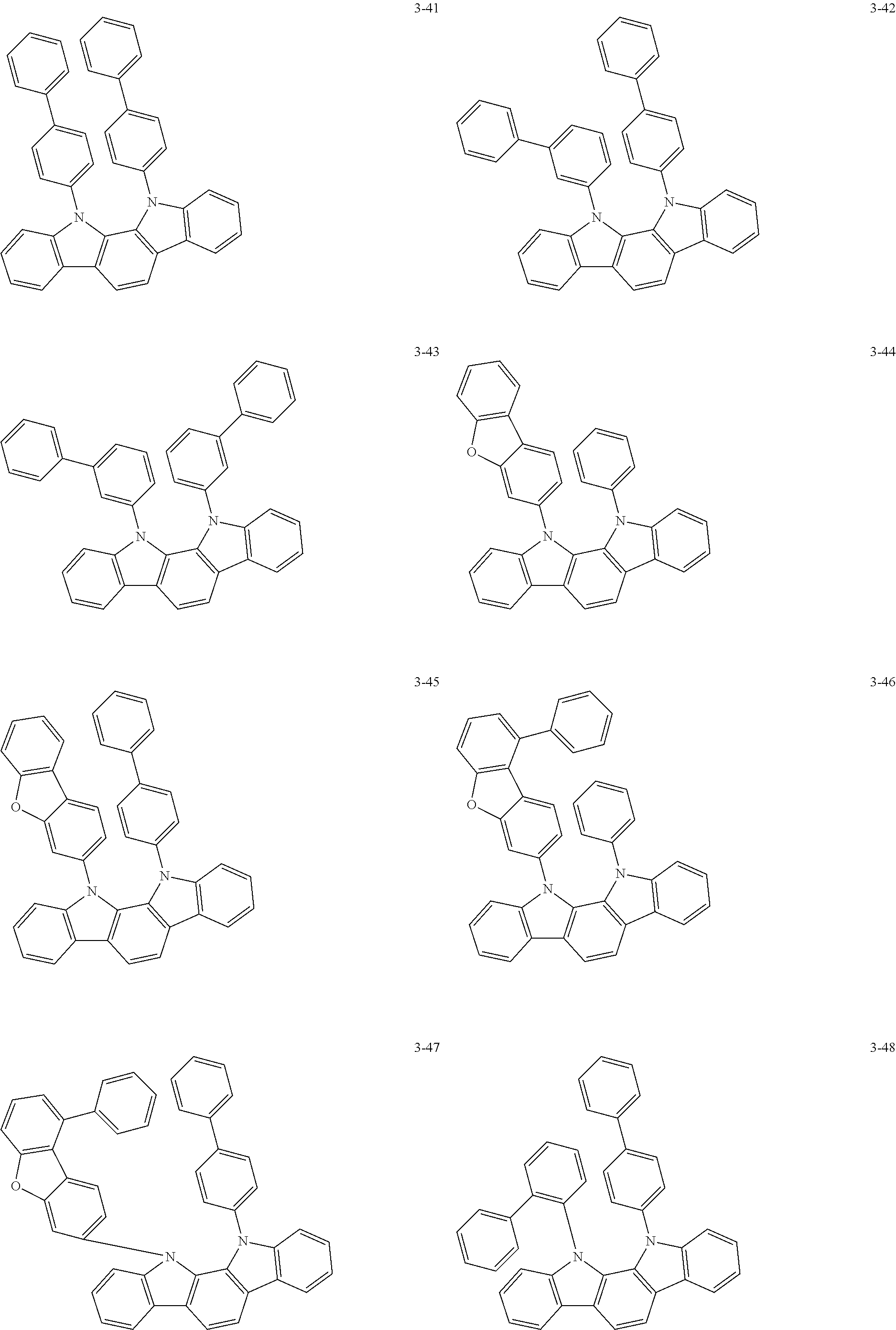

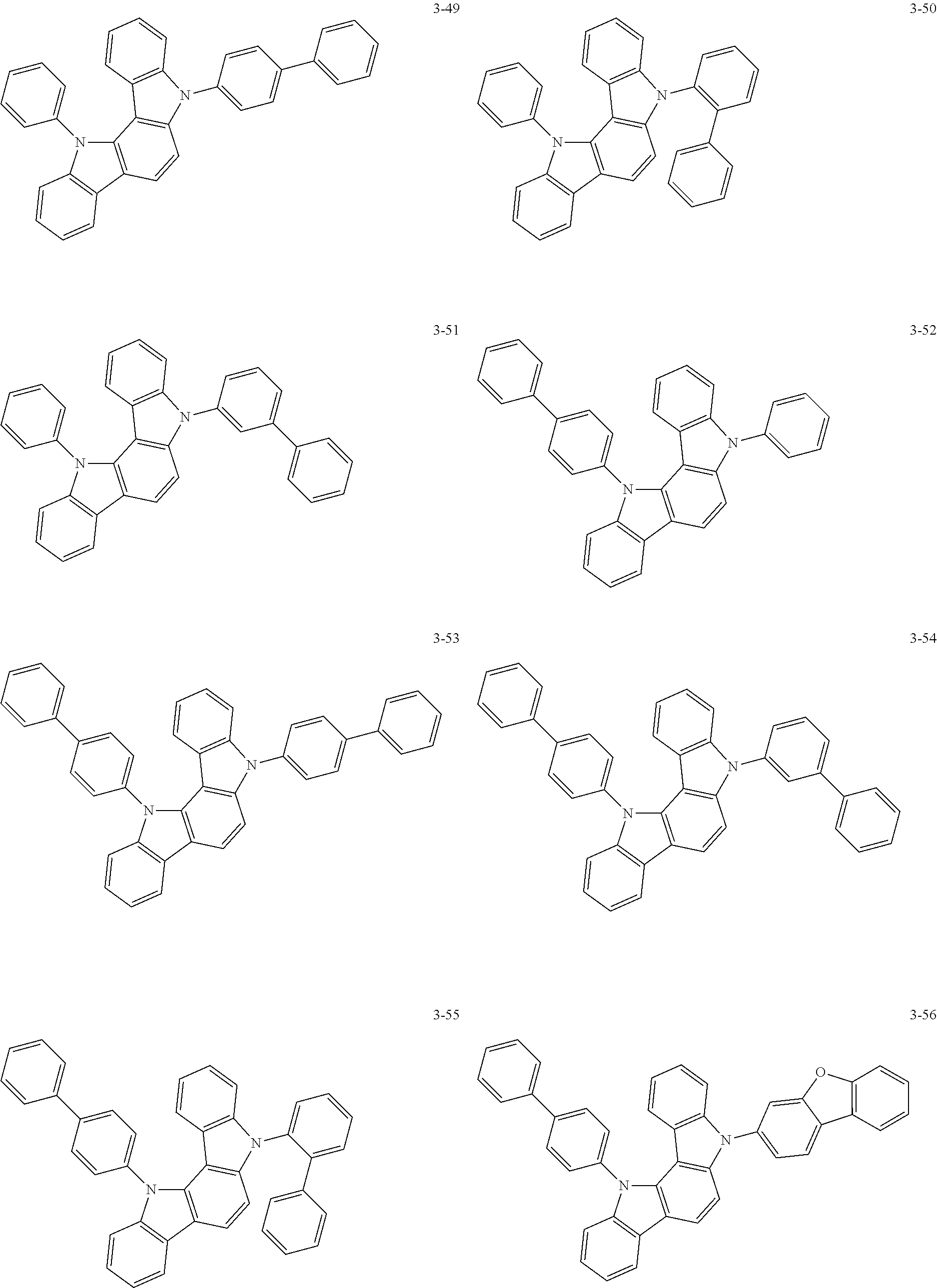

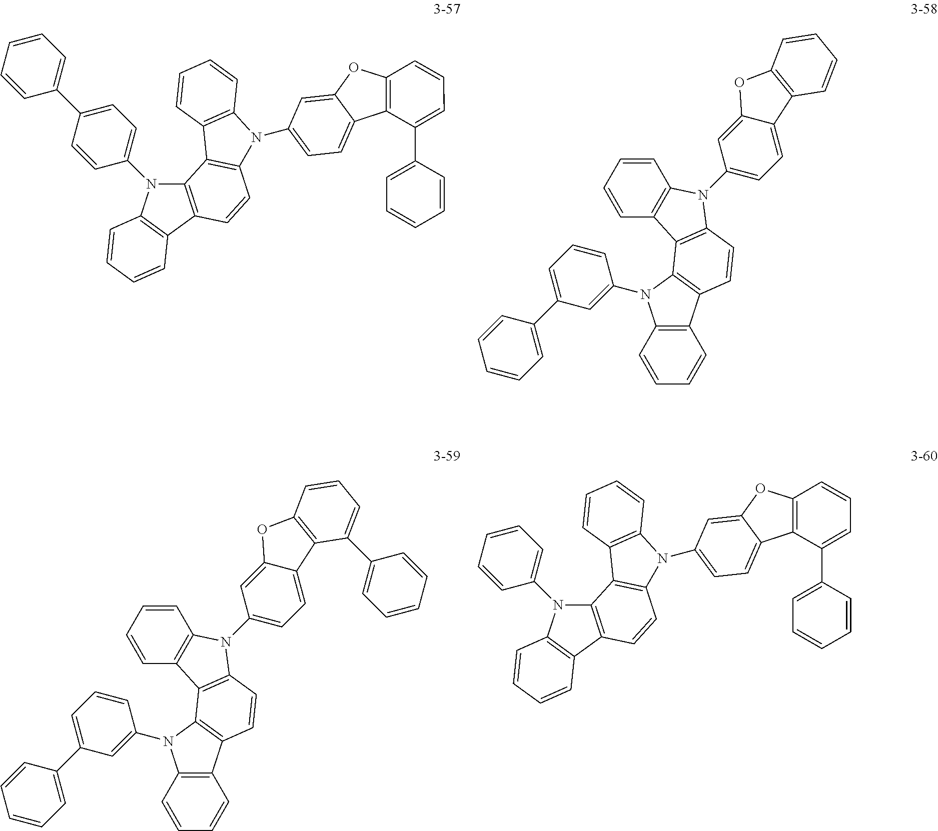 [SMILES: c1ccc(-c2ccc(-n3c4ccccc4c4ccc5c(c6ccccc6n5-c5ccc6c(c5)oc5cccc(-c7ccccc7)c56)c43)cc2)cc1 ; c1ccc(-c2cccc(-n3c4ccccc4c4ccc5c(c6ccccc6n5-c5ccc6c(c5)oc5ccccc56)c43)c2)cc1 ; c1ccc(-c2cccc(-n3c4ccccc4c4ccc5c(c6ccccc6n5-c5ccc6c(c5)oc5cccc(-c7ccccc7)c56)c43)c2)cc1 ; c1ccc(-c2cccc3oc4cc(-n5c6ccccc6c6c5ccc5c7ccccc7n(-c7ccccc7)c56)ccc4c23)cc1]

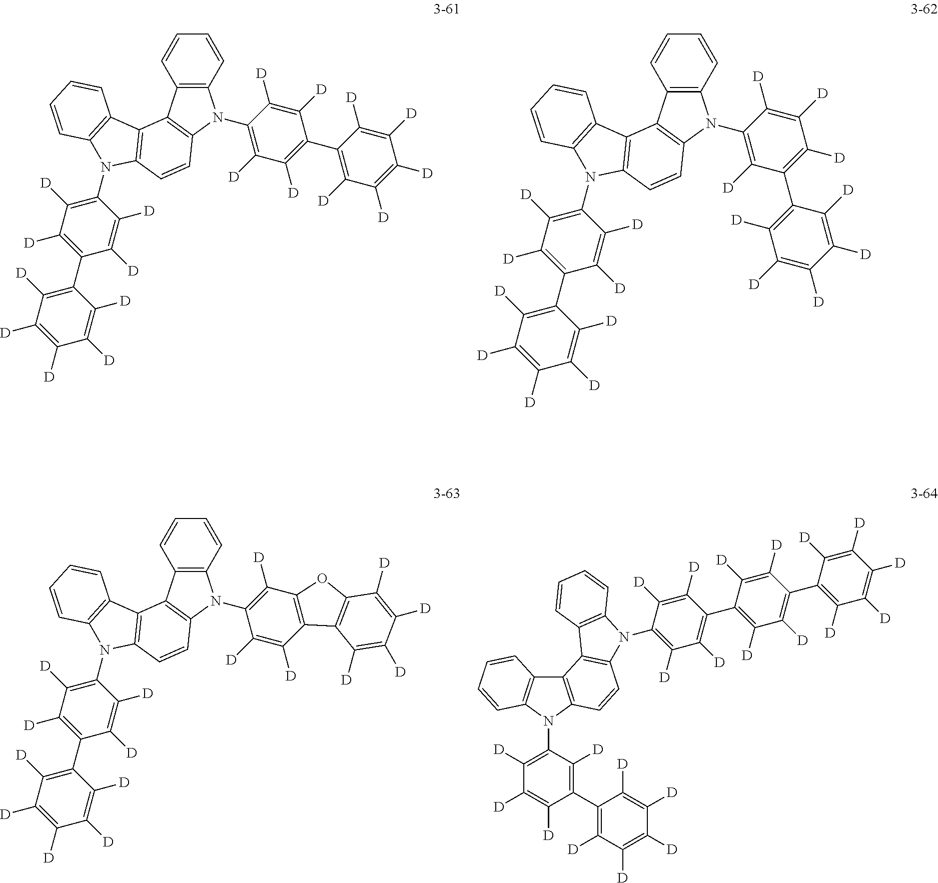

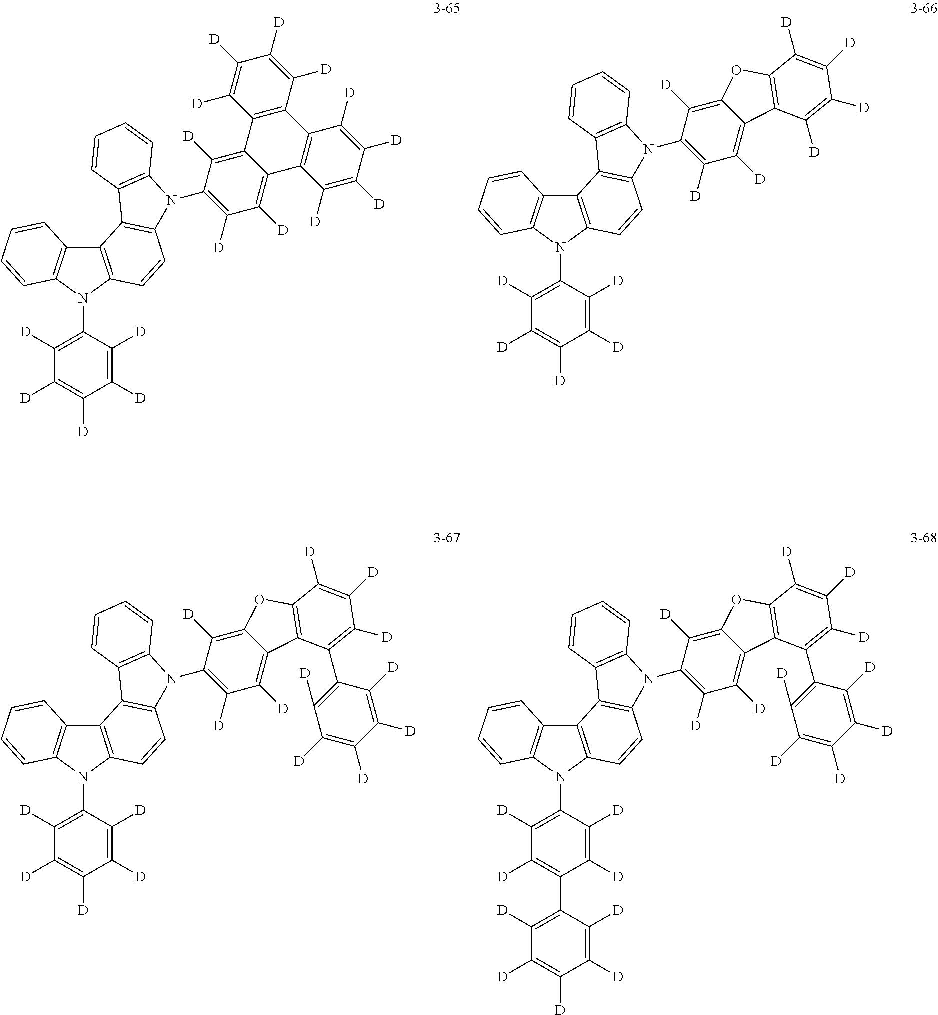

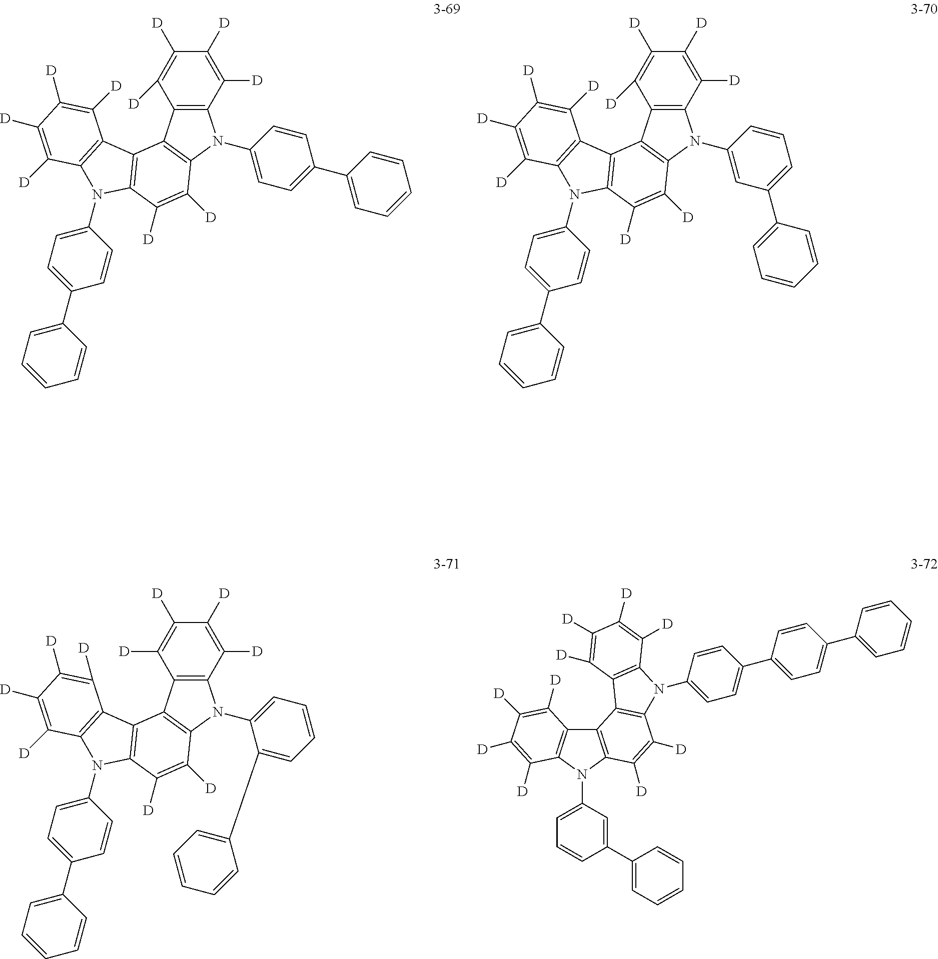

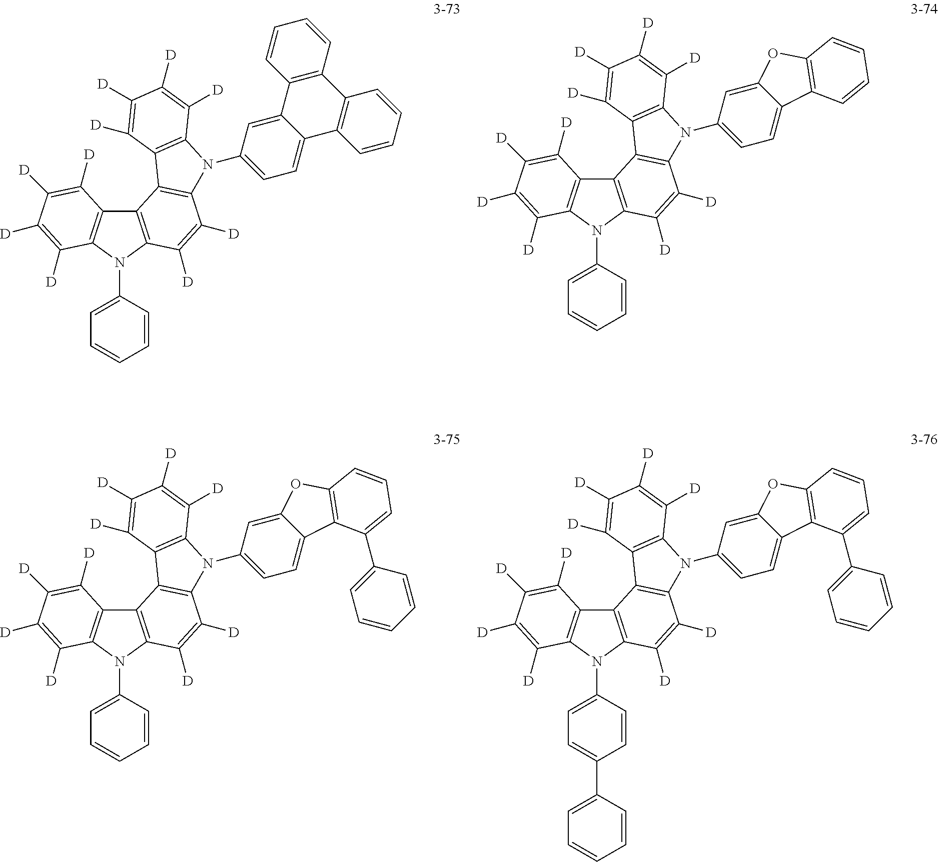

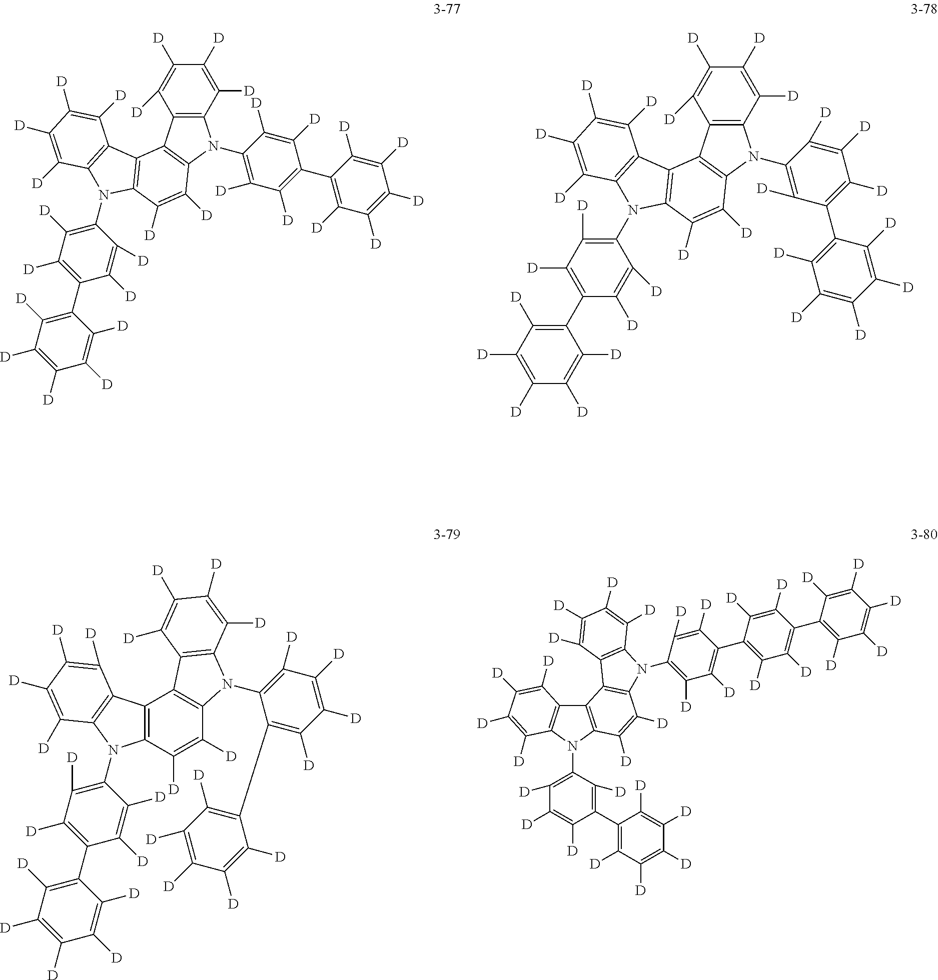

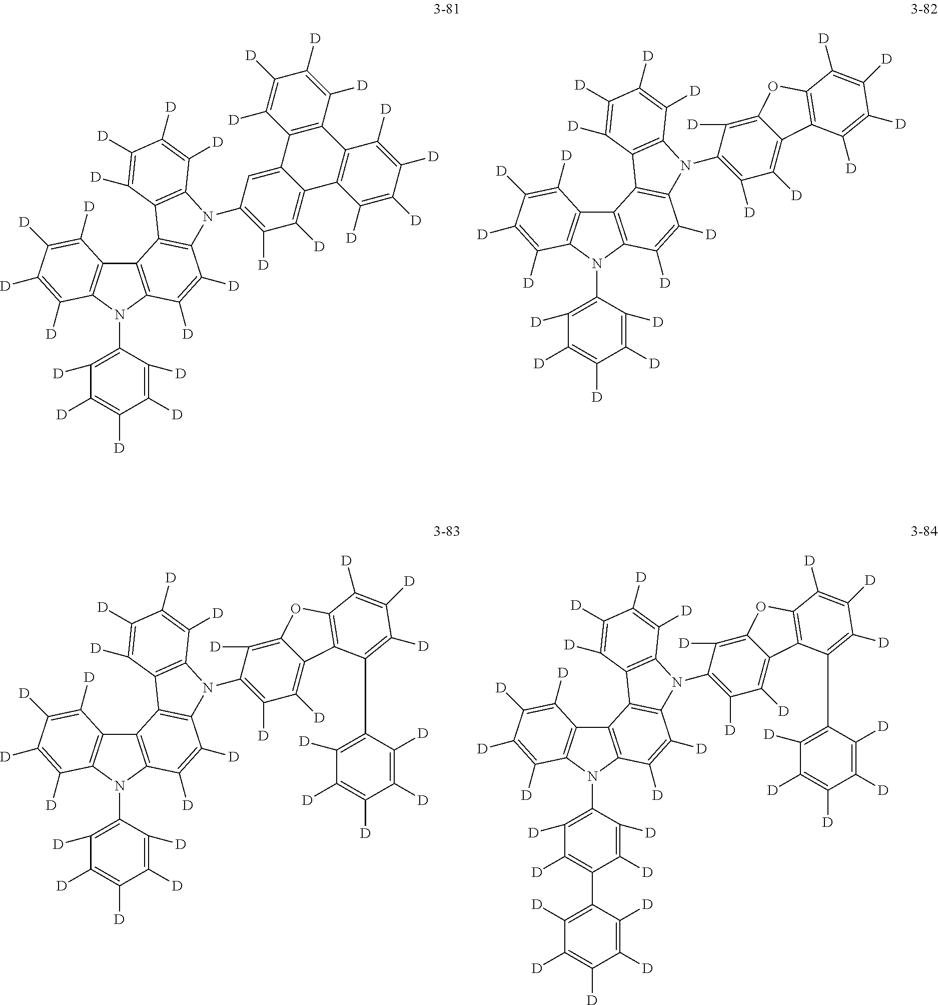

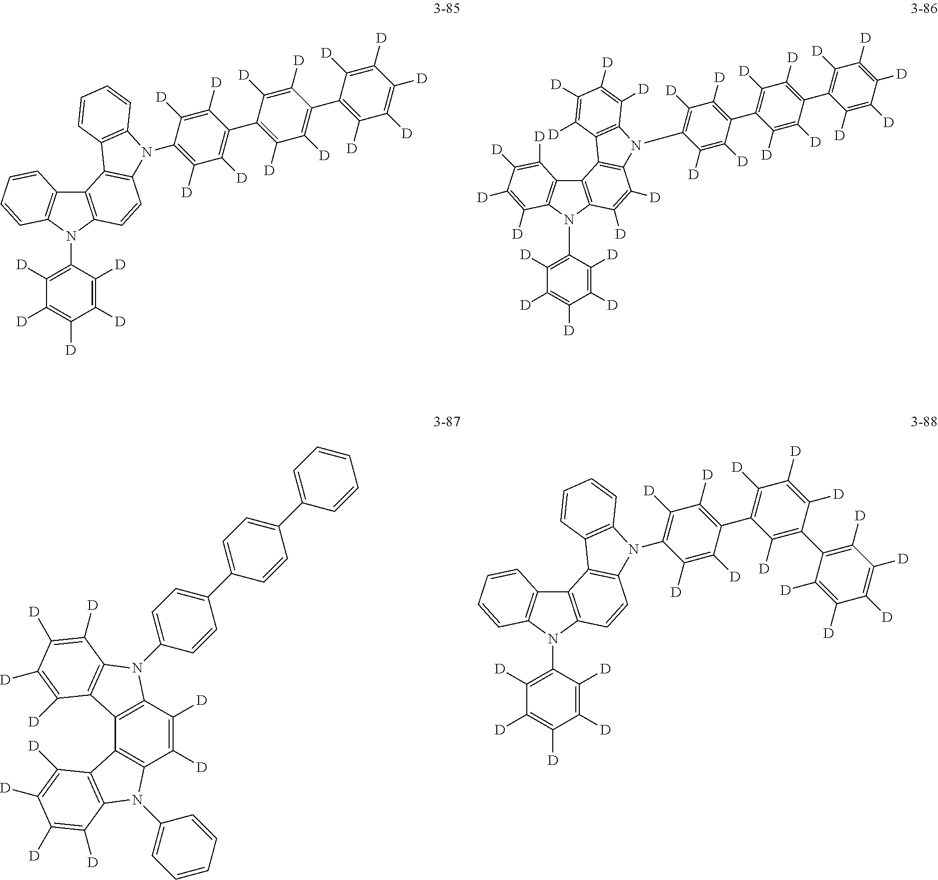

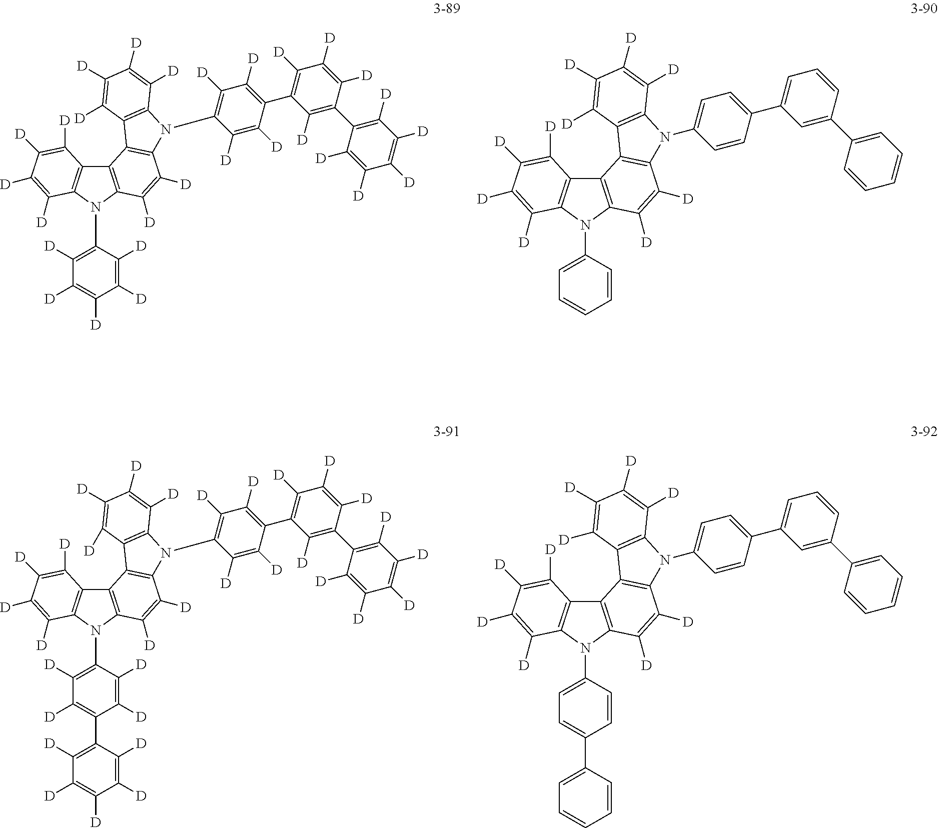

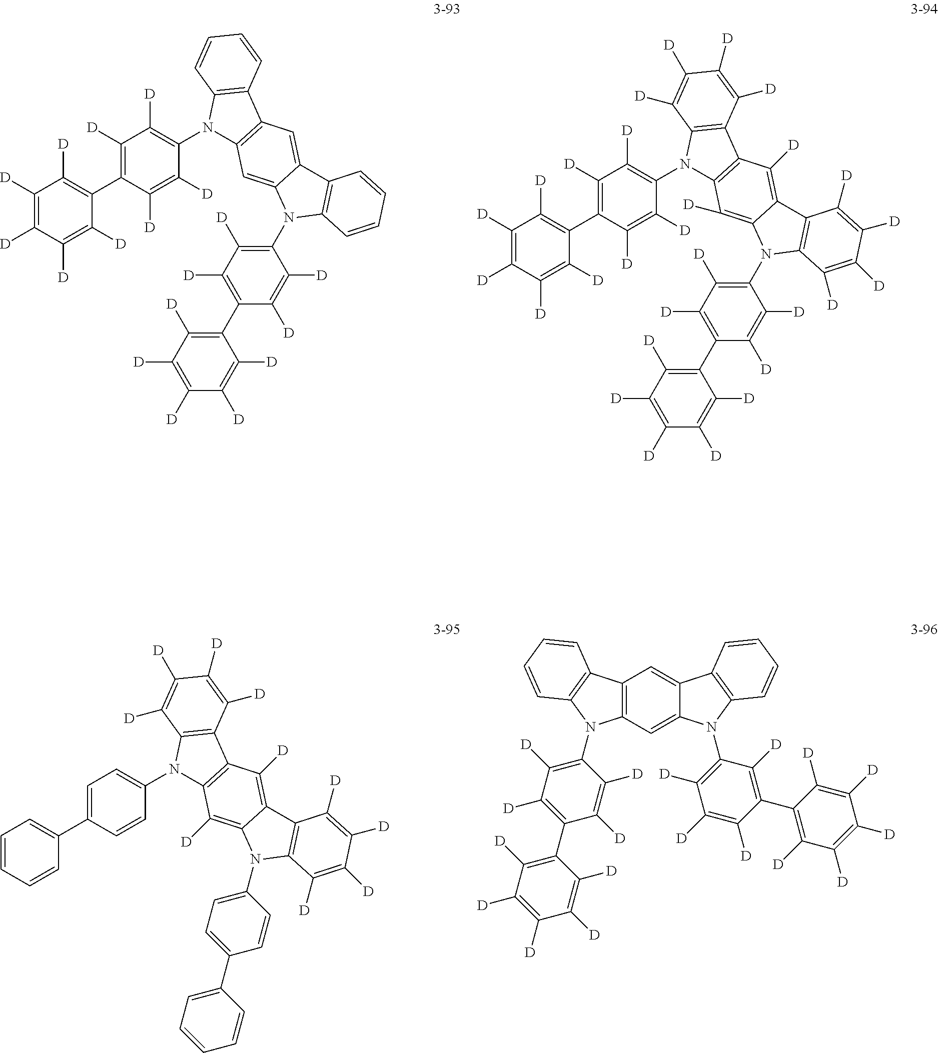

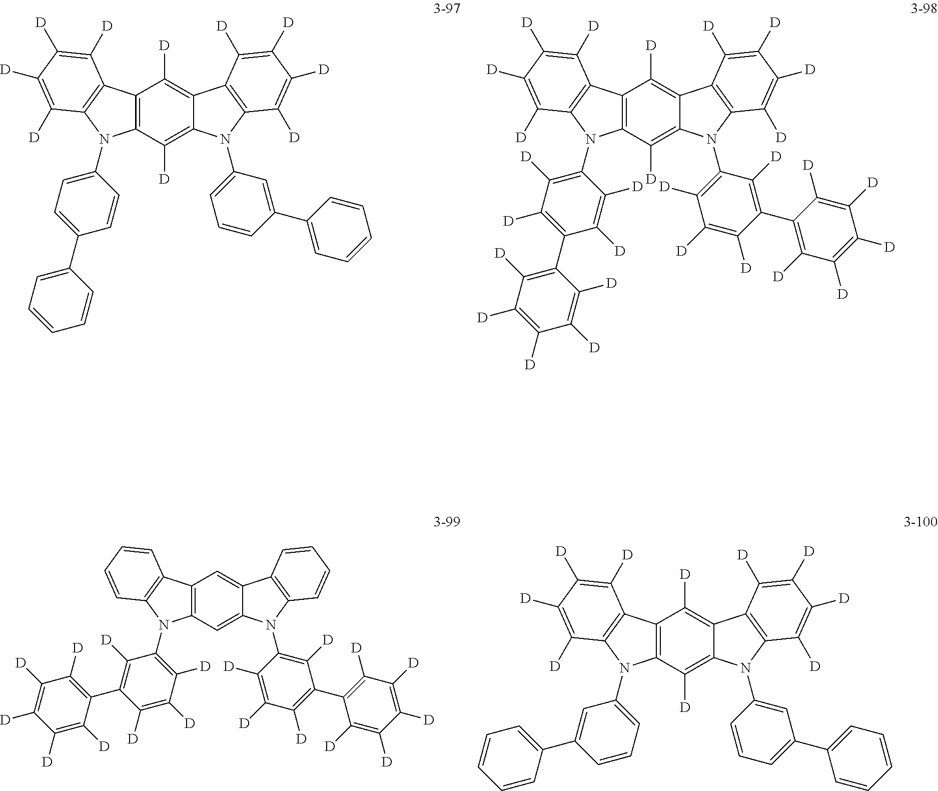

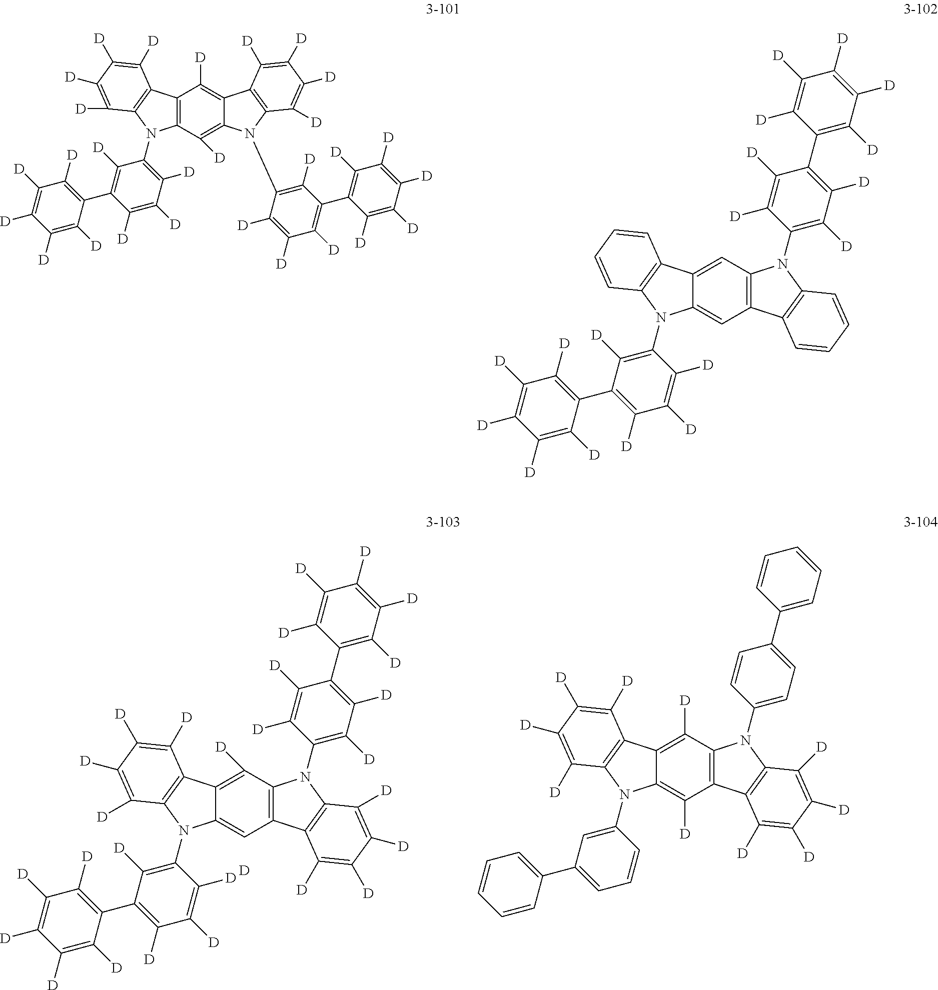

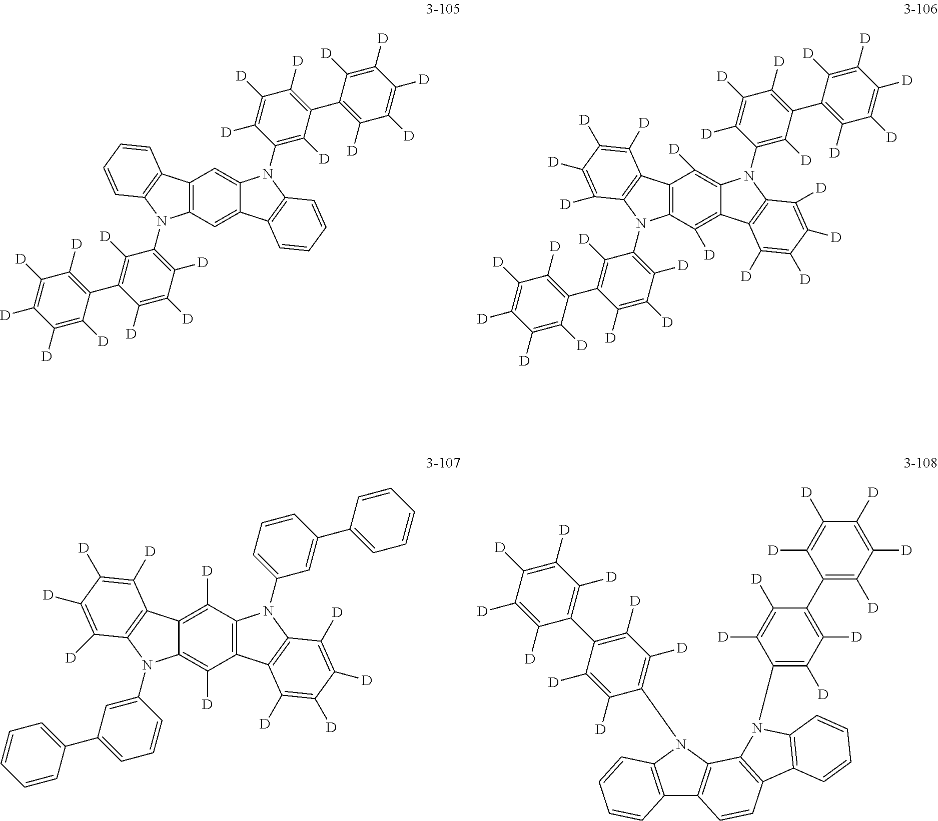

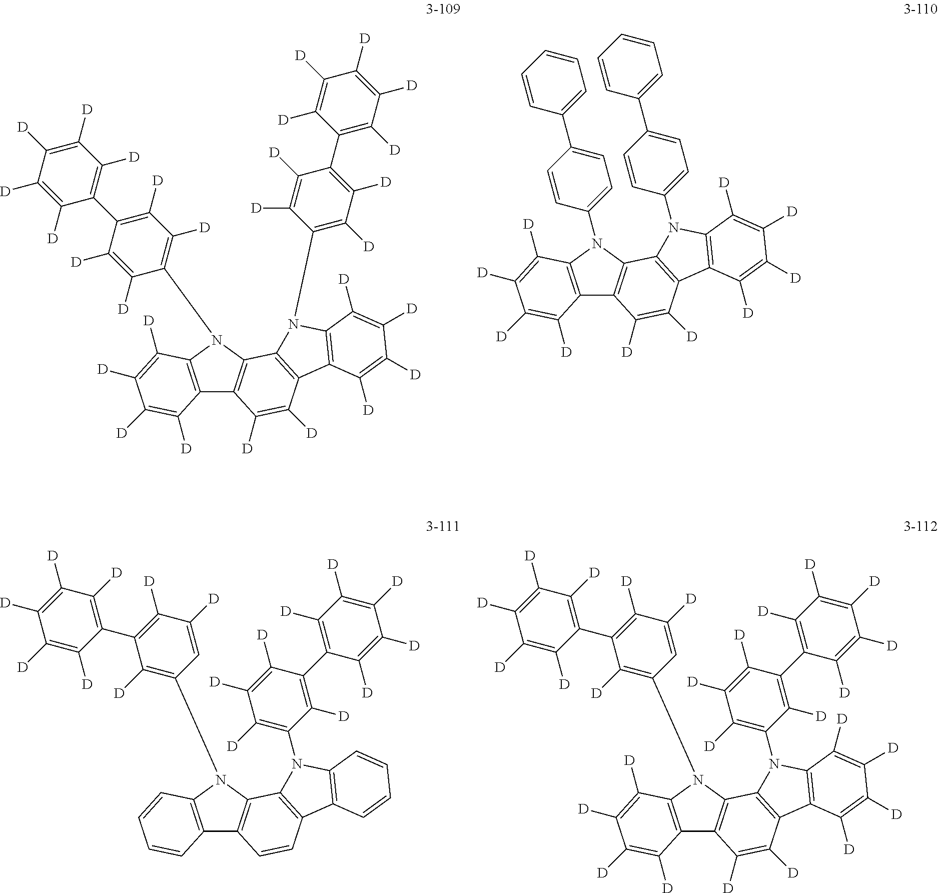

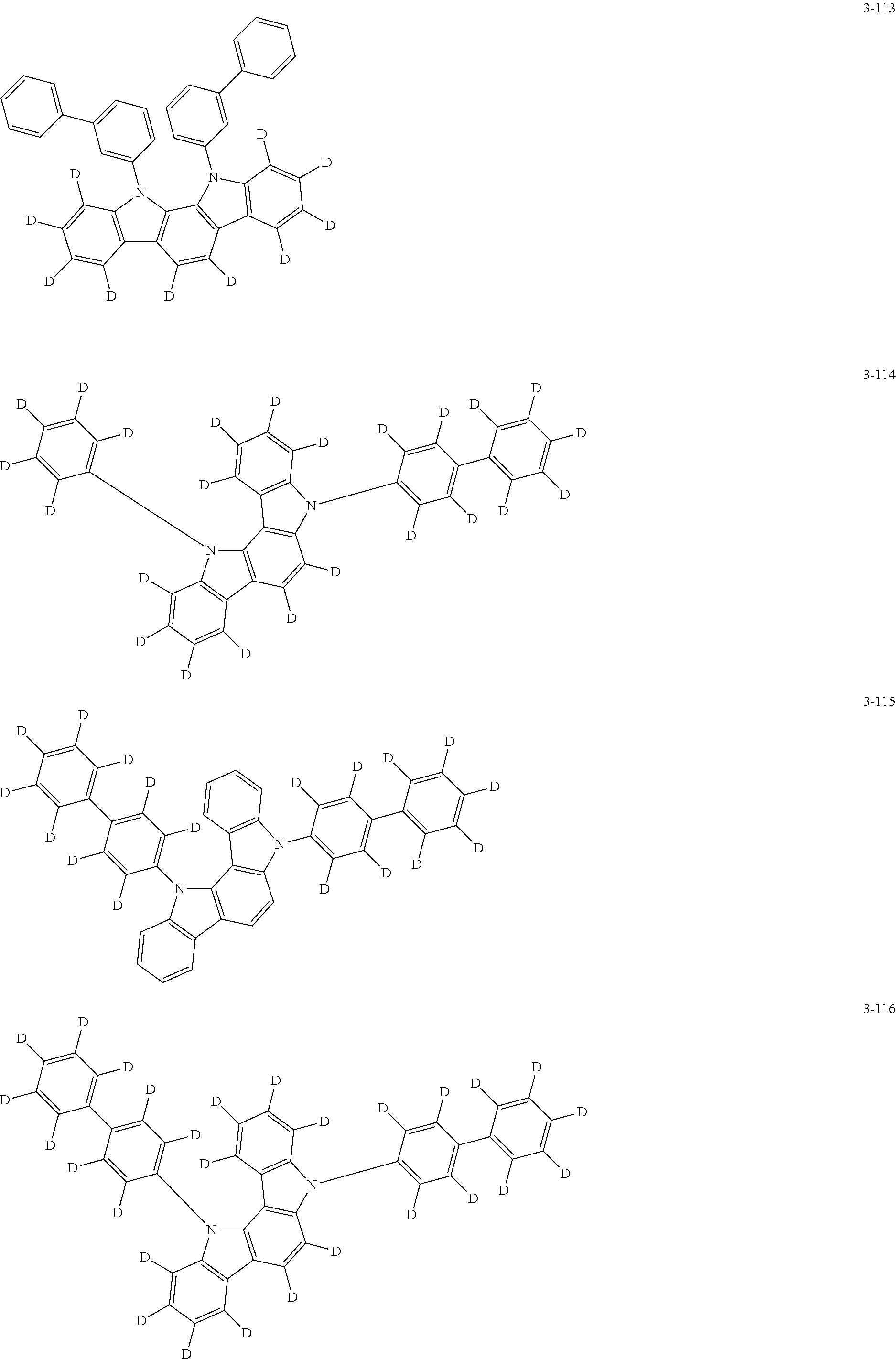

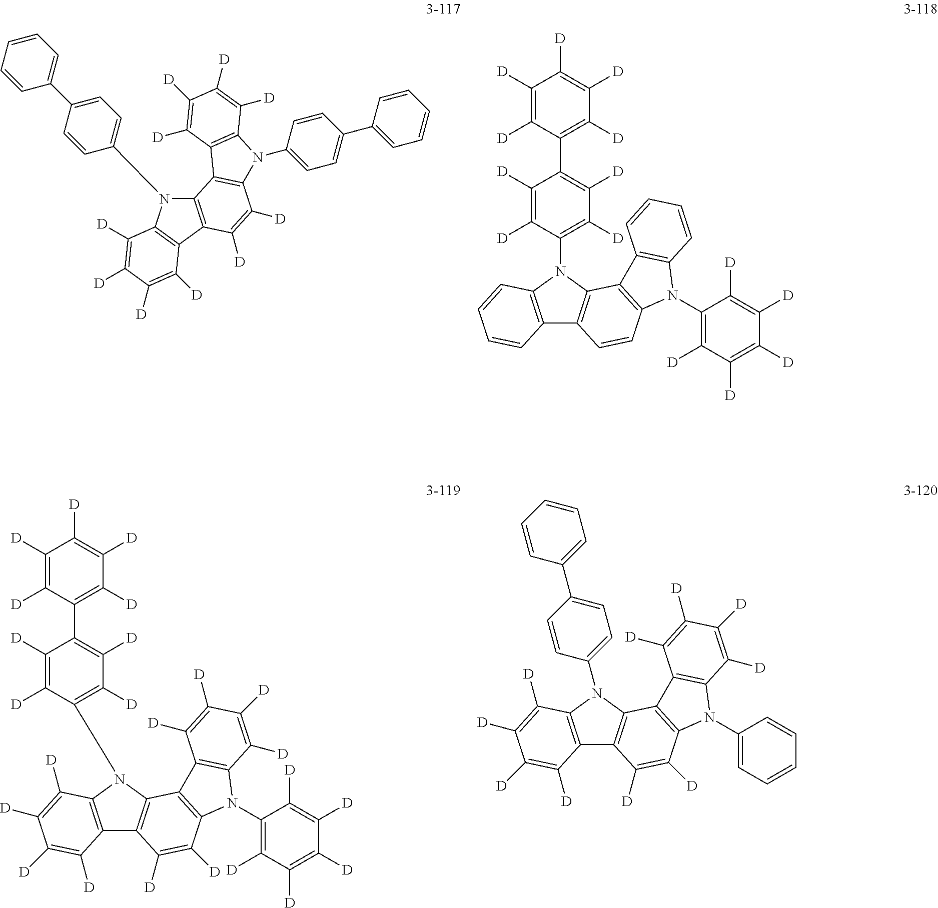

15. A composition for an organic material layer of an organic light emitting device, the composition comprising the heterocyclic compound of claim 1.

16. The composition of claim 15, further comprising a compound of the following Chemical Formula 2 or 3: 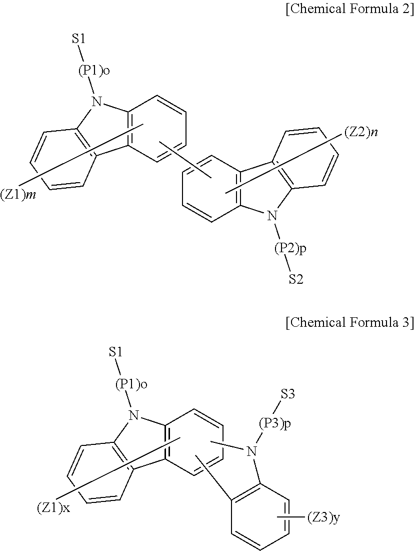
in Chemical Formulae 2 and 3,
P1 to P3 are each independently a direct bond; a substituted or unsubstituted C6 to C60 arylene group; or a substituted or unsubstituted C2 to C60 heteroarylene group,
S1 to S3 are each independently a cyano group; a substituted or unsubstituted triarylsilyl group; a substituted or unsubstituted C6 to C60 aryl group; or a substituted or unsubstituted C2 to C60 heteroaryl group,
Z1 to Z3 are each independently hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
m and n are each an integer of 1 to 7,
x is an integer from 1 to 6,
y is an integer from 1 to 4,
o, p, and q are each an integer from 1 to 3, and
when m, n, x, y, o, p, and g are each 2 or greater, substituents in the parenthesis are the same as or different from each other.

17. The composition of claim 16, wherein the composition comprises the heterocyclic compound and the compound at a weight ratio of 1:10 to 10:1.
